(19) United States
(12) Patent Application Publication
(10) Pub. No.: US 2003/0044935 A1
(43) Pub. Date: Mar. 6, 2003

(54) Secreted proteins and polynucleotides encoding them

(76) Inventors: Kenneth Jacobs, Newton, MA (US); John M. McCoy, Reading, MA (US); Edward R. La Vallie, Harvard, MA (US); Lisa A. Collins-Racie, Acton, MA (US); Cheryl Evans, Germantown, MD (US); David Merberg, Acton, MA (US); Maurice Treacy, Dun Laoghaire (IE); Vikki Spaulding, Lowell, MA (US)

Correspondence Address: LAHIVE & COCKFIELD, 28 STATE STREET, BOSTON, MA, 02109, US

(21) Appl. No.: 09/746,783

(22) Filed: Dec. 21, 2000

Related U.S. Application Data
(63) Continuation-in-part of application No. 09/092,722, filed on Jun. 5, 1998, now abandoned.
(63) Continuation-in-part of application No. 09/096,287, filed on Jun. 11, 1998, now abandoned.
(63) Continuation-in-part of application No. 09/098,588, filed on Jun. 17, 1998, now abandoned.
(63) Continuation-in-part of application No. 08/958,304, filed on Oct. 27, 1997, now abandoned.
(63) Continuation-in-part of application No. 09/130,189, filed on Aug. 4, 1998, now abandoned.
(63) Continuation-in-part of application No. 09/149,633, filed on Sep. 8, 1998, now abandoned.
(63) Continuation-in-part of application No. 09/165,960, filed on Oct. 1, 1998, now abandoned.
(63) Continuation-in-part of application No. 09/185,936, filed on Nov. 4, 1998, now abandoned.
(60) Provisional application No. 60/086,236, filed on Jun. 11, 1997.
(60) Provisional application No. 60/086,234, filed on Jun. 12, 1997.
(60) Provisional application No. 60/092,115, filed on Jul. 8, 1997.
(60) Provisional application No. 60/090,100, filed on Oct. 2, 1997.
(60) Provisional application No. 60/090,111, filed on Nov. 7, 1997.
(60) Provisional application No. 60/093,045, filed on Sep. 8, 1997.

(51) Int. Cl. C07H 21/04; C12P 21/02; C12N 5/06

(52) U.S. Cl. 435/69.5; 530/351; 435/325; 435/320.1; 536/23.5

(57) ABSTRACT

Novel polynucleotides and the proteins encoded thereby are disclosed.

This application is a continuation-in-part of application Ser. No. 09/092,722, filed Jun. 5, 1998, which claims the benefit of Ser. No. 60/086,236 (converted to a provisional application from non-provisional application Ser. No. 08/873,218), filed Jun. 11, 1997. This application is a also a continuation-in-part of application Ser. No. 09/096,287 filed Jun. 11, 1998, which claims the benefit of Ser. No. 60/086,234 (converted to a provisional application from non-provisional application Ser. No. 08/873,488), filed Jun. 12, 1997. This application is also a continuation-in-part of application Ser. No. 09/098,588 filed Jun. 17, 1998, which claims the benefit of Ser. No. ______ (converted to a provisional application from non-provisional application Ser. No. 08/878,715), filed Jun. 19, 1997. This application is also a continuation-in-part of application Ser. No. 08/958,304 filed Oct. 27, 1997, which claims the benefit of Ser. No. 60/092,115 (converted to a provisional application from non-provisional application Ser. No. 08/887,195, filed Jul. 2, 1997). This application is also a continuation-in-part of application Ser. No. 09/130,189 filed Aug. 4, 1998, which claims the benefit of Ser. No. ______ (converted to a provisional application from non-provisional application Ser. No. 08/906,708), filed Aug. 6, 1997. This application is also a continuation-in-part of application Ser. No. 09/149,633 filed Sep. 8, 1998,which claims the benefit of Ser. No. 60/093,045 (converted to a provisional application from non-provisional application Ser. No. 08/929,007), filed Sep. 8, 1997. This application is also a continuation-in-part of application Ser. No. 09/165,960 filed Oct. 1, 1998, which claims the benefit of Ser. No. 60/090,100 (converted to a provisional application from non-provisional application Ser. No. 08/942,813), filed Oct. 2, 1997. This application is also a continuation-in-part of provisional application Ser. No. 09/185,936 filed Nov. 4, 1998, which claims the benefit of Ser. No. 60/090,111 (converted to a provisional application from non-provisional application Ser. No. 08/965,789), filed Nov. 7, 1997. The entire content of all of the above referenced applications are incorporated by reference herein.

### FIELD OF THE INVENTION

The present invention provides novel polynucleotides and proteins encoded by such polynucleotides, along with therapeutic, diagnostic and research utilities for these polynucleotides and proteins.

### BACKGROUND OF THE INVENTION

Technology aimed at the discovery of protein factors (including e.g., cytokines, such as lymphokines, interferons, CSFs and interleukins) has matured rapidly over the past decade. The now routine hybridization cloning and expression cloning techniques clone novel polynucleotides “directly” in the sense that they rely on information directly related to the discovered protein (i.e., partial DNA/amino acid sequence of the protein in the case of hybridization cloning; activity of the protein in the case of expression cloning). More recent “indirect” cloning techniques such as signal sequence cloning, which isolates DNA sequences based on the presence of a now well-recognized secretory leader sequence motif, as well as various PCR-based or low stringency hybridization cloning techniques, have advanced the state of the art by making available large numbers of DNA/amino acid sequences for proteins that are known to have biological activity by virtue of their secreted nature in the case of leader sequence cloning, or by virtue of the cell or tissue source in the case of PCR-based techniques. It is to these proteins and the polynucleotides encoding them that the present invention is directed.

### SUMMARY OF THE INVENTION

In one embodiment, the present invention provides a composition comprising an isolated polynucleotide selected from the group consisting of:

(a) a polynucleotide comprising the nucleotide sequence of SEQ ID NO:1;

(b) a polynucleotide comprising the nucleotide sequence of SEQ ID NO:1 from nucleotide 12 to nucleotide 800;

(c) a polynucleotide comprising the nucleotide sequence of SEQ ID NO:1 from nucleotide 78 to nucleotide 800;

(d) a polynucleotide comprising the nucleotide sequence of SEQ ID NO:1 from nucleotide 1 to nucleotide 547;

(e) a polynucleotide comprising the nucleotide sequence of the full-length protein coding sequence of clone bh389_{—}11 deposited under accession number ATCC 98451;

(f) a polynucleotide encoding the full-length protein encoded by the cDNA insert of clone bh389_{—}11 deposited under accession number ATCC 98451;

(g) a polynucleotide comprising the nucleotide sequence of a mature protein coding sequence of clone bh389_{—}11 deposited under accession number ATCC 98451;

(h) a polynucleotide encoding a mature protein encoded by the cDNA insert of clone bh389_{—}11 deposited under accession number ATCC 98451;

(i) a polynucleotide encoding a protein comprising the amino acid sequence of SEQ ID NO:2;

(j) a polynucleotide encoding a protein comprising a fragment of the amino acid sequence of SEQ ID NO:2 having biological activity, the fragment comprising eight consecutive amino acids of SEQ ID NO:2;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:1 from nucleotide 12 to nucleotide 800; the nucleotide sequence of SEQ ID NO:1 from nucleotide 78 to nucleotide 800; the nucleotide sequence of SEQ ID NO:1 from nucleotide 1 to nucleotide 547; the nucleotide sequence of the full-length protein coding sequence of clone bh389_{—}11 deposited under accession number ATCC 98451; or the nucleotide sequence of a mature protein coding sequence of clone bh389_{—}11 deposited under accession number ATCC 98451. In other preferred embodiments, the polynucleotide encodes the full-length or a mature protein encoded by the cDNA insert of clone bh389_{—}11 deposited under accession number ATCC 98451. In yet other preferred embodiments, the present invention provides a polynucleotide encoding a protein comprising the amino acid sequence of SEQ ID NO:2 from amino acid 1 to amino acid 178. In further preferred embodiments, the present invention provides a polynucleotide encoding a protein comprising a fragment of the amino acid sequence of SEQ ID NO:2 having biological activity, the fragment preferably comprising eight (more preferably twenty, most preferably thirty) consecutive amino acids of SEQ ID NO:2, or a polynucleotide encoding a protein comprising a fragment of the amino acid sequence of SEQ ID NO:2 having biological activity, the fragment comprising the amino acid sequence from amino acid 126 to amino acid 135 of SEQ ID NO:2.

Other embodiments provide the gene corresponding to the cDNA sequence of SEQ ID NO:1.

In other embodiments, the present invention provides a composition comprising a protein, wherein said protein comprises an amino acid sequence selected from the group consisting of:

(a) the amino acid sequence of SEQ ID NO:2;

(b) the amino acid sequence of SEQ ID NO:2 from amino acid 1 to amino acid 178;

(c) fragments of the amino acid sequence of SEQ ID NO:2 comprising eight consecutive amino acids of SEQ ID NO:2; and

(d) the amino acid sequence encoded by the cDNA insert of clone bh389_{—}11 deposited under accession number ATCC 98451;

the protein being substantially free from other mammalian proteins. Preferably such protein comprises the amino acid sequence of SEQ ID NO:2 or the amino acid sequence of SEQ ID NO:2 from amino acid 1 to amino acid 178. In further preferred embodiments, the present invention provides a protein comprising a fragment of the amino acid sequence of SEQ ID NO:2 having biological activity, the fragment preferably comprising eight (more preferably twenty, most preferably thirty) consecutive amino acids of SEQ ID NO:2, or a protein comprising a fragment of the amino acid sequence of SEQ ID NO:2 having biological activity, the fragment comprising the amino acid sequence from amino acid 126 to amino acid 135 of SEQ ID NO:2.

In one embodiment, the present invention provides a composition comprising an isolated polynucleotide selected from the group consisting of:

(a) a polynucleotide comprising the nucleotide sequence of SEQ ID NO:3;

(b) a polynucleotide comprising the nucleotide sequence of SEQ ID NO:3 from nucleotide 100 to nucleotide 882;

(c) a polynucleotide comprising the nucleotide sequence of SEQ ID NO:3 from nucleotide 635 to nucleotide 867;

(d) a polynucleotide comprising the nucleotide sequence of the full-length protein coding sequence of clone bk112_{—}15 deposited under accession number ATCC 98451;

(e) a polynucleotide encoding the full-length protein encoded by the cDNA insert of clone bk112_{—}15 deposited under accession number ATCC 98451;

(f) a polynucleotide comprising the nucleotide sequence of a mature protein coding sequence of clone bk112_{—}15 deposited under accession number ATCC 98451;

(g) a polynucleotide encoding a mature protein encoded by the cDNA insert of clone bk112_{—}15 deposited under accession number ATCC 98451;

(h) a polynucleotide encoding a protein comprising the amino acid sequence of SEQ ID NO:4;

(i) a polynucleotide encoding a protein comprising a fragment of the amino acid sequence of SEQ ID NO:4 having biological activity, the fragment comprising eight consecutive amino acids of SEQ ID NO:4;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:3 from nucleotide 100 to nucleotide 882; the nucleotide sequence of SEQ ID NO:3 from nucleotide 635 to nucleotide 867; the nucleotide sequence of the full-length protein coding sequence of clone bk112_{—}15 deposited under accession number ATCC 98451; or the nucleotide sequence of a mature protein coding sequence of clone bk112_{—}15 deposited under accession number ATCC 98451. In other preferred embodiments, the polynucleotide encodes the full-length or a mature protein encoded by the cDNA insert of clone bk112_{—}15 deposited under accession number ATCC 98451. In yet other preferred embodiments, the present invention provides a polynucleotide encoding a protein comprising the amino acid sequence of SEQ ID NO:4 from amino acid 200 to amino acid 256. In further preferred embodiments, the present invention provides a polynucleotide encoding a protein comprising a fragment of the amino acid sequence of SEQ ID NO:4 having biological activity, the fragment preferably comprising eight (more preferably twenty, most preferably thirty) consecutive amino acids of SEQ ID NO:4, or a polynucleotide encoding a protein comprising a fragment of the amino acid sequence of SEQ ID NO:4 having biological activity, the fragment comprising the amino acid sequence from amino acid 125 to amino acid 134 of SEQ ID NO:4.

Other embodiments provide the gene corresponding to the cDNA sequence of SEQ ID NO:3.

In other embodiments, the present invention provides a composition comprising a protein, wherein said protein comprises an amino acid sequence selected from the group consisting of:

(a) the amino acid sequence of SEQ ID NO:4;

(b) the amino acid sequence of SEQ ID NO:4 from amino acid 200 to amino acid 256;

(c) fragments of the amino acid sequence of SEQ ID NO:4 comprising eight consecutive amino acids of SEQ ID NO:4; and

(d) the amino acid sequence encoded by the cDNA insert of clone bk112_{—}15 deposited under accession number ATCC 98451;

the protein being substantially free from other mammalian proteins. Preferably such protein comprises the amino acid sequence of SEQ ID NO:4 or the amino acid sequence of SEQ ID NO:4 from amino acid 200 to amino acid 256. In further preferred embodiments, the present invention provides a protein comprising a fragment of the amino acid sequence of SEQ ID NO:4 having biological activity, the fragment preferably comprising eight (more preferably twenty, most preferably thirty) consecutive amino acids of SEQ ID NO:4, or a protein comprising a fragment of the amino acid sequence of SEQ ID NO:4 having biological activity, the fragment comprising the amino acid sequence from amino acid 125 to amino acid 134 of SEQ ID NO:4.

In one embodiment, the present invention provides a composition comprising an isolated polynucleotide selected from the group consisting of:

(a) a polynucleotide comprising the nucleotide sequence of SEQ ID NO:5;

(b) a polynucleotide comprising the nucleotide sequence of SEQ ID NO:5 from nucleotide 245 to nucleotide 520;

(c) a polynucleotide comprising the nucleotide sequence of SEQ ID NO:5from nucleotide 181 to nucleotide 527;

(d) a polynucleotide comprising the nucleotide sequence of the full-length protein coding sequence of clone bk200_{—}13 deposited under accession number ATCC 98451;

(e) a polynucleotide encoding the full-length protein encoded by the cDNA insert of clone bk200_{—}13 deposited under accession number ATCC 98451;

(f) a polynucleotide comprising the nucleotide sequence of a mature protein coding sequence of clone bk200_{—}13 deposited under accession number ATCC 98451;

(g) a polynucleotide encoding a mature protein encoded by the cDNA insert of clone bk200_{—}13 deposited under accession number ATCC 98451;

(h) a polynucleotide encoding a protein comprising the amino acid sequence of SEQ ID NO:6;

(i) a polynucleotide encoding a protein comprising a fragment of the amino acid sequence of SEQ ID NO:6 having biological activity, the fragment comprising eight consecutive amino acids of SEQ ID NO:6;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:5 from nucleotide 245 to nucleotide 520; the nucleotide sequence of SEQ ID NO:5 from nucleotide 181 to nucleotide 527; the nucleotide sequence of the full-length protein coding sequence of clone bk200_{—}13 deposited under accession number ATCC 98451; or the nucleotide sequence of a mature protein coding sequence of clone bk200_{—}13 deposited under accession number ATCC 98451. In other preferred embodiments, the polynucleotide encodes the full-length or a mature protein encoded by the cDNA insert of clone bk200_{—}13 deposited under accession number ATCC 98451. In further preferred embodiments, the present invention provides a polynucleotide encoding a protein comprising a fragment of the amino acid sequence of SEQ ID NO:6 having biological activity, the fragment preferably comprising eight (more preferably twenty, most preferably thirty) consecutive amino acids of SEQ ID NO:6, or a polynucleotide encoding a protein comprising a fragment of the amino acid sequence of SEQ ID NO:6 having biological activity, the fragment comprising the amino acid sequence from amino acid 41 to amino acid 50 of SEQ ID NO:6.

Other embodiments provide the gene corresponding to the cDNA sequence of SEQ ID NO:5.

In other embodiments, the present invention provides a composition comprising a protein, wherein said protein comprises an amino acid sequence selected from the group consisting of:

(a) the amino acid sequence of SEQ ID NO:6;

(b) fragments of the amino acid sequence of SEQ ID NO:6 comprising eight consecutive amino acids of SEQ ID NO:6; and

(c) the amino acid sequence encoded by the cDNA insert of clone bk200_{—}13 deposited under accession number ATCC 98451;

the protein being substantially free from other mammalian proteins. Preferably such protein comprises the amino acid sequence of SEQ ID NO:6. In further preferred embodiments, the present invention provides a protein comprising a fragment of the amino acid sequence of SEQ ID NO:6 having biological activity, the fragment preferably comprising eight (more preferably twenty, most preferably thirty) consecutive amino acids of SEQ ID NO:6, or a protein comprising a fragment of the amino acid sequence of SEQ ID NO:6 having biological activity, the fragment comprising the amino acid sequence from amino acid 41 to amino acid 50 of SEQ ID NO:6.

In one embodiment, the present invention provides a composition comprising an isolated polynucleotide selected from the group consisting of:

(a) a polynucleotide comprising the nucleotide sequence of SEQ ID NO:7;

(b) a polynucleotide comprising the nucleotide sequence of SEQ ID NO:7 from nucleotide 365 to nucleotide 784;

(c) a polynucleotide comprising the nucleotide sequence of SEQ ID NO:7 from nucleotide 518 to nucleotide 784;

(d) a polynucleotide comprising the nucleotide sequence of the full-length protein coding sequence of clone di386_{—}3 deposited under accession number ATCC 98451;

(e) a polynucleotide encoding the full-length protein encoded by the cDNA insert of clone di386_{—}3 deposited under accession number ATCC 98451;

(f) a polynucleotide comprising the nucleotide sequence of a mature protein coding sequence of clone di386_{—}3 deposited under accession number ATCC 98451;

(g) a polynucleotide encoding a mature protein encoded by the cDNA insert of clone di386_{—}3 deposited under accession number ATCC 98451;

(h) a polynucleotide encoding a protein comprising the amino acid sequence of SEQ ID NO:8;

(i) a polynucleotide encoding a protein comprising a fragment of the amino acid sequence of SEQ ID NO:8 having biological activity, the fragment comprising eight consecutive amino acids of SEQ ID NO:8;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:7 from nucleotide 365 to nucleotide 784; the nucleotide sequence of SEQ ID NO:7 from nucleotide 518 to nucleotide 784; the nucleotide sequence of the full-length protein coding sequence of clone di386_{—}3 deposited under accession number ATCC 98451; or the nucleotide sequence of a mature protein coding sequence of clone di386_{—}3 deposited under accession number ATCC 98451. In other preferred embodiments, the polynucleotide encodes the full-length or a mature protein encoded by the cDNA insert of clone di386_{—}3 deposited under accession number ATCC 98451. In yet other preferred embodiments, the present invention provides a polynucleotide encoding a protein comprising the amino acid sequence of SEQ ID NO:8 from amino acid 1 to amino acid 140. In further preferred embodiments, the present invention provides a polynucleotide encoding a protein comprising a fragment of the amino acid sequence of SEQ ID NO:8 having biological activity, the fragment preferably comprising eight (more preferably twenty, most preferably thirty) consecutive amino acids of SEQ ID NO:8, or a polynucleotide encoding a protein comprising a fragment of the amino acid sequence of SEQ ID NO:8 having biological activity, the fragment comprising the amino acid sequence from amino acid 65 to amino acid 74 of SEQ ID NO:8.

Other embodiments provide the gene corresponding to the cDNA sequence of SEQ ID NO:7 or SEQ ID NO:9.

In other embodiments, the present invention provides a composition comprising a protein, wherein said protein comprises an amino acid sequence selected from the group consisting of:

(a) the amino acid sequence of SEQ ID NO:8;

(b) the amino acid sequence of SEQ ID NO:8 from amino acid 1 to amino acid 140;

(c) fragments of the amino acid sequence of SEQ ID NO:8 comprising eight consecutive amino acids of SEQ ID NO:8; and

(d) the amino acid sequence encoded by the cDNA insert of clone di386_{—}3 deposited under accession number ATCC 98451;

the protein being substantially free from other mammalian proteins. Preferably such protein comprises the amino acid sequence of SEQ ID NO:8 or the amino acid sequence of SEQ ID NO:8 from amino acid 1 to amino acid 140. In further preferred embodiments, the present invention provides a protein comprising a fragment of the amino acid sequence of SEQ ID NO:8 having biological activity, the fragment preferably comprising eight (more preferably twenty, most preferably thirty) consecutive amino acids of SEQ ID NO:8, or a protein comprising a fragment of the amino acid sequence of SEQ ID NO:8 having biological activity, the fragment comprising the amino acid sequence from amino acid 65 to amino acid 74 of SEQ ID NO:8.

In one embodiment, the present invention provides a composition comprising an isolated polynucleotide selected from the group consisting of:

(a) a polynucleotide comprising the nucleotide sequence of SEQ ID NO:10;

(b) a polynucleotide comprising the nucleotide sequence of SEQ ID NO:10 from nucleotide 191 to nucleotide 781;

(c) a polynucleotide comprising the nucleotide sequence of SEQ ID NO:10 from nucleotide 56 to nucleotide 492;

(d) a polynucleotide comprising the nucleotide sequence of the full-length protein coding sequence of clone em397_{—}2 deposited under accession number ATCC 98451;

(e) a polynucleotide encoding the full-length protein encoded by the cDNA insert of clone em397_{—}2 deposited under accession number ATCC 98451;

(f) a polynucleotide comprising the nucleotide sequence of a mature protein coding sequence of clone em397_{—}2 deposited under accession number ATCC 98451;

(g) a polynucleotide encoding a mature protein encoded by the cDNA insert of clone em397_{—}2 deposited under accession number ATCC 98451;

(h) a polynucleotide encoding a protein comprising the amino acid sequence of SEQ ID NO:11;

(i) a polynucleotide encoding a protein comprising a fragment of the amino acid sequence of SEQ ID NO:11 having biological activity, the fragment comprising eight consecutive amino acids of SEQ ID NO:11;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:10 from nucleotide 191 to nucleotide 781; the nucleotide sequence of SEQ ID NO:10 from nucleotide 56 to nucleotide 492; the nucleotide sequence of the full-length protein coding sequence of clone em397_{—}2 deposited under accession number ATCC 98451; or the nucleotide sequence of a mature protein coding sequence of clone em397_{—}2 deposited under accession number ATCC 98451. In other preferred embodiments, the polynucleotide encodes the full-length or a mature protein encoded by the cDNA insert of clone em397_{—}2 deposited under accession number ATCC 98451. In yet other preferred embodiments, the present invention provides a polynucleotide encoding a protein comprising the amino acid sequence of SEQ ID NO:11 from amino acid 1 to amino acid 101. In further preferred embodiments, the present invention provides a polynucleotide encoding a protein comprising a fragment of the amino acid sequence of SEQ ID NO:11 having biological activity, the fragment preferably comprising eight (more preferably twenty, most preferably thirty) consecutive amino acids of SEQ ID NO:11, or a polynucleotide encoding a protein comprising a fragment of the amino acid sequence of SEQ ID NO:11 having biological activity, the fragment comprising the amino acid sequence from amino acid 93 to amino acid 102 of SEQ ID NO:11.

Other embodiments provide the gene corresponding to the cDNA sequence of SEQ ID NO:10.

In other embodiments, the present invention provides a composition comprising a protein, wherein said protein comprises an amino acid sequence selected from the group consisting of:

(a) the amino acid sequence of SEQ ID NO:11;

(b) the amino acid sequence of SEQ ID NO:11 from amino acid 1 to amino acid 101;

(c) fragments of the amino acid sequence of SEQ ID NO:11 comprising eight consecutive amino acids of SEQ ID NO:11; and

(d) the amino acid sequence encoded by the cDNA insert of clone em397_{—}2 deposited under accession number ATCC 98451;

the protein being substantially free from other mammalian proteins. Preferably such protein comprises the amino acid sequence of SEQ ID NO:11 or the amino acid sequence of SEQ ID NO:11 from amino acid 1 to amino acid 101. In further preferred embodiments, the present invention provides a protein comprising a fragment of the amino acid sequence of SEQ ID NO:11 having biological activity, the fragment preferably comprising eight (more preferably twenty, most preferably thirty) consecutive amino acids of SEQ ID NO:11, or a protein comprising a fragment of the amino acid sequence of SEQ ID NO:11 having biological activity, the fragment comprising the amino acid sequence from amino acid 93 to amino acid 102 of SEQ ID NO:11.

In one embodiment, the present invention provides a composition comprising an isolated polynucleotide selected from the group consisting of:

(a) a polynucleotide comprising the nucleotide sequence of SEQ ID NO:12;

(b) a polynucleotide comprising the nucleotide sequence of SEQ ID NO:12 from nucleotide 65 to nucleotide 1636;

(c) a polynucleotide comprising the nucleotide sequence of SEQ ID NO:12 from nucleotide 482 to nucleotide 1636;

(d) a polynucleotide comprising the nucleotide sequence of SEQ ID NO:12 from nucleotide 487 to nucleotide 1006;

(e) a polynucleotide comprising the nucleotide sequence of the full-length protein coding sequence of clone fh170_{—}7 deposited under accession number ATCC 98451;

(f) a polynucleotide encoding the full-length protein encoded by the cDNA insert of clone fh170_{—}7 deposited under accession number ATCC 98451;

(g) a polynucleotide comprising the nucleotide sequence of a mature protein coding sequence of clone fh170_{—}7 deposited under accession number ATCC 98451;

(h) a polynucleotide encoding a mature protein encoded by the cDNA insert of clone fh170_{—}7 deposited under accession number ATCC 98451;

(i) a polynucleotide encoding a protein comprising the amino acid sequence of SEQ ID NO:13;

(j) a polynucleotide encoding a protein comprising a fragment of the amino acid sequence of SEQ ID NO:13 having biological activity, the fragment comprising eight consecutive amino acids of SEQ ID NO:13;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:12 from nucleotide 65 to nucleotide 1636; the nucleotide sequence of SEQ ID NO:12 from nucleotide 482 to nucleotide 1636; the nucleotide sequence of SEQ ID NO:12 from nucleotide 487 to nucleotide 1006; the nucleotide sequence of the full-length protein coding sequence of clone fh170_{—}7 deposited under accession number ATCC 98451; or the nucleotide sequence of a mature protein coding sequence of clone fh170_{—}7 deposited under accession number ATCC 98451. In other preferred embodiments, the polynucleotide encodes the full-length or a mature protein encoded by the cDNA insert of clone fh170_{—}7 deposited under accession number ATCC 98451. In yet other preferred embodiments, the present invention provides a polynucleotide encoding a protein comprising the amino acid sequence of SEQ ID NO:13 from amino acid 142 to amino acid 314. In further preferred embodiments, the present invention provides a polynucleotide encoding a protein comprising a fragment of the amino acid sequence of SEQ ID NO:13 having biological activity, the fragment preferably comprising eight (more preferably twenty, most preferably thirty) consecutive amino acids of SEQ ID NO:13, or a polynucleotide encoding a protein comprising a fragment of the amino acid sequence of SEQ ID NO:13 having biological activity, the fragment comprising the amino acid sequence from amino acid 257 to amino acid 266 of SEQ ID NO:13.

Other embodiments provide the gene corresponding to the cDNA sequence of SEQ ID NO:12.

In other embodiments, the present invention provides a composition comprising a protein, wherein said protein comprises an amino acid sequence selected from the group consisting of:

(a) the amino acid sequence of SEQ ID NO:13;

(b) the amino acid sequence of SEQ ID NO:13 from amino acid 142 to amino acid 314;

(c) fragments of the amino acid sequence of SEQ ID NO:13 comprising eight consecutive amino acids of SEQ ID NO:13; and

(d) the amino acid sequence encoded by the cDNA insert of clone fh170_{—}7 deposited under accession number ATCC 98451;

the protein being substantially free from other mammalian proteins. Preferably such protein comprises the amino acid sequence of SEQ ID NO:13 or the amino acid sequence of SEQ ID NO:13 from amino acid 142 to amino acid 314. In further preferred embodiments, the present invention provides a protein comprising a fragment of the amino acid sequence of SEQ ID NO:13 having biological activity, the fragment preferably comprising eight (more preferably twenty, most preferably thirty) consecutive amino acids of SEQ ID NO:13, or a protein comprising a fragment of the amino acid sequence of SEQ ID NO:13 having biological activity, the fragment comprising the amino acid sequence from amino acid 257 to amino acid 266 of SEQ ID NO:13.

In one embodiment, the present invention provides a composition comprising an isolated polynucleotide selected from the group consisting of:

(a) a polynucleotide comprising the nucleotide sequence of SEQ ID NO:15;

(b) a polynucleotide comprising the nucleotide sequence of SEQ ID NO:15 from nucleotide 41 to nucleotide 550;

(c) a polynucleotide comprising the nucleotide sequence of the full-length protein coding sequence of clone fn53_{—}4 deposited under accession number ATCC 98451;

(d) a polynucleotide encoding the full-length protein encoded by the cDNA insert of clone fn53_{—}4 deposited under accession number ATCC 98451;

(e) a polynucleotide comprising the nucleotide sequence of a mature protein coding sequence of clone fn53_{—}4 deposited under accession number ATCC 98451;

(f) a polynucleotide encoding a mature protein encoded by the cDNA insert of clone fn53_{—}4 deposited under accession number ATCC 98451;

(g) a polynucleotide encoding a protein comprising the amino acid sequence of SEQ ID NO:16;

(h) a polynucleotide encoding a protein comprising a fragment of the amino acid sequence of SEQ ID NO:16 having biological activity, the fragment comprising eight consecutive amino acids of SEQ ID NO:16;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:15 from nucleotide 41 to nucleotide 550; the nucleotide sequence of the full-length protein coding sequence of clone fn53_{—}4 deposited under accession number ATCC 98451; or the nucleotide sequence of a mature protein coding sequence of clone fn53_{—}4 deposited under accession number ATCC 98451. In other preferred embodiments, the polynucleotide encodes the full-length or a mature protein encoded by the cDNA insert of clone fn53_{—}4 deposited under accession number ATCC 98451. In yet other preferred embodiments, the present invention provides a polynucleotide encoding a protein comprising the amino acid sequence of SEQ ID NO:16 from amino acid 40 to amino acid 170. In further preferred embodiments, the present invention provides a polynucleotide encoding a protein comprising a fragment of the amino acid sequence of SEQ ID NO:16 having biological activity, the fragment preferably comprising eight (more preferably twenty, most preferably thirty) consecutive amino acids of SEQ ID NO:16, or a polynucleotide encoding a protein comprising a fragment of the amino acid sequence of SEQ ID NO:16 having biological activity, the fragment comprising the amino acid sequence from amino acid 80 to amino acid 89 of SEQ ID NO:16.

Other embodiments provide the gene corresponding to the cDNA sequence of SEQ ID NO:15, SEQ ID NO:14 or SEQ ID NO:17.

In other embodiments, the present invention provides a composition comprising a protein, wherein said protein comprises an amino acid sequence selected from the group consisting of:

(a) the amino acid sequence of SEQ ID NO:16;

(b) the amino acid sequence of SEQ ID NO:16 from amino acid 40 to amino acid 170;

(c) fragments of the amino acid sequence of SEQ ID NO:16 comprising eight consecutive amino acids of SEQ ID NO:16; and

(d) the amino acid sequence encoded by the cDNA insert of clone fn53_{—}4 deposited under accession number ATCC 98451;

the protein being substantially free from other mammalian proteins. Preferably such protein comprises the amino acid sequence of SEQ ID NO:16 or the amino acid sequence of SEQ ID NO:16 from amino acid 40 to amino acid 170. In further preferred embodiments, the present invention provides a protein comprising a fragment of the amino acid sequence of SEQ ID NO:16 having biological activity, the fragment preferably comprising eight (more preferably twenty, most preferably thirty) consecutive amino acids of SEQ ID NO:16, or a protein comprising a fragment of the amino acid sequence of SEQ ID NO:16 having biological activity, the fragment comprising the amino acid sequence from amino acid 80 to amino acid 89 of SEQ ID NO:16.

In one embodiment, the present invention provides a composition comprising an isolated polynucleotide selected from the group consisting of:

(a) a polynucleotide comprising the nucleotide sequence of SEQ ID NO:18;

(b) a polynucleotide comprising the nucleotide sequence of SEQ ID NO:18 from nucleotide 84 to nucleotide 404;

(c) a polynucleotide comprising the nucleotide sequence of SEQ ID NO:18 from nucleotide 78 to nucleotide 493;

(d) a polynucleotide comprising the nucleotide sequence of the full-length protein coding sequence of clone fq505_{—}4 deposited under accession number ATCC 98451;

(e) a polynucleotide encoding the full-length protein encoded by the cDNA insert of clone fq505_{—}4 deposited under accession number ATCC 98451;

(f) a polynucleotide comprising the nucleotide sequence of a mature protein coding sequence of clone fq505_{—}4 deposited under accession number ATCC 98451;

(g) a polynucleotide encoding a mature protein encoded by the cDNA insert of clone fq505_{—}4 deposited under accession number ATCC 98451;

(h) a polynucleotide encoding a protein comprising the amino acid sequence of SEQ ID NO:19;

(i) a polynucleotide encoding a protein comprising a fragment of the amino acid sequence of SEQ ID NO:19 having biological activity, the fragment comprising eight consecutive amino acids of SEQ ID NO:19;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:18 from nucleotide 84 to nucleotide 404; the nucleotide sequence of SEQ ID NO:18 from nucleotide 78 to nucleotide 493; the nucleotide sequence of the full-length protein coding sequence of clone fq505_{—}4 deposited under accession number ATCC 98451; or the nucleotide sequence of a mature protein coding sequence of clone fq505_{—}4 deposited under accession number ATCC 98451. In other preferred embodiments, the polynucleotide encodes the full-length or a mature protein encoded by the cDNA insert of clone fq505_{—}4 deposited under accession number ATCC 98451. In yet other preferred embodiments, the present invention provides a polynucleotide encoding a protein comprising the amino acid sequence of SEQ ID NO:19 from amino acid 23 to amino acid 107. In further preferred embodiments, the present invention provides a polynucleotide encoding a protein comprising a fragment of the amino acid sequence of SEQ ID NO:19 having biological activity, the fragment preferably comprising eight (more preferably twenty, most preferably thirty) consecutive amino acids of SEQ ID NO:19, or a polynucleotide encoding a protein comprising a fragment of the amino acid sequence of SEQ ID NO:19 having biological activity, the fragment comprising the amino acid sequence from amino acid 48 to amino acid 57 of SEQ ID NO:19.

Other embodiments provide the gene corresponding to the cDNA sequence of SEQ ID NO:18.

In other embodiments, the present invention provides a composition comprising a protein, wherein said protein comprises an amino acid sequence selected from the group consisting of:

(a) the amino acid sequence of SEQ ID NO:19;

(b) the amino acid sequence of SEQ ID NO:19 from amino acid 23 to amino acid 107;

(c) fragments of the amino acid sequence of SEQ ID NO:19 comprising eight consecutive amino acids of SEQ ID NO:19; and

(d) the amino acid sequence encoded by the cDNA insert of clone fq505_{—}4 deposited under accession number ATCC 98451;

the protein being substantially free from other mammalian proteins. Preferably such protein comprises the amino acid sequence of SEQ ID NO:19 or the amino acid sequence of SEQ ID NO:19 from amino acid 23 to amino acid 107. In further preferred embodiments, the present invention provides a protein comprising a fragment of the amino acid sequence of SEQ ID NO:19 having biological activity, the fragment preferably comprising eight (more preferably twenty, most preferably thirty) consecutive amino acids of SEQ ID NO:19, or a protein comprising a fragment of the amino acid sequence of SEQ ID NO:19 having biological activity, the fragment comprising the amino acid sequence from amino acid 48 to amino acid 57 of SEQ ID NO:19.

In one embodiment, the present invention provides a composition comprising an isolated polynucleotide selected from the group consisting of:

(a) a polynucleotide comprising the nucleotide sequence of SEQ ID NO:20;

(b) a polynucleotide comprising the nucleotide sequence of SEQ ID NO:20 from nucleotide 1439 to nucleotide 1744;

(c) a polynucleotide comprising the nucleotide sequence of SEQ ID NO:20 from nucleotide 1241 to nucleotide 1754;

(d) a polynucleotide comprising the nucleotide sequence of the full-length protein coding sequence of clone fw13_{—}9 deposited under accession number ATCC 98451;

(e) a polynucleotide encoding the full-length protein encoded by the cDNA insert of clone fw13_{—}9 deposited under accession number ATCC 98451;

(f) a polynucleotide comprising the nucleotide sequence of a mature protein coding sequence of clone fw13_{—}9 deposited under accession number ATCC 98451;

(g) a polynucleotide encoding a mature protein encoded by the cDNA insert of clone fw13_{—}9 deposited under accession number ATCC 98451;

(h) a polynucleotide encoding a protein comprising the amino acid sequence of SEQ ID NO:21;

(i) a polynucleotide encoding a protein comprising a fragment of the amino acid sequence of SEQ ID NO:21 having biological activity, the fragment comprising eight consecutive amino acids of SEQ ID NO:21;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:20 from nucleotide 1439 to nucleotide 1744; the nucleotide sequence of SEQ ID NO:20 from nucleotide 1241 to nucleotide 1754; the nucleotide sequence of the full-length protein coding sequence of clone fw13_{—}9 deposited under accession number ATCC 98451; or the nucleotide sequence of a mature protein coding sequence of clone fw13_{—}9 deposited under accession number ATCC 98451. In other preferred embodiments, the polynucleotide encodes the full-length or a mature protein encoded by the cDNA insert of clone fw13_{—}9 deposited under accession number ATCC 98451. In yet other preferred embodiments, the present invention provides a polynucleotide encoding a protein comprising the amino acid sequence of SEQ ID NO:21 from amino acid 1 to amino acid 57. In further preferred embodiments, the present invention provides a polynucleotide encoding a protein comprising a fragment of the amino acid sequence of SEQ ID NO:21 having biological activity, the fragment preferably comprising eight (more preferably twenty, most preferably thirty) consecutive amino acids of SEQ ID NO:21, or a polynucleotide encoding a protein comprising a fragment of the amino acid sequence of SEQ ID NO:21 having biological activity, the fragment comprising the amino acid sequence from amino acid 46 to amino acid 55 of SEQ ID NO:21.

Other embodiments provide the gene corresponding to the cDNA sequence of SEQ ID NO:20.

In other embodiments, the present invention provides a composition comprising a protein, wherein said protein comprises an amino acid sequence selected 2:5 from the group consisting of:

(a) the amino acid sequence of SEQ ID NO:21;

(b) the amino acid sequence of SEQ ID NO:21 from amino acid 1 to amino acid 57;

(c) fragments of the amino acid sequence of SEQ ID NO:21 comprising eight consecutive amino acids of SEQ ID NO:21; and

(d) the amino acid sequence encoded by the cDNA insert of clone fw13_{—}9 deposited under accession number ATCC 98451;

the protein being substantially free from other mammalian proteins. Preferably such protein comprises the amino acid sequence of SEQ ID NO:21 or the amino acid sequence of SEQ ID NO:21 from amino acid 1 to amino acid 57. In further preferred embodiments, the present invention provides a protein comprising a fragment of the amino acid sequence of SEQ ID NO:21 having biological activity, the fragment preferably comprising eight (more preferably twenty, most preferably thirty) consecutive amino acids of SEQ ID NO:21, or a protein comprising a fragment of the amino acid sequence of SEQ ID NO:21 having biological activity, the fragment comprising the amino acid sequence from amino acid 46 to amino acid 55 of SEQ ID NO:21.

In one embodiment, the present invention provides a composition comprising an isolated polynucleotide selected from the group consisting of:

(a) a polynucleotide comprising the nucleotide sequence of SEQ ID NO:22;

(b) a polynucleotide comprising the nucleotide sequence of SEQ ID NO:22 from nucleotide 47 to nucleotide 919;

(c) a polynucleotide comprising the nucleotide sequence of SEQ ID NO:22 from nucleotide 124 to nucleotide 452;

(d) a polynucleotide comprising the nucleotide sequence of the full-length protein coding sequence of clone gg619_{—}2 deposited under accession number ATCC 98451;

(e) a polynucleotide encoding the full-length protein encoded by the cDNA insert of clone gg619_{—}2 deposited under accession number ATCC 98451;

(f) a polynucleotide comprising the nucleotide sequence of a mature protein coding sequence of clone gg619_{—}2 deposited under accession number ATCC 98451;

(g) a polynucleotide encoding a mature protein encoded by the cDNA insert of clone gg619_{—}2 deposited under accession number ATCC 98451;

(h) a polynucleotide encoding a protein comprising the amino acid sequence of SEQ ID NO:23;

(i) a polynucleotide encoding a protein comprising a fragment of the amino acid sequence of SEQ ID NO:23 having biological activity, the fragment comprising eight consecutive amino acids of SEQ ID NO:23;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:22 from nucleotide 47 to nucleotide 919; the nucleotide sequence of SEQ ID NO:22 from nucleotide 124 to nucleotide 452; the nucleotide sequence of the full-length protein coding sequence of clone gg619_{—}2 deposited under accession number ATCC 98451; or the nucleotide sequence of a mature protein coding sequence of clone gg619_{—}2 deposited under accession number ATCC 98451. In other preferred embodiments, the polynucleotide encodes the full-length or a mature protein encoded by the cDNA insert of clone gg619_{—}2 deposited under accession number ATCC 98451. In yet other preferred embodiments, the present invention provides a polynucleotide encoding a protein comprising the amino acid sequence of SEQ ID NO:23 from amino acid 27 to amino acid 135. In further preferred embodiments, the present invention provides a polynucleotide encoding a protein comprising a fragment of the amino acid sequence of SEQ ID NO:23 having biological activity, the fragment preferably comprising eight (more preferably twenty, most preferably thirty) consecutive amino acids of SEQ ID NO:23, or a polynucleotide encoding a protein comprising a fragment of the amino acid sequence of SEQ ID NO:23 having biological activity, the fragment comprising the amino acid sequence from amino acid 140 to amino acid 149 of SEQ ID NO:23.

Other embodiments provide the gene corresponding to the cDNA sequence of SEQ ID NO:22.

In other embodiments, the present invention provides a composition comprising a protein, wherein said protein comprises an amino acid sequence selected from the group consisting of:

(a) the amino acid sequence of SEQ ID NO:23;

(b) the amino acid sequence of SEQ ID NO:23 from amino acid 27 to amino acid 135;

(c) fragments of the amino acid sequence of SEQ ID NO:23 comprising eight consecutive amino acids of SEQ ID NO:23; and

(d) the amino acid sequence encoded by the cDNA insert of clone gg619_{—}2 deposited under accession number ATCC 98451;

the protein being substantially free from other mammalian proteins. Preferably such protein comprises the amino acid sequence of SEQ ID NO:23 or the amino acid sequence of SEQ ID NO:23 from amino acid 27 to amino acid 135. In further preferred embodiments, the present invention provides a protein comprising a fragment of the amino acid sequence of SEQ ID NO:23 having biological activity, the fragment preferably comprising eight (more preferably twenty, most preferably thirty) consecutive amino acids of SEQ ID NO:23, or a protein comprising a fragment of the amino acid sequence of SEQ ID NO:23 having biological activity, the fragment comprising the amino acid sequence from amino acid 140 to amino acid 149 of SEQ ID NO:23.

In one embodiment, the present invention provides a composition comprising an isolated polynucleotide selected from the group consisting of:

(a) a polynucleotide comprising the nucleotide sequence of SEQ ID NO:35;

(b) a polynucleotide comprising the nucleotide sequence of SEQ ID NO:35 from nucleotide 2178 to nucleotide 2513;

(c) a polynucleotide comprising the nucleotide sequence of SEQ ID NO:35 from nucleotide 2364 to nucleotide 2513;

(d) a polynucleotide comprising the nucleotide sequence of SEQ ID NO:35 from nucleotide 1980 to nucleotide 2311;

(e) a polynucleotide comprising the nucleotide sequence of the full-length protein coding sequence of clone cl181_{—}3 deposited under accession number ATCC 98456;

(f) a polynucleotide encoding the full-length protein encoded by the cDNA insert of clone cl1813 deposited under accession number ATCC 98456;

(g) a polynucleotide comprising the nucleotide sequence of a mature protein coding sequence of clone cl181_{—}3 deposited under accession number ATCC 98456;

(h) a polynucleotide encoding a mature protein encoded by the cDNA insert of clone cl181_{—}3 deposited under accession number ATCC 98456;

(i) a polynucleotide encoding a protein comprising the amino acid sequence of SEQ ID NO:36;

(j) a polynucleotide encoding a protein comprising a fragment of the amino acid sequence of SEQ ID NO:36 having biological activity, the fragment comprising eight consecutive amino acids of SEQ ID NO:36;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:35 from nucleotide 2178 to nucleotide 2513; the nucleotide sequence of SEQ ID NO:35 from nucleotide 2364 to nucleotide 2513; the nucleotide sequence of SEQ ID NO:35 from nucleotide 1980 to nucleotide 2311; the nucleotide sequence of the full-length protein coding sequence of clone cl181_{—}3 deposited under accession number ATCC 98456; or the nucleotide sequence of a mature protein coding sequence of clone cl181_{—}3 deposited under accession number ATCC 98456. In other preferred embodiments, the polynucleotide encodes the full-length or a mature protein encoded by the cDNA insert of clone cl181_{—}3 deposited under accession number ATCC 98456. In yet other preferred embodiments, the present invention provides a polynucleotide encoding a protein comprising the amino acid sequence of SEQ ID NO:36 from amino acid 1 to amino acid 67. In further preferred embodiments, the present invention provides a polynucleotide encoding a protein comprising a fragment of the amino acid sequence of SEQ ID NO:36 having biological activity, the fragment preferably comprising eight (more preferably twenty, most preferably thirty) consecutive amino acids of SEQ ID NO:36, or a polynucleotide encoding a protein comprising a fragment of the amino acid sequence of SEQ ID NO:36 having biological activity, the fragment comprising the amino acid sequence from amino acid 51 to amino acid 60 of SEQ ID NO:36.

Other embodiments provide the gene corresponding to the cDNA sequence of SEQ ID NO:35.

In other embodiments, the present invention provides a composition comprising a protein, wherein said protein comprises an amino acid sequence selected from the group consisting of:

(a) the amino acid sequence of SEQ ID NO:36;

(b) the amino acid sequence of SEQ ID NO:36 from amino acid 1 to amino acid 67;

(c) fragments of the amino acid sequence of SEQ ID NO:36 comprising eight consecutive amino acids of SEQ ID NO:36; and

(d) the amino acid sequence encoded by the cDNA insert of clone cl181_{—}3 deposited under accession number ATCC 98456;

the protein being substantially free from other mammalian proteins. Preferably such protein comprises the amino acid sequence of SEQ ID NO:36 or the amino acid sequence of SEQ ID NO:36 from amino acid 1 to amino acid 67. In further preferred embodiments, the present invention provides a protein comprising a fragment of the amino acid sequence of SEQ ID NO:36 having biological activity, the fragment preferably comprising eight (more preferably twenty, most preferably thirty) consecutive amino acids of SEQ ID NO:36, or a protein comprising a fragment of the amino acid sequence of SEQ ID NO:36 having biological activity, the fragment comprising the amino acid sequence from amino acid 51 to amino acid 60 of SEQ ID NO:36.

In one embodiment, the present invention provides a composition comprising an isolated polynucleotide selected from the group consisting of:

(a) a polynucleotide comprising the nucleotide sequence of SEQ ID NO:37;

(b) a polynucleotide comprising the nucleotide sequence of SEQ ID NO:37 from nucleotide 207 to nucleotide 893;

(c) a polynucleotide comprising the nucleotide sequence of SEQ ID NO:37 from nucleotide 1 to nucleotide 527;

(d) a polynucleotide comprising the nucleotide sequence of the full-length protein coding sequence of clone cr1044_{—}1 deposited under accession number ATCC 98456;

(e) a polynucleotide encoding the full-length protein encoded by the cDNA insert of clone cr1044_{—}1 deposited under accession number ATCC 98456;

(f) a polynucleotide comprising the nucleotide sequence of a mature protein coding sequence of clone cr1044_{—}1 deposited under accession number ATCC 98456;

(g) a polynucleotide encoding a mature protein encoded by the cDNA insert of clone cr1044_{—}1 deposited under accession number ATCC 98456;

(h) a polynucleotide encoding a protein comprising the amino acid sequence of SEQ ID NO:38;

(i) a polynucleotide encoding a protein comprising a fragment of the amino acid sequence of SEQ ID NO:38 having biological activity, the fragment comprising eight consecutive amino acids of SEQ ID NO:38;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:37 from nucleotide 207 to nucleotide 893; the nucleotide sequence of SEQ ID NO:37 from nucleotide 1 to nucleotide 527; the nucleotide sequence of the full-length protein coding sequence of clone cr1044_{—}1 deposited under accession number ATCC 98456; or the nucleotide sequence of a mature protein coding sequence of clone cr1044_{—}1 deposited under accession number ATCC 98456. In other preferred embodiments, the polynucleotide encodes the full-length or a mature protein encoded by the cDNA insert of clone cr1044_{—}1 deposited under accession number ATCC 98456. In yet other preferred embodiments, the present invention provides a polynucleotide encoding a protein comprising the amino acid sequence of SEQ ID NO:38 from amino acid 1 to amino acid 107. In further preferred embodiments, the present invention provides a polynucleotide encoding a protein comprising a fragment of the amino acid sequence of SEQ ID NO:38 having biological activity, the fragment preferably comprising eight (more preferably twenty, most preferably thirty) consecutive amino acids of SEQ ID NO:38, or a polynucleotide encoding a protein comprising a fragment of the amino acid sequence of SEQ ID NO:38 having biological activity, the fragment comprising the amino acid sequence from amino acid 109 to amino acid 118 of SEQ ID NO:38.

Other embodiments provide the gene corresponding to the cDNA sequence of SEQ ID NO:37.

In other embodiments, the present invention provides a composition comprising a protein, wherein said protein comprises an amino acid sequence selected from the group consisting of:

(a) the amino acid sequence of SEQ ID NO:38;

(b) the amino acid sequence of SEQ ID NO:38 from amino acid 1 to amino acid 107;

(c) fragments of the amino acid sequence of SEQ ID NO:38 comprising eight consecutive amino acids of SEQ ID NO:38; and

(d) the amino acid sequence encoded by the cDNA insert of clone cr1044_{—}1 deposited under accession number ATCC 98456;

the protein being substantially free from other mammalian proteins. Preferably such protein comprises the amino acid sequence of SEQ ID NO:38 or the amino acid sequence of SEQ ID NO:38 from amino acid 1 to amino acid 107. In further preferred embodiments, the present invention provides a protein comprising a fragment of the amino acid sequence of SEQ ID NO:38 having biological activity, the fragment preferably comprising eight (more preferably twenty, most preferably thirty) consecutive amino acids of SEQ ID NO:38, or a protein comprising a fragment of the amino acid sequence of SEQ ID NO:38 having biological activity, the fragment comprising the amino acid sequence from amino acid 109 to amino acid 118 of SEQ ID NO:38.

In one embodiment, the present invention provides a composition comprising an isolated polynucleotide selected from the group consisting of:

(a) a polynucleotide comprising the nucleotide sequence of SEQ ID NO:39;

(b) a polynucleotide comprising the nucleotide sequence of SEQ ID NO:39 from nucleotide 77 to nucleotide 400;

(c) a polynucleotide comprising the nucleotide sequence of SEQ ID NO:39 from nucleotide 118 to nucleotide 392;

(d) a polynucleotide comprising the nucleotide sequence of the full-length protein coding sequence of clone cz251_{—}1 deposited under accession number ATCC 98456;

(e) a polynucleotide encoding the full-length protein encoded by the cDNA insert of clone cz251_{—}1 deposited under accession number ATCC 98456;

(f) a polynucleotide comprising the nucleotide sequence of a mature protein coding sequence of clone cz251_{—}1 deposited under accession number ATCC 98456;

(g) a polynucleotide encoding a mature protein encoded by the cDNA insert of clone cz251_{—}1 deposited under accession number ATCC 98456;

(h) a polynucleotide encoding a protein comprising the amino acid sequence of SEQ ID NO:40;

(i) a polynucleotide encoding a protein comprising a fragment of the amino acid sequence of SEQ ID NO:40 having biological activity, the fragment comprising eight consecutive amino acids of SEQ ID NO:40;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:39 from nucleotide 77 to nucleotide 400; the nucleotide sequence of SEQ ID NO:39 from nucleotide 118 to nucleotide 392; the nucleotide sequence of the full-length protein coding sequence of clone cz251_{—}1 deposited under accession number ATCC 98456; or the nucleotide sequence of a mature protein coding sequence of clone cz251_{—}1deposited under accession number ATCC 98456. In other preferred embodiments, the polynucleotide encodes the full-length or a mature protein encoded by the cDNA insert of clone cz251_{—}1 deposited under accession number ATCC 98456. In yet other preferred embodiments, the present invention provides a polynucleotide encoding a protein comprising the amino acid sequence of SEQ ID NO:40 from amino acid 15 to amino acid 105. In further preferred embodiments, the present invention provides a polynucleotide encoding a protein comprising a fragment of the amino acid sequence of SEQ ID NO:40 having biological activity, the fragment preferably comprising eight (more preferably twenty, most preferably thirty) consecutive amino acids of SEQ ID NO:40, or a polynucleotide encoding a protein comprising a fragment of the amino acid sequence of SEQ ID NO:40 having biological activity, the fragment comprising the amino acid sequence from amino acid 49 to amino acid 58 of SEQ ID NO:40.

Other embodiments provide the gene corresponding to the cDNA sequence of SEQ ID NO:39.

In other embodiments, the present invention provides a composition comprising a protein, wherein said protein comprises an amino acid sequence selected from the group consisting of:

(a) the amino acid sequence of SEQ ID NO:40;

(b) the amino acid sequence of SEQ ID NO:40 from amino acid 15 to amino acid 105;

(c) fragments of the amino acid sequence of SEQ ID NO:40 comprising eight consecutive amino acids of SEQ ID NO:40; and

(d) the amino acid sequence encoded by the cDNA insert of clone cz251_{—}1 deposited under accession number ATCC 98456;

the protein being substantially free from other mammalian proteins. Preferably such protein comprises the amino acid sequence of SEQ ID NO:40 or the amino acid sequence of SEQ ID NO:40 from amino acid 15 to amino acid 105. In further preferred embodiments, the present invention provides a protein comprising a fragment of the amino acid sequence of SEQ ID NO:40 having biological activity, the fragment preferably comprising eight (more preferably twenty, most preferably thirty) consecutive amino acids of SEQ ID NO:40, or a protein comprising a fragment of the amino acid sequence of SEQ ID NO:40 having biological activity, the fragment comprising the amino acid sequence from amino acid 49 to amino acid 58 of SEQ ID NO:40.

In one embodiment, the present invention provides a composition comprising an isolated polynucleotide selected from the group consisting of:

(a) a polynucleotide comprising the nucleotide sequence of SEQ ID NO:41;

(b) a polynucleotide comprising the nucleotide sequence of SEQ ID NO:41 from nucleotide 13 to nucleotide 501;

(c) a polynucleotide comprising the nucleotide sequence of SEQ ID NO:41 from nucleotide 1 to nucleotide 506;

(d) a polynucleotide comprising the nucleotide sequence of the full-length protein coding sequence of clone dd12_{—}7 deposited under accession number ATCC 98456;

(e) a polynucleotide encoding the full-length protein encoded by the cDNA insert of clone dd12_{—}7 deposited under accession number ATCC 98456;

(f) a polynucleotide comprising the nucleotide sequence of a mature protein coding sequence of clone dd12_{—}7 deposited under accession number ATCC 98456;

(g) a polynucleotide encoding a mature protein encoded by the cDNA insert of clone dd12_{—}7 deposited under accession number ATCC 98456;

(h) a polynucleotide encoding a protein comprising the amino acid sequence of SEQ ID NO:42;

(i) a polynucleotide encoding a protein comprising a fragment of the amino acid sequence of SEQ ID NO:42 having biological activity, the fragment comprising eight consecutive amino acids of SEQ ID NO:42;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:41 from nucleotide 13 to nucleotide 501; the nucleotide sequence of SEQ ID NO:41 from nucleotide 1 to nucleotide 506; the nucleotide sequence of the full-length protein coding sequence of clone dd12_{—}7 deposited under accession number ATCC 98456; or the nucleotide sequence of a mature protein coding sequence of clone dd12_{—}7 deposited under accession number ATCC 98456. In other preferred embodiments, the polynucleotide encodes the full-length or a mature protein encoded by the cDNA insert of clone dd12_{—}7 deposited under accession number ATCC 98456. In further preferred embodiments, the present invention provides a polynucleotide encoding a protein comprising a fragment of the amino acid sequence of SEQ ID NO:42 having biological activity, the fragment preferably comprising eight (more preferably twenty, most preferably thirty) consecutive amino acids of SEQ ID NO:42, or a polynucleotide encoding a protein comprising a fragment of the amino acid sequence of SEQ ID NO:42 having biological activity, the fragment comprising the amino acid sequence from amino acid 76 to amino acid 85 of SEQ ID NO:42.

Other embodiments provide the gene corresponding to the cDNA sequence of SEQ ID NO:41.

In other embodiments, the present invention provides a composition comprising a protein, wherein said protein comprises an amino acid sequence selected from the group consisting of:

(a) the amino acid sequence of SEQ ID NO:42;

(b) fragments of the amino acid sequence of SEQ ID NO:42 comprising eight consecutive amino acids of SEQ ID NO:42; and

(c) the amino acid sequence encoded by the cDNA insert of clone dd12_{—}7 deposited under accession number ATCC 98456;

the protein being substantially free from other mammalian proteins. Preferably such protein comprises the amino acid sequence of SEQ ID NO:42. In further preferred embodiments, the present invention provides a protein comprising a fragment of the amino acid sequence of SEQ ID NO:42 having biological activity, the fragment preferably comprising eight (more preferably twenty, most preferably thirty) consecutive amino acids of SEQ ID NO:42, or a protein comprising a fragment of the amino acid sequence of SEQ ID NO:42 having biological activity, the fragment comprising the amino acid sequence from amino acid 76 to amino acid 85 of SEQ ID NO:42.

In one embodiment, the present invention provides a composition comprising an isolated polynucleotide selected from the group consisting of:

(a) a polynucleotide comprising the nucleotide sequence of SEQ ID NO:43;

(b) a polynucleotide comprising the nucleotide sequence of SEQ ID NO:43 from nucleotide 778 to nucleotide 1083;

(c) a polynucleotide comprising the nucleotide sequence of SEQ ID NO:9 from nucleotide 931 to nucleotide 1083;

(d) a polynucleotide comprising the nucleotide sequence of SEQ ID NO:43 from nucleotide 802 to nucleotide 1056;

(e) a polynucleotide comprising the nucleotide sequence of the full-length protein coding sequence of clone fn191_{—}3 deposited under accession number ATCC 98456;

(f) a polynucleotide encoding the full-length protein encoded by the cDNA insert of clone fn191_{—}3 deposited under accession number ATCC 98456;

(g) a polynucleotide comprising the nucleotide sequence of a mature protein coding sequence of clone fn191_{—}3 deposited under accession number ATCC 98456;

(h) a polynucleotide encoding a mature protein encoded by the cDNA insert of clone fn191_{—}3 deposited under accession number ATCC 98456;

(i) a polynucleotide encoding a protein comprising the amino acid sequence of SEQ ID NO:44;

(j) a polynucleotide encoding a protein comprising a fragment of the amino acid sequence of SEQ ID NO:44 having biological activity, the fragment comprising eight consecutive amino acids of SEQ ID NO:44;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:43 from nucleotide 778 to nucleotide 1083; the nucleotide sequence of SEQ ID NO:43 from nucleotide 931 to nucleotide 1083; the nucleotide sequence of SEQ ID NO:43 from nucleotide 802 to nucleotide 1056; the nucleotide sequence of the full-length protein coding sequence of clone fn191_{—}3 deposited under accession number ATCC 98456; or the nucleotide sequence of a mature protein coding sequence of clone fn191_{—}3 deposited under accession number ATCC 98456. In other preferred embodiments, the polynucleotide encodes the full-length or a mature protein encoded by the cDNA insert of clone fn191_{—}3 deposited under accession number ATCC 98456. In yet other preferred embodiments, the present invention provides a polynucleotide encoding a protein comprising the amino acid sequence of SEQ ID NO:10 from amino acid 1 to amino acid 93. In further preferred embodiments, the present invention provides a polynucleotide encoding a protein comprising a fragment of the amino acid sequence of SEQ ID NO:44 having biological activity, the fragment preferably comprising eight (more preferably twenty, most preferably thirty) consecutive amino acids of SEQ ID NO:44, or a polynucleotide encoding a protein comprising a fragment of the amino acid sequence of SEQ ID NO:44 having biological activity, the fragment comprising the amino acid sequence from amino acid 46 to amino acid 55 of SEQ ID NO:44.

Other embodiments provide the gene corresponding to the cDNA sequence of SEQ ID NO:43.

In other embodiments, the present invention provides a composition comprising a protein, wherein said protein comprises an amino acid sequence selected from the group consisting of:

(a) the amino acid sequence of SEQ ID NO:43;

(b) the amino acid sequence of SEQ ID NO:43 from amino acid 1 to amino acid 93;

(c) fragments of the amino acid sequence of SEQ ID NO:43 comprising eight consecutive amino acids of SEQ ID NO:43; and

(d) the amino acid sequence encoded by the cDNA insert of clone fn191_{—}3 deposited under accession number ATCC 98456;

the protein being substantially free from other mammalian proteins. Preferably such protein comprises the amino acid sequence of SEQ ID NO:44 or the amino acid sequence of SEQ ID NO:44 from amino acid 1 to amino acid 93. In further preferred embodiments, the present invention provides a protein comprising a fragment of the amino acid sequence of SEQ ID NO:44 having biological activity, the fragment preferably comprising eight (more preferably twenty, most preferably thirty) consecutive amino acids of SEQ ID NO:44, or a protein comprising a fragment of the amino acid sequence of SEQ ID NO:44 having biological activity, the fragment comprising the amino acid sequence from amino acid 46 to amino acid 55 of SEQ ID NO:44.

In one embodiment, the present invention provides a composition comprising an isolated polynucleotide selected from the group consisting of:

(a) a polynucleotide comprising the nucleotide sequence of SEQ ID NO:45;

(b) a polynucleotide comprising the nucleotide sequence of SEQ ID NO:45 from nucleotide 390 to nucleotide 1355;

(c) a polynucleotide comprising the nucleotide sequence of SEQ ID NO:45 from nucleotide 1384 to nucleotide 1736;

(d) a polynucleotide comprising the nucleotide sequence of the full-length protein coding sequence of clone gm196_{—}4 deposited under accession number ATCC 98456;

(e) a polynucleotide encoding the full-length protein encoded by the cDNA insert of clone gm196_{—}4 deposited under accession number ATCC 98456;

(f) a polynucleotide comprising the nucleotide sequence of a mature protein coding sequence of clone gm196_{—}4 deposited under accession number ATCC 98456;

(g) a polynucleotide encoding a mature protein encoded by the cDNA insert of clone gm196_{—}4 deposited under accession number ATCC 98456;

(h) a polynucleotide encoding a protein comprising the amino acid sequence of SEQ ID NO:46;

(i) a polynucleotide encoding a protein comprising a fragment of the amino acid sequence of SEQ ID NO:46 having biological activity, the fragment comprising eight consecutive amino acids of SEQ ID NO:46;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:45 from nucleotide 390 to nucleotide 1355; the nucleotide sequence of SEQ ID NO:45 from nucleotide 1384 to nucleotide 1736; the nucleotide sequence of the full-length protein coding sequence of clone gm196_{—}4 deposited under accession number ATCC 98456; or the nucleotide sequence of a mature protein coding sequence of clone gm196_{—}4 deposited under accession number ATCC 98456. In other preferred embodiments, the polynucleotide encodes the full-length or a mature protein encoded by the cDNA insert of clone gm196_{—}4 deposited under accession number ATCC 98456. In further preferred embodiments, the present invention provides a polynucleotide encoding a protein comprising a fragment of the amino acid sequence of SEQ ID NO:46 having biological activity, the fragment preferably comprising eight (more preferably twenty, most preferably thirty) consecutive amino acids of SEQ ID NO:46, or a polynucleotide encoding a protein comprising a fragment of the amino acid sequence of SEQ ID NO:46 having biological activity, the fragment comprising the amino acid sequence from amino acid 156 to amino acid 165 of SEQ ID NO:46.

Other embodiments provide the gene corresponding to the cDNA sequence of SEQ ID NO:45.

In other embodiments, the present invention provides a composition comprising a protein, wherein said protein comprises an amino acid sequence selected from the group consisting of:

(a) the amino acid sequence of SEQ ID NO:46;

(b) fragments of the amino acid sequence of SEQ ID NO:46 comprising eight consecutive amino acids of SEQ ID NO:46; and

(c) the amino acid sequence encoded by the cDNA insert of clone gm196_{—}4 deposited under accession number ATCC 98456;

the protein being substantially free from other mammalian proteins. Preferably such protein comprises the amino acid sequence of SEQ ID NO:46. In further preferred embodiments, the present invention provides a protein comprising a fragment of the amino acid sequence of SEQ ID NO:46 having biological activity, the fragment preferably comprising eight (more preferably twenty, most preferably thirty) consecutive amino acids of SEQ ID NO:46, or a protein comprising a fragment of the amino acid sequence of SEQ ID NO:46 having biological activity, the fragment comprising the amino acid sequence from amino acid 156 to amino acid 165 of SEQ ID NO:46.

In one embodiment, the present invention provides a composition comprising an isolated polynucleotide selected from the group consisting of:

(a) a polynucleotide comprising the nucleotide sequence of SEQ ID NO:47;

(b) a polynucleotide comprising the nucleotide sequence of SEQ ID NO:47 from nucleotide 879 to nucleotide 1391;

(c) a polynucleotide comprising the nucleotide sequence of SEQ ID NO:47 from nucleotide 519 to nucleotide 1074;

(d) a polynucleotide comprising the nucleotide sequence of the full-length protein coding sequence of clone gn114_{—}1 deposited under accession number ATCC 98456;

(e) a polynucleotide encoding the full-length protein encoded by the cDNA insert of clone gn114_{—}1 deposited under accession number ATCC 98456;

(f) a polynucleotide comprising the nucleotide sequence of a mature protein coding sequence of clone gn114_{—}1 deposited under accession number ATCC 98456;

(g) a polynucleotide encoding a mature protein encoded by the cDNA insert of clone gn114_{—}1 deposited under accession number ATCC 98456;

(h) a polynucleotide encoding a protein comprising the amino acid sequence of SEQ ID NO:48;

(i) a polynucleotide encoding a protein comprising a fragment of the amino acid sequence of SEQ ID NO:48 having biological activity, the fragment comprising eight consecutive amino acids of SEQ ID NO:48;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:47 from nucleotide 879 to nucleotide 1391; the nucleotide sequence of SEQ ID NO:47 from nucleotide 519 to nucleotide 1074; the nucleotide sequence of the full-length protein coding sequence of clone gn114_{—}1 deposited under accession number ATCC 98456; or the nucleotide sequence of a mature protein coding sequence of clone gn114_{—}1 deposited under accession number ATCC 98456. In other preferred embodiments, the polynucleotide encodes the full-length or a mature protein encoded by the cDNA insert of clone gn114_{—}1 deposited under accession number ATCC 98456. In yet other preferred embodiments, the present invention provides a polynucleotide encoding a protein comprising the amino acid sequence of SEQ ID NO:48 from amino acid 1 to amino acid 65. In further preferred embodiments, the present invention provides a polynucleotide encoding a protein comprising a fragment of the amino acid sequence of SEQ ID NO:48 having biological activity, the fragment preferably comprising eight (more preferably twenty, most preferably thirty) consecutive amino acids of SEQ ID NO:48, or a polynucleotide encoding a protein comprising a fragment of the amino acid sequence of SEQ ID NO:48 having biological activity, the fragment comprising the amino acid sequence from amino acid 80 to amino acid 89 of SEQ ID NO:48.

Other embodiments provide the gene corresponding to the cDNA sequence of SEQ ID NO:47.

In other embodiments, the present invention provides a composition comprising a protein, wherein said protein comprises an amino acid sequence selected from the group consisting of:

(a) the amino acid sequence of SEQ ID NO:48;

(b) the amino acid sequence of SEQ ID NO:48 from amino acid 1 to amino acid 65;

(c) fragments of the amino acid sequence of SEQ ID NO:48 comprising eight consecutive amino acids of SEQ ID NO:48; and

(d) the amino acid sequence encoded by the cDNA insert of clone gn114_{—}1 deposited under accession number ATCC 98456;

the protein being substantially free from other mammalian proteins. Preferably such protein comprises the amino acid sequence of SEQ ID NO:48 or the amino acid sequence of SEQ ID NO:48 from amino acid 1 to amino acid 65. In further preferred embodiments, the present invention provides a protein comprising a fragment of the amino acid sequence of SEQ ID NO:48 having biological activity, the fragment preferably comprising eight (more preferably twenty, most preferably thirty) consecutive amino acids of SEQ ID NO:48, or a protein comprising a fragment of the amino acid sequence of SEQ ID NO:48 having biological activity, the fragment comprising the amino acid sequence from amino acid 80 to amino acid 89 of SEQ ID NO:48.

In one embodiment, the present invention provides a composition comprising an isolated polynucleotide selected from the group consisting of:

(a) a polynucleotide comprising the nucleotide sequence of SEQ ID NO:49;

(b) a polynucleotide comprising the nucleotide sequence of SEQ ID NO:49 from nucleotide 225 to nucleotide 1508;

(c) a polynucleotide comprising the nucleotide sequence of SEQ ID NO:49 from nucleotide 252 to nucleotide 1508;

(d) a polynucleotide comprising the nucleotide sequence of SEQ ID NO:49 from nucleotide 1 to nucleotide 302;

(e) a polynucleotide comprising the nucleotide sequence of the full-length protein coding sequence of clone hj968_{—}2 deposited under accession number ATCC 98456;

(f) a polynucleotide encoding the full-length protein encoded by the cDNA insert of clone hj968_{—}2 deposited under accession number ATCC 98456;

(g) a polynucleotide comprising the nucleotide sequence of a mature protein coding sequence of clone hj968_{—}2 deposited under accession number ATCC 98456;

(h) a polynucleotide encoding a mature protein encoded by the cDNA insert of clone hj968_{—}2 deposited under accession number ATCC 98456;

(i) a polynucleotide encoding a protein comprising the amino acid sequence of SEQ ID NO:50;

(j) a polynucleotide encoding a protein comprising a fragment of the amino acid sequence of SEQ ID NO:50 having biological activity, the fragment comprising eight consecutive amino acids of SEQ ID NO:50;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:49 from nucleotide 225 to nucleotide 1508; the nucleotide sequence of SEQ ID NO49 from nucleotide 252 to nucleotide 1508; the nucleotide sequence of SEQ ID NO:49 from nucleotide 1 to nucleotide 302; the nucleotide sequence of the full-length protein coding sequence of clone hj968_{—}2 deposited under accession number ATCC 98456; or the nucleotide sequence of a mature protein coding sequence of clone hj968_{—}2 deposited under accession number ATCC 98456. In other preferred embodiments, the polynucleotide encodes the full-length or a mature protein encoded by the cDNA insert of clone hj968_{—}2 deposited under accession number ATCC 98456. In yet other preferred embodiments, the present invention provides a polynucleotide encoding a protein comprising the amino acid sequence of SEQ ID NO:50 from amino acid 1 to amino acid 26. In further preferred embodiments, the present invention provides a polynucleotide encoding a protein comprising a fragment of the amino acid sequence of SEQ ID NO:50 having biological activity, the fragment preferably comprising eight (more preferably twenty, most preferably thirty) consecutive amino acids of SEQ ID NO:50, or a polynucleotide encoding a protein comprising a fragment of the amino acid sequence of SEQ ID NO:50 having biological activity, the fragment comprising the amino acid sequence from amino acid 209 to amino acid 218 of SEQ ID NO:50.

Other embodiments provide the gene corresponding to the cDNA sequence of SEQ ID NO:50.

In other embodiments, the present invention provides a composition comprising a protein, wherein said protein comprises an amino acid sequence selected from the group consisting of:

(a) the amino acid sequence of SEQ ID NO:50;

(b) the amino acid sequence of SEQ ID NO:50 from amino acid 1 to amino acid 26;

(c) fragments of the amino acid sequence of SEQ ID NO:50 comprising eight consecutive amino acids of SEQ ID NO:50; and

(d) the amino acid sequence encoded by the cDNA insert of clone hj968_{—}2 deposited under accession number ATCC 98456;

the protein being substantially free from other mammalian proteins. Preferably such protein comprises the amino acid sequence of SEQ ID NO:50 or the amino acid sequence of SEQ ID NO:50 from amino acid 1 to amino acid 26. In further preferred embodiments, the present invention provides a protein comprising a fragment of the amino acid sequence of SEQ ID NO:50 having biological activity, the fragment preferably comprising eight (more preferably twenty, most preferably thirty) consecutive amino acids of SEQ ID NO:50, or a protein comprising a fragment of the amino acid sequence of SEQ ID NO:50 having biological activity, the fragment comprising the amino acid sequence from amino acid 209 to amino acid 218 of SEQ ID NO:50.

In one embodiment, the present invention provides a composition comprising an isolated polynucleotide selected from the group consisting of:

(a) a polynucleotide comprising the nucleotide sequence of SEQ ID NO:51;

(b) a polynucleotide comprising the nucleotide sequence of SEQ ID NO:51 from nucleotide 1113 to nucleotide 1274;

(c) a polynucleotide comprising the nucleotide sequence of SEQ ID NO:51 from nucleotide 1233 to nucleotide 1274;

(d) a polynucleotide comprising the nucleotide sequence of SEQ ID NO:51 from nucleotide 894 to nucleotide 1309;

(e) a polynucleotide comprising the nucleotide sequence of the full-length protein coding sequence of clone hk10_{—}3 deposited under accession number ATCC 98456;

(f) a polynucleotide encoding the full-length protein encoded by the cDNA insert of clone hk10_{—}3 deposited under accession number ATCC 98456;

(g) a polynucleotide comprising the nucleotide sequence of a mature protein coding sequence of clone hk10_{—}3 deposited under accession number ATCC 98456;

(h) a polynucleotide encoding a mature protein encoded by the cDNA insert of clone hk10_{—}3 deposited under accession number ATCC 98456;

(i) a polynucleotide encoding a protein comprising the amino acid sequence of SEQ ID NO:52;

(j) a polynucleotide encoding a protein comprising a fragment of the amino acid sequence of SEQ ID NO:52 having biological activity, the fragment comprising eight consecutive amino acids of SEQ ID NO:52;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:51 from nucleotide 1113 to nucleotide 1274; the nucleotide sequence of SEQ ID NO:51 from nucleotide 1233 to nucleotide 1274; the nucleotide sequence of SEQ ID NO:51 from nucleotide 894 to nucleotide 1309; the nucleotide sequence of the full-length protein coding sequence of clone hk10_{—}3 deposited under accession number ATCC 98456; or the nucleotide sequence of a mature protein coding sequence of clone hk10_{—}3 deposited under accession number ATCC 98456. In other preferred embodiments, the polynucleotide encodes the full-length or a mature protein encoded by the cDNA insert of clone hk10_{—}3 deposited under accession number ATCC 98456. In further preferred embodiments, the present invention provides a polynucleotide encoding a protein comprising a fragment of the amino acid sequence of SEQ ID NO:52 having biological activity, the fragment preferably comprising eight (more preferably twenty, most preferably thirty) consecutive amino acids of SEQ ID NO:52, or a polynucleotide encoding a protein comprising a fragment of the amino acid sequence of SEQ ID NO:52 having biological activity, the fragment comprising the amino acid sequence from amino acid 22 to amino acid 31 of SEQ ID NO:52.

Other embodiments provide the gene corresponding to the cDNA sequence of SEQ ID NO:51.

In other embodiments, the present invention provides a composition comprising a protein, wherein said protein comprises an amino acid sequence selected from the group consisting of:

(a) the amino acid sequence of SEQ ID NO:52;

(b) fragments of the amino acid sequence of SEQ ID NO:52 comprising eight consecutive amino acids of SEQ ID NO:52; and

(c) the amino acid sequence encoded by the cDNA insert of clone hk10_{—}3 deposited under accession number ATCC 98456;

the protein being substantially free from other mammalian proteins. Preferably such protein comprises the amino acid sequence of SEQ ID NO:52. In further preferred embodiments, the present invention provides a protein comprising a fragment of the amino acid sequence of SEQ ID NO:52 having biological activity, the fragment preferably comprising eight (more preferably twenty, most preferably thirty) consecutive amino acids of SEQ ID NO:52, or a protein comprising a fragment of the amino acid sequence of SEQ ID NO:52 having biological activity, the fragment comprising the amino acid sequence from amino acid 22 to amino acid 31 of SEQ ID NO:52.

In one embodiment, the present invention provides a composition comprising an isolated polynucleotide selected from the group consisting of:

(a) a polynucleotide comprising the nucleotide sequence of SEQ ID NO:53;

(b) a polynucleotide comprising the nucleotide sequence of SEQ ID NO:53 from nucleotide 96 to nucleotide 1145;

(c) a polynucleotide comprising the nucleotide sequence of SEQ ID NO:53 from nucleotide 109 to nucleotide 539;

(d) a polynucleotide comprising the nucleotide sequence of the full-length protein coding sequence of clone hm236_{—}1 deposited under accession number ATCC 98456;

(e) a polynucleotide encoding the full-length protein encoded by the cDNA insert of clone hm236_{—}1 deposited under accession number ATCC 98456;

(f) a polynucleotide comprising the nucleotide sequence of a mature protein coding sequence of clone hm236_{—}1 deposited under accession number ATCC 98456;

(g) a polynucleotide encoding a mature protein encoded by the cDNA insert of clone hm236_{—}1 deposited under accession number ATCC 98456;

(h) a polynucleotide encoding a protein comprising the amino acid sequence of SEQ ID NO:54;

(i) a polynucleotide encoding a protein comprising a fragment of the amino acid sequence of SEQ ID NO:54 having biological activity, the fragment comprising eight consecutive amino acids of SEQ ID NO:54;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:54 from nucleotide 96 to nucleotide 1145; the nucleotide sequence of SEQ ID NO:54 from nucleotide 109 to nucleotide 539; the nucleotide sequence of the full-length protein coding sequence of clone hm236_{—}1 deposited under accession number ATCC 98456; or the nucleotide sequence of a mature protein coding sequence of clone hm236_{—}1 deposited under accession number ATCC 98456. In other preferred embodiments, the polynucleotide encodes the full-length or a mature protein encoded by the cDNA insert of clone hm236_{—}1 deposited under accession number ATCC 98456. In yet other preferred embodiments, the present invention provides a polynucleotide encoding a protein comprising the amino acid sequence of SEQ ID NO:54 from amino acid 6 to amino acid 148. In further preferred embodiments, the present invention provides a polynucleotide encoding a protein comprising a fragment of the amino acid sequence of SEQ ID NO:54 having biological activity, the fragment preferably comprising eight (more preferably twenty, most preferably thirty) consecutive amino acids of SEQ ID NO:54, or a polynucleotide encoding a protein comprising a fragment of the amino acid sequence of SEQ ID NO:54 having biological activity, the fragment comprising the amino acid sequence from amino acid 170 to amino acid 179 of SEQ ID NO:54.

Other embodiments provide the gene corresponding to the cDNA sequence of SEQ ID NO:53.

In other embodiments, the present invention provides a composition comprising a protein, wherein said protein comprises an amino acid sequence selected from the group consisting of:

(a) the amino acid sequence of SEQ ID NO:54;

(b) the amino acid sequence of SEQ ID NO:54 from amino acid 6 to amino acid 148;

(c) fragments of the amino acid sequence of SEQ ID NO:54 comprising eight consecutive amino acids of SEQ ID NO:54; and

(d) the amino acid sequence encoded by the cDNA insert of clone hm236_{—}1 deposited under accession number ATCC 98456;

the protein being substantially free from other mammalian proteins. Preferably such protein comprises the amino acid sequence of SEQ ID NO:54 or the amino acid sequence of SEQ ID NO:43 from amino acid 6 to amino acid 148. In further preferred embodiments, the present invention provides a protein comprising a fragment of the amino acid sequence of SEQ ID NO:54 having biological activity, the fragment preferably comprising eight (more preferably twenty, most preferably thirty) consecutive amino acids of SEQ ID NO:54, or a protein comprising a fragment of the amino acid sequence of SEQ ID NO:54 having biological activity, the fragment comprising the amino acid sequence from amino acid 170 to amino acid 179 of SEQ ID NO:54.

In one embodiment, the present invention provides a composition comprising an isolated polynucleotide selected from the group consisting of:

(a) a polynucleotide comprising the nucleotide sequence of SEQ ID NO:67;

(b) a polynucleotide comprising the nucleotide sequence of SEQ ID NO:67 from nucleotide 185 to nucleotide 1600

(c) a polynucleotide comprising the nucleotide sequence of SEQ ID NO:67 from nucleotide 1403 to nucleotide 1600;

(d) a polynucleotide comprising the nucleotide sequence of SEQ ID NO:67 from nucleotide 1 to nucleotide 850;

(e) a polynucleotide comprising the nucleotide sequence of the full-length protein coding sequence of clone do15_{—}4 deposited under accession number ATCC 98468;

(f) a polynucleotide encoding the full-length protein encoded by the cDNA insert of clone do15_{—}4 deposited under accession number ATCC 98468;

(g) a polynucleotide comprising the nucleotide sequence of a mature protein coding sequence of clone do15_{—}4 deposited under accession number ATCC 98468;

(h) a polynucleotide encoding a mature protein encoded by the cDNA insert of clone do15_{—}4 deposited under accession number ATCC 98468;

(i) a polynucleotide encoding a protein comprising the amino acid sequence of SEQ ID NO:68;

(j) a polynucleotide encoding a protein comprising a fragment of the amino acid sequence of SEQ ID NO:68 having biological activity, the fragment comprising eight consecutive amino acids of SEQ ID NO:68;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:67 from nucleotide 185 to nucleotide 1600; the nucleotide sequence of SEQ ID NO:67 from nucleotide 1403 to nucleotide 1600; the nucleotide sequence of SEQ ID NO:67 from nucleotide 1 to nucleotide 850; the nucleotide sequence of the full-length protein coding sequence of clone do15_{—}4 deposited under accession number ATCC 98468; or the nucleotide sequence of a mature protein coding sequence of clone do15_{—}4 deposited under accession number ATCC 98468. In other preferred embodiments, the polynucleotide encodes the full-length or a mature protein encoded by the cDNA insert of clone do15_{—}4 deposited under accession number ATCC 98468. In yet other preferred embodiments, the present invention provides a polynucleotide encoding a protein comprising the amino acid sequence of SEQ ID NO:2 from amino acid 1 to amino acid 222. In further preferred embodiments, the present invention provides a polynucleotide encoding a protein comprising a fragment of the amino acid sequence of SEQ ID NO:68 having biological activity, the fragment preferably comprising eight (more preferably twenty, most preferably thirty) consecutive amino acids of SEQ ID NO:68, or a polynucleotide encoding a protein comprising a fragment of the amino acid sequence of SEQ ID NO:68 having biological activity, the fragment comprising the amino acid sequence from amino acid 231 to amino acid 240 of SEQ ID NO:68.

Other embodiments provide the gene corresponding to the cDNA sequence of SEQ ID NO:67.

In other embodiments, the present invention provides a composition comprising a protein, wherein said protein comprises an amino acid sequence selected from the group consisting of:

(a) the amino acid sequence of SEQ ID NO:68;

(b) the amino acid sequence of SEQ ID NO:68 from amino acid 1 to amino acid 222;

(c) fragments of the amino acid sequence of SEQ ID NO:68 comprising eight consecutive amino acids of SEQ ID NO:68; and

(d) the amino acid sequence encoded by the cDNA insert of clone do15_{—}4 deposited under accession number ATCC 98468;

the protein being substantially free from other mammalian proteins. Preferably such protein comprises the amino acid sequence of SEQ ID NO:68 or the amino acid sequence of SEQ ID NO:68 from amino acid 1 to amino acid 222. In further preferred embodiments, the present invention provides a protein comprising a fragment of the amino acid sequence of SEQ ID NO:68 having biological activity, the fragment preferably comprising eight (more preferably twenty, most preferably thirty) consecutive amino acids of SEQ ID NO:68, or a protein comprising a fragment of the amino acid sequence of SEQ ID NO:68 having biological activity, the fragment comprising the amino acid sequence from amino acid 231 to amino acid 240 of SEQ ID NO:68.

In one embodiment, the present invention provides a composition comprising an isolated polynucleotide selected from the group consisting of:

(a) a polynucleotide comprising the nucleotide sequence of SEQ ID NO:69;

(b) a polynucleotide comprising the nucleotide sequence of SEQ ID NO:69 from nucleotide 47 to nucleotide 2065;

(c) a polynucleotide comprising the nucleotide sequence of SEQ ID NO:69 from nucleotide 1086 to nucleotide 1848;

(d) a polynucleotide comprising the nucleotide sequence of the full-length protein coding sequence of clone dx290_{—}1 deposited under accession number ATCC 98468;

(e) a polynucleotide encoding the full-length protein encoded by the cDNA insert of clone dx290_{—}1 deposited under accession number ATCC 98468;

(f) a polynucleotide comprising the nucleotide sequence of a mature protein coding sequence of clone dx290_{—}1 deposited under accession number ATCC 98468;

(g) a polynucleotide encoding a mature protein encoded by the cDNA insert of clone dx290_{—}1 deposited under accession number ATCC 98468;

(h) a polynucleotide encoding a protein comprising the amino acid sequence of SEQ ID NO:70;

(i) a polynucleotide encoding a protein comprising a fragment of the amino acid sequence of SEQ ID NO:70 having biological activity, the fragment comprising eight consecutive amino acids of SEQ ID NO:70;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:69 from nucleotide 47 to nucleotide 2065; the nucleotide sequence of SEQ ID NO:69 from nucleotide 1086 to nucleotide 1848; the nucleotide sequence of the full-length protein coding sequence of clone dx290_{—}1 deposited under accession number ATCC 98468; or the nucleotide sequence of a mature protein coding sequence of clone dx290_{—}1 deposited under accession number ATCC 98468. In other preferred embodiments, the polynucleotide encodes the full-length or a mature protein encoded by the cDNA insert of clone dx290_{—}1 deposited under accession number ATCC 98468. In yet other preferred embodiments, the present invention provides a polynucleotide encoding a protein comprising the amino acid sequence of SEQ ID NO:70 from amino acid 312 to amino acid 600. In further preferred embodiments, the present invention provides a polynucleotide encoding a protein comprising a fragment of the amino acid sequence of SEQ ID NO:70 having biological activity, the fragment preferably comprising eight (more preferably twenty, most preferably thirty) consecutive amino acids of SEQ ID NO:70, or a polynucleotide encoding a protein comprising a fragment of the amino acid sequence of SEQ ID NO:70 having biological activity, the fragment comprising the amino acid sequence from amino acid 331 to amino acid 340 of SEQ ID NO:70.

Other embodiments provide the gene corresponding to the cDNA sequence of SEQ ID NO:70.

In other embodiments, the present invention provides a composition comprising a protein, wherein said protein comprises an amino acid sequence selected from the group consisting of:

(a) the amino acid sequence of SEQ ID NO:70;

(b) the amino acid sequence of SEQ ID NO:70 from amino acid 312 to amino acid 600;

(c) fragments of the amino acid sequence of SEQ ID NO:70 comprising eight consecutive amino acids of SEQ ID NO:70; and

(d) the amino acid sequence encoded by the cDNA insert of clone dx290_{—}1 deposited under accession number ATCC 98468;

the protein being substantially free from other mammalian proteins. Preferably such protein comprises the amino acid sequence of SEQ ID NO:70 or the amino acid sequence of SEQ ID NO:4 from amino acid 312 to amino acid 600. In further preferred embodiments, the present invention provides a protein comprising a fragment of the amino acid sequence of SEQ ID NO:70 having biological activity, the fragment preferably comprising eight (more preferably twenty, most preferably thirty) consecutive amino acids of SEQ ID NO:70, or a protein comprising a fragment of the amino acid sequence of SEQ ID NO:70 having biological activity, the fragment comprising the amino acid sequence from amino acid 331 to amino acid 340 of SEQ ID NO:70.

In one embodiment, the present invention provides a composition comprising an isolated polynucleotide selected from the group consisting of:

(a) a polynucleotide comprising the nucleotide sequence of SEQ ID NO:71;

(b) a polynucleotide comprising the nucleotide sequence of SEQ ID NO:71 from nucleotide 107 to nucleotide 724;

(c) a polynucleotide comprising the nucleotide sequence of SEQ ID NO:71 from nucleotide 218 to nucleotide 724;

(d) a polynucleotide comprising the nucleotide sequence of SEQ ID NO:71 from nucleotide 536 to nucleotide 866;

(e) a polynucleotide comprising the nucleotide sequence of the full-length protein coding sequence of clone ek390_{—}4 deposited under accession number ATCC 98468;

(f) a polynucleotide encoding the full-length protein encoded by the cDNA insert of clone ek390_{—}4 deposited under accession number ATCC 98468;

(g) a polynucleotide comprising the nucleotide sequence of a mature protein coding sequence of clone ek390_{—}4 deposited under accession number ATCC 98468;

(h) a polynucleotide encoding a mature protein encoded by the cDNA insert of clone ek390_{—}4 deposited under accession number ATCC 98468;

(i) a polynucleotide encoding a protein comprising the amino acid sequence of SEQ ID NO:72;

(j) a polynucleotide encoding a protein comprising a fragment of the amino acid sequence of SEQ ID NO:72 having biological activity, the fragment comprising eight consecutive amino acids of SEQ ID NO:72;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:71 from nucleotide 107 to nucleotide 724; the nucleotide sequence of SEQ ID NO:71 from nucleotide 218 to nucleotide 724; the nucleotide sequence of SEQ ID NO:71 from nucleotide 536 to nucleotide 866; the nucleotide sequence of the full-length protein coding sequence of clone ek390_{—}4 deposited under accession number ATCC 98468; or the nucleotide sequence of a mature protein coding sequence of clone ek390_{—}4 deposited under accession number ATCC 98468. In other preferred embodiments, the polynucleotide encodes the full-length or a mature protein encoded by the cDNA insert of clone ek390_{—}4 deposited under accession number ATCC 98468. In yet other preferred embodiments, the present invention provides a polynucleotide encoding a protein comprising the amino acid sequence of SEQ ID NO:6 from amino acid 6 to amino acid 92. In further preferred embodiments, the present invention provides a polynucleotide encoding a protein comprising a fragment of the amino acid sequence of SEQ ID NO:72 having biological activity, the fragment preferably comprising eight (more preferably twenty, most preferably thirty) consecutive amino acids of SEQ ID NO:72, or a polynucleotide encoding a protein comprising a fragment of the amino acid sequence of SEQ ID NO:72 having biological activity, the fragment comprising the amino acid sequence from amino acid 97 to amino acid 106 of SEQ ID NO:72.

Other embodiments provide the gene corresponding to the cDNA sequence of SEQ ID NO:71.

In other embodiments, the present invention provides a composition comprising a protein, wherein said protein comprises an amino acid sequence selected from the group consisting of:

(a) the amino acid sequence of SEQ ID NO:72;

(b) the amino acid sequence of SEQ ID NO:6 from amino acid 6 to amino acid 92;

(c) fragments of the amino acid sequence of SEQ ID NO:72 comprising eight consecutive amino acids of SEQ ID NO:72; and

(d) the amino acid sequence encoded by the cDNA insert of clone ek390_{—}4 deposited under accession number ATCC 98468;

the protein being substantially free from other mammalian proteins. Preferably such protein comprises the amino acid sequence of SEQ ID NO:72 or the amino acid sequence of SEQ ID NO:72 from amino acid 6 to amino acid 92. In further preferred embodiments, the present invention provides a protein comprising a fragment of the amino acid sequence of SEQ ID NO:72 having biological activity, the fragment preferably comprising eight (more preferably twenty, most preferably thirty) consecutive amino acids of SEQ ID NO:72, or a protein comprising a fragment of the amino acid sequence of SEQ ID NO:72 having biological activity, the fragment comprising the amino acid sequence from amino acid 97 to amino acid 106 of SEQ ID NO:72.

In one embodiment, the present invention provides a composition comprising an isolated polynucleotide selected from the group consisting of:

(a) a polynucleotide comprising the nucleotide sequence of SEQ ID NO:73;

(b) a polynucleotide comprising the nucleotide sequence of SEQ ID NO:73 from nucleotide 31 to nucleotide 1230;

(c) a polynucleotide comprising the nucleotide sequence of SEQ ID NO:73from nucleotide 289 to nucleotide 1230;

(d) a polynucleotide comprising the nucleotide sequence of SEQ ID NO:73 from nucleotide 344 to nucleotide 1119;

(e) a polynucleotide comprising the nucleotide sequence of the full-length protein coding sequence of clone er471_{—}7 deposited under accession number ATCC 98468;

(f) a polynucleotide encoding the full-length protein encoded by the cDNA insert of clone er471_{—}7 deposited under accession number ATCC 98468;

(g) a polynucleotide comprising the nucleotide sequence of a mature protein coding sequence of clone er471_{—}7 deposited under accession number ATCC 98468;

(h) a polynucleotide encoding a mature protein encoded by the cDNA insert of clone er471_{—}7 deposited under accession number ATCC 98468;

(i) a polynucleotide encoding a protein comprising the amino acid sequence of SEQ ID NO:74;

(j) a polynucleotide encoding a protein comprising a fragment of the amino acid sequence of SEQ ID NO:74 having biological activity, the fragment comprising eight consecutive amino acids of SEQ ID NO:74;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:73 from nucleotide 31 to nucleotide 1230; the nucleotide sequence of SEQ ID NO:73from nucleotide 289 to nucleotide 1230; the nucleotide sequence of SEQ ID NO:73 from nucleotide 344 to nucleotide 1119; the nucleotide sequence of the full-length protein coding sequence of clone er471_{—}7 deposited under accession number ATCC 98468; or the nucleotide sequence of a mature protein coding sequence of clone er471_{—}7 deposited under accession number ATCC 98468. In other preferred embodiments, the polynucleotide encodes the full-length or a mature protein encoded by the cDNA insert of clone er471_{—}7 deposited under accession number ATCC 98468. In yet other preferred embodiments, the present invention provides a polynucleotide encoding a protein comprising the amino acid sequence of SEQ ID NO:74 from amino acid 111 to amino acid 363. In further preferred embodiments, the present invention provides a polynucleotide encoding a protein comprising a fragment of the amino acid sequence of SEQ ID NO:74 having biological activity, the fragment preferably comprising eight (more preferably twenty, most preferably thirty) consecutive amino acids of SEQ ID NO:74, or a polynucleotide encoding a protein comprising a fragment of the amino acid sequence of SEQ ID NO:74 having biological activity, the fragment comprising the amino acid sequence from amino acid 195 to amino acid 204 of SEQ ID NO:74.

Other embodiments provide the gene corresponding to the cDNA sequence of SEQ ID NO:73.

In other embodiments, the present invention provides a composition comprising a protein, wherein said protein comprises an amino acid sequence selected from the group consisting of:

(a) the amino acid sequence of SEQ ID NO51;

(b) the amino acid sequence of SEQ ID NO:74 from amino acid 111 to amino acid 363;

(c) fragments of the amino acid sequence of SEQ ID NO:8 comprising eight consecutive amino acids of SEQ ID NO:74; and

(d) the amino acid sequence encoded by the cDNA insert of clone er471_{—}7 deposited under accession number ATCC 98468;

the protein being substantially free from other mammalian proteins. Preferably such protein comprises the amino acid sequence of SEQ ID NO:74 or the amino acid sequence of SEQ ID NO:74 from amino acid 111 to amino acid 363. In further preferred embodiments, the present invention provides a protein comprising a fragment of the amino acid sequence of SEQ ID NO:74 having biological activity, the fragment preferably comprising eight (more preferably twenty, most preferably thirty) consecutive amino acids of SEQ ID NO:74, or a protein comprising a fragment of the amino acid sequence of SEQ ID NO:74 having biological activity, the fragment comprising the amino acid sequence from amino acid 195 to amino acid 204 of SEQ ID NO:74.

In one embodiment, the present invention provides a composition comprising an isolated polynucleotide selected from the group consisting of:

(a) a polynucleotide comprising the nucleotide sequence of SEQ ID NO:75;

(b) a polynucleotide comprising the nucleotide sequence of SEQ ID NO:75 from nucleotide 62 to nucleotide 322;

(c) a polynucleotide comprising the nucleotide sequence of SEQ ID NO:75 from nucleotide 571 to nucleotide 878;

(d) a polynucleotide comprising the nucleotide sequence of the full-length protein coding sequence of clone fs40_{—}3 deposited under accession number ATCC 98468;

(e) a polynucleotide encoding the full-length protein encoded by the cDNA insert of clone fs40_{—}3 deposited under accession number ATCC 98468;

(f) a polynucleotide comprising the nucleotide sequence of a mature protein coding sequence of clone fs40_{—}3 deposited under accession number ATCC 98468;

(g) a polynucleotide encoding a mature protein encoded by the cDNA insert of clone fs40_{—}3 deposited under accession number ATCC 98468;

(h) a polynucleotide encoding a protein comprising the amino acid sequence of SEQ ID NO:76;

(i) a polynucleotide encoding a protein comprising a fragment of the amino acid sequence of SEQ ID NO:76 having biological activity, the fragment comprising eight consecutive amino acids of SEQ ID NO:76;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:75 from nucleotide 62 to nucleotide 322; the nucleotide sequence of SEQ ID NO:75 from nucleotide 571 to nucleotide 878; the nucleotide sequence of the full-length protein coding sequence of clone fs40_{—}3 deposited under accession number ATCC 98468; or the nucleotide sequence of a mature protein coding sequence of clone fs40_{—}3 deposited under accession number ATCC 98468. In other preferred embodiments, the polynucleotide encodes the full-length or a mature protein encoded by the cDNA insert of clone fs40_{—}3 deposited under accession number ATCC 98468. In further preferred embodiments, the present invention provides a polynucleotide encoding a protein comprising a fragment of the amino acid sequence of SEQ ID NO:76 having biological activity, the fragment preferably comprising eight (more preferably twenty, most preferably thirty) consecutive amino acids of SEQ ID NO:76, or a polynucleotide encoding a protein comprising a fragment of the amino acid sequence of SEQ ID NO:76 having biological activity, the fragment comprising the amino acid sequence from amino acid 38 to amino acid 47 of SEQ ID NO:76.

Other embodiments provide the gene corresponding to the cDNA sequence of SEQ ID NO:75.

In other embodiments, the present invention provides a composition comprising a protein, wherein said protein comprises an amino acid sequence selected from the group consisting of:

(a) the amino acid sequence of SEQ ID NO:76;

(b) fragments of the amino acid sequence of SEQ ID NO:76 comprising eight consecutive amino acids of SEQ ID NO:76; and

(c) the amino acid sequence encoded by the cDNA insert of clone fs40_{—}3 deposited under accession number ATCC 98468;

the protein being substantially free from other mammalian proteins. Preferably such protein comprises the amino acid sequence of SEQ ID NO:76. In further preferred embodiments, the present invention provides a protein comprising a fragment of the amino acid sequence of SEQ ID NO:76 having biological activity, the fragment preferably comprising eight (more preferably twenty, most preferably thirty) consecutive amino acids of SEQ ID NO:76, or a protein comprising a fragment of the amino acid sequence of SEQ ID NO:76 having biological activity, the fragment comprising the amino acid sequence from amino acid 38 to amino acid 47 of SEQ ID NO:76.

In one embodiment, the present invention provides a composition comprising an isolated polynucleotide selected from the group consisting of:

(a) a polynucleotide comprising the nucleotide sequence of SEQ ID NO:77;

(b) a polynucleotide comprising the nucleotide sequence of SEQ ID NO:77 from nucleotide 43 to nucleotide 1671;

(c) a polynucleotide comprising the nucleotide sequence of SEQ ID NO:77 from nucleotide 112 to nucleotide 1671;

(d) a polynucleotide comprising the nucleotide sequence of SEQ ID NO:77 from nucleotide 224 to nucleotide 679;

(e) a polynucleotide comprising the nucleotide sequence of the full-length protein coding sequence of clone ga63_{—}6 deposited under accession number ATCC 98468;

(f) a polynucleotide encoding the full-length protein encoded by the cDNA insert of clone ga63_{—}6 deposited under accession number ATCC 98468;

(g) a polynucleotide comprising the nucleotide sequence of a mature protein coding sequence of clone ga63_{—}6 deposited under accession number ATCC 98468;

(h) a polynucleotide encoding a mature protein encoded by the cDNA insert 3′ 5 of clone ga63_{—}6 deposited under accession number ATCC 98468;

(i) a polynucleotide encoding a protein comprising the amino acid sequence of SEQ ID NO:78;

(j) a polynucleotide encoding a protein comprising a fragment of the amino acid sequence of SEQ ID NO:78 having biological activity, the fragment comprising eight consecutive amino acids of SEQ ID NO:78;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:77 from nucleotide 43 to nucleotide 1671; the nucleotide sequence of SEQ ID NO:77 from nucleotide 112 to nucleotide 1671; the nucleotide sequence of SEQ ID NO:77 from nucleotide 224 to nucleotide 679; the nucleotide sequence of the full-length protein coding sequence of clone ga63_{—}6 deposited under accession number ATCC 98468; or the nucleotide sequence of a mature protein coding sequence of clone ga63_{—}6 deposited under accession number ATCC 98468. In other preferred embodiments, the polynucleotide encodes the full-length or a mature protein encoded by the cDNA insert of clone ga63_{—}6 deposited under accession number ATCC 98468. In yet other preferred embodiments, the present invention provides a polynucleotide encoding a protein comprising the amino acid sequence of SEQ ID NO:78 from amino acid 62 to amino acid 212. In further preferred embodiments, the present invention provides a polynucleotide encoding a protein comprising a fragment of the amino acid sequence of SEQ ID NO:78 having biological activity, the fragment preferably comprising eight (more preferably twenty, most preferably thirty) consecutive amino acids of SEQ ID NO:78, or a polynucleotide encoding a protein comprising a fragment of the amino acid sequence of SEQ ID NO:78 having biological activity, the fragment comprising the amino acid sequence from amino acid 266 to amino acid 275 of SEQ ID NO:78.

Other embodiments provide the gene corresponding to the cDNA sequence of SEQ ID NO:77.

In other embodiments, the present invention provides a composition comprising a protein, wherein said protein comprises an amino acid sequence selected from the group consisting of:

(a) the amino acid sequence of SEQ ID NO:78;

(b) the amino acid sequence of SEQ ID NO:78 from amino acid 62 to amino acid 212;

(c) fragments of the amino acid sequence of SEQ ID NO:78 comprising eight consecutive amino acids of SEQ ID NO:12; and

(d) the amino acid sequence encoded by the cDNA insert of clone ga63_{—}6 deposited under accession number ATCC 98468;

the protein being substantially free from other mammalian proteins. Preferably such protein comprises the amino acid sequence of SEQ ID NO:78 or the amino acid sequence of SEQ ID NO:78 from amino acid 62 to amino acid 212. In further preferred embodiments, the present invention provides a protein comprising a fragment of the amino acid sequence of SEQ ID NO:78 having biological activity, the fragment preferably comprising eight (more preferably twenty, most preferably thirty) consecutive amino acids of SEQ ID NO:78, or a protein comprising a fragment of the amino acid sequence of SEQ ID NO:78 having biological activity, the fragment comprising the amino acid sequence from amino acid 266 to amino acid 275 of SEQ ID NO:78.

In one embodiment, the present invention provides a composition comprising an isolated polynucleotide selected from the group consisting of:

(a) a polynucleotide comprising the nucleotide sequence of SEQ ID NO:79;

(b) a polynucleotide comprising the nucleotide sequence of SEQ ID NO:79 from nucleotide 17 to nucleotide 523;

(c) a polynucleotide comprising the nucleotide sequence of SEQ ID NO:79 from nucleotide 77 to nucleotide 523;

(d) a polynucleotide comprising the nucleotide sequence of SEQ ID NO:79 from nucleotide 1 to nucleotide 392;

(e) a polynucleotide comprising the nucleotide sequence of the full-length protein coding sequence of clone gm335_{—}4 deposited under accession number ATCC 98468;

(f) a polynucleotide encoding the full-length protein encoded by the cDNA insert of clone gm335_{—}4 deposited under accession number ATCC 98468;

(g) a polynucleotide comprising the nucleotide sequence of a mature protein coding sequence of clone gm335_{—}4 deposited under accession number ATCC 98468;

(h) a polynucleotide encoding a mature protein encoded by the cDNA insert of clone gm335_{—}4 deposited under accession number ATCC 98468;

(i) a polynucleotide encoding a protein comprising the amino acid sequence of SEQ ID NO:80;

(j) a polynucleotide encoding a protein comprising a fragment of the amino acid sequence of SEQ ID NO:80 having biological activity, the fragment comprising eight consecutive amino acids of SEQ ID NO:80;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:79 from nucleotide 17 to nucleotide 523; the nucleotide sequence of SEQ ID NO:79 from nucleotide 77 to nucleotide 523; the nucleotide sequence of SEQ ID NO:79 from nucleotide 1 to nucleotide 392; the nucleotide sequence of the full-length protein coding sequence of clone gm335_{—}4 deposited under accession number ATCC 98468; or the nucleotide sequence of a mature protein coding sequence of clone gm335_{—}4 deposited under accession number ATCC 98468. In other preferred embodiments, the polynucleotide encodes the full-length or a mature protein encoded by the cDNA insert of clone gm335_{—}4 deposited under accession number ATCC 98468. In yet other preferred embodiments, the present invention provides a polynucleotide encoding a protein comprising the amino acid sequence of SEQ ID NO:80 from amino acid 1 to amino acid 125. In further preferred embodiments, the present invention provides a polynucleotide encoding a protein comprising a fragment of the amino acid sequence of SEQ ID NO:80 having biological activity, the fragment preferably comprising eight (more preferably twenty, most preferably thirty) consecutive amino acids of SEQ ID NO:80, or a polynucleotide encoding a protein comprising a fragment of the amino acid sequence of SEQ ID NO:80 having biological activity, the fragment comprising the amino acid sequence from amino acid 79 to amino acid 88 of SEQ ID NO:80.

Other embodiments provide the gene corresponding to the cDNA sequence of SEQ ID NO:79.

In other embodiments, the present invention provides a composition comprising a protein, wherein said protein comprises an amino acid sequence selected from the group consisting of:

(a) the amino acid sequence of SEQ ID NO:80;

(b) the amino acid sequence of SEQ ID NO:80 from amino acid 1 to amino acid 125;

(c) fragments of the amino acid sequence of SEQ ID NO:80 comprising eight consecutive amino acids of SEQ ID NO:80; and

(d) the amino acid sequence encoded by the cDNA insert of clone gm335_{—}4 deposited under accession number ATCC 98468;

the protein being substantially free from other mammalian proteins. Preferably such protein comprises the amino acid sequence of SEQ ID NO:80 or the amino acid sequence of SEQ ID NO:80 from amino acid 1 to amino acid 125. In further preferred embodiments, the present invention provides a protein comprising a fragment of the amino acid sequence of SEQ ID NO:80 having biological activity, the fragment preferably comprising eight (more preferably twenty, most preferably thirty) consecutive amino acids of SEQ ID NO:80, or a protein comprising a fragment of the amino acid sequence of SEQ ID NO:80 having biological activity, the fragment comprising the amino acid sequence from amino acid 79 to amino acid 88 of SEQ ID NO:80.

In one embodiment, the present invention provides a composition comprising an isolated polynucleotide selected from the group consisting of:

(a) a polynucleotide comprising the nucleotide sequence of SEQ ID NO:81;

(b) a polynucleotide comprising the nucleotide sequence of SEQ ID NO:81 from nucleotide 2 to nucleotide 991;

(c) a polynucleotide comprising the nucleotide sequence of SEQ ID NO:81 from nucleotide 62 to nucleotide 991;

(d) a polynucleotide comprising the nucleotide sequence of SEQ ID NO:81from nucleotide 2 to nucleotide 504;

(e) a polynucleotide comprising the nucleotide sequence of the full-length protein coding sequence of clone hy370_{—}9 deposited under accession number ATCC 98468;

(f) a polynucleotide encoding the full-length protein encoded by the cDNA insert of clone hy370_{—}9 deposited under accession number ATCC 98468;

(g) a polynucleotide comprising the nucleotide sequence of a mature protein coding sequence of clone hy370_{—}9 deposited under accession number ATCC 98468;

(h) a polynucleotide encoding a mature protein encoded by the cDNA insert of clone hy370_{—}9 deposited under accession number ATCC 98468;

(i) a polynucleotide encoding a protein comprising the amino acid sequence of SEQ ID NO:82;

(j) a polynucleotide encoding a protein comprising a fragment of the amino acid sequence of SEQ ID NO:82 having biological activity, the fragment comprising eight consecutive amino acids of SEQ ID NO:82;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:81 from nucleotide 2 to nucleotide 991; the nucleotide sequence of SEQ ID NO:81 from nucleotide 62 to nucleotide 991; the nucleotide sequence of SEQ ID NO:81 from nucleotide 2 to nucleotide 504; the nucleotide sequence of the full-length protein coding sequence of clone hy370_{—}9 deposited under accession number ATCC 98468; or the nucleotide sequence of a mature protein coding sequence of clone hy370_{—}9 deposited under accession number ATCC 98468. In other preferred embodiments, the polynucleotide encodes the full-length or a mature protein encoded by the cDNA insert of clone hy370_{—}9 deposited under accession number ATCC 98468. In yet other preferred embodiments, the present invention provides a polynucleotide encoding a protein comprising the amino acid sequence of SEQ ID NO:82 from amino acid 1 to amino acid 167. In further preferred embodiments, the present invention provides a polynucleotide encoding a protein comprising a fragment of the amino acid sequence of SEQ ID NO:82 having biological activity, the fragment preferably comprising eight (more preferably twenty, most preferably thirty) consecutive amino acids of SEQ ID NO:82, or a polynucleotide encoding a protein comprising a fragment of the amino acid sequence of SEQ ID NO:82 having biological activity, the fragment comprising the amino acid sequence from amino acid 160 to amino acid 169 of SEQ ID NO:82.

Other embodiments provide the gene corresponding to the cDNA sequence of SEQ ID NO:81.

In other embodiments, the present invention provides a composition comprising a protein, wherein said protein comprises an amino acid sequence selected from the group consisting of:

(a) the amino acid sequence of SEQ ID NO:82;

(b) the amino acid sequence of SEQ ID NO:82 from amino acid 1 to amino acid 167;

(c) fragments of the amino acid sequence of SEQ ID NO:82 comprising eight consecutive amino acids of SEQ ID NO:82; and

(d) the amino acid sequence encoded by the cDNA insert of clone hy370_{—}9 deposited under accession number ATCC 98468;

the protein being substantially free from other mammalian proteins. Preferably such protein comprises the amino acid sequence of SEQ ID NO:82 or the amino acid sequence of SEQ ID NO:82 from amino acid 1 to amino acid 167. In further preferred embodiments, the present invention provides a protein comprising a fragment of the amino acid sequence of SEQ ID NO:82 having biological activity, the fragment preferably comprising eight (more preferably twenty, most preferably thirty) consecutive amino acids of SEQ ID NO:82, or a protein comprising a fragment of the amino acid sequence of SEQ ID NO:82 having biological activity, the fragment comprising the amino acid sequence from amino acid 160 to amino acid 169 of SEQ ID NO:82.

In one embodiment, the present invention provides a composition comprising an isolated polynucleotide selected from the group consisting of:

(a) a polynucleotide comprising the nucleotide sequence of SEQ ID NO:83;

(b) a polynucleotide comprising the nucleotide sequence of SEQ ID NO:83from nucleotide 77 to nucleotide 616;

(c) a polynucleotide comprising the nucleotide sequence of SEQ ID NO:83 from nucleotide 164 to nucleotide 616;

(d) a polynucleotide comprising the nucleotide sequence of SEQ ID NO:83 from nucleotide 1 to nucleotide 415;

(e) a polynucleotide comprising the nucleotide sequence of the full-length protein coding sequence of clone ie47_{—}4 deposited under accession number ATCC 98468;

(f) a polynucleotide encoding the full-length protein encoded by the cDNA insert of clone ie47_{—}4 deposited under accession number ATCC 98468;

(g) a polynucleotide comprising the nucleotide sequence of a mature protein coding sequence of clone ie47_{—}4 deposited under accession number ATCC 98468;

(h) a polynucleotide encoding a mature protein encoded by the cDNA insert of clone ie47_{—}4 deposited under accession number ATCC 98468;

(i) a polynucleotide encoding a protein comprising the amino acid sequence of SEQ ID NO:84;

(j) a polynucleotide encoding a protein comprising a fragment of the amino acid sequence of SEQ ID NO:84 having biological activity, the fragment comprising eight consecutive amino acids of SEQ ID NO:84;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:83 from nucleotide 77 to nucleotide 616; the nucleotide sequence of SEQ ID NO:83 from nucleotide 164 to nucleotide 616; the nucleotide sequence of SEQ ID NO:83 from nucleotide 1 to nucleotide 415; the nucleotide sequence of the full-length protein coding sequence of clone ie47_{—}4 deposited under accession number ATCC 98468; or the nucleotide sequence of a mature protein coding sequence of clone ie47_{—}4 deposited under accession number ATCC 98468. In other preferred embodiments, the polynucleotide encodes the full-length or a mature protein encoded by the cDNA insert of clone ie47_{—}4 deposited under accession number ATCC 98468. In yet other preferred embodiments, the present invention provides a polynucleotide encoding a protein comprising the amino acid sequence of SEQ ID NO:84 from amino acid 1 to amino acid 113. In further preferred embodiments, the present invention provides a polynucleotide encoding a protein comprising a fragment of the amino acid sequence of SEQ ID NO:84 having biological activity, the fragment preferably comprising eight (more preferably twenty, most preferably thirty) consecutive amino acids of SEQ ID NO:84, or a polynucleotide encoding a protein comprising a fragment of the amino acid sequence of SEQ ID NO:84 having biological activity, the fragment comprising the amino acid sequence from amino acid 85 to amino acid 94 of SEQ ID NO:84.

Other embodiments provide the gene corresponding to the cDNA sequence of SEQ ID NO:83.

In other embodiments, the present invention provides a composition comprising a protein, wherein said protein comprises an amino acid sequence selected from the group consisting of:

(a) the amino acid sequence of SEQ ID NO:84;

(b) the amino acid sequence of SEQ ID NO:84 from amino acid 1 to amino acid 113;

(c) fragments of the amino acid sequence of SEQ ID NO:84 comprising eight consecutive amino acids of SEQ ID NO:84; and

(d) the amino acid sequence encoded by the cDNA insert of clone ie47_{—}4 deposited under accession number ATCC 98468;

the protein being substantially free from other mammalian proteins. Preferably such protein comprises the amino acid sequence of SEQ ID NO:84 or the amino acid sequence of SEQ ID NO:84 from amino acid 1 to amino acid 113. In further preferred embodiments, the present invention provides a protein comprising a fragment of the amino acid sequence of SEQ ID NO:84 having biological activity, the fragment preferably comprising eight (more preferably twenty, most preferably thirty) consecutive amino acids of SEQ ID NO:84, or a protein comprising a fragment of the amino acid sequence of SEQ ID NO:84 having biological activity, the fragment comprising the amino acid sequence from amino acid 85 to amino acid 94 of SEQ ID NO:84.

In one embodiment, the present invention provides a composition comprising an isolated polynucleotide selected from the group consisting of:

(a) a polynucleotide comprising the nucleotide sequence of SEQ ID NO:85;

(b) a polynucleotide comprising the nucleotide sequence of SEQ ID NO:85 from nucleotide 564 to nucleotide 2813;

(c) a polynucleotide comprising the nucleotide sequence of SEQ ID NO:85 from nucleotide 705 to nucleotide 2813;

(d) a polynucleotide comprising the nucleotide sequence of SEQ ID NO:85 from nucleotide 793 to nucleotide 1628;

(e) a polynucleotide comprising the nucleotide sequence of the full-length protein coding sequence of clone s195_{—}10 deposited under accession number ATCC 98468;

(f) a polynucleotide encoding the full-length protein encoded by the cDNA insert of clone s195_{—}10 deposited under accession number ATCC 98468;

(g) a polynucleotide comprising the nucleotide sequence of a mature protein coding sequence of clone s195_{—}10 deposited under accession number ATCC 98468;

(h) a polynucleotide encoding a mature protein encoded by the cDNA insert of clone s195_{—}10 deposited under accession number ATCC 98468;

(i) a polynucleotide encoding a protein comprising the amino acid sequence of SEQ ID NO:86;

(j) a polynucleotide encoding a protein comprising a fragment of the amino acid sequence of SEQ ID NO:86 having biological activity, the fragment comprising eight consecutive amino acids of SEQ ID NO:86;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:85 from nucleotide 564 to nucleotide 2813; the nucleotide sequence of SEQ ID NO:85 from nucleotide 705 to nucleotide 2813; the nucleotide sequence of SEQ ID NO:85 from nucleotide 793 to nucleotide 1628; the nucleotide sequence of the full-length protein coding sequence of clone s195_{—}10 deposited under accession number ATCC 98468; or the nucleotide sequence of a mature protein coding sequence of clone s195_{—}10 deposited under accession number ATCC 98468. In other preferred embodiments, the polynucleotide encodes the full-length or a mature protein encoded by the cDNA insert of clone s195_{—}10 deposited under accession number ATCC 98468. In yet other preferred embodiments, the present invention provides a polynucleotide encoding a protein comprising the amino acid sequence of SEQ ID NO:86 from amino acid 78 to amino acid 355. In further preferred embodiments, the present invention provides a polynucleotide encoding a protein comprising a fragment of the amino acid sequence of SEQ ID NO:86 having biological activity, the fragment preferably comprising eight (more preferably twenty, most preferably thirty) consecutive amino acids of SEQ ID NO:86, or a polynucleotide encoding a protein comprising a fragment of the amino acid sequence of SEQ ID NO:86 having biological activity, the fragment comprising the amino acid sequence from amino acid 370 to amino acid 379 of SEQ ID NO:86.

Other embodiments provide the gene corresponding to the cDNA sequence of SEQ ID NO:85.

In other embodiments, the present invention provides a composition comprising a protein, wherein said protein comprises an amino acid sequence selected from the group consisting of:

(a) the amino acid sequence of SEQ ID NO:86;

(b) the amino acid sequence of SEQ ID NO:86 from amino acid 78 to amino acid 355;

(c) fragments of the amino acid sequence of SEQ ID NO:86 comprising eight consecutive amino acids of SEQ ID NO:86; and

(d) the amino acid sequence encoded by the cDNA insert of clone s195_{—}10 deposited under accession number ATCC 98468;

the protein being substantially free from other mammalian proteins. Preferably such protein comprises the amino acid sequence of SEQ ID NO:86 or the amino acid sequence of SEQ ID NO:86 from amino acid 78 to amino acid 355. In further preferred embodiments, the present invention provides a protein comprising a fragment of the amino acid sequence of SEQ ID NO:86 having biological activity, the fragment preferably comprising eight (more preferably twenty, most preferably thirty) consecutive amino acids of SEQ ID NO:86, or a protein comprising a fragment of the amino acid sequence of SEQ ID NO:86 having biological activity, the fragment comprising the amino acid sequence from amino acid 370 to amino acid 379 of SEQ ID NO:86.

In one embodiment, the present invention provides a composition comprising an isolated polynucleotide selected from the group consisting of:

(a) a polynucleotide comprising the nucleotide sequence of SEQ ID NO:97;

(b) a polynucleotide comprising the nucleotide sequence of SEQ ID NO:97 from nucleotide 516 to nucleotide 797;

(c) a polynucleotide comprising the nucleotide sequence of SEQ ID NO:97 from nucleotide 606 to nucleotide 797;

(d) a polynucleotide comprising the nucleotide sequence of SEQ ID NO:97 from nucleotide 1 to nucleotide 773;

(e) a polynucleotide comprising the nucleotide sequence of the full-length protein coding sequence of clone bf228_{—}14 deposited under accession number ATCC 98482;

(f) a polynucleotide encoding the full-length protein encoded by the cDNA insert of clone bf228_{—}14 deposited under accession number ATCC 98482;

(g) a polynucleotide comprising the nucleotide sequence of the mature protein coding sequence of clone bf228_{—}14 deposited under accession number ATCC 98482;

(h) a polynucleotide encoding the mature protein encoded by the cDNA insert of clone bf228_{—}14 deposited under accession number ATCC 98482;

(i) a polynucleotide encoding a protein comprising the amino acid sequence of SEQ ID NO:98;

(j) a polynucleotide encoding a protein comprising a fragment of the amino acid sequence of SEQ ID NO:98 having biological activity;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:97 from nucleotide 516 to nucleotide 797; the nucleotide sequence of SEQ ID NO:97 from nucleotide 606 to nucleotide 797; the nucleotide sequence of SEQ ID NO:97 from nucleotide 1 to nucleotide 773; the nucleotide sequence of the full-length protein coding sequence of clone bf228_{—}14 deposited under accession number ATCC 98482; or the nucleotide sequence of the mature protein coding sequence of clone bf228_{—}14 deposited under accession number ATCC 98482. In other preferred embodiments, the polynucleotide encodes the full-length or mature protein encoded by the cDNA insert of clone bf228_{—}14 deposited under accession number ATCC 98482. In yet other preferred embodiments, the present invention provides a polynucleotide encoding a protein comprising the amino acid sequence of SEQ ID NO:98 from amino acid 1 to amino acid 86.

Other embodiments provide the gene corresponding to the cDNA sequence of SEQ ID NO:97.

In other embodiments, the present invention provides a composition comprising a protein, wherein said protein comprises an amino acid sequence selected from the group consisting of:

(a) the amino acid sequence of SEQ ID NO:98;

(b) the amino acid sequence of SEQ ID NO:98 from amino acid 1 to amino acid 86;

(c) fragments of the amino acid sequence of SEQ ID NO:98; and

(d) the amino acid sequence encoded by the cDNA insert of clone bf228_{—}14 deposited under accession number ATCC 98482;

the protein being substantially free from other mammalian proteins. Preferably such protein comprises the amino acid sequence of SEQ ID NO:98 or the amino acid sequence of SEQ ID NO:98 from amino acid 1 to amino acid 86.

In one embodiment, the present invention provides a composition comprising an isolated polynucleotide selected from the group consisting of:

(a) a polynucleotide comprising the nucleotide sequence of SEQ ID NO:99;

(b) a polynucleotide comprising the nucleotide sequence of SEQ ID NO:99 from nucleotide 137 to nucleotide 1240;

(c) a polynucleotide comprising the nucleotide sequence of SEQ ID NO:99 from nucleotide 1 to nucleotide 1153;

(d) a polynucleotide comprising the nucleotide sequence of the full-length protein coding sequence of clone bg249_{—}1 deposited under accession number ATCC 1_{—}5 98482;

(e) a polynucleotide encoding the full-length protein encoded by the cDNA insert of clone bg249_{—}1 deposited under accession number ATCC 98482;

(f) a polynucleotide comprising the nucleotide sequence of the mature protein coding sequence of clone bg249_{—}1 deposited under accession number ATCC 98482;

(g) a polynucleotide encoding the mature protein encoded by the cDNA insert of clone bg249_{—}1 deposited under accession number ATCC 98482;

(h) a polynucleotide encoding a protein comprising the amino acid sequence of SEQ ID NO:100;

(i) a polynucleotide encoding a protein comprising a fragment of the amino acid sequence of SEQ ID NO:100 having biological activity;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:99 from nucleotide 137 to nucleotide 1240; the nucleotide sequence of SEQ ID NO:99 from nucleotide 1 to nucleotide 1153; the nucleotide sequence of the full-length protein coding sequence of clone bg249_{—}1 deposited under accession number ATCC 98482; or the nucleotide sequence of the mature protein coding sequence of clone bg249_{—}1 deposited under accession number ATCC 98482. In other preferred embodiments, the polynucleotide encodes the full-length or mature protein encoded by the cDNA insert of clone bg249_{—}1 deposited under accession number ATCC 98482. In yet other preferred embodiments, the present invention provides a polynucleotide encoding a protein comprising the amino acid sequence of SEQ ID NO:100 from amino acid 1 to amino acid 339.

Other embodiments provide the gene corresponding to the cDNA sequence of SEQ ID NO:99.

In other embodiments, the present invention provides a composition comprising a protein, wherein said protein comprises an amino acid sequence selected from the group consisting of:

(a) the amino acid sequence of SEQ ID NO:100;

(b) the amino acid sequence of SEQ ID NO:100 from amino acid 1 to amino acid 339;

(c) fragments of the amino acid sequence of SEQ ID NO:100; and

(d) the amino acid sequence encoded by the cDNA insert of clone bg249_{—}1 deposited under accession number ATCC 98482;

the protein being substantially free from other mammalian proteins. Preferably such protein comprises the amino acid sequence of SEQ ID NO:100 or the amino acid sequence of SEQ ID NO:100 from amino acid 1 to amino acid 339.

In one embodiment, the present invention provides a composition comprising an isolated polynucleotide selected from the group consisting of:

(a) a polynucleotide comprising the nucleotide sequence of SEQ ID NO:101;

(b) a polynucleotide comprising the nucleotide sequence of SEQ ID NO:101 from nucleotide 26 to nucleotide 301;

(c) a polynucleotide comprising the nucleotide sequence of SEQ ID NO:101 from nucleotide 104 to nucleotide 301;

(d) a polynucleotide comprising the nucleotide sequence of SEQ ID NO:5 from nucleotide 1 to nucleotide 119;

(e) a polynucleotide comprising the nucleotide sequence of the full-length protein coding sequence of clone bv286_{—}1 deposited under accession number ATCC 98482;

(f) a polynucleotide encoding the full-length protein encoded by the cDNA insert of clone bv286_{—}1 deposited under accession number ATCC 98482;

(g) a polynucleotide comprising the nucleotide sequence of the mature protein coding sequence of clone bv286_{—}1 deposited under accession number ATCC 98482;

(h) a polynucleotide encoding the mature protein encoded by the cDNA insert of clone bv286_{—}1 deposited under accession number ATCC 98482;

(i) a polynucleotide encoding a protein comprising the amino acid sequence of SEQ ID NO:102;

(j) a polynucleotide encoding a protein comprising a fragment of the amino acid sequence of SEQ ID NO:102 having biological activity;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:101 from nucleotide 26 to nucleotide 301; the nucleotide sequence of SEQ ID NO:101 from nucleotide 104 to nucleotide 301; the nucleotide sequence of SEQ ID NO:101 from nucleotide 1 to nucleotide 119; the nucleotide sequence of the full-length protein coding sequence of clone bv286_{—}1 deposited under accession number ATCC 98482; or the nucleotide sequence of the mature protein coding sequence of clone bv286_{—}1 deposited under accession number ATCC 98482. In other preferred embodiments, the polynucleotide encodes the full-length or mature protein encoded by the cDNA insert of clone bv286_{—}1 deposited under accession number ATCC 98482. In yet other preferred embodiments, the present invention provides a polynucleotide encoding a protein comprising the amino acid sequence of SEQ ID NO:102 from amino acid 1 to amino acid 31.

Other embodiments provide the gene corresponding to the cDNA sequence of SEQ ID NO:101.

In other embodiments, the present invention provides a composition comprising a protein, wherein said protein comprises an amino acid sequence selected from the group consisting of:

(a) the amino acid sequence of SEQ ID NO:102;

(b) the amino acid sequence of SEQ ID NO:102 from amino acid 1 to amino acid 31;

(c) fragments of the amino acid sequence of SEQ ID NO:102; and

(d) the amino acid sequence encoded by the cDNA insert of clone bv286_{—}1 deposited under accession number ATCC 98482;

the protein being substantially free from other mammalian proteins. Preferably such protein comprises the amino acid sequence of SEQ ID NO:102 or the amino acid sequence of SEQ ID NO:102 from amino acid 1 to amino acid 31.

In one embodiment, the present invention provides a composition comprising an isolated polynucleotide selected from the group consisting of:

(a) a polynucleotide comprising the nucleotide sequence of SEQ ID NO:103;

(b) a polynucleotide comprising the nucleotide sequence of SEQ ID NO:103 from nucleotide 663 to nucleotide 755;

(c) a polynucleotide comprising the nucleotide sequence of SEQ ID NO:103from nucleotide 1 to nucleotide 850;

(d) a polynucleotide comprising the nucleotide sequence of the full-length protein coding sequence of clone co36_{—}1 deposited under accession number ATCC 98482;

(e) a polynucleotide encoding the full-length protein encoded by the cDNA insert of clone co36_{—}1 deposited under accession number ATCC 98482;

(f) a polynucleotide comprising the nucleotide sequence of the mature protein coding sequence of clone co36_{—}1 deposited under accession number ATCC 98482;

(g) a polynucleotide encoding the mature protein encoded by the cDNA insert of clone co36_{—}1 deposited under accession number ATCC 98482;

(h) a polynucleotide encoding a protein comprising the amino acid sequence of SEQ ID NO:104;

(i) a polynucleotide encoding a protein comprising a fragment of the amino acid sequence of SEQ ID NO:104 having biological activity;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:103 from nucleotide 663 to nucleotide 755; the nucleotide sequence of SEQ ID NO:103 from nucleotide 1 to nucleotide 850; the nucleotide sequence of the full-length protein coding sequence of clone co36_{—}1 deposited under accession number ATCC 98482; or the nucleotide sequence of the mature protein coding sequence of clone co36_{—}1 deposited under accession number ATCC 98482. In other preferred embodiments, the polynucleotide encodes the full-length or mature protein encoded by the cDNA insert of clone co36_{—}1 deposited under accession number ATCC 98482. In yet other preferred embodiments, the present invention provides a polynucleotide encoding a protein comprising the amino acid sequence of SEQ ID NO:104 from amino acid 1 to amino acid 22.

Other embodiments provide the gene corresponding to the cDNA sequence of SEQ ID NO:103.

In other embodiments, the present invention provides a composition comprising a protein, wherein said protein comprises an amino acid sequence selected from the group consisting of:

(a) the amino acid sequence of SEQ ID NO:104;

(b) the amino acid sequence of SEQ ID NO:8 from amino acid 1 to amino acid 22;

(c) fragments of the amino acid sequence of SEQ ID NO:104; and

(d) the amino acid sequence encoded by the cDNA insert of clone co36_{—}1 deposited under accession number ATCC 98482;

the protein being substantially free from other mammalian proteins. Preferably such protein comprises the amino acid sequence of SEQ ID NO:104 or the amino acid sequence of SEQ ID NO:104 from amino acid 1 to amino acid 22.

In one embodiment, the present invention provides a composition comprising an isolated polynucleotide selected from the group consisting of:

(a) a polynucleotide comprising the nucleotide sequence of SEQ ID NO:105;

(b) a polynucleotide comprising the nucleotide sequence of SEQ ID NO:105 from nucleotide 127 to nucleotide 783;

(c) a polynucleotide comprising the nucleotide sequence of SEQ ID NO:105 from nucleotide 172 to nucleotide 783;

(d) a polynucleotide comprising the nucleotide sequence of SEQ ID NO:105 from nucleotide 7 to nucleotide 462;

(e) a polynucleotide comprising the nucleotide sequence of the full-length protein coding sequence of clone cp116_{—}1 deposited under accession number ATCC 98482;

(f) a polynucleotide encoding the full-length protein encoded by the cDNA insert of clone cp116_{—}1 deposited under accession number ATCC 98482;

(g) a polynucleotide comprising the nucleotide sequence of the mature protein coding sequence of clone cp116_{—}1 deposited under accession number ATCC 98482;

(h) a polynucleotide encoding the mature protein encoded by the cDNA insert of clone cp116_{—}1 deposited under accession number ATCC 98482;

(i) a polynucleotide encoding a protein comprising the amino acid sequence of SEQ ID NO:106;

(j) a polynucleotide encoding a protein comprising a fragment of the amino acid sequence of SEQ ID NO:106 having biological activity;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:105 from nucleotide 127 to nucleotide 783; the nucleotide sequence of SEQ ID NO:105 from nucleotide 172 to nucleotide 783; the nucleotide sequence of SEQ ID NO:105 from nucleotide 7 to nucleotide 462; the nucleotide sequence of the full-length protein coding sequence of clone cp116_{—}1 deposited under accession number ATCC 98482; or the nucleotide sequence of the mature protein coding sequence of clone cp116_{—}1 deposited under accession number ATCC 98482. In other preferred embodiments, the polynucleotide encodes the full-length or mature protein encoded by the cDNA insert of clone cp116_{—}1 deposited under accession number ATCC 98482. In yet other preferred embodiments, the present invention provides a polynucleotide encoding a protein comprising the amino acid sequence of SEQ ID NO:106 from amino acid 1 to amino acid 112.

Other embodiments provide the gene corresponding to the cDNA sequence of SEQ ID NO:105.

In other embodiments, the present invention provides a composition comprising a protein, wherein said protein comprises an amino acid sequence selected from the group consisting of:

(a) the amino acid sequence of SEQ ID NO:106;

(b) the amino acid sequence of SEQ ID NO:106 from amino acid 1 to amino acid 112;

(c) fragments of the amino acid sequence of SEQ ID NO:106; and

(d) the amino acid sequence encoded by the cDNA insert of clone cp116_{—}1 deposited under accession number ATCC 98482;

the protein being substantially free from other mammalian proteins. Preferably such protein comprises the amino acid sequence of SEQ ID NO:106 or the amino acid sequence of SEQ ID NO:106 from amino acid 1 to amino acid 112.

In one embodiment, the present invention provides a composition comprising an isolated polynucleotide selected from the group consisting of:

(a) a polynucleotide comprising the nucleotide sequence of SEQ ID NO:108;

(b) a polynucleotide comprising the nucleotide sequence of SEQ ID NO:108 from nucleotide 231 to nucleotide 533;

(c) a polynucleotide comprising the nucleotide sequence of the full-length protein coding sequence of clone cw1195_{—}2 deposited under accession number ATCC 98482;

(d) a polynucleotide encoding the full-length protein encoded by the cDNA insert of clone cw1195_{—}2 deposited under accession number ATCC 98482;

(e) a polynucleotide comprising the nucleotide sequence of the mature protein coding sequence of clone cw1195_{—}2 deposited under accession number ATCC 98482;

(f) a polynucleotide encoding the mature protein encoded by the cDNA insert of clone cw1195_{—}2 deposited under accession number ATCC 98482;

(g) a polynucleotide encoding a protein comprising the amino acid sequence of SEQ ID NO:109;

(h) a polynucleotide encoding a protein comprising a fragment of the amino acid sequence of SEQ ID NO:109 having biological activity;

(i) a polynucleotide which is an allelic variant of a polynucleotide of (a)-(f) above;

(j) a polynucleotide which encodes a species homologue of the protein of (g) or (h) above; and

(k) a polynucleotide capable of hybridizing under stringent conditions to any one of the polynucleotides specified in (a)-(h).

Preferably, such polynucleotide comprises the nucleotide sequence of SEQ ID NO75 from nucleotide 231 to nucleotide 533; the nucleotide sequence of the full-length protein coding sequence of clone cw1195_{—}2 deposited under accession number ATCC 98482; or the nucleotide sequence of the mature protein coding sequence of clone cw1195_{—}2 deposited under accession number ATCC 98482. In other preferred embodiments, the polynucleotide encodes the full-length or mature protein encoded by the cDNA insert of clone cw1195_{—}2 deposited under accession number ATCC 98482.

Other embodiments provide the gene corresponding to the cDNA sequence of SEQ ID NO:108, SEQ ID NO74 or SEQ ID NO:110.

In other embodiments, the present invention provides a composition comprising a protein, wherein said protein comprises an amino acid sequence selected from the group consisting of:

(a) the amino acid sequence of SEQ ID NO:109;

(b) fragments of the amino acid sequence of SEQ ID NO:109; and

(c) the amino acid sequence encoded by the cDNA insert of clone cw1195_{—}2 deposited under accession number ATCC 98482;

the protein being substantially free from other mammalian proteins. Preferably such protein comprises the amino acid sequence of SEQ ID NO:109.

In one embodiment, the present invention provides a composition comprising an isolated polynucleotide selected from the group consisting of:

(a) a polynucleotide comprising the nucleotide sequence of SEQ ID NO:111;

(b) a polynucleotide comprising the nucleotide sequence of SEQ ID NO:111 from nucleotide 645 to nucleotide 782;

(c) a polynucleotide comprising the nucleotide sequence of SEQ ID NO:111 from nucleotide 10 to nucleotide 773;

(d) a polynucleotide comprising the nucleotide sequence of the full-length protein coding sequence of clone fh13_{—}10 deposited under accession number ATCC 98482;

(e) a polynucleotide encoding the full-length protein encoded by the cDNA insert of clone fh13_{—}10 deposited under accession number ATCC 98482;

(f) a polynucleotide comprising the nucleotide sequence of the mature protein coding sequence of clone fh13_{—}10 deposited under accession number ATCC 98482;

(g) a polynucleotide encoding the mature protein encoded by the cDNA insert of clone fh13_{—}10 deposited under accession number ATCC 98482;

(h) a polynucleotide encoding a protein comprising the amino acid sequence of SEQ ID NO:112;

(i) a polynucleotide encoding a protein comprising a fragment of the amino acid sequence of SEQ ID NO:112 having biological activity;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:111 from nucleotide 645 to nucleotide 782; the nucleotide sequence of SEQ ID NO:111 from nucleotide 10 to nucleotide 773; the nucleotide sequence of the full-length protein coding sequence of clone fh13_{—}10 deposited under accession number ATCC 98482; or the nucleotide sequence of the mature protein coding sequence of clone fh13_{—}10 deposited under accession number ATCC 98482. In other preferred embodiments, the polynucleotide encodes the full-length or mature protein encoded by the cDNA insert of clone fh13_{—}10 deposited under accession number ATCC 98482. In yet other preferred embodiments, the present invention provides a polynucleotide encoding a protein comprising the amino acid sequence of SEQ ID NO:112 from amino acid 1 to amino acid 43.

Other embodiments provide the gene corresponding to the cDNA sequence of SEQ ID NO:111.

In other embodiments, the present invention provides a composition comprising a protein, wherein said protein comprises an amino acid sequence selected from the group consisting of:

(a) the amino acid sequence of SEQ ID NO:112;

(b) the amino acid sequence of SEQ ID NO:16 from amino acid 1 to amino acid 43;

(c) fragments of the amino acid sequence of SEQ ID NO:112; and

(d) the amino acid sequence encoded by the cDNA insert of clone fh13_{—}10 deposited under accession number ATCC 98482;

the protein being substantially free from other mammalian proteins. Preferably such protein comprises the amino acid sequence of SEQ ID NO:112 or the amino acid sequence of SEQ ID NO:112 from amino acid 1 to amino acid 43.

In one embodiment, the present invention provides a composition comprising an isolated polynucleotide selected from the group consisting of:

(a) a polynucleotide comprising the nucleotide sequence of SEQ ID NO:113;

(b) a polynucleotide comprising the nucleotide sequence of SEQ ID NO:113 from nucleotide 94 to nucleotide 216;

(c) a polynucleotide comprising the nucleotide sequence of SEQ ID NO:17 from nucleotide 160 to nucleotide 216;

(d) a polynucleotide comprising the nucleotide sequence of SEQ ID NO:113 from nucleotide 20 to nucleotide 193;

(e) a polynucleotide comprising the nucleotide sequence of the full-length protein coding sequence of clone gc57_{—}4 deposited under accession number ATCC 98482;

(f) a polynucleotide encoding the full-length protein encoded by the cDNA insert of clone gc57_{—}4 deposited under accession number ATCC 98482;

(g) a polynucleotide comprising the nucleotide sequence of the mature protein coding sequence of clone gc57_{—}4 deposited under accession number ATCC 98482;

(h) a polynucleotide encoding the mature protein encoded by the cDNA insert of clone gc57_{—}4 deposited under accession number ATCC 98482;

(i) a polynucleotide encoding a protein comprising the amino acid sequence of SEQ ID NO:114;

(j) a polynucleotide encoding a protein comprising a fragment of the amino acid sequence of SEQ ID NO:114 having biological activity;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:113 from nucleotide 94 to nucleotide 216; the nucleotide sequence of SEQ ID NO:113 from nucleotide 160 to nucleotide 216; the nucleotide sequence of SEQ ID NO:113 from nucleotide 20 to nucleotide 193; the nucleotide sequence of the full-length protein coding sequence of clone gc57_{—}4 deposited under accession number ATCC 98482; or the nucleotide sequence of the mature protein coding sequence of clone gc57_{—}4 deposited under accession number ATCC 98482. In other preferred embodiments, the polynucleotide encodes the full-length or mature protein encoded by the cDNA insert of clone gc57_{—}4 deposited under accession number ATCC 98482. In yet other preferred embodiments, the present invention provides a polynucleotide encoding a protein comprising the amino acid sequence of SEQ ID NO:18 from amino acid 1 to amino acid 33.

Other embodiments provide the gene corresponding to the cDNA sequence of SEQ ID NO:113.

In other embodiments, the present invention provides a composition comprising a protein, wherein said protein comprises an amino acid sequence selected from the group consisting of:

(a) the amino acid sequence of SEQ ID NO:114;

(b) the amino acid sequence of SEQ ID NO:114 from amino acid 1 to amino acid 33;

(c) fragments of the amino acid sequence of SEQ ID NO:114; and

(d) the amino acid sequence encoded by the cDNA insert of clone gc57_{—}4 deposited under accession number ATCC 98482;

the protein being substantially free from other mammalian proteins. Preferably such protein comprises the amino acid sequence of SEQ ID NO:114 or the amino acid sequence of SEQ ID NO:114 from amino acid 1 to amino acid 33.

In one embodiment, the present invention provides a composition comprising an isolated polynucleotide selected from the group consisting of:

(a) a polynucleotide comprising the nucleotide sequence of SEQ ID NO:115;

(b) a polynucleotide comprising the nucleotide sequence of SEQ ID NO:115 from nucleotide 2 to nucleotide 943;

(c) a polynucleotide comprising the nucleotide sequence of SEQ ID NO:115 from nucleotide 2 to nucleotide 670;

(d) a polynucleotide comprising the nucleotide sequence of the full-length protein coding sequence of clone h1165_{—}3 deposited under accession number ATCC 98482;

(e) a polynucleotide encoding the full-length protein encoded by the cDNA insert of clone h1165_{—}3 deposited under accession number ATCC 98482;

(f) a polynucleotide comprising the nucleotide sequence of the mature protein coding sequence of clone h1165_{—}3 deposited under accession number ATCC 98482;

(g) a polynucleotide encoding the mature protein encoded by the cDNA insert of clone h1165_{—}3 deposited under accession number ATCC 98482;

(h) a polynucleotide encoding a protein comprising the amino acid sequence of SEQ ID NO:116;

(i) a polynucleotide encoding a protein comprising a fragment of the amino acid sequence of SEQ ID NO:116 having biological activity;

(j) a polynucleotide which is an allelic variant of a polynucleotide of (a)-(g) above;

(k) a polynucleotide which encodes a species homologue of the protein of (h) or (i) above; and

(l) a polynucleotide capable of hybridizing under stringent conditions to any one of the polynucleotides specified in (a)-(i).

Preferably, such polynucleotide comprises the nucleotide sequence of SEQ ID NO:115 from nucleotide 2 to nucleotide 943; the nucleotide sequence of SEQ ID NO:115 from nucleotide 2 to nucleotide 670; the nucleotide sequence of the full-length protein coding sequence of clone h1165_{—}3 deposited under accession number ATCC 98482; or the nucleotide sequence of the mature protein coding sequence of clone h1165_{—}3 deposited under accession number ATCC 98482. In other preferred embodiments, the polynucleotide encodes the full-length or mature protein encoded by the cDNA insert of clone h1165_{—}3 deposited under accession number ATCC 98482. In yet other preferred embodiments, the present invention provides a polynucleotide encoding a protein comprising the amino acid sequence of SEQ ID NO:116 from amino acid 1 to amino acid 223.

Other embodiments provide the gene corresponding to the cDNA sequence of SEQ ID NO:115.

In other embodiments, the present invention provides a composition comprising a protein, wherein said protein comprises an amino acid sequence selected from the group consisting of:

(a) the amino acid sequence of SEQ ID NO:116;

(b) the amino acid sequence of SEQ ID NO:116 from amino acid 1 to amino acid 223;

(c) fragments of the amino acid sequence of SEQ ID NO:116; and

(d) the amino acid sequence encoded by the cDNA insert of clone h1165_{—}3 deposited under accession number ATCC 98482;

the protein being substantially free from other mammalian proteins. Preferably such protein comprises the amino acid sequence of SEQ ID NO:116 or the amino acid sequence of SEQ ID NO:116 from amino acid 1 to amino acid 223.

In one embodiment, the present invention provides a composition comprising an isolated polynucleotide selected from the group consisting of:

(a) a polynucleotide comprising the nucleotide sequence of SEQ ID NO:117;

(b) a polynucleotide comprising the nucleotide sequence of SEQ ID NO:117 from nucleotide 1242 to nucleotide 1457;

(c) a polynucleotide comprising the nucleotide sequence of SEQ ID NO:21 from nucleotide 1326 to nucleotide 1457;

(d) a polynucleotide comprising the nucleotide sequence of SEQ ID NO:117 from nucleotide 869 to nucleotide 1544;

(e) a polynucleotide comprising the nucleotide sequence of the full-length protein coding sequence of clone hb752_{—}1 deposited under accession number ATCC 98482;

(f) a polynucleotide encoding the full-length protein encoded by the cDNA insert of clone hb752_{—}1 deposited under accession number ATCC 98482;

(g) a polynucleotide comprising the nucleotide sequence of the mature protein coding sequence of clone hb752_{—}1 deposited under accession number ATCC 98482;

(h) a polynucleotide encoding the mature protein encoded by the cDNA insert of clone hb752_{—}1 deposited under accession number ATCC 98482;

(i) a polynucleotide encoding a protein comprising the amino acid sequence of SEQ ID NO:118;

(j) a polynucleotide encoding a protein comprising a fragment of the amino acid sequence of SEQ ID NO:118 having biological activity;

(k) a polynucleotide which is an allelic variant of a polynucleotide of (a)-(h) above;

(l) a polynucleotide which encodes a species homologue of the protein of (i) or (j) above; and

(m) a polynucleotide capable of hybridizing under stringent conditions to any one of the polynucleotides specified in (a)-(j).

Preferably, such polynucleotide comprises the nucleotide sequence of SEQ ID NO:117 from nucleotide 1242 to nucleotide 1457; the nucleotide sequence of SEQ ID NO:117 from nucleotide 1326 to nucleotide 1457; the nucleotide sequence of SEQ ID NO:117 from nucleotide 869 to nucleotide 1544; the nucleotide sequence of the full-length protein coding sequence of clone hb752_{—}1 deposited under accession number ATCC 98482; or the nucleotide sequence of the mature protein coding sequence of clone hb752_{—}1 deposited under accession number ATCC 98482. In other preferred embodiments, the polynucleotide encodes the full-length or mature protein encoded by the cDNA insert of clone hb752_{—}1 deposited under accession number ATCC 98482. In yet other preferred embodiments, the present invention provides a polynucleotide encoding a protein comprising the amino acid sequence of SEQ ID NO:118 from amino acid 1 to amino acid 69.

Other embodiments provide the gene corresponding to the cDNA sequence of SEQ ID NO:117.

In other embodiments, the present invention provides a composition comprising a protein, wherein said protein comprises an amino acid sequence selected from the group consisting of:

(a) the amino acid sequence of SEQ ID NO:118;

(b) the amino acid sequence of SEQ ID NO:118 from amino acid 1 to amino acid 69;

(c) fragments of the amino acid sequence of SEQ ID NO:118; and

(d) the amino acid sequence encoded by the cDNA insert of clone hb752_{—}1 deposited under accession number ATCC 98482;

the protein being substantially free from other mammalian proteins. Preferably such protein comprises the amino acid sequence of SEQ ID NO:118 or the amino acid sequence of SEQ ID NO:118 from amino acid 1 to amino acid 69.

In one embodiment, the present invention provides a composition comprising an isolated polynucleotide selected from the group consisting of:

(a) a polynucleotide comprising the nucleotide sequence of SEQ ID NO:129;

(b) a polynucleotide comprising the nucleotide sequence of SEQ ID NO:129 from nucleotide 864 to nucleotide 1340;

(c) a polynucleotide comprising the nucleotide sequence of SEQ ID NO:129 from nucleotide 1 to nucleotide 1175;

(d) a polynucleotide comprising the nucleotide sequence of the full-length protein coding sequence of clone bi127_{—}5 deposited under accession number ATCC 98501;

(e) a polynucleotide encoding the full-length protein encoded by the cDNA insert of clone bi127_{—}5 deposited under accession number ATCC 98501;

(f) a polynucleotide comprising the nucleotide sequence of a mature protein coding sequence of clone bi127_{—}5 deposited under accession number ATCC 98501;

(g) a polynucleotide encoding a mature protein encoded by the cDNA insert of clone bi127_{—}5 deposited under accession number ATCC 98501;

(h) a polynucleotide encoding a protein comprising the amino acid sequence of SEQ ID NO:130;

(i) a polynucleotide encoding a protein comprising a fragment of the amino acid sequence of SEQ ID NO:130 having biological activity, the fragment comprising eight consecutive amino acids of SEQ ID NO:130;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:129 from nucleotide 864 to nucleotide 1340; the nucleotide sequence of SEQ ID NO:129 from nucleotide 1 to nucleotide 1175; the nucleotide sequence of the full-length protein coding sequence of clone bi127_{—}5 deposited under accession number ATCC 98501; or the nucleotide sequence of a mature protein coding sequence of clone bi127_{—}5 deposited under accession number ATCC 98501. In other preferred embodiments, the polynucleotide encodes the full-length or a mature protein encoded by the cDNA insert of clone bi127_{—}5 deposited under accession number ATCC 98501. In yet other preferred embodiments, the present invention provides a polynucleotide encoding a protein comprising the amino acid sequence of SEQ ID NO:130 from amino acid 1 to amino acid 104. In further preferred embodiments, the present invention provides a polynucleotide encoding a protein comprising a fragment of the amino acid sequence of SEQ ID NO:130 having biological activity, the fragment preferably comprising eight (more preferably twenty, most preferably thirty) consecutive amino acids of SEQ ID NO:130, or a polynucleotide encoding a protein comprising a fragment of the amino acid sequence of SEQ ID NO:130 having biological activity, the fragment comprising the amino acid sequence from amino acid 74 to amino acid 83 of SEQ ID NO:130.

Other embodiments provide the gene corresponding to the cDNA sequence of SEQ ID NO:129.

In other embodiments, the present invention provides a composition comprising a protein, wherein said protein comprises an amino acid sequence selected from the group consisting of:

(a) the amino acid sequence of SEQ ID NO:130;

(b) the amino acid sequence of SEQ ID NO:130 from amino acid 1 to amino acid 104;

(c) fragments of the amino acid sequence of SEQ ID NO:130 comprising eight consecutive amino acids of SEQ ID NO:130; and

(d) the amino acid sequence encoded by the cDNA insert of clone bi127_{—}5 deposited under accession number ATCC 98501;

the protein being substantially free from other mammalian proteins. Preferably such protein comprises the amino acid sequence of SEQ ID NO:130 or the amino acid sequence of SEQ ID NO:130 from amino acid 1 to amino acid 104. In further preferred embodiments, the present invention provides a protein comprising a fragment of the amino acid sequence of SEQ ID NO:130 having biological activity, the fragment preferably comprising eight (more preferably twenty, most preferably thirty) consecutive amino acids of SEQ ID NO:130, or a protein comprising a fragment of the amino acid sequence of SEQ ID NO:130 having biological activity, the fragment comprising the amino acid sequence from amino acid 74 to amino acid 83 of SEQ ID NO:130.

In one embodiment, the present invention provides a composition comprising an isolated polynucleotide selected from the group consisting of:

(a) a polynucleotide comprising the nucleotide sequence of SEQ ID NO:131;

(b) a polynucleotide comprising the nucleotide sequence of SEQ ID NO:131 from nucleotide 46 to nucleotide 738;

(c) a polynucleotide comprising the nucleotide sequence of SEQ ID NO:131 from nucleotide 346 to nucleotide 738;

(d) a polynucleotide comprising the nucleotide sequence of SEQ ID NO:3 from nucleotide 688 to nucleotide 1425;

(e) a polynucleotide comprising the nucleotide sequence of the full-length protein coding sequence of clone bl194_{—}2 deposited under accession number ATCC 98501;

(f) a polynucleotide encoding the full-length protein encoded by the cDNA insert of clone bl194_{—}2 deposited under accession number ATCC 98501;

(g) a polynucleotide comprising the nucleotide sequence of a mature protein coding sequence of clone bl194_{—}2 deposited under accession number ATCC 98501;

(h) a polynucleotide encoding a mature protein encoded by the cDNA insert of clone bl194_{—}2 deposited under accession number ATCC 98501;

(i) a polynucleotide encoding a protein comprising the amino acid sequence of SEQ ID NO:132;

(j) a polynucleotide encoding a protein comprising a fragment of the amino acid sequence of SEQ ID NO:132 having biological activity, the fragment comprising eight consecutive amino acids of SEQ ID NO:132;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:131 from nucleotide 46 to nucleotide 738; the nucleotide sequence of SEQ ID NO:131 from nucleotide 346 to nucleotide 738; the nucleotide sequence of SEQ ID NO:131 from nucleotide 688 to nucleotide 1425; the nucleotide sequence of the full-length protein coding sequence of clone bl194_{—}2 deposited under accession number ATCC 98501; or the nucleotide sequence of a mature protein coding sequence of clone bl194_{—}2 deposited under accession number ATCC 98501. In other preferred embodiments, the polynucleotide encodes the full-length or a mature protein encoded by the cDNA insert of clone bl194_{—}2 deposited under accession number ATCC 98501. In yet other preferred embodiments, the present invention provides a polynucleotide encoding a protein comprising the amino acid sequence of SEQ ID NO:4 from amino acid 1 to amino acid 171. In further preferred embodiments, the present invention provides a polynucleotide encoding a protein comprising a fragment of the amino acid sequence of SEQ ID NO:132 having biological activity, the fragment preferably comprising eight (more preferably twenty, most preferably thirty) consecutive amino acids of SEQ ID NO:132, or a polynucleotide encoding a protein comprising a fragment of the amino acid sequence of SEQ ID NO:132 having biological activity, the fragment comprising the amino acid sequence from amino acid 110 to amino acid 119 of SEQ ID NO:132.

Other embodiments provide the gene corresponding to the cDNA sequence of SEQ ID NO:131.

In other embodiments, the present invention provides a composition comprising a protein, wherein said protein comprises an amino acid sequence selected from the group consisting of:

(a) the amino acid sequence of SEQ ID NO:132;

(b) the amino acid sequence of SEQ ID NO:132 from amino acid 1 to amino acid 171;

(c) fragments of the amino acid sequence of SEQ ID NO:132 comprising eight consecutive amino acids of SEQ ID NO:132; and

(d) the amino acid sequence encoded by the cDNA insert of clone bl194_{—}2 deposited under accession number ATCC 98501;

the protein being substantially free from other mammalian proteins. Preferably such protein comprises the amino acid sequence of SEQ ID NO:132 or the amino acid sequence of SEQ ID NO:132 from amino acid 1 to amino acid 171. In further preferred embodiments, the present invention provides a protein comprising a fragment of the amino acid sequence of SEQ ID NO:132 having biological activity, the fragment preferably comprising eight (more preferably twenty, most preferably thirty) consecutive amino acids of SEQ ID NO:132, or a protein comprising a fragment of the amino acid sequence of SEQ ID NO:132 having biological activity, the fragment comprising the amino acid sequence from amino acid 110 to amino acid 119 of SEQ ID NO:132.

In one embodiment, the present invention provides a composition comprising an isolated polynucleotide selected from the group consisting of:

(a) a polynucleotide comprising the nucleotide sequence of SEQ ID NO:133;

(b) a polynucleotide comprising the nucleotide sequence of SEQ ID NO:133 from nucleotide 234 to nucleotide 1235;

(c) a polynucleotide comprising the nucleotide sequence of SEQ ID NO:133 from nucleotide 291 to nucleotide 1235;

(d) a polynucleotide comprising the nucleotide sequence of SEQ ID NO:133 from nucleotide 209 to nucleotide 1050;

(e) a polynucleotide comprising the nucleotide sequence of the full-length protein coding sequence of clone cc130_{—}1 deposited under accession number ATCC 98501;

(f) a polynucleotide encoding the full-length protein encoded by the cDNA insert of clone cc130_{—}1 deposited under accession number ATCC 98501;

(g) a polynucleotide comprising the nucleotide sequence of a mature protein coding sequence of clone cc130_{—}1 deposited under accession number ATCC 98501;

(h) a polynucleotide encoding a mature protein encoded by the cDNA insert of clone cc130_{—}1 deposited under accession number ATCC 98501;

(i) a polynucleotide encoding a protein comprising the amino acid sequence of SEQ ID NO:134;

(j) a polynucleotide encoding a protein comprising a fragment of the amino acid sequence of SEQ ID NO:134 having biological activity, the fragment comprising eight consecutive amino acids of SEQ ID NO:134;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:133 from nucleotide 234 to nucleotide 1235; the nucleotide sequence of SEQ ID NO:133 from nucleotide 291 to nucleotide 1235; the nucleotide sequence of SEQ ID NO:133 from nucleotide 209 to nucleotide 1050; the nucleotide sequence of the full-length protein coding sequence of clone cc1301 deposited under accession number ATCC 98501; or the nucleotide sequence of a mature protein coding sequence of clone cc130_{—}1 deposited under accession number ATCC 98501. In other preferred embodiments, the polynucleotide encodes the full-length or a mature protein encoded by the cDNA insert of clone cc1301 deposited under accession number ATCC 98501. In yet other preferred embodiments, the present invention provides a polynucleotide encoding a protein comprising the amino acid sequence of SEQ ID NO:6 from amino acid 1 to amino acid 272. In further preferred embodiments, the present invention provides a polynucleotide encoding a protein comprising a fragment of the amino acid sequence of SEQ ID NO:134 having biological activity, the fragment preferably comprising eight (more preferably twenty, most preferably thirty) consecutive amino acids of SEQ ID NO:134, or a polynucleotide encoding a protein comprising a fragment of the amino acid sequence of SEQ ID NO:134 having biological activity, the fragment comprising the amino acid sequence from amino acid 162 to amino acid 171 of SEQ ID NO:134.

Other embodiments provide the gene corresponding to the cDNA sequence of SEQ ID NO:133.

In other embodiments, the present invention provides a composition comprising a protein, wherein said protein comprises an amino acid sequence selected from the group consisting of:

(a) the amino acid sequence of SEQ ID NO:134;

(b) the amino acid sequence of SEQ ID NO:134 from amino acid 1 to amino acid 272;

(c) fragments of the amino acid sequence of SEQ ID NO:134 comprising eight consecutive amino acids of SEQ ID NO:134; and

(d) the amino acid sequence encoded by the cDNA insert of clone cc130_{—}1 deposited under accession number ATCC 98501;

the protein being substantially free from other mammalian proteins. Preferably such protein comprises the amino acid sequence of SEQ ID NO:134 or the amino acid sequence of SEQ ID NO:134 from amino acid 1 to amino acid 272. In further preferred embodiments, the present invention provides a protein comprising a fragment of the amino acid sequence of SEQ ID NO:134 having biological activity, the fragment preferably comprising eight (more preferably twenty, most preferably thirty) consecutive amino acids of SEQ ID NO:134, or a protein comprising a fragment of the amino acid sequence of SEQ ID NO:134 having biological activity, the fragment comprising the amino acid sequence from amino acid 162 to amino acid 171 of SEQ ID NO:134.

In one embodiment, the present invention provides a composition comprising an isolated polynucleotide selected from the group consisting of:

(a) a polynucleotide comprising the nucleotide sequence of SEQ ID NO:135;

(b) a polynucleotide comprising the nucleotide sequence of SEQ ID NO:135 from nucleotide 1554 to nucleotide 1784;

(c) a polynucleotide comprising the nucleotide sequence of SEQ ID NO:135 from nucleotide 1659 to nucleotide 1784;

(d) a polynucleotide comprising the nucleotide sequence of SEQ ID NO:135 from nucleotide 1508 to nucleotide 1865;

(e) a polynucleotide comprising the nucleotide sequence of the full-length protein coding sequence of clone ch582_{—}1 deposited under accession number ATCC 98501;

(f) a polynucleotide encoding the full-length protein encoded by the cDNA insert of clone ch582_{—}1 deposited under accession number ATCC 98501;

(g) a polynucleotide comprising the nucleotide sequence of a mature protein coding sequence of clone ch582_{—}1 deposited under accession number ATCC 98501;

(h) a polynucleotide encoding a mature protein encoded by the cDNA insert of clone ch582_{—}1 deposited under accession number ATCC 98501;

(i) a polynucleotide encoding a protein comprising the amino acid sequence of SEQ ID NO:136;

(j) a polynucleotide encoding a protein comprising a fragment of the amino acid sequence of SEQ ID NO:136 having biological activity, the fragment comprising eight consecutive amino acids of SEQ ID NO:136;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:135 from nucleotide 1554 to nucleotide 1784; the nucleotide sequence of SEQ ID NO:135 from nucleotide 1659 to nucleotide 1784; the nucleotide sequence of SEQ ID NO:135 from nucleotide 1508 to nucleotide 1865; the nucleotide sequence of the full-length protein coding sequence of clone ch582_{—}1 deposited under accession number ATCC 98501; or the nucleotide sequence of a mature protein coding sequence of clone ch582_{—}1 deposited under accession number ATCC 98501. In other preferred embodiments, the polynucleotide encodes the full-length or a mature protein encoded by the cDNA insert of clone ch582_{—}1 deposited under accession number ATCC 98501. In further preferred embodiments, the present invention provides a polynucleotide encoding a protein comprising a fragment of the amino acid sequence of SEQ ID NO:136 having biological activity, the fragment preferably comprising eight (more preferably twenty, most preferably thirty) consecutive amino acids of SEQ ID NO:136, or a polynucleotide encoding a protein comprising a fragment of the amino acid sequence of SEQ ID NO:136 having biological activity, the fragment comprising the amino acid sequence from amino acid 33 to amino acid 42 of SEQ ID NO:136.

Other embodiments provide the gene corresponding to the cDNA sequence of SEQ ID NO:135.

In other embodiments, the present invention provides a composition comprising a protein, wherein said protein comprises an amino acid sequence selected from the group consisting of:

(a) the amino acid sequence of SEQ ID NO:136;

(b) fragments of the amino acid sequence of SEQ ID NO:136 comprising eight consecutive amino acids of SEQ ID NO:136; and

(c) the amino acid sequence encoded by the cDNA insert of clone ch582_{—}1 deposited under accession number ATCC 98501;

the protein being substantially free from other mammalian proteins. Preferably such protein comprises the amino acid sequence of SEQ ID NO:136. In further preferred embodiments, the present invention provides a protein comprising a fragment of the amino acid sequence of SEQ ID NO:136 having biological activity, the fragment preferably comprising eight (more preferably twenty, most preferably thirty) consecutive amino acids of SEQ ID NO:136, or a protein comprising a fragment of the amino acid sequence of SEQ ID NO:136 having biological activity, the fragment comprising the amino acid sequence from amino acid 33 to amino acid 42 of SEQ ID NO:136.

In one embodiment, the present invention provides a composition comprising an isolated polynucleotide selected from the group consisting of:

(a) a polynucleotide comprising the nucleotide sequence of SEQ ID NO:137;

(b) a polynucleotide comprising the nucleotide sequence of SEQ ID NO:137 from nucleotide 1375 to nucleotide 1605;

(c) a polynucleotide comprising the nucleotide sequence of SEQ ID NO:137 from nucleotide 1107 to nucleotide 1539;

(d) a polynucleotide comprising the nucleotide sequence of the full-length protein coding sequence of clone cq294_{—}14 deposited under accession number ATCC 98501;

(e) a polynucleotide encoding the full-length protein encoded by the cDNA insert of clone cq294_{—}14 deposited under accession number ATCC 98501;

(f) a polynucleotide comprising the nucleotide sequence of a mature protein coding sequence of clone cq294_{—}14 deposited under accession number ATCC 98501;

(g) a polynucleotide encoding a mature protein encoded by the cDNA insert of clone cq294_{—}14 deposited under accession number ATCC 98501;

(h) a polynucleotide encoding a protein comprising the amino acid sequence of SEQ ID NO:138;

(i) a polynucleotide encoding a protein comprising a fragment of the amino acid sequence of SEQ ID NO:138 having biological activity, the fragment comprising eight consecutive amino acids of SEQ ID NO:138;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:137 from nucleotide 1375 to nucleotide 1605; the nucleotide sequence of SEQ ID NO:137 from nucleotide 1107 to nucleotide 1539; the nucleotide sequence of the full-length protein coding sequence of clone cq294_{—}14 deposited under accession number ATCC 98501; or the nucleotide sequence of a mature protein coding sequence of clone cq294_{—}14 deposited under accession number ATCC 98501. In other preferred embodiments, the polynucleotide encodes the full-length or a mature protein encoded by the cDNA insert of clone cq294_{—}14 deposited under accession number ATCC 98501. In yet other preferred embodiments, the present invention provides a polynucleotide encoding a protein comprising the amino acid sequence of SEQ ID NO:138 from amino acid 1 to amino acid 55. In further preferred embodiments, the present invention provides a polynucleotide encoding a protein comprising a fragment of the amino acid sequence of SEQ ID NO:138 having biological activity, the fragment preferably comprising eight (more preferably twenty, most preferably thirty) consecutive amino acids of SEQ ID NO:138, or a polynucleotide encoding a protein comprising a fragment of the amino acid sequence of SEQ ID NO:138 having biological activity, the fragment comprising the amino acid sequence from amino acid 33 to amino acid 42 of SEQ ID NO:138.

Other embodiments provide the gene corresponding to the cDNA sequence of SEQ ID NO:137.

In other embodiments, the present invention provides a composition comprising a protein, wherein said protein comprises an amino acid sequence selected from the group consisting of:

(a) the amino acid sequence of SEQ ID NO:138;

(b) the amino acid sequence of SEQ ID NO:138 from amino acid 1 to amino acid 55;

(c) fragments of the amino acid sequence of SEQ ID NO:138 comprising eight consecutive amino acids of SEQ ID NO:138; and

(d) the amino acid sequence encoded by the cDNA insert of clone cq294_{—}14 deposited under accession number ATCC 98501;

the protein being substantially free from other mammalian proteins. Preferably such protein comprises the amino acid sequence of SEQ ID NO:138 or the amino acid sequence of SEQ ID NO:138 from amino acid 1 to amino acid 55. In further preferred embodiments, the present invention provides a protein comprising a fragment of the amino acid sequence of SEQ ID NO:138 having biological activity, the fragment preferably comprising eight (more preferably twenty, most preferably thirty) consecutive amino acids of SEQ ID NO:138, or a protein comprising a fragment of the amino acid sequence of SEQ ID NO:138 having biological activity, the fragment comprising the amino acid sequence from amino acid 33 to amino acid 42 of SEQ ID NO:138.

In one embodiment, the present invention provides a composition comprising an isolated polynucleotide selected from the group consisting of:

(a) a polynucleotide comprising the nucleotide sequence of SEQ ID NO:139;

(b) a polynucleotide comprising the nucleotide sequence of SEQ ID NO:139 from nucleotide 66 to nucleotide 1880;

(c) a polynucleotide comprising the nucleotide sequence of SEQ ID NO:139 from nucleotide 1 to nucleotide 581;

(d) a polynucleotide comprising the nucleotide sequence of the full-length protein coding sequence of clone dd454_{—}1 deposited under accession number ATCC 98501;

(e) a polynucleotide encoding the full-length protein encoded by the cDNA insert of clone dd454_{—}1 deposited under accession number ATCC 98501;

(f) a polynucleotide comprising the nucleotide sequence of a mature protein coding sequence of clone dd454_{—}1 deposited under accession number ATCC 98501;

(g) a polynucleotide encoding a mature protein encoded by the cDNA insert of clone dd454_{—}1 deposited under accession number ATCC 98501;

(h) a polynucleotide encoding a protein comprising the amino acid sequence of SEQ ID NO:140;

(i) a polynucleotide encoding a protein comprising a fragment of the amino acid sequence of SEQ ID NO:140 having biological activity, the fragment comprising eight consecutive amino acids of SEQ ID NO:140;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:139 from nucleotide 66 to nucleotide 1880; the nucleotide sequence of SEQ ID NO:139 from nucleotide 1 to nucleotide 581; the nucleotide sequence of the full-length protein coding sequence of clone dd454_{—}1 deposited under accession number ATCC 98501; or the nucleotide sequence of a mature protein coding sequence of clone dd454_{—}1 deposited under accession number ATCC 98501. In other preferred embodiments, the polynucleotide encodes the full-length or a mature protein encoded by the cDNA insert of clone dd454_{—}1 deposited under accession number ATCC 98501. In yet other preferred embodiments, the present invention provides a polynucleotide encoding a protein comprising the amino acid sequence of SEQ ID NO:140 from amino acid 1 to amino acid 172. In further preferred embodiments, the present invention provides a polynucleotide encoding a protein comprising a fragment of the amino acid sequence of SEQ ID NO:140 having biological activity, the fragment preferably comprising eight (more preferably twenty, most preferably thirty) consecutive amino acids of SEQ ID NO:140, or a polynucleotide encoding a protein comprising a fragment of the amino acid sequence of SEQ ID NO:140 having biological activity, the fragment comprising the amino acid sequence from amino acid 297 to amino acid 306 of SEQ ID NO:140.

Other embodiments provide the gene corresponding to the cDNA sequence of SEQ ID NO:139.

In other embodiments, the present invention provides a composition comprising a protein, wherein said protein comprises an amino acid sequence selected from the group consisting of:

(a) the amino acid sequence of SEQ ID NO:140;

(b) the amino acid sequence of SEQ ID NO:140 from amino acid 1 to amino acid 172;

(c) fragments of the amino acid sequence of SEQ ID NO:140 comprising eight consecutive amino acids of SEQ ID NO:140; and

(d) the amino acid sequence encoded by the cDNA insert of clone dd454_{—}1 deposited under accession number ATCC 98501;

the protein being substantially free from other mammalian proteins. Preferably such protein comprises the amino acid sequence of SEQ ID NO:140 or the amino acid sequence of SEQ ID NO:140 from amino acid 1 to amino acid 172. In further preferred embodiments, the present invention provides a protein comprising a fragment of the amino acid sequence of SEQ ID NO:140 having biological activity, the fragment preferably comprising eight (more preferably twenty, most preferably thirty) consecutive amino acids of SEQ ID NO:140, or a protein comprising a fragment of the amino acid sequence of SEQ ID NO:140 having biological activity, the fragment comprising the amino acid sequence from amino acid 297 to amino acid 306 of SEQ ID NO:140.

In one embodiment, the present invention provides a composition comprising an isolated polynucleotide selected from the group consisting of:

(a) a polynucleotide comprising the nucleotide sequence of SEQ ID NO:141;

(b) a polynucleotide comprising the nucleotide sequence of SEQ ID NO:141 from nucleotide 462 to nucleotide 3170;

(c) a polynucleotide comprising the nucleotide sequence of SEQ ID NO:141 from nucleotide 1188 to nucleotide 1517;

(d) a polynucleotide comprising the nucleotide sequence of the full-length protein coding sequence of clone du157_{—}12 deposited under accession number ATCC 98724;

(e) a polynucleotide encoding the full-length protein encoded by the cDNA insert of clone du157_{—}12 deposited under accession number ATCC 98724;

(f) a polynucleotide comprising the nucleotide sequence of a mature protein coding sequence of clone du157_{—}12 deposited under accession number ATCC 98724;

(g) a polynucleotide encoding a mature protein encoded by the cDNA insert of clone du157_{—}12 deposited under accession number ATCC 98724;

(h) a polynucleotide encoding a protein comprising the amino acid sequence of SEQ ID NO:142;

(i) a polynucleotide encoding a protein comprising a fragment of the amino acid sequence of SEQ ID NO:142 having biological activity, the fragment comprising eight consecutive amino acids of SEQ ID NO:142;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:141 from nucleotide 462 to nucleotide 3170; the nucleotide sequence of SEQ ID NO:141 from nucleotide 1188 to nucleotide 1517; the nucleotide sequence of the full-length protein coding sequence of clone du157_{—}12 deposited under accession number ATCC 98724; or the nucleotide sequence of a mature protein coding sequence of clone du157_{—}12 deposited under accession number ATCC 98724. In other preferred embodiments, the polynucleotide encodes the full-length or a mature protein encoded by the cDNA insert of clone du157_{—}12 deposited under accession number ATCC 98724. In yet other preferred embodiments, the present invention provides a polynucleotide encoding a protein comprising the amino acid sequence of SEQ ID NO:142 from amino acid 251 to amino acid 352. In further preferred embodiments, the present invention provides a polynucleotide encoding a protein comprising a fragment of the amino acid sequence of SEQ ID NO:142 having biological activity, the fragment preferably comprising eight (more preferably twenty, most preferably thirty) consecutive amino acids of SEQ ID NO:142, or a polynucleotide encoding a protein comprising a fragment of the amino acid sequence of SEQ ID NO:142 having biological activity, the fragment comprising the amino acid sequence from amino acid 446 to amino acid 455 of SEQ ID NO:142.

Other embodiments provide the gene corresponding to the cDNA sequence of SEQ ID NO:141.

In other embodiments, the present invention provides a composition comprising a protein, wherein said protein comprises an amino acid sequence selected from the group consisting of:

(a) the amino acid sequence of SEQ ID NO:142;

(b) the amino acid sequence of SEQ ID NO:142 from amino acid 251 to amino acid 352;

(c) fragments of the amino acid sequence of SEQ ID NO:142 comprising eight consecutive amino acids of SEQ ID NO:142; and

(d) the amino acid sequence encoded by the cDNA insert of clone du157_{—}12 deposited under accession number ATCC 98724;

the protein being substantially free from other mammalian proteins. Preferably such protein comprises the amino acid sequence of SEQ ID NO:142 or the amino acid sequence of SEQ ID NO:142 from amino acid 251 to amino acid 352. In further preferred embodiments, the present invention provides a protein comprising a fragment of the amino acid sequence of SEQ ID NO:142 having biological activity, the fragment preferably comprising eight (more preferably twenty, most preferably thirty) consecutive amino acids of SEQ ID NO:142, or a protein comprising a fragment of the amino acid sequence of SEQ ID NO:142 having biological activity, the fragment comprising the amino acid sequence from amino acid 446 to amino acid 455 of SEQ ID NO:142.

In one embodiment, the present invention provides a composition comprising an isolated polynucleotide selected from the group consisting of:

(a) a polynucleotide comprising the nucleotide sequence of SEQ ID NO:143;

(b) a polynucleotide comprising the nucleotide sequence of SEQ ID NO:143 from nucleotide 865 to nucleotide 1158;

(c) a polynucleotide comprising the nucleotide sequence of SEQ ID NO:143 from nucleotide 1108 to nucleotide 1158;

(d) a polynucleotide comprising the nucleotide sequence of SEQ ID NO:143 from nucleotide 1 to nucleotide 764;

(e) a polynucleotide comprising the nucleotide sequence of the full-length protein coding sequence of clone du372_{—}1 deposited under accession number ATCC 98501;

(f) a polynucleotide encoding the full-length protein encoded by the cDNA insert of clone du372_{—}1 deposited under accession number ATCC 98501;

(g) a polynucleotide comprising the nucleotide sequence of a mature protein coding sequence of clone du372_{—}1 deposited under accession number ATCC 98501;

(h) a polynucleotide encoding a mature protein encoded by the cDNA insert of clone du372_{—}1 deposited under accession number ATCC 98501;

(i) a polynucleotide encoding a protein comprising the amino acid sequence of SEQ ID NO:144;

(j) a polynucleotide encoding a protein comprising a fragment of the amino acid sequence of SEQ ID NO:144 having biological activity, the fragment comprising eight consecutive amino acids of SEQ ID NO:144;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:143 from nucleotide 865 to nucleotide 1158; the nucleotide sequence of SEQ ID NO:143 from nucleotide 1108 to nucleotide 1158; the nucleotide sequence of SEQ ID NO:143 from nucleotide 1 to nucleotide 764; the nucleotide sequence of the full-length protein coding sequence of clone du372_{—}1 deposited under accession number ATCC 98501; or the nucleotide sequence of a mature protein coding sequence of clone du372_{—}1 deposited under accession number ATCC 98501. In other preferred embodiments, the polynucleotide encodes the full-length or a mature protein encoded by the cDNA insert of clone du372_{—}1 deposited under accession number ATCC 98501. In yet other preferred embodiments, the present invention provides a polynucleotide encoding a protein comprising the amino acid sequence of SEQ ID NO:144 from amino acid 69 to amino acid 98. In further preferred embodiments, the present invention provides a polynucleotide encoding a protein comprising a fragment of the amino acid sequence of SEQ ID NO:144 having biological activity, the fragment preferably comprising eight (more preferably twenty, most preferably thirty) consecutive amino acids of SEQ ID NO:144, or a polynucleotide encoding a protein comprising a fragment of the amino acid sequence of SEQ ID NO:144 having biological activity, the fragment comprising the amino acid sequence from amino acid 44 to amino acid 53 of SEQ ID NO:144.

Other embodiments provide the gene corresponding to the cDNA sequence of SEQ ID NO:143.

In other embodiments, the present invention provides a composition comprising a protein, wherein said protein comprises an amino acid sequence selected from the group consisting of:

(a) the amino acid sequence of SEQ ID NO:144;

(b) the amino acid sequence of SEQ ID NO:144 from amino acid 69 to amino acid 98;

(c) fragments of the amino acid sequence of SEQ ID NO:144 comprising eight consecutive amino acids of SEQ ID NO:144; and

(d) the amino acid sequence encoded by the cDNA insert of clone du372_{—}1 deposited under accession number ATCC 98501;

the protein being substantially free from other mammalian proteins. Preferably such protein comprises the amino acid sequence of SEQ ID NO:144 or the amino acid sequence of SEQ ID NO:144 from amino acid 69 to amino acid 98. In further preferred embodiments, the present invention provides a protein comprising a fragment of the amino acid sequence of SEQ ID NO:144 having biological activity, the fragment preferably comprising eight (more preferably twenty, most preferably thirty) consecutive amino acids of SEQ ID NO:144, or a protein comprising a fragment of the amino acid sequence of SEQ ID NO:144 having biological activity, the fragment comprising the amino acid sequence from amino acid 44 to amino acid 53 of SEQ ID NO:144.

In one embodiment, the present invention provides a composition comprising an isolated polynucleotide selected from the group consisting of:

(a) a polynucleotide comprising the nucleotide sequence of SEQ ID NO:145;

(b) a polynucleotide comprising the nucleotide sequence of SEQ ID NO:145 from nucleotide 32 to nucleotide 586;

(c) a polynucleotide comprising the nucleotide sequence of SEQ ID NO:145 from nucleotide 92 to nucleotide 586;

(d) a polynucleotide comprising the nucleotide sequence of SEQ ID NO:145 from nucleotide 1 to nucleotide 481;

(e) a polynucleotide comprising the nucleotide sequence of the full-length protein coding sequence of clone ej90_{—}5 deposited under accession number ATCC 98501;

(f) a polynucleotide encoding the full-length protein encoded by the cDNA insert of clone ej90_{—}5 deposited under accession number ATCC 98501;

(g) a polynucleotide comprising the nucleotide sequence of a mature protein coding sequence of clone ej90_{—}5 deposited under accession number ATCC 98501;

(h) a polynucleotide encoding a mature protein encoded by the cDNA insert of clone ej90_{—}5 deposited under accession number ATCC 98501;

(i) a polynucleotide encoding a protein comprising the amino acid sequence of SEQ ID NO:146;

(j) a polynucleotide encoding a protein comprising a fragment of the amino acid sequence of SEQ ID NO:146 having biological activity, the fragment comprising eight consecutive amino acids of SEQ ID NO:146;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:145 from nucleotide 32 to nucleotide 586; the nucleotide sequence of SEQ ID NO:145 from nucleotide 92 to nucleotide 586; the nucleotide sequence of SEQ ID NO:145 from nucleotide 1 to nucleotide 481; the nucleotide sequence of the full-length protein coding sequence of clone ej90_{—}5 deposited under accession number ATCC 98501; or the nucleotide sequence of a mature protein coding sequence of clone ej90_{—}5 deposited under accession number ATCC 98501. In other preferred embodiments, the polynucleotide encodes the full-length or a mature protein encoded by the cDNA insert of clone ej90_{—}5 deposited under accession number ATCC 98501. In yet other preferred embodiments, the present invention provides a polynucleotide encoding a protein comprising the amino acid sequence of SEQ ID NO:146 from amino acid 1 to amino acid 150. In further preferred embodiments, the present invention provides a polynucleotide encoding a protein comprising a fragment of the amino acid sequence of SEQ ID NO:146 having biological activity, the fragment preferably comprising eight (more preferably twenty, most preferably thirty) consecutive amino acids of SEQ ID NO:146, or a polynucleotide encoding a protein comprising a fragment of the amino acid sequence of SEQ ID NO:146 having biological activity, the fragment comprising the amino acid sequence from amino acid 87 to amino acid 96 of SEQ ID NO:146.

Other embodiments provide the gene corresponding to the cDNA sequence of SEQ ID NO:145.

In other embodiments, the present invention provides a composition comprising a protein, wherein said protein comprises an amino acid sequence selected from the group consisting of:

(a) the amino acid sequence of SEQ ID NO:146;

(b) the amino acid sequence of SEQ ID NO:146 from amino acid 1 to amino acid 150;

(c) fragments of the amino acid sequence of SEQ ID NO:146 comprising eight consecutive amino acids of SEQ ID NO:146; and

(d) the amino acid sequence encoded by the cDNA insert of clone ej90_{—}5 deposited under accession number ATCC 98501;

the protein being substantially free from other mammalian proteins. Preferably such protein comprises the amino acid sequence of SEQ ID NO:146 or the amino acid sequence of SEQ ID NO:146 from amino acid 1 to amino acid 150. In further preferred embodiments, the present invention provides a protein comprising a fragment of the amino acid sequence of SEQ ID NO:146 having biological activity, the fragment preferably comprising eight (more preferably twenty, most preferably thirty) consecutive amino acids of SEQ ID NO:146, or a protein comprising a fragment of the amino acid sequence of SEQ ID NO:146 having biological activity, the fragment comprising the amino acid sequence from amino acid 87 to amino acid 96 of SEQ ID NO:146.

In one embodiment, the present invention provides a composition comprising an isolated polynucleotide selected from the group consisting of:

(a) a polynucleotide comprising the nucleotide sequence of SEQ ID NO:147;

(b) a polynucleotide comprising the nucleotide sequence of SEQ ID NO:147 from nucleotide 281 to nucleotide 1786;

(c) a polynucleotide comprising the nucleotide sequence of SEQ ID NO:147 from nucleotide 332 to nucleotide 1786;

(d) a polynucleotide comprising the nucleotide sequence of SEQ ID NO:147 from nucleotide 1 to nucleotide 574;

(e) a polynucleotide comprising the nucleotide sequence of the full-length protein coding sequence of clone ic2_{—}6 deposited under accession number ATCC 98501;

(f) a polynucleotide encoding the full-length protein encoded by the cDNA insert of clone ic2_{—}6 deposited under accession number ATCC 98501;

(g) a polynucleotide comprising the nucleotide sequence of a mature protein coding sequence of clone ic2_{—}6 deposited under accession number ATCC 98501;

(h) a polynucleotide encoding a mature protein encoded by the cDNA insert of clone ic2_{—}6 deposited under accession number ATCC 98501;

(i) a polynucleotide encoding a protein comprising the amino acid sequence of SEQ ID NO:148;

(j) a polynucleotide encoding a protein comprising a fragment of the amino acid sequence of SEQ ID NO:148 having biological activity, the fragment comprising eight consecutive amino acids of SEQ ID NO:148;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:147 from nucleotide 281 to nucleotide 1786; the nucleotide sequence of SEQ ID NO:147 from nucleotide 332 to nucleotide 1786; the nucleotide sequence of SEQ ID NO:147 from nucleotide 1 to nucleotide 574; the nucleotide sequence of the full-length protein coding sequence of clone ic2_{—}6 deposited under accession number ATCC 98501; or the nucleotide sequence of a mature protein coding sequence of clone ic2_{—}6 deposited under accession number ATCC 98501. In other preferred embodiments, the polynucleotide encodes the full-length or a mature protein encoded by the cDNA insert of clone ic2_{—}6 deposited under accession number ATCC 98501. In yet other preferred embodiments, the present invention provides a polynucleotide encoding a protein comprising the amino acid sequence of SEQ ID NO:148 from amino acid 1 to amino acid 98. In further preferred embodiments, the present invention provides a polynucleotide encoding a protein comprising a fragment of the amino acid sequence of SEQ ID NO:148 having biological activity, the fragment preferably comprising eight (more preferably twenty, most preferably thirty) consecutive amino acids of SEQ ID NO:148, or a polynucleotide encoding a protein comprising a fragment of the amino acid sequence of SEQ ID NO:148 having biological activity, the fragment comprising the amino acid sequence from amino acid 246 to amino acid 255 of SEQ ID NO:148.

Other embodiments provide the gene corresponding to the cDNA sequence of SEQ ID NO:147.

In other embodiments, the present invention provides a composition comprising a protein, wherein said protein comprises an amino acid sequence selected from the group consisting of:

(a) the amino acid sequence of SEQ ID NO:148;

(b) the amino acid sequence of SEQ ID NO:148 from amino acid 1 to amino acid 98;

(c) fragments of the amino acid sequence of SEQ ID NO:148 comprising eight consecutive amino acids of SEQ ID NO:148; and

(d) the amino acid sequence encoded by the cDNA insert of clone ic2_{—}6 deposited under accession number ATCC 98501;

the protein being substantially free from other mammalian proteins. Preferably such protein comprises the amino acid sequence of SEQ ID NO:148 or the amino acid sequence of SEQ ID NO:148 from amino acid 1 to amino acid 98. In further preferred embodiments, the present invention provides a protein comprising a fragment of the amino acid sequence of SEQ ID NO:148 having biological activity, the fragment preferably comprising eight (more preferably twenty, most preferably thirty) consecutive amino acids of SEQ ID NO:148, or a protein comprising a fragment of the amino acid sequence of SEQ ID NO:148 having biological activity, the fragment comprising the amino acid sequence from amino acid 246 to amino acid 255 of SEQ ID NO:148.

In one embodiment, the present invention provides a composition comprising an isolated polynucleotide selected from the group consisting of:

(a) a polynucleotide comprising the nucleotide sequence of SEQ ID NO:159;

(b) a polynucleotide comprising the nucleotide sequence of SEQ ID NO:159 from nucleotide 69 to nucleotide 908;

(c) a polynucleotide comprising the nucleotide sequence of SEQ ID NO:159 from nucleotide 270 to nucleotide 908;

(d) a polynucleotide comprising the nucleotide sequence of the full-length protein coding sequence of clone bn97_{—}1 deposited under accession number ATCC 98535;

(e) a polynucleotide encoding the full-length protein encoded by the cDNA insert of clone bn97_{—}1 deposited under accession number ATCC 98535;

(f) a polynucleotide comprising the nucleotide sequence of a mature protein coding sequence of clone bn97_{—}1 deposited under accession number ATCC 98535;

(g) a polynucleotide encoding a mature protein encoded by the cDNA insert of clone bn97_{—}1 deposited under accession number ATCC 98535;

(h) a polynucleotide encoding a protein comprising the amino acid sequence of SEQ ID NO:160;

(i) a polynucleotide encoding a protein comprising a fragment of the amino acid sequence of SEQ ID NO:160 having biological activity, the fragment comprising eight consecutive amino acids of SEQ ID NO:160;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:159 from nucleotide 69 to nucleotide 908; the nucleotide sequence of SEQ ID NO:159 from nucleotide 270 to nucleotide 908; the nucleotide sequence of the full-length protein coding sequence of clone bn97_{—}1 deposited under accession number ATCC 98535; or the nucleotide sequence of a mature protein coding sequence of clone bn97_{—}1 deposited under accession number ATCC 98535. In other preferred embodiments, the polynucleotide encodes the full-length or a mature protein encoded by the cDNA insert of clone bn97_{—}1 deposited under accession number ATCC 98535. In yet other preferred embodiments, the present invention provides a polynucleotide encoding a protein comprising the amino acid sequence of SEQ ID NO:160 from amino acid 1 to amino acid 83. In further preferred embodiments, the present invention provides a polynucleotide encoding a protein comprising a fragment of the amino acid sequence of SEQ ID NO:160 having biological activity, the fragment preferably comprising eight (more preferably twenty, most preferably thirty) consecutive amino acids of SEQ ID NO:160, or a polynucleotide encoding a protein comprising a fragment of the amino acid sequence of SEQ ID NO:160 having biological activity, the fragment comprising the amino acid sequence from amino acid 135 to amino acid 144 of SEQ ID NO:160.

Other embodiments provide the gene corresponding to the cDNA sequence of SEQ ID NO:159.

In other embodiments, the present invention provides a composition comprising a protein, wherein said protein comprises an amino acid sequence selected from the group consisting of:

(a) the amino acid sequence of SEQ ID NO:160;

(b) the amino acid sequence of SEQ ID NO:160 from amino acid 1 to amino acid 83;

(c) fragments of the amino acid sequence of SEQ ID NO:160 comprising eight consecutive amino acids of SEQ ID NO:160; and

(d) the amino acid sequence encoded by the cDNA insert of clone bn97_{—}1 deposited under accession number ATCC 98535;

the protein being substantially free from other mammalian proteins. Preferably such protein comprises the amino acid sequence of SEQ ID NO:160 or the amino acid sequence of SEQ ID NO:160 from amino acid 1 to amino acid 83. In further preferred embodiments, the present invention provides a protein comprising a fragment of the amino acid sequence of SEQ ID NO:160 having biological activity, the fragment preferably comprising eight (more preferably twenty, most preferably thirty) consecutive amino acids of SEQ ID NO:160, or a protein comprising a fragment of the amino acid sequence of SEQ ID NO:160 having biological activity, the fragment comprising the amino acid sequence from amino acid 135 to amino acid 144 of SEQ ID NO:160.

In one embodiment, the present invention provides a composition comprising an isolated polynucleotide selected from the group consisting of:

(a) a polynucleotide comprising the nucleotide sequence of SEQ ID NO:161;

(b) a polynucleotide comprising the nucleotide sequence of SEQ ID NO:161 from nucleotide 562 to nucleotide 777;

(c) a polynucleotide comprising the nucleotide sequence of SEQ ID NO:161 from nucleotide 236 to nucleotide 673;

(d) a polynucleotide comprising the nucleotide sequence of the full-length protein coding sequence of clone bn268_{—}11 deposited under accession number ATCC 98535;

(e) a polynucleotide encoding the full-length protein encoded by the cDNA insert of clone bn268_{—}11 deposited under accession number ATCC 98535;

(f) a polynucleotide comprising the nucleotide sequence of a mature protein coding sequence of clone bn268_{—}11 deposited under accession number ATCC 98535;

(g) a polynucleotide encoding a mature protein encoded by the cDNA insert of clone bn268_{—}11 deposited under accession number ATCC 98535;

(h) a polynucleotide encoding a protein comprising the amino acid sequence of SEQ ID NO:162;

(i) a polynucleotide encoding a protein comprising a fragment of the amino acid sequence of SEQ ID NO:162 having biological activity, the fragment comprising eight consecutive amino acids of SEQ ID NO:162;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:161 from nucleotide 562 to nucleotide 777; the nucleotide sequence of SEQ ID NO:161 from nucleotide 236 to nucleotide 673; the nucleotide sequence of the full-length protein coding sequence of clone bn268_{—}11 deposited under accession number ATCC 98535; or the nucleotide sequence of a mature protein coding sequence of clone bn268_{—}11 deposited under accession number ATCC 98535. In other preferred embodiments, the polynucleotide encodes the full-length or a mature protein encoded by the cDNA insert of clone bn268_{—}11 deposited under accession number ATCC 98535. In yet other preferred embodiments, the present invention provides a polynucleotide encoding a protein comprising the amino acid sequence of SEQ ID NO:162 from amino acid 1 to amino acid 37. In further preferred embodiments, the present invention provides a polynucleotide encoding a protein comprising a fragment of the amino acid sequence of SEQ ID NO:162 having biological activity, the fragment preferably comprising eight (more preferably twenty, most preferably thirty) consecutive amino acids of SEQ ID NO:4, or a polynucleotide encoding a protein comprising a fragment of the amino acid sequence of SEQ ID NO:4 having biological activity, the fragment comprising the amino acid sequence from amino acid 31 to amino acid 40 of SEQ ID NO:162.

Other embodiments provide the gene corresponding to the cDNA sequence of SEQ ID NO:161.

In other embodiments, the present invention provides a composition comprising a protein, wherein said protein comprises an amino acid sequence selected from the group consisting of:

(a) the amino acid sequence of SEQ ID NO:162;

(b) the amino acid sequence of SEQ ID NO:162 from amino acid 1 to amino acid 37;

(c) fragments of the amino acid sequence of SEQ ID NO:162 comprising eight consecutive amino acids of SEQ ID NO:162; and

(d) the amino acid sequence encoded by the cDNA insert of clone bn268_{—}11 deposited under accession number ATCC 98535;

the protein being substantially free from other mammalian proteins. Preferably such protein comprises the amino acid sequence of SEQ ID NO:162 or the amino acid sequence of SEQ ID NO:162 from amino acid 1 to amino acid 37. In further preferred embodiments, the present invention provides a protein comprising a fragment of the amino acid sequence of SEQ ID NO:162 having biological activity, the fragment preferably comprising eight (more preferably twenty, most preferably thirty) consecutive amino acids of SEQ ID NO:162, or a protein comprising a fragment of the amino acid sequence of SEQ ID NO:162 having biological activity, the fragment comprising the amino acid sequence from amino acid 31 to amino acid 40 of SEQ ID NO:162.

In one embodiment, the present invention provides a composition comprising an isolated polynucleotide selected from the group consisting of:

(a) a polynucleotide comprising the nucleotide sequence of SEQ ID NO:163;

(b) a polynucleotide comprising the nucleotide sequence of SEQ ID NO:163 from nucleotide 286 to nucleotide 1686;

(c) a polynucleotide comprising the nucleotide sequence of SEQ ID NO:163 from nucleotide 544 to nucleotide 1686;

(d) a polynucleotide comprising the nucleotide sequence of SEQ ID NO:163 from nucleotide 365 to nucleotide 1160;

(e) a polynucleotide comprising the nucleotide sequence of the full-length protein coding sequence of clone cb96_{—}10 deposited under accession number ATCC 98535;

(f) a polynucleotide encoding the full-length protein encoded by the cDNA insert of clone cb96_{—}10 deposited under accession number ATCC 98535;

(g) a polynucleotide comprising the nucleotide sequence of a mature protein coding sequence of clone cb96_{—}10 deposited under accession number ATCC 98535;

(h) a polynucleotide encoding a mature protein encoded by the cDNA insert of clone cb96_{—}10 deposited under accession number ATCC 98535;

(i) a polynucleotide encoding a protein comprising the amino acid sequence of SEQ ID NO:164;

(j) a polynucleotide encoding a protein comprising a fragment of the amino acid sequence of SEQ ID NO:164 having biological activity, the fragment comprising eight consecutive amino acids of SEQ ID NO:164;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:163 from nucleotide 286 to nucleotide 1686; the nucleotide sequence of SEQ ID NO:163 from nucleotide 544 to nucleotide 1686; the nucleotide sequence of SEQ ID NO:163 from nucleotide 365 to nucleotide 1160; the nucleotide sequence of the full-length protein coding sequence of clone cb96_{—}10 deposited under accession number ATCC 98535; or the nucleotide sequence of a mature protein coding sequence of clone cb96_{—}10 deposited under accession number ATCC 98535. In other preferred embodiments, the polynucleotide encodes the full-length or a mature protein encoded by the cDNA insert of clone cb96_{—}10 deposited under accession number ATCC 98535. In yet other preferred embodiments, the present invention provides a polynucleotide encoding a protein comprising the amino acid sequence of SEQ ID NO:164 from amino acid 28 to amino acid 395. In further preferred embodiments, the present invention provides a polynucleotide encoding a protein comprising a fragment of the amino acid sequence of SEQ ID NO:164 having biological activity, the fragment preferably comprising eight (more preferably twenty, most preferably thirty) consecutive amino acids of SEQ ID NO:164, or a polynucleotide encoding a protein comprising a fragment of the amino acid sequence of SEQ ID NO:164 having biological activity, the fragment comprising the amino acid sequence from amino acid 228 to amino acid 237 of SEQ ID NO:164.

Other embodiments provide the gene corresponding to the cDNA sequence of SEQ ID NO:163.

In other embodiments, the present invention provides a composition comprising a protein, wherein said protein comprises an amino acid sequence selected from the group consisting of:

(a) the amino acid sequence of SEQ ID NO:164;

(b) the amino acid sequence of SEQ ID NO:164 from amino acid 28 to amino acid 395;

(c) fragments of the amino acid sequence of SEQ ID NO:164 comprising eight consecutive amino acids of SEQ ID NO:164; and

(d) the amino acid sequence encoded by the cDNA insert of clone cb96_{—}10 deposited under accession number ATCC 98535;

the protein being substantially free from other mammalian proteins. Preferably such protein comprises the amino acid sequence of SEQ ID NO:164 or the amino acid sequence of SEQ ID NO:164 from amino acid 28 to amino acid 395. In further preferred embodiments, the present invention provides a protein comprising a fragment of the amino acid sequence of SEQ ID NO:164 having biological activity, the fragment preferably comprising eight (more preferably twenty, most preferably thirty) consecutive amino acids of SEQ ID NO:164, or a protein comprising a fragment of the amino acid sequence of SEQ ID NO:164 having biological activity, the fragment comprising the amino acid sequence from amino acid 228 to amino acid 237 of SEQ ID NO:164.

In one embodiment, the present invention provides a composition comprising an isolated polynucleotide selected from the group consisting of:

(a) a polynucleotide comprising the nucleotide sequence of SEQ ID NO:165;

(b) a polynucleotide comprising the nucleotide sequence of SEQ ID NO:165 from nucleotide 99 to nucleotide 1049;

(c) a polynucleotide comprising the nucleotide sequence of SEQ ID NO:165 from nucleotide 222 to nucleotide 1049;

(d) a polynucleotide comprising the nucleotide sequence of SEQ ID NO:165 from nucleotide 632 to nucleotide 998;

(e) a polynucleotide comprising the nucleotide sequence of the full-length protein coding sequence of clone cb213_{—}11 deposited under accession number ATCC 98535;

(f) a polynucleotide encoding the full-length protein encoded by the cDNA insert of clone cb213_{—}11 deposited under accession number ATCC 98535;

(g) a polynucleotide comprising the nucleotide sequence of a mature protein coding sequence of clone cb213_{—}11 deposited under accession number ATCC 98535;

(h) a polynucleotide encoding a mature protein encoded by the cDNA insert of clone cb213_{—}11 deposited under accession number ATCC 98535;

(i) a polynucleotide encoding a protein comprising the amino acid sequence of SEQ ID NO:166;

(j) a polynucleotide encoding a protein comprising a fragment of the amino acid sequence of SEQ ID NO:166 having biological activity, the fragment comprising eight consecutive amino acids of SEQ ID NO:166;

(k) a polynucleotide which is an allelic variant of a polynucleotide of (a)-(h) above;

(l) a polynucleotide which encodes a species homologue of the protein of (i) or 0(j) above; and

(m) a polynucleotide that hybridizes under stringent conditions to any one of the polynucleotides specified in (a)-(j).

Preferably, such polynucleotide comprises the nucleotide sequence of SEQ ID NO:166 from nucleotide 99 to nucleotide 1049; the nucleotide sequence of SEQ ID NO:166 from nucleotide 222 to nucleotide 1049; the nucleotide sequence of SEQ ID NO:166 from nucleotide 632 to nucleotide 998; the nucleotide sequence of the full-length protein coding sequence of clone cb213_{—}11 deposited under accession number ATCC 98535; or the nucleotide sequence of a mature protein coding sequence of clone cb213_{—}11 deposited under accession number ATCC 98535. In other preferred embodiments, the polynucleotide encodes the full-length or a mature protein encoded by the cDNA insert of clone cb213_{—}11 deposited under accession number ATCC 98535. In yet other preferred embodiments, the present invention provides a polynucleotide encoding a protein comprising the amino acid sequence of SEQ ID NO:166 from amino acid 187 to amino acid 300. In further preferred embodiments, the present invention provides a polynucleotide encoding a protein comprising a fragment of the amino acid sequence of SEQ ID NO:166 having biological activity, the fragment preferably comprising eight (more preferably twenty, most preferably thirty) consecutive amino acids of SEQ ID NO:166, or a polynucleotide encoding a protein comprising a fragment of the amino acid sequence of SEQ ID NO:166 having biological activity, the fragment comprising the amino acid sequence from amino acid 153 to amino acid 162 of SEQ ID NO:166.

Other embodiments provide the gene corresponding to the cDNA sequence of SEQ ID NO:165.

In other embodiments, the present invention provides a composition comprising a protein, wherein said protein comprises an amino acid sequence selected from the group consisting of:

(a) the amino acid sequence of SEQ ID NO:166;

(b) the amino acid sequence of SEQ ID NO:166 from amino acid 187 to amino acid 300;

(c) fragments of the amino acid sequence of SEQ ID NO:166 comprising eight consecutive amino acids of SEQ ID NO:166; and

(d) the amino acid sequence encoded by the cDNA insert of clone cb213_{—}11 deposited under accession number ATCC 98535;

the protein being substantially free from other mammalian proteins. Preferably such protein comprises the amino acid sequence of SEQ ID NO:166 or the amino acid sequence of SEQ ID NO:166 from amino acid 187 to amino acid 300. In further preferred embodiments, the present invention provides a protein comprising a fragment of the amino acid sequence of SEQ ID NO:166 having biological activity, the fragment preferably comprising eight (more preferably twenty, most preferably thirty) consecutive amino acids of SEQ ID NO:166, or a protein comprising a fragment of the amino acid sequence of SEQ ID NO:166 having biological activity, the fragment comprising the amino acid sequence from amino acid 153 to amino acid 162 of SEQ ID NO:166.

In one embodiment, the present invention provides a composition comprising an isolated polynucleotide selected from the group consisting of:

(a) a polynucleotide comprising the nucleotide sequence of SEQ ID NO:167;

(b) a polynucleotide comprising the nucleotide sequence of SEQ ID NO:167 from nucleotide 3003 to nucleotide 3137;

(c) a polynucleotide comprising the nucleotide sequence of SEQ ID NO:167 from nucleotide 3072 to nucleotide 3137;

(d) a polynucleotide comprising the nucleotide sequence of SEQ ID NO:167 from nucleotide 2713 to nucleotide 3114;

(e) a polynucleotide comprising the nucleotide sequence of the full-length protein coding sequence of clone cj457_{—}4 deposited under accession number ATCC 98535;

(f) a polynucleotide encoding the full-length protein encoded by the cDNA insert of clone cj457_{—}4 deposited under accession number ATCC 98535;

(g) a polynucleotide comprising the nucleotide sequence of a mature protein coding sequence of clone cj457_{—}4 deposited under accession number ATCC 98535;

(h) a polynucleotide encoding a mature protein encoded by the cDNA insert of clone cj457_{—}4 deposited under accession number ATCC 98535;

(i) a polynucleotide encoding a protein comprising the amino acid sequence of SEQ ID NO:168;

(j) a polynucleotide encoding a protein comprising a fragment of the amino acid sequence of SEQ ID NO:168 having biological activity, the fragment comprising eight consecutive amino acids of SEQ ID NO:168;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:167 from nucleotide 3003 to nucleotide 3137; the nucleotide sequence of SEQ ID NO:167 from nucleotide 3072 to nucleotide 3137; the nucleotide sequence of SEQ ID NO:167 from nucleotide 2713 to nucleotide 3114; the nucleotide sequence of the full-length protein coding sequence of clone cj457_{—}4 deposited under accession number ATCC 98535; or the nucleotide sequence of a mature protein coding sequence of clone cj457_{—}4 deposited under accession number ATCC 98535. In other preferred embodiments, the polynucleotide encodes the full-length or a mature protein encoded by the cDNA insert of clone cj457_{—}4 deposited under accession number ATCC 98535. In yet other preferred embodiments, the present invention provides a polynucleotide encoding a protein comprising the amino acid sequence of SEQ ID NO:168 from amino acid 1 to amino acid 37. In further preferred embodiments, the present invention provides a polynucleotide encoding a protein comprising a fragment of the amino acid sequence of SEQ ID NO:168 having biological activity, the fragment preferably comprising eight (more preferably twenty, most preferably thirty) consecutive amino acids of SEQ ID NO:168, or a polynucleotide encoding a protein comprising a fragment of the amino acid sequence of SEQ ID NO:168 having biological activity, the fragment comprising the amino acid sequence from amino acid 17 to amino acid 26 of SEQ ID NO:168.

Other embodiments provide the gene corresponding to the cDNA sequence of SEQ ID NO:167.

In other embodiments, the present invention provides a composition comprising a protein, wherein said protein comprises an amino acid sequence selected from the group consisting of:

(a) the amino acid sequence of SEQ ID NO:168;

(b) the amino acid sequence of SEQ ID NO:168 from amino acid 1 to amino acid 37;

(c) fragments of the amino acid sequence of SEQ ID NO:168 comprising eight consecutive amino acids of SEQ ID NO:168; and

(d) the amino acid sequence encoded by the cDNA insert of clone cj457_{—}4 deposited under accession number ATCC 98535;

the protein being substantially free from other mammalian proteins. Preferably such protein comprises the amino acid sequence of SEQ ID NO:168 or the amino acid sequence of SEQ ID NO:168 from amino acid 1 to amino acid 37. In further preferred embodiments, the present invention provides a protein comprising a fragment of the amino acid sequence of SEQ ID NO:168 having biological activity, the fragment preferably comprising eight (more preferably twenty, most preferably thirty) consecutive amino acids of SEQ ID NO:168, or a protein comprising a fragment of the amino acid sequence of SEQ ID NO:168 having biological activity, the fragment comprising the amino acid sequence from amino acid 17 to amino acid 26 of SEQ ID NO:168.

In one embodiment, the present invention provides a composition comprising an isolated polynucleotide selected from the group consisting of:

(a) a polynucleotide comprising the nucleotide sequence of SEQ ID NO:169;

(b) a polynucleotide comprising the nucleotide sequence of SEQ ID NO:169 from nucleotide 284 to nucleotide 1357;

(c) a polynucleotide comprising the nucleotide sequence of SEQ ID NO:169 from nucleotide 603 to nucleotide 1233;

(d) a polynucleotide comprising the nucleotide sequence of the full-length protein coding sequence of clone cz653_{—}11 deposited under accession number ATCC 98535;

(e) a polynucleotide encoding the full-length protein encoded by the cDNA insert of clone cz653_{—}11 deposited under accession number ATCC 98535;

(f) a polynucleotide comprising the nucleotide sequence of a mature protein coding sequence of clone cz653_{—}11 deposited under accession number ATCC 98535;

(g) a polynucleotide encoding a mature protein encoded by the cDNA insert of clone cz653_{—}11 deposited under accession number ATCC 98535;

(h) a polynucleotide encoding a protein comprising the amino acid sequence of SEQ ID NO:170;

(i) a polynucleotide encoding a protein comprising a fragment of the amino acid sequence of SEQ ID NO:12 having biological activity, the fragment comprising eight consecutive amino acids of SEQ ID NO:170;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:169 from nucleotide 284 to nucleotide 1357; the nucleotide sequence of SEQ ID NO:169 from nucleotide 603 to nucleotide 1233; the nucleotide sequence of the full-length protein coding sequence of clone cz653_{—}11 deposited under accession number ATCC 98535; or the nucleotide sequence of a mature protein coding sequence of clone cz653_{—}11 deposited under accession number ATCC 98535. In other preferred embodiments, the polynucleotide encodes the full-length or a mature protein encoded by the cDNA insert of clone cz653_{—}11 deposited under accession number ATCC 98535. In yet other preferred embodiments, the present invention provides a polynucleotide encoding a protein comprising the amino acid sequence of SEQ ID NO:170 from amino acid 147 to amino acid 358. In further preferred embodiments, the present invention provides a polynucleotide encoding a protein comprising a fragment of the amino acid sequence of SEQ ID NO:170 having biological activity, the fragment preferably comprising eight (more preferably twenty, most preferably thirty) consecutive amino acids of SEQ ID NO:170, or a polynucleotide encoding a protein comprising a fragment of the amino acid sequence of SEQ ID NO:170 having biological activity, the fragment comprising the amino acid sequence from amino acid 174 to amino acid 183 of SEQ ID NO:170.

Other embodiments provide the gene corresponding to the cDNA sequence of SEQ ID NO:169.

In other embodiments, the present invention provides a composition comprising a protein, wherein said protein comprises an amino acid sequence selected from the group consisting of:

(a) the amino acid sequence of SEQ ID NO:170;

(b) the amino acid sequence of SEQ ID NO:170 from amino acid 147 to amino acid 358;

(c) fragments of the amino acid sequence of SEQ ID NO:170 comprising eight consecutive amino acids of SEQ ID NO:170; and

(d) the amino acid sequence encoded by the cDNA insert of clone cz653_{—}11 deposited under accession number ATCC 98535;

the protein being substantially free from other mammalian proteins. Preferably such protein comprises the amino acid sequence of SEQ ID NO:170 or the amino acid sequence of SEQ ID NO:170 from amino acid 147 to amino acid 358. In further preferred embodiments, the present invention provides a protein comprising a fragment of the amino acid sequence of SEQ ID NO:170 having biological activity, the fragment preferably comprising eight (more preferably twenty, most preferably thirty) consecutive amino acids of SEQ ID NO:170, or a protein comprising a fragment of the amino acid sequence of SEQ ID NO:170 having biological activity, the fragment comprising the amino acid sequence from amino acid 174 to amino acid 183 of SEQ ID NO:170.

In one embodiment, the present invention provides a composition comprising an isolated polynucleotide selected from the group consisting of:

(a) a polynucleotide comprising the nucleotide sequence of SEQ ID NO:171;

(b) a polynucleotide comprising the nucleotide sequence of SEQ ID NO:171 from nucleotide 621 to nucleotide 1763;

(c) a polynucleotide comprising the nucleotide sequence of SEQ ID NO:171 from nucleotide 1461 to nucleotide 1763;

(d) a polynucleotide comprising the nucleotide sequence of the full-length protein coding sequence of clone dx138_{—}4 deposited under accession number ATCC 98535;

(e) a polynucleotide encoding the full-length protein encoded by the cDNA insert of clone dx138_{—}4 deposited under accession number ATCC 98535;

(f) a polynucleotide comprising the nucleotide sequence of a mature protein coding sequence of clone dx138_{—}4 deposited under accession number ATCC 98535;

(g) a polynucleotide encoding a mature protein encoded by the cDNA insert of clone dx138_{—}4 deposited under accession number ATCC 98535;

(h) a polynucleotide encoding a protein comprising the amino acid sequence of SEQ ID NO:172;

(i) a polynucleotide encoding a protein comprising a fragment of the amino acid sequence of SEQ ID NO:172 having biological activity, the fragment comprising eight consecutive amino acids of SEQ ID NO:172;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:171 from nucleotide 621 to nucleotide 1763; the nucleotide sequence of SEQ ID NO:171 from nucleotide 1461 to nucleotide 1763; the nucleotide sequence of the full-length protein coding sequence of clone dx138_{—}4 deposited under accession number ATCC 98535; or the nucleotide sequence of a mature protein coding sequence of clone dx138_{—}4 deposited under accession number ATCC 98535. In other preferred embodiments, the polynucleotide encodes the full-length or a mature protein encoded by the cDNA insert of clone dx138_{—}4 deposited under accession number ATCC 98535. In yet other preferred embodiments, the present invention provides a polynucleotide encoding a protein comprising the amino acid sequence of SEQ ID NO:172 from amino acid 83 to amino acid 229. In further preferred embodiments, the present invention provides a polynucleotide encoding a protein comprising a fragment of the amino acid sequence of SEQ ID NO:172 having biological activity, the fragment preferably comprising eight (more preferably twenty, most preferably thirty) consecutive amino acids of SEQ ID NO:172, or a polynucleotide encoding a protein comprising a fragment of the amino acid sequence of SEQ ID NO:172 having biological activity, the fragment comprising the amino acid sequence from amino acid 185 to amino acid 194 of SEQ ID NO:172.

Other embodiments provide the gene corresponding to the cDNA sequence of SEQ ID NO:171.

In other embodiments, the present invention provides a composition comprising a protein, wherein said protein comprises an amino acid sequence selected from the group consisting of:

(a) the amino acid sequence of SEQ ID NO:172;

(b) the amino acid sequence of SEQ ID NO:172 from amino acid 83 to amino acid 229;

(c) fragments of the amino acid sequence of SEQ ID NO:172 comprising eight consecutive amino acids of SEQ ID NO:172; and

(d) the amino acid sequence encoded by the cDNA insert of clone dx138_{—}4 deposited under accession number ATCC 98535;

the protein being substantially free from other mammalian proteins. Preferably such protein comprises the amino acid sequence of SEQ ID NO:172 or the amino acid sequence of SEQ ID NO:172 from amino acid 83 to amino acid 229. In further preferred embodiments, the present invention provides a protein comprising a fragment of the amino acid sequence of SEQ ID NO:172 having biological activity, the fragment preferably comprising eight (more preferably twenty, most preferably thirty) consecutive amino acids of SEQ ID NO:172, or a protein comprising a fragment of the amino acid sequence of SEQ ID NO:172 having biological activity, the fragment comprising the amino acid sequence from amino acid 185 to amino acid 194 of SEQ ID NO:172.

In one embodiment, the present invention provides a composition comprising an isolated polynucleotide selected from the group consisting of:

(a) a polynucleotide comprising the nucleotide sequence of SEQ ID NO:173;

(b) a polynucleotide comprising the nucleotide sequence of SEQ ID NO:173 from nucleotide 119 to nucleotide 295;

(c) a polynucleotide comprising the nucleotide sequence of SEQ ID NO:173 from nucleotide 191 to nucleotide 295;

(d) a polynucleotide comprising the nucleotide sequence of the full-length protein coding sequence of clone ij167_{—}5 deposited under accession number ATCC 98535;

(e) a polynucleotide encoding the full-length protein encoded by the cDNA insert of clone ij167_{—}5 deposited under accession number ATCC 98535;

(f) a polynucleotide comprising the nucleotide sequence of a mature protein coding sequence of clone ij167_{—}5 deposited under accession number ATCC 98535;

(g) a polynucleotide encoding a mature protein encoded by the cDNA insert of clone ij167_{—}5 deposited under accession number ATCC 98535;

(h) a polynucleotide encoding a protein comprising the amino acid sequence of SEQ ID NO:174;

(i) a polynucleotide encoding a protein comprising a fragment of the amino acid sequence of SEQ ID NO:174 having biological activity, the fragment comprising eight consecutive amino acids of SEQ ID NO:174;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:174 from nucleotide 119 to nucleotide 295; the nucleotide sequence of SEQ ID NO:174 from nucleotide 191 to nucleotide 295; the nucleotide sequence of the full-length protein coding sequence of clone ij167_{—}5 deposited under accession number ATCC 98535; or the nucleotide sequence of a mature protein coding sequence of clone ij167_{—}5 deposited under accession number ATCC 98535. In other preferred embodiments, the polynucleotide encodes the full-length or a mature protein encoded by the cDNA insert of clone ij167_{—}5 deposited under accession number ATCC 98535. In yet other preferred embodiments, the present invention provides a polynucleotide encoding a protein comprising the amino acid sequence of SEQ ID NO:16 from amino acid 1 to amino acid 26. In further preferred embodiments, the present invention provides a polynucleotide encoding a protein comprising a fragment of the amino acid sequence of SEQ ID NO:174 having biological activity, the fragment preferably comprising eight (more preferably twenty, most preferably thirty) consecutive amino acids of SEQ ID NO:174, or a polynucleotide encoding a protein comprising a fragment of the amino acid sequence of SEQ ID NO:174 having biological activity, the fragment comprising the amino acid sequence from amino acid 24 to amino acid 33 of SEQ ID NO:174.

Other embodiments provide the gene corresponding to the cDNA sequence of SEQ ID NO:174.

In other embodiments, the present invention provides a composition comprising a protein, wherein said protein comprises an amino acid sequence selected from the group consisting of:

(a) the amino acid sequence of SEQ ID NO:174;

(b) the amino acid sequence of SEQ ID NO:174 from amino acid 1 to amino acid 26;

(c) fragments of the amino acid sequence of SEQ ID NO:174 comprising eight consecutive amino acids of SEQ ID NO:174; and

(d) the amino acid sequence encoded by the cDNA insert of clone ij167_{—}5 deposited under accession number ATCC 98535;

the protein being substantially free from other mammalian proteins. Preferably such protein comprises the amino acid sequence of SEQ ID NO:174 or the amino acid sequence of SEQ ID NO:174 from amino acid 1 to amino acid 26. In further preferred embodiments, the present invention provides a protein comprising a fragment of the amino acid sequence of SEQ ID NO:174 having biological activity, the fragment preferably comprising eight (more preferably twenty, most preferably thirty) consecutive amino acids of SEQ ID NO:174, or a protein comprising a fragment of the amino acid sequence of SEQ ID NO:174 having biological activity, the fragment comprising the amino acid sequence from amino acid 24 to amino acid 33 of SEQ ID NO:174.

In one embodiment, the present invention provides a composition comprising an isolated polynucleotide selected from the group consisting of:

(a) a polynucleotide comprising the nucleotide sequence of SEQ ID NO:183;

(b) a polynucleotide comprising the nucleotide sequence of SEQ ID NO:183 from nucleotide 25 to nucleotide 1458;

(c) a polynucleotide comprising the nucleotide sequence of SEQ ID NO:183 from nucleotide 21 to nucleotide 730;

(d) a polynucleotide comprising the nucleotide sequence of the full-length protein coding sequence of clone bd107_{—}16 deposited under accession number ATCC 98898;

(e) a polynucleotide encoding the full-length protein encoded by the cDNA insert of clone bd107_{—}16 deposited under accession number ATCC 98898;

(f) a polynucleotide comprising the nucleotide sequence of a mature protein coding sequence of clone bd107_{—}16 deposited under accession number ATCC 98898;

(g) a polynucleotide encoding a mature protein encoded by the cDNA insert of clone bd107_{—}16 deposited under accession number ATCC 98898;

(h) a polynucleotide encoding a protein comprising the amino acid sequence of SEQ ID NO:184;

(i) a polynucleotide encoding a protein comprising a fragment of the amino acid sequence of SEQ ID NO:184 having biological activity, the fragment comprising eight consecutive amino acids of SEQ ID NO:184;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:183 from nucleotide 25 to nucleotide 1458; the nucleotide sequence of SEQ ID NO:183 from nucleotide 21 to nucleotide 730; the nucleotide sequence of the full-length protein coding sequence of clone bd107_{—}16 deposited under accession number ATCC 98898; or the nucleotide sequence of a mature protein coding sequence of clone bd107_{—}16 deposited under accession number ATCC 98898. In other preferred embodiments, the polynucleotide encodes the full-length or a mature protein encoded by the cDNA insert of clone bd107_{—}16 deposited under accession number ATCC 98898. In yet other preferred embodiments, the present invention provides a polynucleotide encoding a protein comprising the amino acid sequence of SEQ ID NO:184 from amino acid 2 to amino acid 118. In further preferred embodiments, the present invention provides a polynucleotide encoding a protein comprising a fragment of the amino acid sequence of SEQ ID NO:184 having biological activity, the fragment preferably comprising eight (more preferably twenty, most preferably thirty) consecutive amino acids of SEQ ID NO:184, or a polynucleotide encoding a protein comprising a fragment of the amino acid sequence of SEQ ID NO:184 having biological activity, the fragment comprising the amino acid sequence from amino acid 234 to amino acid 243 of SEQ ID NO:184.

Other embodiments provide the gene corresponding to the cDNA sequence of SEQ ID NO:183.

In other embodiments, the present invention provides a composition comprising a protein, wherein said protein comprises an amino acid sequence selected from the group consisting of:

(a) the amino acid sequence of SEQ ID NO:184;

(b) the amino acid sequence of SEQ ID NO:184 from amino acid 2 to amino acid 118;

(c) fragments of the amino acid sequence of SEQ ID NO:184 comprising eight consecutive amino acids of SEQ ID NO:184; and

(d) the amino acid sequence encoded by the cDNA insert of clone bd107_{—}16 deposited under accession number ATCC 98898;

the protein being substantially free from other mammalian proteins. Preferably such protein comprises the amino acid sequence of SEQ ID NO:184 or the amino acid sequence of SEQ ID NO:184 from amino acid 2 to amino acid 118. In further preferred embodiments, the present invention provides a protein comprising a fragment of the amino acid sequence of SEQ ID NO:184 having biological activity, the fragment preferably comprising eight (more preferably twenty, most preferably thirty) consecutive amino acids of SEQ ID NO:184, or a protein comprising a fragment of the amino acid sequence of SEQ ID NO:184 having biological activity, the fragment comprising the amino acid sequence from amino acid 234 to amino acid 243 of SEQ ID NO:184.

In one embodiment, the present invention provides a composition comprising an isolated polynucleotide selected from the group consisting of:

(a) a polynucleotide comprising the nucleotide sequence of SEQ ID NO:185;

(b) a polynucleotide comprising the nucleotide sequence of SEQ ID NO:185 from nucleotide 6 to nucleotide 977;

(c) a polynucleotide comprising the nucleotide sequence of SEQ ID NO:185 from nucleotide 87 to nucleotide 977;

(d) a polynucleotide comprising the nucleotide sequence of SEQ ID NO:185 from nucleotide 8 to nucleotide 630;

(e) a polynucleotide comprising the nucleotide sequence of the full-length protein coding sequence of clone bm41_{—}7 deposited under accession number ATCC 98898;

(f) a polynucleotide encoding the full-length protein encoded by the cDNA insert of clone bm41_{—}7 deposited under accession number ATCC 98898;

(g) a polynucleotide comprising the nucleotide sequence of a mature protein coding sequence of clone bm41_{—}7 deposited under accession number ATCC 98898;

(h) a polynucleotide encoding a mature protein encoded by the cDNA insert of clone bm41_{—}7 deposited under accession number ATCC 98898;

(i) a polynucleotide encoding a protein comprising the amino acid sequence of SEQ ID NO:186;

(j) a polynucleotide encoding a protein comprising a fragment of the amino acid sequence of SEQ ID NO:186 having biological activity, the fragment comprising eight consecutive amino acids of SEQ ID NO:4;

(k) a polynucleotide which is an allelic variant of a polynucleotide of (a)-(h) above;

(l) a polynucleotide which encodes a species homologue of the protein of (i) or (j) above; and

(m) a polynucleotide that hybridizes under stringent conditions to any one of the polynucleotides specified in (a)-(j).

Preferably, such polynucleotide comprises the nucleotide sequence of SEQ ID NO:184 from nucleotide 6 to nucleotide 977; the nucleotide sequence of SEQ ID NO:184 from nucleotide 87 to nucleotide 977; the nucleotide sequence of SEQ ID NO:184 from nucleotide 8 to nucleotide 630; the nucleotide sequence of the full-length protein coding sequence of clone bm41_{—}7 deposited under accession number ATCC 98898; or the nucleotide sequence of a mature protein coding sequence of clone bm41_{—}7 deposited under accession number ATCC 98898. In other preferred embodiments, the polynucleotide encodes the full-length or a mature protein encoded by the cDNA insert of clone bm41_{—}7 deposited under accession number ATCC 98898. In yet other preferred embodiments, the present invention provides a polynucleotide encoding a protein comprising the amino acid sequence of SEQ ID NO:4 from amino acid 211 to amino acid 315. In further preferred embodiments, the present invention provides a polynucleotide encoding a protein comprising a fragment of the amino acid sequence of SEQ ID NO:186 having biological activity, the fragment preferably comprising eight (more preferably twenty, most preferably thirty) consecutive amino acids of SEQ ID NO:186, or a polynucleotide encoding a protein comprising a fragment of the amino acid sequence of SEQ ID NO:186 having biological activity, the fragment comprising the amino acid sequence from amino acid 157 to amino acid 166 of SEQ ID NO:186.

Other embodiments provide the gene corresponding to the cDNA sequence of SEQ ID NO:185.

In other embodiments, the present invention provides a composition comprising a protein, wherein said protein comprises an amino acid sequence selected from the group consisting of:

(a) the amino acid sequence of SEQ ID NO:186;

(b) the amino acid sequence of SEQ ID NO:186 from amino acid 211 to amino acid 315;

(c) fragments of the amino acid sequence of SEQ ID NO:186 comprising eight consecutive amino acids of SEQ ID NO:186; and

(d) the amino acid sequence encoded by the cDNA insert of clone bm41_{—}7 deposited under accession number ATCC 98898;

the protein being substantially free from other mammalian proteins. Preferably such protein comprises the amino acid sequence of SEQ ID NO:186 or the amino acid sequence of SEQ ID NO:4 from amino acid 211 to amino acid 315. In further preferred embodiments, the present invention provides a protein comprising a fragment of the amino acid sequence of SEQ ID NO:186 having biological activity, the fragment preferably comprising eight (more preferably twenty, most preferably thirty) consecutive amino acids of SEQ ID NO:186, or a protein comprising a fragment of the amino acid sequence of SEQ ID NO:186 having biological activity, the fragment comprising the amino acid sequence from amino acid 157 to amino acid 166 of SEQ ID NO:186.

In one embodiment, the present invention provides a composition comprising an isolated polynucleotide selected from the group consisting of:

(a) a polynucleotide comprising the nucleotide sequence of SEQ ID NO:187;

(b) a polynucleotide comprising the nucleotide sequence of SEQ ID NO:187 from nucleotide 168 to nucleotide 962;

(c) a polynucleotide comprising the nucleotide sequence of SEQ ID NO:187 from nucleotide 351 to nucleotide 962;

(d) a polynucleotide comprising the nucleotide sequence of the full-length protein coding sequence of clone br342_{—}11 deposited under accession number ATCC 98551;

(e) a polynucleotide encoding the full-length protein encoded by the cDNA insert of clone br342_{—}11 deposited under accession number ATCC 98551;

(f) a polynucleotide comprising the nucleotide sequence of a mature protein coding sequence of clone br342_{—}11 deposited under accession number ATCC 98551;

(g) a polynucleotide encoding a mature protein encoded by the cDNA insert of clone br342_{—}11 deposited under accession number ATCC 98551;

(h) a polynucleotide encoding a protein comprising the amino acid sequence of SEQ ID NO:188;

(i) a polynucleotide encoding a protein comprising a fragment of the amino acid sequence of SEQ ID NO:188 having biological activity, the fragment comprising eight consecutive amino acids of SEQ ID NO:188;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:187 from nucleotide 168 to nucleotide 962; the nucleotide sequence of SEQ ID NO:187 from nucleotide 351 to nucleotide 962; the nucleotide sequence of the full-length protein coding sequence of clone br342_{—}11 deposited under accession number ATCC 98551; or the nucleotide sequence of a mature protein coding sequence of clone br342_{—}11 deposited under accession number ATCC 98551. In other preferred embodiments, the polynucleotide encodes the full-length or a mature protein encoded by the cDNA insert of clone br342_{—}11 deposited under accession number ATCC 98551. In yet other preferred embodiments, the present invention provides a polynucleotide encoding a protein comprising the amino acid sequence of SEQ ID NO:188 from amino acid 1 to amino acid 78. In further preferred embodiments, the present invention provides a polynucleotide encoding a protein comprising a fragment of the amino acid sequence of SEQ ID NO:188 having biological activity, the fragment preferably comprising eight (more preferably twenty, most preferably thirty) consecutive amino acids of SEQ ID NO:188, or a polynucleotide encoding a protein comprising a fragment of the amino acid sequence of SEQ ID NO:188 having biological activity, the fragment comprising the amino acid sequence from amino acid 127 to amino acid 136 of SEQ ID NO:188.

Other embodiments provide the gene corresponding to the cDNA sequence of SEQ ID NO:187.

In other embodiments, the present invention provides a composition comprising a protein, wherein said protein comprises an amino acid sequence selected from the group consisting of:

(a) the amino acid sequence of SEQ ID NO:188;

(b) the amino acid sequence of SEQ ID NO:188 from amino acid 1 to amino acid 78;

(c) fragments of the amino acid sequence of SEQ ID NO:188 comprising eight consecutive amino acids of SEQ ID NO:188; and

(d) the amino acid sequence encoded by the cDNA insert of clone br342_{—}11 deposited under accession number ATCC 98551;

the protein being substantially free from other mammalian proteins. Preferably such protein comprises the amino acid sequence of SEQ ID NO:188 or the amino acid sequence of SEQ ID NO:188 from amino acid 1 to amino acid 78. In further preferred embodiments, the present invention provides a protein comprising a fragment of the amino acid sequence of SEQ ID NO:188 having biological activity, the fragment preferably comprising eight (more preferably twenty, most preferably thirty) consecutive amino acids of SEQ ID NO:188, or a protein comprising a fragment of the amino acid sequence of SEQ ID NO:188 having biological activity, the fragment comprising the amino acid sequence from amino acid 127 to amino acid 136 of SEQ ID NO:188.

In one embodiment, the present invention provides a composition comprising an isolated polynucleotide selected from the group consisting of:

(a) a polynucleotide comprising the nucleotide sequence of SEQ ID NO:189;

(b) a polynucleotide comprising the nucleotide sequence of SEQ ID NO:189 from nucleotide 134 to nucleotide 493;

(c) a polynucleotide comprising the nucleotide sequence of the full-length protein coding sequence of clone ej258_{—}11 deposited under accession number ATCC 98551;

(d) a polynucleotide encoding the full-length protein encoded by the cDNA insert of clone ej258_{—}11 deposited under accession number ATCC 98551;

(e) a polynucleotide comprising the nucleotide sequence of a mature protein coding sequence of clone ej258_{—}11 deposited under accession number ATCC 98551;

(f) a polynucleotide encoding a mature protein encoded by the cDNA insert of clone ej258_{—}11 deposited under accession number ATCC 98551;

(g) a polynucleotide encoding a protein comprising the amino acid sequence of SEQ ID NO:190;

(h) a polynucleotide encoding a protein comprising a fragment of the amino acid sequence of SEQ ID NO:190 having biological activity, the fragment comprising eight consecutive amino acids of SEQ ID NO:190;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:189 from nucleotide 134 to nucleotide 493; the nucleotide sequence of the full-length protein coding sequence of clone ej258_{—}11 deposited under accession number ATCC 98551; or the nucleotide sequence of a mature protein coding sequence of clone ej258_{—}11 deposited under accession number ATCC 98551. In other preferred embodiments, the polynucleotide encodes the full-length or a mature protein encoded by the cDNA insert of clone ej258_{—}11 deposited under accession number ATCC 98551. In yet other preferred embodiments, the present invention provides a polynucleotide encoding a protein comprising the amino acid sequence of SEQ ID NO:190 from amino acid 1 to amino acid 64. In further preferred embodiments, the present invention provides a polynucleotide encoding a protein comprising a fragment of the amino acid sequence of SEQ ID NO:190 having biological activity, the fragment preferably comprising eight (more preferably twenty, most preferably thirty) consecutive amino acids of SEQ ID NO:190, or a polynucleotide encoding a protein comprising a fragment of the amino acid sequence of SEQ ID NO:190 having biological activity, the fragment comprising the amino acid sequence from amino acid 55 to amino acid 64 of SEQ ID NO:190.

Other embodiments provide the gene corresponding to the cDNA sequence of SEQ ID NO:189.

In other embodiments, the present invention provides a composition comprising a protein, wherein said protein comprises an amino acid sequence selected from the group consisting of:

(a) the amino acid sequence of SEQ ID NO:190;

(b) the amino acid sequence of SEQ ID NO:8 from amino acid 1 to amino acid 64;

(c) fragments of the amino acid sequence of SEQ ID NO:190 comprising eight consecutive amino acids of SEQ ID NO:190; and

(d) the amino acid sequence encoded by the cDNA insert of clone ej258_{—}11 deposited under accession number ATCC 98551;

the protein being substantially free from other mammalian proteins. Preferably such protein comprises the amino acid sequence of SEQ ID NO:190 or the amino acid sequence of SEQ ID NO:190 from amino acid 1 to amino acid 64. In further preferred embodiments, the present invention provides a protein comprising a fragment of the amino acid sequence of SEQ ID NO:190 having biological activity, the fragment preferably comprising eight (more preferably twenty, most preferably thirty) consecutive amino acids of SEQ ID NO:190, or a protein comprising a fragment of the amino acid sequence of SEQ ID NO:190 having biological activity, the fragment comprising the amino acid sequence from amino acid 55 to amino acid 64 of SEQ ID NO:190.

In one embodiment, the present invention provides a composition comprising an isolated polynucleotide selected from the group consisting of:

(a) a polynucleotide comprising the nucleotide sequence of SEQ ID NO:191;

(b) a polynucleotide comprising the nucleotide sequence of SEQ ID NO:191 from nucleotide 14 to nucleotide 406;

(c) a polynucleotide comprising the nucleotide sequence of SEQ ID NO:191 from nucleotide 62 to nucleotide 406;

(d) a polynucleotide comprising the nucleotide sequence of the full-length protein coding sequence of clone k232_{—}2x deposited under accession number ATCC 98551;

(e) a polynucleotide encoding the full-length protein encoded by the cDNA insert of clone k232_{—}2x deposited under accession number ATCC 98551;

(f) a polynucleotide comprising the nucleotide sequence of a mature protein coding sequence of clone k232_{—}2x deposited under accession number ATCC 98551;

(g) a polynucleotide encoding a mature protein encoded by the cDNA insert of clone k232_{—}2x deposited under accession number ATCC 98551;

(h) a polynucleotide encoding a protein comprising the amino acid sequence of SEQ ID NO:192;

(i) a polynucleotide encoding a protein comprising a fragment of the amino acid sequence of SEQ ID NO:192 having biological activity, the fragment comprising eight consecutive amino acids of SEQ ID NO:192;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:191 from nucleotide 14 to nucleotide 406; the nucleotide sequence of SEQ ID NO:191 from nucleotide 62 to nucleotide 406; the nucleotide sequence of the full-length protein coding sequence of clone k232_{—}2x deposited under accession number ATCC 98551; or the nucleotide sequence of a mature protein coding sequence of clone k232_{—}2x deposited under accession number ATCC 98551. In other preferred embodiments, the polynucleotide encodes the full-length or a mature protein encoded by the cDNA insert of clone k232_{—}2x deposited under accession number ATCC 98551. In yet other preferred embodiments, the present invention provides a polynucleotide encoding a protein comprising the amino acid sequence of SEQ ID NO:192 from amino acid 1 to amino acid 81. In further preferred embodiments, the present invention provides a polynucleotide encoding a protein comprising a fragment of the amino acid sequence of SEQ ID NO:192 having biological activity, the fragment preferably comprising eight (more preferably twenty, most preferably thirty) consecutive amino acids of SEQ ID NO:192, or a polynucleotide encoding a protein comprising a fragment of the amino acid sequence of SEQ ID NO:192 having biological activity, the fragment comprising the amino acid sequence from amino acid 60 to amino acid 69 of SEQ ID NO:192.

Other embodiments provide the gene corresponding to the cDNA sequence of SEQ ID NO:191.

In other embodiments, the present invention provides a composition comprising a protein, wherein said protein comprises an amino acid sequence selected from the group consisting of:

(a) the amino acid sequence of SEQ ID NO:192;

(b) the amino acid sequence of SEQ ID NO:192 from amino acid 1 to amino acid 81;

(c) fragments of the amino acid sequence of SEQ ID NO:192 comprising eight consecutive amino acids of SEQ ID NO:192; and

(d) the amino acid sequence encoded by the cDNA insert of clone k232_{—}2x deposited under accession number ATCC 98551;

the protein being substantially free from other mammalian proteins. Preferably such protein comprises the amino acid sequence of SEQ ID NO:192 or the amino acid sequence of SEQ ID NO:192 from amino acid 1 to amino acid 81. In further preferred embodiments, the present invention provides a protein comprising a fragment of the amino acid sequence of SEQ ID NO:192 having biological activity, the fragment preferably comprising eight (more preferably twenty, most preferably thirty) consecutive amino acids of SEQ ID NO:192, or a protein comprising a fragment of the amino acid sequence of SEQ ID NO:192 having biological activity, the fragment comprising the amino acid sequence from amino acid 60 to amino acid 69 of SEQ ID NO:192.

In one embodiment, the present invention provides a composition comprising an isolated polynucleotide selected from the group consisting of:

(a) a polynucleotide comprising the nucleotide sequence of SEQ ID NO:193;

(b) a polynucleotide comprising the nucleotide sequence of SEQ ID NO:193 from nucleotide 580 to nucleotide 816;

(c) a polynucleotide comprising the nucleotide sequence of the full-length protein coding sequence of clone lf307_{—}5 deposited under accession number ATCC 98551;

(d) a polynucleotide encoding the full-length protein encoded by the cDNA insert of clone lf307_{—}5 deposited under accession number ATCC 98551;

(e) a polynucleotide comprising the nucleotide sequence of a mature protein coding sequence of clone lf307_{—}5 deposited under accession number ATCC 98551;

(f) a polynucleotide encoding a mature protein encoded by the cDNA insert of clone lf307_{—}5 deposited under accession number ATCC 98551;

(g) a polynucleotide encoding a protein comprising the amino acid sequence of SEQ ID NO:194;

(h) a polynucleotide encoding a protein comprising a fragment of the amino acid sequence of SEQ ID NO:194 having biological activity, the fragment comprising eight consecutive amino acids of SEQ ID NO:194;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:193 from nucleotide 580 to nucleotide 816; the nucleotide sequence of the full-length protein coding sequence of clone lf307_{—}5 deposited under accession number ATCC 98551; or the nucleotide sequence of a mature protein coding sequence of clone lf307_{—}5 deposited under accession number ATCC 98551. In other preferred embodiments, the polynucleotide encodes the full-length or a mature protein encoded by the cDNA insert of clone lf307_{—}5 deposited under accession number ATCC 98551. In further preferred embodiments, the present invention provides a polynucleotide encoding a protein comprising a fragment of the amino acid sequence of SEQ ID NO:194 having biological activity, the fragment preferably comprising eight (more preferably twenty, most preferably thirty) consecutive amino acids of SEQ ID NO:194, or a polynucleotide encoding a protein comprising a fragment of the amino acid sequence of SEQ ID NO:194 having biological activity, the fragment comprising the amino acid sequence from amino acid 34 to amino acid 43 of SEQ ID NO:194.

Other embodiments provide the gene corresponding to the cDNA sequence of SEQ ID NO:193 or SEQ ID NO:195.

In other embodiments, the present invention provides a composition comprising a protein, wherein said protein comprises an amino acid sequence selected from the group consisting of:

(a) the amino acid sequence of SEQ ID NO:194;

(b) fragments of the amino acid sequence of SEQ ID NO:194 comprising eight consecutive amino acids of SEQ ID NO:194; and

(c) the amino acid sequence encoded by the cDNA insert of clone lf307_{—}5 deposited under accession number ATCC 98551;

the protein being substantially free from other mammalian proteins. Preferably such protein comprises the amino acid sequence of SEQ ID NO:194. In further preferred embodiments, the present invention provides a protein comprising a fragment of the amino acid sequence of SEQ ID NO:194 having biological activity, the fragment preferably comprising eight (more preferably twenty, most preferably thirty) consecutive amino acids of SEQ ID NO:194, or a protein comprising a fragment of the amino acid sequence of SEQ ID NO:194 having biological activity, the fragment comprising the amino acid sequence from amino acid 34 to amino acid 43 of SEQ ID NO:194.

In one embodiment, the present invention provides a composition comprising an isolated polynucleotide selected from the group consisting of:

(a) a polynucleotide comprising the nucleotide sequence of SEQ ID NO:196;

(b) a polynucleotide comprising the nucleotide sequence of SEQ ID NO:196 from nucleotide 127 to nucleotide 627;

(c) a polynucleotide comprising the nucleotide sequence of SEQ ID NO:196 from nucleotide 250 to nucleotide 627;

(d) a polynucleotide comprising the nucleotide sequence of the full-length protein coding sequence of clone lr204_{—}1 deposited under accession number ATCC 98551;

(e) a polynucleotide encoding the full-length protein encoded by the cDNA insert of clone lr204_{—}1 deposited under accession number ATCC 98551;

(f) a polynucleotide comprising the nucleotide sequence of a mature protein coding sequence of clone lr204_{—}1 deposited under accession number ATCC 98551;

(g) a polynucleotide encoding a mature protein encoded by the cDNA insert of clone lr204_{—}1 deposited under accession number ATCC 98551;

(h) a polynucleotide encoding a protein comprising the amino acid sequence of SEQ ID NO:197;

(i) a polynucleotide encoding a protein comprising a fragment of the amino acid sequence of SEQ ID NO:197 having biological activity, the fragment comprising eight consecutive amino acids of SEQ ID NO:197;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:196 from nucleotide 127 to nucleotide 627; the nucleotide sequence of SEQ ID NO:196 from nucleotide 250 to nucleotide 627; the nucleotide sequence of the full-length protein coding sequence of clone lr204_{—}1 deposited under accession number ATCC 98551; or the nucleotide sequence of a mature protein coding sequence of clone lr204_{—}1 deposited under accession number ATCC 98551. In other preferred embodiments, the polynucleotide encodes the full-length or a mature protein encoded by the cDNA insert of clone lr204_{—}1 deposited under accession number ATCC 98551. In yet other preferred embodiments, the present invention provides a polynucleotide encoding a protein comprising the amino acid sequence of SEQ ID NO:197 from amino acid 23 to amino acid 106. In further preferred embodiments, the present invention provides a polynucleotide encoding a protein comprising a fragment of the amino acid sequence of SEQ ID NO:197 having biological activity, the fragment preferably comprising eight (more preferably twenty, most preferably thirty) consecutive amino acids of SEQ ID NO:197, or a polynucleotide encoding a protein comprising a fragment of the amino acid sequence of SEQ ID NO:197 having biological activity, the fragment comprising the amino acid sequence from amino acid 78 to amino acid 87 of SEQ ID NO:197.

Other embodiments provide the gene corresponding to the cDNA sequence of SEQ ID NO:196.

In other embodiments, the present invention provides a composition comprising a protein, wherein said protein comprises an amino acid sequence selected from the group consisting of:

(a) the amino acid sequence of SEQ ID NO:197;

(b) the amino acid sequence of SEQ ID NO:197 from amino acid 23 to amino acid 106;

(c) fragments of the amino acid sequence of SEQ ID NO:197 comprising eight consecutive amino acids of SEQ ID NO:197; and

(d) the amino acid sequence encoded by the cDNA insert of clone lr204_{—}1 deposited under accession number ATCC 98551;

the protein being substantially free from other mammalian proteins. Preferably such protein comprises the amino acid sequence of SEQ ID NO:197 or the amino acid sequence of SEQ ID NO:197 from amino acid 23 to amino acid 106. In further preferred embodiments, the present invention provides a protein comprising a fragment of the amino acid sequence of SEQ ID NO:197 having biological activity, the fragment preferably comprising eight (more preferably twenty, most preferably thirty) consecutive amino acids of SEQ ID NO:197, or a protein comprising a fragment of the amino acid sequence of SEQ ID NO:197 having biological activity, the fragment comprising the amino acid sequence from amino acid 78 to amino acid 87 of SEQ ID NO:197.

In one embodiment, the present invention provides a composition comprising an isolated polynucleotide selected from the group consisting of:

(a) a polynucleotide comprising the nucleotide sequence of SEQ ID NO:205;

(b) a polynucleotide comprising the nucleotide sequence of SEQ ID NO:205 from nucleotide 876 to nucleotide 1190;

(c) a polynucleotide comprising the nucleotide sequence of SEQ ID NO:205 from nucleotide 963 to nucleotide 1190;

(d) a polynucleotide comprising the nucleotide sequence of the full-length protein coding sequence of clone as20_{—}2 deposited under accession number ATCC 98580;

(e) a polynucleotide encoding the full-length protein encoded by the cDNA insert of clone as20_{—}2 deposited under accession number ATCC 98580;

(f) a polynucleotide comprising the nucleotide sequence of a mature protein coding sequence of clone as20_{—}2 deposited under accession number ATCC 98580;

(g) a polynucleotide encoding a mature protein encoded by the cDNA insert of clone as20_{—}2 deposited under accession number ATCC 98580;

(h) a polynucleotide encoding a protein comprising the amino acid sequence of SEQ ID NO:206;

(i) a polynucleotide encoding a protein comprising a fragment of the amino acid sequence of SEQ ID NO:206 having biological activity, the fragment comprising eight consecutive amino acids of SEQ ID NO:206;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:37 from nucleotide 876 to nucleotide 1190; the nucleotide sequence of SEQ ID NO:205 from nucleotide 963 to nucleotide 1190; the nucleotide sequence of the full-length protein coding sequence of clone as20_{—}2 deposited under accession number ATCC 98580; or the nucleotide sequence of a mature protein coding sequence of clone as20_{—}2 deposited under accession number ATCC 98580. In other preferred embodiments, the polynucleotide encodes the full-length or a mature protein encoded by the cDNA insert of clone as20_{—}2 deposited under accession number ATCC 98580. In yet other preferred embodiments, the present invention provides a polynucleotide encoding a protein comprising the amino acid sequence of SEQ ID NO:2 from amino acid 1 to amino acid 60. In further preferred embodiments, the present invention provides a polynucleotide encoding a protein comprising a fragment of the amino acid sequence of SEQ ID NO:206 having biological activity, the fragment preferably comprising eight (more preferably twenty, most preferably thirty) consecutive amino acids of SEQ ID NO:206, or a polynucleotide encoding a protein comprising a fragment of the amino acid sequence of SEQ ID NO:206 having biological activity, the fragment comprising the amino acid sequence from amino acid 47 to amino acid 56 of SEQ ID NO:206.

Other embodiments provide the gene corresponding to the cDNA sequence of SEQ ID NO:205.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:205, but excluding the poly(A) tail at the 3′ end of SEQ ID NO:205; and

(ab) the nucleotide sequence of the cDNA insert of clone as20_{—}2 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:205, but excluding the poly(A) tail at the 3′ end of SEQ ID NO:205; and

(bb) the nucleotide sequence of the cDNA insert of clone as20_{—}2 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:205, and extends contiguously from a nucleotide sequence corresponding to the 5′ end of SEQ ID NO:205 to a nucleotide sequence corresponding to the 3′ end of SEQ ID NO:1 but excluding the poly(A) tail at the 3′ end of SEQ ID NO:205. In another preferred embodiment, the nucleotide sequence of said isolated polynucleotide corresponds to the cDNA sequence of SEQ ID NO:205 from nucleotide 876 to nucleotide 1190, and extends contiguously from a nucleotide sequence corresponding to the 5′ end of said sequence of SEQ ID NO:205 from nucleotide 876 to nucleotide 1190, to a nucleotide sequence corresponding to the 3′ end of said sequence of SEQ ID NO:205 from nucleotide 876 to nucleotide 1190. In a further preferred embodiment, the nucleotide sequence of said isolated polynucleotide corresponds to the cDNA sequence of SEQ ID NO:205 from nucleotide 963 to nucleotide 1190, and extends contiguously from a nucleotide sequence corresponding to the 5′ end of said sequence of SEQ ID NO:205 from nucleotide 963 to nucleotide 1190, to a nucleotide sequence corresponding to the 3′ end of said sequence of SEQ ID NO:1 from nucleotide 963 to nucleotide 1190.

In other embodiments, the present invention provides a composition comprising a protein, wherein said protein comprises an amino acid sequence selected from the group consisting of:

(a) the amino acid sequence of SEQ ID NO:206;

(b) the amino acid sequence of SEQ ID NO:206 from amino acid 1 to amino acid 60;

(c) fragments of the amino acid sequence of SEQ ID NO:206 comprising eight consecutive amino acids of SEQ ID NO:206; and

(d) the amino acid sequence encoded by the cDNA insert of clone as20_{—}2 deposited under accession number ATCC 98580;

the protein being substantially free from other mammalian proteins. Preferably such protein comprises the amino acid sequence of SEQ ID NO:206 or the amino acid sequence of SEQ ID NO:206 from amino acid 1 to amino acid 60. In further preferred embodiments, the present invention provides a protein comprising a fragment of the amino acid sequence of SEQ ID NO:206 having biological activity, the fragment preferably comprising eight (more preferably twenty, most preferably thirty) consecutive amino acids of SEQ ID NO:206, or a protein comprising a fragment of the amino acid sequence of SEQ ID NO:206 having biological activity, the fragment comprising the amino acid sequence from amino acid 47 to amino acid 56 of SEQ ID NO:206.

In one embodiment, the present invention provides a composition comprising an isolated polynucleotide selected from the group consisting of:

(a) a polynucleotide comprising the nucleotide sequence of SEQ ID NO:207;

(b) a polynucleotide comprising the nucleotide sequence of SEQ ID NO:207 from nucleotide 946 to nucleotide 1095;

(c) a polynucleotide comprising the nucleotide sequence of the full-length protein coding sequence of clone bf227_{—}8 deposited under accession number ATCC 98580;

(d) a polynucleotide encoding the full-length protein encoded by the cDNA insert of clone bf227_{—}8 deposited under accession number ATCC 98580;

(e) a polynucleotide comprising the nucleotide sequence of a mature protein coding sequence of clone bf227_{—}8 deposited under accession number ATCC 98580;

(f) a polynucleotide encoding a mature protein encoded by the cDNA insert of clone bf227_{—}8 deposited under accession number ATCC 98580;

(g) a polynucleotide encoding a protein comprising the amino acid sequence of SEQ ID NO:208;

(h) a polynucleotide encoding a protein comprising a fragment of the amino acid sequence of SEQ ID NO:208 having biological activity, the fragment comprising eight consecutive amino acids of SEQ ID NO:208;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:207 from nucleotide 946 to nucleotide 1095; the nucleotide sequence of the full-length protein coding sequence of clone bf227_{—}8 deposited under accession number ATCC 98580; or the nucleotide sequence of a mature protein coding sequence of clone bf227_{—}8 deposited under accession number ATCC 98580. In other preferred embodiments, the polynucleotide encodes the full-length or a mature protein encoded by the cDNA insert of clone bf227_{—}8 deposited under accession number ATCC 98580. In yet other preferred embodiments, the present invention provides a polynucleotide encoding a protein comprising the amino acid sequence of SEQ ID NO:208 from amino acid 1 to amino acid 34. In further preferred embodiments, the present invention provides a polynucleotide encoding a protein comprising a fragment of the amino acid sequence of SEQ ID NO:208 having biological activity, the fragment preferably comprising eight (more preferably twenty, most preferably thirty) consecutive amino acids of SEQ ID NO:208, or a polynucleotide encoding a protein comprising a fragment of the amino acid sequence of SEQ ID NO:208 having biological activity, the fragment comprising the amino acid sequence from amino acid 19 to amino acid 28 of SEQ ID NO:208.

Other embodiments provide the gene corresponding to the cDNA sequence of SEQ ID NO:207.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:207, but excluding the poly(A) tail at the 3′ end of SEQ ID NO:207; and

(ab) the nucleotide sequence of the cDNA insert of clone bf227_{—}8 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:207, but excluding the poly(A) tail at the 3′ end of SEQ ID NO:207; and

(bb) the nucleotide sequence of the cDNA insert of clone bf227_{—}8 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:207, and extends contiguously from a nucleotide sequence corresponding to the 5′ end of SEQ ID NO:207 to a nucleotide sequence corresponding to the 3′ end of SEQ ID NO:3 but excluding the poly(A) tail at the 3′ end of SEQ ID NO:207. In another preferred embodiment, the nucleotide sequence of said isolated polynucleotide corresponds to the cDNA sequence of SEQ ID NO:207 from nucleotide 946 to nucleotide 1095, and extends contiguously from a nucleotide sequence corresponding to the 5′ end of said sequence of SEQ ID NO:207 from nucleotide 946 to nucleotide 1095, to a nucleotide sequence corresponding to the 3′ end of said sequence of SEQ ID NO:207 from nucleotide 946 to nucleotide 1095.

In other embodiments, the present invention provides a composition comprising a protein, wherein said protein comprises an amino acid sequence selected from the group consisting of:

(a) the amino acid sequence of SEQ ID NO:208;

(b) the amino acid sequence of SEQ ID NO:208 from amino acid 1 to amino acid 34;

(c) fragments of the amino acid sequence of SEQ ID NO:208 comprising eight consecutive amino acids of SEQ ID NO:208; and

(d) the amino acid sequence encoded by the cDNA insert of clone bf227_{—}8 deposited under accession number ATCC 98580;

the protein being substantially free from other mammalian proteins. Preferably such protein comprises the amino acid sequence of SEQ ID NO:208 or the amino acid sequence of SEQ ID NO:208 from amino acid 1 to amino acid 34. In further preferred embodiments, the present invention provides a protein comprising a fragment of the amino acid sequence of SEQ ID NO:208 having biological activity, the fragment preferably comprising eight (more preferably twenty, most preferably thirty) consecutive amino acids of SEQ ID NO:208, or a protein comprising a fragment of the amino acid sequence of SEQ ID NO:208 having biological activity, the fragment comprising the amino acid sequence from amino acid 19 to amino acid 28 of SEQ ID NO:208.

In one embodiment, the present invention provides a composition comprising an isolated polynucleotide selected from the group consisting of:

(a) a polynucleotide comprising the nucleotide sequence of SEQ ID NO:209;

(b) a polynucleotide comprising the nucleotide sequence of SEQ ID NO:209 from nucleotide 183 to nucleotide 911;

(c) a polynucleotide comprising the nucleotide sequence of the full-length protein coding sequence of clone bh157_{—}7 deposited under accession number ATCC 98580;

(d) a polynucleotide encoding the full-length protein encoded by the cDNA insert of clone bh157_{—}7 deposited under accession number ATCC 98580;

(e) a polynucleotide comprising the nucleotide sequence of a mature protein coding sequence of clone bh157_{—}7 deposited under accession number ATCC 98580;

(f) a polynucleotide encoding a mature protein encoded by the cDNA insert of clone bh157_{—}7 deposited under accession number ATCC 98580;

(g) a polynucleotide encoding a protein comprising the amino acid sequence of SEQ ID NO:6;

(h) a polynucleotide encoding a protein comprising a fragment of the amino acid sequence of SEQ ID NO:6 having biological activity, the fragment comprising eight consecutive amino acids of SEQ ID NO:6;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:209 from nucleotide 183 to nucleotide 911; the nucleotide sequence of the full-length protein coding sequence of clone bh157_{—}7 deposited under accession number ATCC 98580; or the nucleotide sequence of a mature protein coding sequence of clone bh157_{—}7 deposited under accession number ATCC 98580. In other preferred embodiments, the polynucleotide encodes the full-length or a mature protein encoded by the cDNA insert of clone bh157_{—}7 deposited under accession number ATCC 98580. In yet other preferred embodiments, the present invention provides a polynucleotide encoding a protein comprising the amino acid sequence of SEQ ID NO:210 from amino acid 1 to amino acid 76. In further preferred embodiments, the present invention provides a polynucleotide encoding a protein comprising a fragment of the amino acid sequence of SEQ ID NO:210 having biological activity, the fragment preferably comprising eight (more preferably twenty, most preferably thirty) consecutive amino acids of SEQ ID NO:210, or a polynucleotide encoding a protein comprising a fragment of the amino acid sequence of SEQ ID NO:210 having biological activity, the fragment comprising the amino acid sequence from amino acid 116 to amino acid 125 of SEQ ID NO:210.

Other embodiments provide the gene corresponding to the cDNA sequence of SEQ ID NO:209.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:209, but excluding the poly(A) tail at the 3′ end of SEQ ID NO:209; and

(ab) the nucleotide sequence of the cDNA insert of clone bh157_{—}7 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:209, but excluding the poly(A) tail at the 3′ end of SEQ ID NO:209; and

(bb) the nucleotide sequence of the cDNA insert of clone bh157_{—}7 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:209, and extends contiguously from a nucleotide sequence corresponding to the 5′ end of SEQ ID NO:209 to a nucleotide sequence corresponding to the 3′ end of SEQ ID NO:209 but excluding the poly(A) tail at the 3′ end of SEQ ID NO:209. In another preferred embodiment, the nucleotide sequence of said isolated polynucleotide corresponds to the cDNA sequence of SEQ ID NO:209 from nucleotide 183 to nucleotide 911, and extends contiguously from a nucleotide sequence corresponding to the 5′ end of said sequence of SEQ ID NO:209 from nucleotide 183 to nucleotide 911, to a nucleotide sequence corresponding to the 3′ end of said sequence of SEQ ID NO:209 from nucleotide 183 to nucleotide 911.

In other embodiments, the present invention provides a composition comprising a protein, wherein said protein comprises an amino acid sequence selected from the group consisting of:

(a) the amino acid sequence of SEQ ID NO:210;

(b) the amino acid sequence of SEQ ID NO:210 from amino acid 1 to amino acid 76;

(c) fragments of the amino acid sequence of SEQ ID NO:210 comprising eight consecutive amino acids of SEQ ID NO:210; and

(d) the amino acid sequence encoded by the cDNA insert of clone bh157_{—}7 deposited under accession number ATCC 98580;

the protein being substantially free from other mammalian proteins. Preferably such protein comprises the amino acid sequence of SEQ ID NO:210 or the amino acid sequence of SEQ ID NO:210 from amino acid 1 to amino acid 76. In further preferred embodiments, the present invention provides a protein comprising a fragment of the amino acid sequence of SEQ ID NO:210 having biological activity, the fragment preferably comprising eight (more preferably twenty, most preferably thirty) consecutive amino acids of SEQ ID NO:210, or a protein comprising a fragment of the amino acid sequence of SEQ ID NO:210 having biological activity, the fragment comprising the amino acid sequence from amino acid 116 to amino acid 125 of SEQ ID NO:210.

In one embodiment, the present invention provides a composition comprising an isolated polynucleotide selected from the group consisting of:

(a) a polynucleotide comprising the nucleotide sequence of SEQ ID NO:211;

(b) a polynucleotide comprising the nucleotide sequence of SEQ ID NO:211 from nucleotide 1391 to nucleotide 1609;

(c) a polynucleotide comprising the nucleotide sequence of SEQ ID NO:211 from nucleotide 1439 to nucleotide 1609;

(d) a polynucleotide comprising the nucleotide sequence of the full-length protein coding sequence of clone cg426_{—}8 deposited under accession number ATCC 98580;

(e) a polynucleotide encoding the full-length protein encoded by the cDNA insert of clone cg426_{—}8 deposited under accession number ATCC 98580;

(f) a polynucleotide comprising the nucleotide sequence of a mature protein coding sequence of clone cg426_{—}8 deposited under accession number ATCC 98580;

(g) a polynucleotide encoding a mature protein encoded by the cDNA insert of clone cg426_{—}8 deposited under accession number ATCC 98580;

(h) a polynucleotide encoding a protein comprising the amino acid sequence of SEQ ID NO:212;

(i) a polynucleotide encoding a protein comprising a fragment of the amino acid sequence of SEQ ID NO:212 having biological activity, the fragment comprising eight consecutive amino acids of SEQ ID NO:212;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:211 from nucleotide 1391 to nucleotide 1609; the nucleotide sequence of SEQ ID NO:211 from nucleotide 1439 to nucleotide 1609; the nucleotide sequence of the full-length protein coding sequence of clone cg426_{—}8 deposited under accession number ATCC 98580; or the nucleotide sequence of a mature protein coding sequence of clone cg426_{—}8 deposited under accession number ATCC 98580. In other preferred embodiments, the polynucleotide encodes the full-length or a mature protein encoded by the cDNA insert of clone cg426_{—}8 deposited under accession number ATCC 98580. In further preferred embodiments, the present invention provides a polynucleotide encoding a protein comprising a fragment of the amino acid sequence of SEQ ID NO:212 having biological activity, the fragment preferably comprising eight (more preferably twenty, most preferably thirty) consecutive amino acids of SEQ ID NO:212, or a polynucleotide encoding a protein comprising a fragment of the amino acid sequence of SEQ ID NO:212 having biological activity, the fragment comprising the amino acid sequence from amino acid 31 to amino acid 40 of SEQ ID NO:212.

Other embodiments provide the gene corresponding to the cDNA sequence of SEQ ID NO:211.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:211, but excluding the poly(A) tail at the 3′ end of SEQ ID NO:211; and

(ab) the nucleotide sequence of the cDNA insert of clone cg426_{—}8 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:211, but excluding the poly(A) tail at the 3′ end of SEQ ID NO:211; and

(bb) the nucleotide sequence of the cDNA insert of clone cg426_{—}8 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:211, and extends contiguously from a nucleotide sequence corresponding to the 5′ end of SEQ ID NO:211 to a nucleotide sequence corresponding to the 3′ end of SEQ ID NO:211 but excluding the poly(A) tail at the 3′ end of SEQ ID NO:211. In another preferred embodiment, the nucleotide sequence of said isolated polynucleotide corresponds to the cDNA sequence of SEQ ID NO:211 from nucleotide 1391 to nucleotide 1609, and extends contiguously from a nucleotide sequence corresponding to the 5′ end of said sequence of SEQ ID NO:211 from nucleotide 1391 to nucleotide 1609, to a nucleotide sequence corresponding to the 3′ end of said sequence of SEQ ID NO:211 from nucleotide 1391 to nucleotide 1609. In a further preferred embodiment, the nucleotide sequence of said isolated polynucleotide corresponds to the cDNA sequence of SEQ ID NO:211 from nucleotide 1439 to nucleotide 1609, and extends contiguously from a nucleotide sequence corresponding to the 5′ end of said sequence of SEQ ID NO:211 from nucleotide 1439 to nucleotide 1609, to a nucleotide sequence corresponding to the 3′ end of said sequence of SEQ ID NO:211 from nucleotide 1439 to nucleotide 1609.

In other embodiments, the present invention provides a composition comprising a protein, wherein said protein comprises an amino acid sequence selected from the group consisting of:

(a) the amino acid sequence of SEQ ID NO:212;

(b) fragments of the amino acid sequence of SEQ ID NO:212 comprising eight consecutive amino acids of SEQ ID NO:212; and

(c) the amino acid sequence encoded by the cDNA insert of clone cg426_{—}8 deposited under accession number ATCC 98580;

the protein being substantially free from other mammalian proteins. Preferably such protein comprises the amino acid sequence of SEQ ID NO:212. In further preferred embodiments, the present invention provides a protein comprising a fragment of the amino acid sequence of SEQ ID NO:212 having biological activity, the fragment preferably comprising eight (more preferably twenty, most preferably thirty) consecutive amino acids of SEQ ID NO:212, or a protein comprising a fragment of the amino acid sequence of SEQ ID NO:212 having biological activity, the fragment comprising the amino acid sequence from amino acid 31 to amino acid 40 of SEQ ID NO:212.

In one embodiment, the present invention provides a composition comprising an isolated polynucleotide selected from the group consisting of:

(a) a polynucleotide comprising the nucleotide sequence of SEQ ID NO:213;

(b) a polynucleotide comprising the nucleotide sequence of SEQ ID NO:213 from nucleotide 185 to nucleotide 586;

(c) a polynucleotide comprising the nucleotide sequence of SEQ ID NO:213 from nucleotide 578 to nucleotide 586;

(d) a polynucleotide comprising the nucleotide sequence of the full-length protein coding sequence of clone ck48_{—}12 deposited under accession number ATCC 98580;

(e) a polynucleotide encoding the full-length protein encoded by the cDNA insert of clone ck48_{—}12 deposited under accession number ATCC 98580;

(f) a polynucleotide comprising the nucleotide sequence of a mature protein coding sequence of clone ck48_{—}12 deposited under accession number ATCC 98580;

(g) a polynucleotide encoding a mature protein encoded by the cDNA insert of clone ck48_{—}12 deposited under accession number ATCC 98580;

(h) a polynucleotide encoding a protein comprising the amino acid sequence of SEQ ID NO:214;

(i) a polynucleotide encoding a protein comprising a fragment of the amino acid sequence of SEQ ID NO:214 having biological activity, the fragment comprising eight consecutive amino acids of SEQ ID NO:214;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:213 from nucleotide 185 to nucleotide 586; the nucleotide sequence of SEQ ID NO:213 from nucleotide 578 to nucleotide 586; the nucleotide sequence of the full-length protein coding sequence of clone ck48_{—}12 deposited under accession number ATCC 98580; or the nucleotide sequence of a mature protein coding sequence of clone ck48_{—}12 deposited under accession number ATCC 98580. In other preferred embodiments, the polynucleotide encodes the full-length or a mature protein encoded by the cDNA insert of clone ck48_{—}12 deposited under accession number ATCC 98580. In further preferred embodiments, the present invention provides a polynucleotide encoding a protein comprising a fragment of the amino acid sequence of SEQ ID NO:214 having biological activity, the fragment preferably comprising eight (more preferably twenty, most preferably thirty) consecutive amino acids of SEQ ID NO:214, or a polynucleotide encoding a protein comprising a fragment of the amino acid sequence of SEQ ID NO:214 having biological activity, the fragment comprising the amino acid sequence from amino acid 62 to amino acid 71 of SEQ ID NO:214.

Other embodiments provide the gene corresponding to the cDNA sequence of SEQ ID NO:213.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:213, but excluding the poly(A) tail at the 3′ end of SEQ ID NO:213; and

(ab) the nucleotide sequence of the cDNA insert of clone ck48_{—}12 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:213, but excluding the poly(A) tail at the 3′ end of SEQ ID NO:213; and

(bb) the nucleotide sequence of the cDNA insert of clone ck48_{—}12 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:213, and extends contiguously from a nucleotide sequence corresponding to the 5′ end of SEQ ID NO:213 to a nucleotide sequence corresponding to the 3′ end of SEQ ID NO:9 but excluding the poly(A) tail at the 3′ end of SEQ ID NO:213. In another preferred embodiment, the nucleotide sequence of said isolated polynucleotide corresponds to the cDNA sequence of SEQ ID NO:213 from nucleotide 185 to nucleotide 586, and extends contiguously from a nucleotide sequence corresponding to the 5′ end of said sequence of SEQ ID NO:213 from nucleotide 185 to nucleotide 586, to a nucleotide sequence corresponding to the 3′ end of said sequence of SEQ ID NO:213 from nucleotide 185 to nucleotide 586.

In other embodiments, the present invention provides a composition comprising a protein, wherein said protein comprises an amino acid sequence selected from the group consisting of:

(a) the amino acid sequence of SEQ ID NO:214;

(b) fragments of the amino acid sequence of SEQ ID NO:214 comprising eight consecutive amino acids of SEQ ID NO:214; and

(c) the amino acid sequence encoded by the cDNA insert of clone ck48_{—}12 deposited under accession number ATCC 98580;

the protein being substantially free from other mammalian proteins. Preferably such protein comprises the amino acid sequence of SEQ ID NO:214. In further preferred embodiments, the present invention provides a protein comprising a fragment of the amino acid sequence of SEQ ID NO:214 having biological activity, the fragment preferably comprising eight (more preferably twenty, most preferably thirty) consecutive amino acids of SEQ ID NO:214, or a protein comprising a fragment of the amino acid sequence of SEQ ID NO:214 having biological activity, the fragment comprising the amino acid sequence from amino acid 62 to amino acid 71 of SEQ ID NO:214.

In one embodiment, the present invention provides a composition comprising an isolated polynucleotide selected from the group consisting of:

(a) a polynucleotide comprising the nucleotide sequence of SEQ ID NO:215;

(b) a polynucleotide comprising the nucleotide sequence of SEQ ID NO:215 from nucleotide 554 to nucleotide 1012;

(c) a polynucleotide comprising the nucleotide sequence of SEQ ID NO:215 from nucleotide 632 to nucleotide 1012;

(d) a polynucleotide comprising the nucleotide sequence of the full-length protein coding sequence of clone co1000_{—}1 deposited under accession number ATCC 98580;

(e) a polynucleotide encoding the full-length protein encoded by the cDNA insert of clone co1000_{—}1 deposited under accession number ATCC 98580;

(f) a polynucleotide comprising the nucleotide sequence of a mature protein coding sequence of clone co1000_{—}1 deposited under accession number ATCC 98580;

(g) a polynucleotide encoding a mature protein encoded by the cDNA insert of clone co1000_{—}1 deposited under accession number ATCC 98580;

(h) a polynucleotide encoding a protein comprising the amino acid sequence of SEQ ID NO:216;

(i) a polynucleotide encoding a protein comprising a fragment of the amino acid sequence of SEQ ID NO:216 having biological activity, the fragment comprising eight consecutive amino acids of SEQ ID NO:216;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:215 from nucleotide 554 to nucleotide 1012; the nucleotide sequence of SEQ ID NO:215 from nucleotide 632 to nucleotide 1012; the nucleotide sequence of the full-length protein coding sequence of clone co1000_{—}1 deposited under accession number ATCC 98580; or the nucleotide sequence of a mature protein coding sequence of clone co1000_{—}1 deposited under accession number ATCC 98580. In other preferred embodiments, the polynucleotide encodes the full-length or a mature protein encoded by the cDNA insert of clone co1000_{—}1 deposited under accession number ATCC 98580. In yet other preferred embodiments, the present invention provides a polynucleotide encoding a protein comprising the amino acid sequence of SEQ ID NO:216 from amino acid 1 to amino acid 63. In further preferred embodiments, the present invention provides a polynucleotide encoding a protein comprising a fragment of the amino acid sequence of SEQ ID NO:216 having biological activity, the fragment preferably comprising eight (more preferably twenty, most preferably thirty) consecutive amino acids of SEQ ID NO:216, or a polynucleotide encoding a protein comprising a fragment of the amino acid sequence of SEQ ID NO:216 having biological activity, the fragment comprising the amino acid sequence from amino acid 71 to amino acid 80 of SEQ ID NO:216.

Other embodiments provide the gene corresponding to the cDNA sequence of SEQ ID NO:215.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:215, but excluding the poly(A) tail at the 3′ end of SEQ ID NO:215; and

(ab) the nucleotide sequence of the cDNA insert of clone co1000_{—}1 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:215, but excluding the poly(A) tail at the 3′ end of SEQ ID NO:215; and

(bb) the nucleotide sequence of the cDNA insert of clone co1000_{—}1 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:215, and extends contiguously from a nucleotide sequence corresponding to the 5′ end of SEQ ID NO:215 to a nucleotide sequence corresponding to the 3′ end of SEQ ID NO:215 but excluding the poly(A) tail at the 3′ end of SEQ ID NO:215. In another preferred embodiment, the nucleotide sequence of said isolated polynucleotide corresponds to the cDNA sequence of SEQ ID NO:215 from nucleotide 554 to nucleotide 1012, and extends contiguously from a nucleotide sequence corresponding to the 5′ end of said sequence of SEQ ID NO:215 from nucleotide 554 to nucleotide 1012, to a nucleotide sequence corresponding to the 3′ end of said sequence of SEQ ID NO:215 from nucleotide 554 to nucleotide 1012. In a further preferred embodiment, the nucleotide sequence of said isolated polynucleotide corresponds to the cDNA sequence of SEQ ID NO:215 from nucleotide 632 to nucleotide 1012, and extends contiguously from a nucleotide sequence corresponding to the 5′ end of said sequence of SEQ ID NO:215 from nucleotide 632 to nucleotide 1012, to a nucleotide sequence corresponding to the 3′ end of said sequence of SEQ ID NO:215 from nucleotide 632 to nucleotide 1012.

In other embodiments, the present invention provides a composition comprising a protein, wherein said protein comprises an amino acid sequence selected from the group consisting of:

(a) the amino acid sequence of SEQ ID NO:216;

(b) the amino acid sequence of SEQ ID NO:216 from amino acid 1 to amino acid 63;

(c) fragments of the amino acid sequence of SEQ ID NO:216 comprising eight consecutive amino acids of SEQ ID NO:216; and

(d) the amino acid sequence encoded by the cDNA insert of clone co1000_{—}1 deposited under accession number ATCC 98580;

the protein being substantially free from other mammalian proteins. Preferably such protein comprises the amino acid sequence of SEQ ID NO:216 or the amino acid sequence of SEQ ID NO:216 from amino acid 1 to amino acid 63. In further preferred embodiments, the present invention provides a protein comprising a fragment of the amino acid sequence of SEQ ID NO:216 having biological activity, the fragment preferably comprising eight (more preferably twenty, most preferably thirty) consecutive amino acids of SEQ ID NO:216, or a protein comprising a fragment of the amino acid sequence of SEQ ID NO:216 having biological activity, the fragment comprising the amino acid sequence from amino acid 71 to amino acid 80 of SEQ ID NO:216.

In one embodiment, the present invention provides a composition comprising an isolated polynucleotide selected from the group consisting of:

(a) a polynucleotide comprising the nucleotide sequence of SEQ ID NO:217;

(b) a polynucleotide comprising the nucleotide sequence of SEQ ID NO:217 from nucleotide 83 to nucleotide 1111;

(c) a polynucleotide comprising the nucleotide sequence of SEQ ID NO:217 from nucleotide 155 to nucleotide 1111;

(d) a polynucleotide comprising the nucleotide sequence of the full-length protein coding sequence of clone ct489_{—}14 deposited under accession number ATCC 98580;

(e) a polynucleotide encoding the full-length protein encoded by the cDNA insert of clone ct489_{—}14 deposited under accession number ATCC 98580;

(f) a polynucleotide comprising the nucleotide sequence of a mature protein coding sequence of clone ct489_{—}14 deposited under accession number ATCC 98580;

(g) a polynucleotide encoding a mature protein encoded by the cDNA insert of clone ct489_{—}14 deposited under accession number ATCC 98580;

(h) a polynucleotide encoding a protein comprising the amino acid sequence of SEQ ID NO:218;

(i) a polynucleotide encoding a protein comprising a fragment of the amino acid sequence of SEQ ID NO:218 having biological activity, the fragment comprising eight consecutive amino acids of SEQ ID NO:218;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:217 from nucleotide 83 to nucleotide 1111; the nucleotide sequence of SEQ ID NO:217 from nucleotide 155 to nucleotide 1111; the nucleotide sequence of the full-length protein coding sequence of clone ct489_{—}14 deposited under accession number ATCC 98580; or the nucleotide sequence of a mature protein coding sequence of clone ct489_{—}14 deposited under accession number ATCC 98580. In other preferred embodiments, the polynucleotide encodes the full-length or a mature protein encoded by the cDNA insert of clone ct489_{—}14 deposited under accession number ATCC 98580. In further preferred embodiments, the present invention provides a polynucleotide encoding a protein comprising a fragment of the amino acid sequence of SEQ ID NO:218 having biological activity, the fragment preferably comprising eight (more preferably twenty, most preferably thirty) consecutive amino acids of SEQ ID NO:218, or a polynucleotide encoding a protein comprising a fragment of the amino acid sequence of SEQ ID NO:218 having biological activity, the fragment comprising the amino acid sequence from amino acid 166 to amino acid 175 of SEQ ID NO:218.

Other embodiments provide the gene corresponding to the cDNA sequence of SEQ ID NO:217.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:217, but excluding the poly(A) tail at the 3′ end of SEQ ID NO:217; and

(ab) the nucleotide sequence of the cDNA insert of clone ct489_{—}14 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:217, but excluding the poly(A) tail at the 3′ end of SEQ ID NO:217; and

(bb) the nucleotide sequence of the cDNA insert of clone ct489_{—}14 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:217, and extends contiguously from a nucleotide sequence corresponding to the 5′ end of SEQ ID NO:217 to a nucleotide sequence corresponding to the 3′ end of SEQ ID NO:217 but excluding the poly(A) tail at the 3′ end of SEQ ID NO:217. In another preferred embodiment, the nucleotide sequence of said isolated polynucleotide corresponds to the cDNA sequence of SEQ ID NO:217 from nucleotide 83 to nucleotide 1111, and extends contiguously from a nucleotide sequence corresponding to the 5′ end of said sequence of SEQ ID NO:217 from nucleotide 83 to nucleotide 1111, to a nucleotide sequence corresponding to the 3′ end of said sequence of SEQ ID NO:217 from nucleotide 83 to nucleotide 1111.

In a further preferred embodiment, the nucleotide sequence of said isolated polynucleotide corresponds to the cDNA sequence of SEQ ID NO:217 from nucleotide 155 to nucleotide 1111, and extends contiguously from a nucleotide sequence corresponding to the 5′ end of said sequence of SEQ ID NO:217 from nucleotide 155 to nucleotide 1111, to a nucleotide sequence corresponding to the 3′ end of said sequence of SEQ ID NO:217 from nucleotide 155 to nucleotide 1111.

In other embodiments, the present invention provides a composition comprising a protein, wherein said protein comprises an amino acid sequence selected from the group consisting of:

(a) the amino acid sequence of SEQ ID NO:218;

(b) fragments of the amino acid sequence of SEQ ID NO:218 comprising eight consecutive amino acids of SEQ ID NO:218; and

(c) the amino acid sequence encoded by the cDNA insert of clone ct489_{—}14 deposited under accession number ATCC 98580;

the protein being substantially free from other mammalian proteins. Preferably such protein comprises the amino acid sequence of SEQ ID NO:218. In further preferred embodiments, the present invention provides a protein comprising a fragment of the amino acid sequence of SEQ ID NO:218 having biological activity, the fragment preferably comprising eight (more preferably twenty, most preferably thirty) consecutive amino acids of SEQ ID NO:218, or a protein comprising a fragment of the amino acid sequence of SEQ ID NO:218 having biological activity, the fragment comprising the amino acid sequence from amino acid 166 to amino acid 175 of SEQ ID NO:218.

In one embodiment, the present invention provides a composition comprising an isolated polynucleotide selected from the group consisting of:

(a) a polynucleotide comprising the nucleotide sequence of SEQ ID NO:219;

(b) a polynucleotide comprising the nucleotide sequence of SEQ ID NO:219 from nucleotide 26 to nucleotide 490;

(c) a polynucleotide comprising the nucleotide sequence of the full-length protein coding sequence of clone df821_{—}1 deposited under accession number ATCC 98580;

(d) a polynucleotide encoding the full-length protein encoded by the cDNA insert of clone df821_{—}1 deposited under accession number ATCC 98580;

(e) a polynucleotide comprising the nucleotide sequence of a mature protein coding sequence of clone df821_{—}1 deposited under accession number ATCC 98580;

(f) a polynucleotide encoding a mature protein encoded by the cDNA insert of clone df821_{—}1 deposited under accession number ATCC 98580;

(g) a polynucleotide encoding a protein comprising the amino acid sequence of SEQ ID NO:220;

(h) a polynucleotide encoding a protein comprising a fragment of the amino acid sequence of SEQ ID NO:220 having biological activity, the fragment comprising eight consecutive amino acids of SEQ ID NO:220;

(i) a polynucleotide which is an allelic variant of a polynucleotide of (a)-(f) above;

(j) a polynucleotide which encodes a species homologue of the protein of (g) or (h) above; and

(k) a polynucleotide that hybridizes under stringent conditions to any one of the polynucleotides specified in (a)-(h).

Preferably, such polynucleotide comprises the nucleotide sequence of SEQ ID NO:219 from nucleotide 26 to nucleotide 490; the nucleotide sequence of the full-length protein coding sequence of clone df821_{—}1 deposited under accession number ATCC 98580; or the nucleotide sequence of a mature protein coding sequence of clone df821_{—}1 deposited under accession number ATCC 98580. In other preferred embodiments, the polynucleotide encodes the full-length or a mature protein encoded by the cDNA insert of clone df821_{—}1 deposited under accession number ATCC 98580. In yet other preferred embodiments, the present invention provides a polynucleotide encoding a protein comprising the amino acid sequence of SEQ ID NO:220 from amino acid 92 to amino acid 152. In further preferred embodiments, the present invention provides a polynucleotide encoding a protein comprising a fragment of the amino acid sequence of SEQ ID NO:220 having biological activity, the fragment preferably comprising eight (more preferably twenty, most preferably thirty) consecutive amino acids of SEQ ID NO:220, or a polynucleotide encoding a protein comprising a fragment of the amino acid sequence of SEQ ID NO:220 having biological activity, the fragment comprising the amino acid sequence from amino acid 72 to amino acid 81 of SEQ ID NO:220.

Other embodiments provide the gene corresponding to the cDNA sequence of SEQ ID NO:219.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:219, but excluding the poly(A) tail at the 3′ end of SEQ ID NO:219; and

(ab) the nucleotide sequence of the cDNA insert of clone df821_{—}1 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:219, but excluding the poly(A) tail at the 3′ end of SEQ ID NO:15; and

(bb) the nucleotide sequence of the cDNA insert of clone df821_{—}1 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:219, and extends contiguously from a nucleotide sequence corresponding to the 5′ end of SEQ ID NO:219 to a nucleotide sequence corresponding to the 3′ end of SEQ ID NO:219 but excluding the poly(A) tail at the 3′ end of SEQ ID NO:219. In another preferred embodiment, the nucleotide sequence of said isolated polynucleotide corresponds to the cDNA sequence of SEQ ID NO:219 from nucleotide 26 to nucleotide 490, and extends contiguously from a nucleotide sequence corresponding to the 5′ end of said sequence of SEQ ID NO:219 from nucleotide 26 to nucleotide 490, to a nucleotide sequence corresponding to the 3′ end of said sequence of SEQ ID NO:219 from nucleotide 26 to nucleotide 490.

In other embodiments, the present invention provides a composition comprising a protein, wherein said protein comprises an amino acid sequence selected from the group consisting of:

(a) the amino acid sequence of SEQ ID NO:220;

(b) the amino acid sequence of SEQ ID NO:220 from amino acid 92 to amino acid 152;

(c) fragments of the amino acid sequence of SEQ ID NO:220 comprising eight consecutive amino acids of SEQ ID NO:220; and

(d) the amino acid sequence encoded by the cDNA insert of clone df821_{—}1 deposited under accession number ATCC 98580;

the protein being substantially free from other mammalian proteins. Preferably such protein comprises the amino acid sequence of SEQ ID NO:220 or the amino acid sequence of SEQ ID NO:220 from amino acid 92 to amino acid 152. In further preferred embodiments, the present invention provides a protein comprising a fragment of the amino acid sequence of SEQ ID NO:220 having biological activity, the fragment preferably comprising eight (more preferably twenty, most preferably thirty) consecutive amino acids of SEQ ID NO:220, or a protein comprising a fragment of the amino acid sequence of SEQ ID NO:220 having biological activity, the fragment comprising the amino acid sequence from amino acid 72 to amino acid 81 of SEQ ID NO:220.

In one embodiment, the present invention provides a composition comprising an isolated polynucleotide selected from the group consisting of:

(a) a polynucleotide comprising the nucleotide sequence of SEQ ID NO:221;

(b) a polynucleotide comprising the nucleotide sequence of SEQ ID NO:221 from nucleotide 65 to nucleotide 412;

(c) a polynucleotide comprising the nucleotide sequence of SEQ ID NO:221 from nucleotide 197 to nucleotide 412;

(d) a polynucleotide comprising the nucleotide sequence of the full-length protein coding sequence of clone dy41_{—}2 deposited under accession number ATCC 98580;

(e) a polynucleotide encoding the full-length protein encoded by the cDNA insert of clone dy41_{—}2 deposited under accession number ATCC 98580;

(f) a polynucleotide comprising the nucleotide sequence of a mature protein coding sequence of clone dy412 deposited under accession number ATCC 98580;

(g) a polynucleotide encoding a mature protein encoded by the cDNA insert of clone dy41_{—}2 deposited under accession number ATCC 98580;

(h) a polynucleotide encoding a protein comprising the amino acid sequence of SEQ ID NO:222;

(i) a polynucleotide encoding a protein comprising a fragment of the amino acid sequence of SEQ ID NO:222 having biological activity, the fragment comprising eight consecutive amino acids of SEQ ID NO:222;

(j) a polynucleotide which is an allelic variant of a polynucleotide of (a)-(g) above;

(k) a polynucleotide which encodes a species homologue of the protein of (h) or (i) above; and

(l) a polynucleotide that hybridizes under stringent conditions to any one of the polynucleotides specified in (a)-(i).

Preferably, such polynucleotide comprises the nucleotide sequence of SEQ ID NO:221 from nucleotide 65 to nucleotide 412; the nucleotide sequence of SEQ ID NO:221 from nucleotide 197 to nucleotide 412; the nucleotide sequence of the full-length protein coding sequence of clone dy412 deposited under accession number ATCC 98580; or the nucleotide sequence of a mature protein coding sequence of clone dy412 deposited under accession number ATCC 98580. In other preferred embodiments, the polynucleotide encodes the full-length or a mature protein encoded by the cDNA insert of clone dy412 deposited under accession number ATCC 98580. In further preferred embodiments, the present invention provides a polynucleotide encoding a protein comprising a fragment of the amino acid sequence of SEQ ID NO:222 having biological activity, the fragment preferably comprising eight (more preferably twenty, most preferably thirty) consecutive amino acids of SEQ ID NO:222, or a polynucleotide encoding a protein comprising a fragment of the amino acid sequence of SEQ ID NO:222 having biological activity, the fragment comprising the amino acid sequence from amino acid 53 to amino acid 62 of SEQ ID NO:222.

Other embodiments provide the gene corresponding to the cDNA sequence of SEQ ID NO:221.

A further embodiment of the invention provides a process for producing an isolated polynucleotide, wherein the process is selected from the group consisting of:

(a) a process comprising the steps of:

(i) preparing one or more polynucleotide probes that hybridize in 6× SSC at 65 degrees C. to a nucleotide sequence selected from the group consisting of:

(aa) SEQ ID NO:221, but excluding the poly(A) tail at the 3′ end of SEQ ID NO:221; and

(ab) the nucleotide sequence of the cDNA insert of clone dy412 deposited under ATCC 98580;

(ii) hybridizing said probe(s) to human DNA; and

(iii) isolating the DNA polynucleotide detected with the probe(s); and

(b) a process comprising the steps of:

(i) preparing one or more polynucleotide primers that hybridize in 6× SSC at 65 degrees C. to a nucleotide sequence selected from the group consisting of:

(ba) SEQ ID NO:221, but excluding the poly(A) tail at the 3′ end of SEQ ID NO:221; and

(bb) the nucleotide sequence of the cDNA insert of clone dy412 deposited under ATCC 98580;

(ii) hybridizing said primer(s) to human DNA;

(iii) amplifying human DNA sequences; and

(iv) isolating the polynucleotide product of step (b)(iii).

Preferably, the nucleotide sequence of the polynucleotide isolated according to this method corresponds to the cDNA sequence of SEQ ID NO:221, and extends contiguously from a nucleotide sequence corresponding to the 5′ end of SEQ ID NO:221 to a nucleotide sequence corresponding to the 3′ end of SEQ ID NO:221 but excluding the poly(A) tail at the 3′ end of SEQ ID NO:221. In another preferred embodiment, the nucleotide sequence of said isolated polynucleotide corresponds to the cDNA sequence of SEQ ID NO:221 from nucleotide 65 to nucleotide 412, and extends contiguously from a nucleotide sequence corresponding to the 5′ end of said sequence of SEQ ID NO:221 from nucleotide 65 to nucleotide 412, to a nucleotide sequence corresponding to the 3′ end of said sequence of SEQ ID NO:221 from nucleotide 65 to nucleotide 412.

In other embodiments, the present invention provides a composition comprising a protein, wherein said protein comprises an amino acid sequence selected from the group consisting of:

(a) the amino acid sequence of SEQ ID NO:222;

(b) fragments of the amino acid sequence of SEQ ID NO:222 comprising eight consecutive amino acids of SEQ ID NO:222; and

(c) the amino acid sequence encoded by the cDNA insert of clone dy41_{—}2 deposited under accession number ATCC 98580;

the protein being substantially free from other mammalian proteins. Preferably such protein comprises the amino acid sequence of SEQ ID NO:222. In further preferred embodiments, the present invention provides a protein comprising a fragment of the amino acid sequence of SEQ ID NO:222 having biological activity, the fragment preferably comprising eight (more preferably twenty, most preferably thirty) consecutive amino acids of SEQ ID NO:222, or a protein comprising a fragment of the amino acid sequence of SEQ ID NO:222 having biological activity, the fragment comprising the amino acid sequence from amino acid 53 to amino acid 62 of SEQ ID NO:222.

In certain preferred embodiments, the polynucleotide is operably linked to an expression control sequence. The invention also provides a host cell, including bacterial, yeast, insect and mammalian cells, transformed with such polynucleotide compositions. Also provided by the present invention are organisms that have enhanced, reduced, or modified expression of the gene(s) corresponding to the polynucleotide sequences disclosed herein.

Processes are also provided for producing a protein encoded by polynucleotides of the present invention, which comprise:

(a) growing a culture of the host cell transformed with such polynucleotides in a suitable culture medium; and

(b) purifying the protein from the culture.

The protein produced according to such methods is also provided by the present invention, as are isolated polynucleotides encoding the proteins produced according to such methods.

Protein compositions of the present invention may further comprise a pharmaceutically acceptable carrier. Compositions comprising an antibody which specifically reacts with such protein are also provided by the present invention.

Methods are also provided for preventing, treating or ameliorating a medical condition which comprises administering to a mammalian subject a therapeutically effective amount of a composition comprising a protein of the present invention and a pharmaceutically acceptable carrier.

### BRIEF DESCRIPTION OF THE DRAWINGS

FIGS. 1A and 1B are schematic representations of the pED6 and pNOTs vectors, respectively, used for deposit of clones disclosed herein.

### DETAILED DESCRIPTION

Isolated Proteins and Polynucleotides

Nucleotide and amino acid sequences, as presently determined, are reported below for each clone and protein disclosed in the present application. The nucleotide sequence of each clone can readily be determined by sequencing of the deposited clone in accordance with known methods. The predicted amino acid sequence (both full-length and mature forms) can then be determined from such nucleotide sequence. The amino acid sequence of the protein encoded by a particular clone can also be determined by expression of the clone in a suitable host cell, collecting the protein and determining its sequence. For each disclosed protein applicants have identified what they have determined to be the reading frame best identifiable with sequence information available at the time of filing.

As used herein a “secreted” protein is one which, when expressed in a suitable host cell, is transported across or through a membrane, including transport as a result of signal sequences in its amino acid sequence. “Secreted” proteins include without limitation proteins secreted wholly (e.g., soluble proteins) or partially (e.g., receptors) from the cell in which they are expressed. “Secreted” proteins also include without limitation proteins which are transported across the membrane of the endoplasmic reticulum.

Clone “bh389_{—}11”

A polynucleotide of the present invention has been identified as clone “bh389_{—}11”. bh389_{—}11 was isolated from a human adult thyroid cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bh389_{—}11 is a full-length clone, including the entire coding sequence of a secreted protein (also referred to herein as “bh389_{—}11 protein”).

The nucleotide sequence of bh389_{—}11 as presently determined is reported in SEQ ID NO:1. What applicants presently believe to be the proper reading frame and the predicted amino acid sequence of the bh389_{—}11 protein corresponding to the foregoing nucleotide sequence is reported in SEQ ID NO:2. Amino acids 10 to 22 of SEQ ID NO:2 are a predicted leader/signal sequence, with the predicted mature amino acid sequence beginning at amino acid 23, or are a transmembrane domain. The TopPredII computer program predicts a potential transmembrane domain within the bh389_{—}11 protein sequence centered around amino acid 68 of SEQ ID NO:2.

Another potential bh389_{—}11 reading frame and predicted amino acid sequence is encoded by basepairs 757 to 1833 of SEQ ID NO:1 and is reported in SEQ ID NO:34. A frameshift in the nucleotide sequence of SEQ ID NO:1 between about nucleotide 754 to about nucleotide 803 could join the reading frames of SEQ ID NO:1 and SEQ ID NO:34. The TopPredII computer program predicts a potential transmembrane domain within the amino acid sequence of SEQ ID NO:34, centered around amino acid 357 of SEQ ID NO:34.

The EcoRI/NotI restriction fragment obtainable from the deposit containing clone bh389_{—}11 should be approximately 1700 bp.

The nucleotide sequence disclosed herein for bh389_{—}11 was searched against the GenBank and GeneSeq nucleotide sequence databases using BLASTN/BLASTX and FASTA search protocols. bh389_{—}11 demonstrated at least some similarity with sequences identified as AA307880 (EST178733 Colon carcinoma (HCC) cell line Homo sapiens cDNA 5′ end), AA442426 (zv70f06.rl Soares total fetus Nb2HF8 9w Homo sapiens cDNA clone 759011 5′), H70103 (yr92f04.rl Homo sapiens cDNA clone 212767 5′), R19820 (yg37f12.rl Homo sapiens cDNA clone 34771 5′), and W46238 (zc30e10.s1 Soares senescent fibroblasts NbHSF Homo sapiens cDNA clone 323850 3′). Based upon sequence similarity, bh389_{—}11 proteins and each similar protein or peptide may share at least some activity.

Clone “bk112_{—}15”

A polynucleotide of the present invention has been identified as clone “bk112_{—}15”. bk112_{—}15 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bk112_{—}15 is a full-length clone, including the entire coding sequence of a secreted protein (also referred to herein as “bk112_{—}15 protein”).

The nucleotide sequence of bk112_{—}15 as presently determined is reported in SEQ ID NO:3. What applicants presently believe to be the proper reading frame and the predicted amino acid sequence of the bk112_{—}15 protein corresponding to the foregoing nucleotide sequence is reported in SEQ ID NO:4.

The EcoRI/NotI restriction fragment obtainable from the deposit containing clone bk112_{—}15 should be approximately 1300 bp.

The nucleotide sequence disclosed herein for bk112_{—}15 was searched against the GenBank and GeneSeq nucleotide sequence databases using BLASTN/BLASTX and FASTA search protocols. bk112_{—}15 demonstrated at least some similarity with sequences identified as AA307119 (EST178031 Colon carcinoma (HCC) cell line Homo sapiens cDNA 5′ end), AA318352 (EST20422 Retina II Homo sapiens cDNA 5′ end similar to similar to C. elegans hypothetical protein, cosmid ZK688.2), L20941 (Human ferritin heavy chain mRNA, complete cds), M97164 (Human ferritin heavy chain mRNA, complete cds), N25339 (yx55d08.s1 Homo sapiens cDNA clone 265647 3′), N31453 (yx55d08.rl Homo sapiens cDNA clone 265647 5′), and N33227 (yy07d02.s1 Homo sapiens cDNA clone 270531 3′ similar to gb:L20941 FERRITIN HEAVY CHAIN (HUMAN)). The predicted amino acid sequence disclosed herein for bk112_{—}15 was searched against the GenPept and GeneSeq amino acid sequence databases using the BLASTX search protocol. The predicted bk112_{—}15 protein demonstrated at least some similarity to sequences identified as Z68335 (C29F4.2 [Caenorhabditis elegans]). Based upon sequence similarity, bk112_{—}15 proteins and each similar protein or peptide may share at least some activity.

Clone “bk200_{—}13”

A polynucleotide of the present invention has been identified as clone “bk200_{—}13”. bk200_{—}13 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bk200_{—}13 is a full-length clone, including the entire coding sequence of a secreted protein (also referred to herein as “bk200_{—}13 protein”).

The nucleotide sequence of bk200_{—}13 as presently determined is reported in SEQ ID NO:5. What applicants presently believe to be the proper reading frame and the predicted amino acid sequence of the bk200_{—}13 protein corresponding to the foregoing nucleotide sequence is reported in SEQ ID NO:6.

The EcoRI/NotI restriction fragment obtainable from the deposit containing clone bk200_{—}13 should be approximately 1000 bp.

The nucleotide sequence disclosed herein for bk200_{—}13 was searched against the GenBank and GeneSeq nucleotide sequence databases using BLASTN/BLASTX and FASTA search protocols. bk200_{—}13 demonstrated at least some similarity with sequences identified as AA098915 zk84f06.s1 Soares pregnant uterus NbHPU Homo sapiens cDNA clone 489539 3′), AA150367 z107b06.rl Soares pregnant uterus NbHPU Homo sapiens cDNA clone 491603 5′), AA235904 (zs40h05.rl Soares NhHMPu S1 Homo sapiens cDNA clone 687705 5′), N32487 (yx79g10.rl Homo sapiens cDNA clone 268002 5′), and T47862 (yb17g03.rl Homo sapiens cDNA clone 71476 5′). Based upon sequence similarity, bk200_{—}13 proteins and each similar protein or peptide may share at least some activity. The nucleotide sequence of bk200_{—}13 may contain CAAAAA repeat-like elements.

Clone “di386_{—}3”

A polynucleotide of the present invention has been identified as clone “di386_{—}3”. di386_{—}3 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. di386_{—}3 is a full-length clone, including the entire coding sequence of a secreted protein (also referred to herein as “di386_{—}3 protein”).

The nucleotide sequence of the 5′ portion of di386_{—}3 as presently determined is reported in SEQ ID NO:7. What applicants presently believe is the proper reading frame for the coding region is indicated in SEQ ID NO:8. The predicted amino acid sequence of the di386_{—}3 protein corresponding to the foregoing nucleotide sequence is reported in SEQ ID NO:8. Amino acids 39 to 51 are a predicted leader/signal sequence, with the predicted mature amino acid sequence beginning at amino acid 52, or are a transmembrane domain. Amino acids 17 to 29° F. SEQ ID NO:8 are also a possible leader/signal sequence, with the predicted mature amino acid sequence beginning at amino acid 30, or are a transmembrane domain. Additional nucleotide sequence from the 3′ portion of di386_{—}3, including the polyA tail, is reported in SEQ ID NO:9.

The EcoRI/NotI restriction fragment obtainable from the deposit containing clone di386_{—}3 should be approximately 2000 bp.

The nucleotide sequence disclosed herein for di386_{—}3 was searched against the GenBank and GeneSeq nucleotide sequence databases using BLASTN/BLASTX and FASTA search protocols. di386_{—}3 demonstrated no similarity with any known sequences in those databases.

Clone “em397_{—}2”

A polynucleotide of the present invention has been identified as clone “em397_{—}2”. em397_{—}2 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. em397_{—}2 is a full-length clone, including the entire coding sequence of a secreted protein (also referred to herein as “em397_{—}2 protein”).

The nucleotide sequence of em397_{—}2 as presently determined is reported in SEQ ID NO:10. What applicants presently believe to be the proper reading frame and the predicted amino acid sequence of the em397_{—}2 protein corresponding to the foregoing nucleotide sequence is reported in SEQ ID NO:11.

The EcoRI/NotI restriction fragment obtainable from the deposit containing clone em397_{—}2 should be approximately 1250 bp.

The nucleotide sequence disclosed herein for em397_{—}2 was searched against the GenBank and GeneSeq nucleotide sequence databases using BLASTN/BLASTX and FASTA search protocols. em397_{—}2 demonstrated at least some similarity with sequences identified as AA092876 (m0851.seq.F Fetal heart, Lambda ZAP Express Homo sapiens cDNA 5′), AA180952 (zp41b06.rl Stratagene muscle 937209 Homo sapiens cDNA clone 611987 5′), AA463323 (zx71f10.rl Soares total fetus Nb2HF8 9w Homo sapiens), H87081 (ys74f01.rl Homo sapiens cDNA clone 220537 5′), W56381 (zc57a01.rl Soares parathyroid tumor NbHPA Homo sapiens cDNA clone 326376 5′), W88527 (zh73g02.s1 Soares fetal liver spleen 1NFLS S1 Homo sapiens cDNA clone 417746 3′), and Z64565 (H.sapiens CpG island DNA genomic Mse1 fragment, clone 13d12, reverse read cpg13d12.rt1c). Based upon sequence similarity, em397_{—}2 proteins and each similar protein or peptide may share at least some activity.

Clone “fh170_{—}7”

A polynucleotide of the present invention has been identified as clone “fh170_{—}7”. fh170_{—}7 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fh170_{—}7 is a full-length clone, including the entire coding sequence of a secreted protein (also referred to herein as “fh170_{—}7 protein”).

The nucleotide sequence of fh170_{—}7 as presently determined is reported in SEQ ID NO:12. What applicants presently believe to be the proper reading frame and the predicted amino acid sequence of the fh170_{—}7 protein corresponding to the foregoing nucleotide sequence is reported in SEQ ID NO:13. Amino acids 127 to 139 are a predicted leader/signal sequence, with the predicted mature amino acid sequence beginning at amino acid 140, or are a transmembrane domain.

The EcoRI/NotI restriction fragment obtainable from the deposit containing clone fh170_{—}7 should be approximately 2200 bp.

The nucleotide sequence disclosed herein for fh170_{—}7 was searched against the GenBank and GeneSeq nucleotide sequence databases using BLASTN/BLASTX and FASTA search protocols. fh170_{—}7 demonstrated at least some similarity with sequences identified as AA112479 (zn69a02.s1 Stratagene HeLa cell s3 937216 Homo sapiens cDNA clone 563402 3′), AA593402 (nn57g10.s1 NCI_CGAP_Kid6 Homo sapiens cDNA clone IMAGE:1088034), Q76795 (Human genome fragment), T26136 (Human gene signature HUMGS08373), and Z19759 (H. sapiens putatively transcribed partial sequence; UK-HGMP sequence ID AAAALWX; single read). The predicted amino acid sequence disclosed herein for fh170_{—}7 was searched against the GenPept and GeneSeq amino acid sequence databases using the BLASTX search protocol. The predicted fh170_{—}7 protein demonstrated at least some similarity to sequences identified as D32253 (MagA [Magnetospirillum sp.]) and W01520 (MagA protein). The predicted fh170_{—}7 protein also demonstrated at least some similarity to other prokaryotic membrane transport proteins: potassium-efflux system protein kefB and NaH-antiporter protein. Based upon sequence similarity, fh170_{—}7 proteins and each similar protein or peptide may share at least some activity. The TopPredII computer program predicts ten potential transmembrane domains within the fh170_{—}7 protein sequence, centered around amino acids 130, 160, 210, 230, 280, 310, 360, 380, 420, and 500 of SEQ ID NO:13, respectively.

Clone “fn53_{—}4”

A polynucleotide of the present invention has been identified as clone “fn53_{—}4”. fn53_{—}4 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fn53_{—}4 is a full-length clone, including the entire coding sequence of a secreted protein (also referred to herein as “fn53_{—}4 protein”).

The nucleotide sequence of the 5′ portion of fn53_{—}4 as presently determined is reported in SEQ ID NO:14. An additional internal nucleotide sequence from fn53_{—}4 as presently determined is reported in SEQ ID NO:15. What applicants believe is the proper reading frame and the predicted amino acid sequence encoded by such internal sequence is reported in SEQ ID NO:16. Additional nucleotide sequence from the 3′ portion of fn53_{—}4, including the polyA tail, is reported in SEQ ID NO:17.

The EcoRI/NotI restriction fragment obtainable from the deposit containing clone fn53_{—}4 should be approximately 4100 bp.

The nucleotide sequence disclosed herein for fn53_{—}4 was searched against the GenBank and GeneSeq nucleotide sequence databases using BLASTN/BLASTX and FASTA search protocols. fn53_{—}4 demonstrated at least some similarity with sequences identified as AA179207 (zp46c11.s1 Stratagene HeLa cell s3 937216 Homo sapiens cDNA clone 612500 3′), AA279207 (zs83e06.s1 NCI_CGAP_GCB1 Homo sapiens cDNA clone IMAGE:704098 3′, mRNA sequence), H87151 (yw15a06.s1 Homo sapiens cDNA clone 252274 3′), and H83373 (ys90a09.rl Homo sapiens cDNA clone 222040 5′ similar to SP:BICD_DROME P16568 CYTOSKELETON-LIKE BICAUDAL D). The predicted amino acid sequence disclosed herein for fn53_{—}4 was searched against the GenPept and GeneSeq amino acid sequence databases using the BLASTX search protocol. The predicted fn53_{—}4 protein demonstrated at least some similarity to sequences identified as M3 1684 and X5 1652 (bicaudalD protein [Drosophila melanogaster]) and R66930 (AMML chromosome inv(16) product). Based upon sequence similarity, fn53_{—}4 proteins and each similar protein or peptide may share at least some activity.

Clone “fq505_{—}4”

A polynucleotide of the present invention has been identified as clone “fq505_{—}4”. fq505_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fq505_{—}4 is a full-length clone, including the entire coding sequence of a secreted protein (also referred to herein as “fq505_{—}4 protein”).

The nucleotide sequence of fq505_{—}4 as presently determined is reported in SEQ ID NO:18. What applicants presently believe to be the proper reading frame and the predicted amino acid sequence of the fq505_{—}4 protein corresponding to the foregoing nucleotide sequence is reported in SEQ ID NO:19.

The EcoRI/NotI restriction fragment obtainable from the deposit containing clone fq505_{—}4 should be approximately 512 bp.

The nucleotide sequence disclosed herein for fq505_{—}4 was searched against the GenBank and GeneSeq nucleotide sequence databases using BLASTN/BLASTX and FASTA search protocols. fq505_{—}4 demonstrated at least some similarity with sequences identified as Z71861 (C.hircus mRNA for EST2-31). The predicted amino acid sequence disclosed herein for fq505_{—}4 was searched against the GenPept and GeneSeq amino acid sequence databases using the BLASTX search protocol. The predicted fq505_{—}4 protein demonstrated at least some similarity to sequences identified as P92141 (Recombinant human adult T cell leukaemia derived factor polypeptide), X54539 (thioredoxin [Homo sapiens]), and X77584 (ATL-derived factor/thioredoxin [Homo sapiens]). The predicted fq505_{—}4 protein also demonstrated at least some similarity to sequences identified as surface associated sulphydryl protein (GenProt accession number135773). The similarity between these proteins includes a WCGPC catalytic site, which is present as RCGPC at amino acids 31 to 35 of the predicted fq505_{—}4 protein. In addition to having thioredoxin catalytic activity, at least one thioredoxin-related protein has also been reported to be “an IL-2 receptor/Tac inducer” (Tagaya et al., 1989, EMBO J. 8(3): 757-764). At least one thioredoxin-related protein is reported to be associated with the plasma membrane, “indicating that the protein may be a member of this [thioredoxin] family and function as an essential growth factor” (Martin and Dean, 1991, Biochem. Biophys. Res. Commun. 175(1): 123-128). Based upon sequence similarity, fq505_{—}4 proteins and each similar protein or peptide may share at least some activity.

Clone “fw13_{—}9”

A polynucleotide of the present invention has been identified as clone “fw13_{—}9”. fw13_{—}9 was isolated from a human adult testes (teratocarcinoma NCCIT) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fw13_{—}9 is a full-length clone, including the entire coding sequence of a secreted protein (also referred to herein as “fw13_{—}9 protein”).

The nucleotide sequence of fw13_{—}9 as presently determined is reported in SEQ ID NO:20. What applicants presently believe to be the proper reading frame and the predicted amino acid sequence of the fw13_{—}9 protein corresponding to the foregoing nucleotide sequence is reported in SEQ ID NO:21.

The EcoRI/NotI restriction fragment obtainable from the deposit containing clone fw13_{—}9 should be approximately 1900 bp.

The nucleotide sequence disclosed herein for fw13_{—}9 was searched against the GenBank and GeneSeq nucleotide sequence databases using BLASTN/BLASTX and FASTA search protocols. fw13_{—}9 demonstrated at least some similarity with sequences identified as AA047557 (zf13f08.rl Soares fetal heart NbHH19W Homo sapiens cDNA clone 376839 5′), AA284524 (zt20d07.s1 Soares ovary tumor NbHOT Homo sapiens cDNA clone 713677 3′), AA502778 (ne43e04.s1 NCI_CGAP_Co3 Homo sapiens cDNA clone IMAGE:900126), J04743 (M.musculus Ms6-hm locus, repeat elements), R35040 (yh86a10.rl Homo sapiens cDNA clone 136602 5′), T21414 (Human gene signature HUMGS02783), and U91318 (Human chromosome 16p13 BAC clone CIT987SK-962B4 complete sequence). Based upon sequence similarity, fw13_{—}9 proteins and each similar protein or peptide may share at least some activity. The TopPredII computer program predicts a potential transmembrane domain within the fw13_{—}9 protein sequence centered around amino acid 30 of SEQ ID NO:21.

Clone “gg619_{—}2”

A polynucleotide of the present invention has been identified as clone “gg619_{—}2”. gg619_{—}2 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gg619_{—}2 is a full-length clone, including the entire coding sequence of a secreted protein (also referred to herein as “gg619_{—}2 protein”).

The nucleotide sequence of gg619_{—}2 as presently determined is reported in SEQ ID NO:22. What applicants presently believe to be the proper reading frame and the predicted amino acid sequence of the gg619_{—}2 protein corresponding to the foregoing nucleotide sequence is reported in SEQ ID NO:23.

The EcoRI/NotI restriction fragment obtainable from the deposit containing clone gg619_{—}2 should be approximately 1350 bp.

The nucleotide sequence disclosed herein for gg619_{—}2 was searched against the GenBank and GeneSeq nucleotide sequence databases using BLASTN/BLASTX and FASTA search protocols. gg619_{—}2 demonstrated at least some similarity with sequences identified as N42957 (yy12b12.rl Homo sapiens cDNA clone 271007 5′ similar to SW:ALG5_YEAST P40350 dolichyl-phosphate beta-glucosyltransferase), N50844 (yy91g05.s1 Homo sapiens cDNA clone 280952 3′ similar to SW:ALG5_YEAST P40350 dolichyl-phosphate beta-glucosyltransferase), and N62597 (yz75a06.s1 Homo sapiens cDNA clone 288850 3′ similar to SW:ALG5_YEAST P40350 Dolichyl-phosphate beta-glucosyltransferase). The predicted amino acid sequence disclosed herein for gg619_{—}2 was searched against the GenPept and GeneSeq amino acid sequence databases using the BLASTX search protocol. The predicted gg619_{—}2 protein demonstrated at least some similarity to sequences identified as R38093 (nodC N-terminal portion [Bradyrhizobium sp. (Parasponia)]) and X77573 (dolichyl-phosphate beta-glucosyl-transferase [Saccharomyces cerevisiae]). The enzyme UDP-glucose:dolichyl-phosphate glucosyltransferase is a transmembrane-bound enzyme of the endoplasmic reticulum involved in protein N-linked glycosylation, and catalyzes the transfer of glucose from UDP-glucose to dolichyl phosphate. Based upon sequence similarity, gg619_{—}2 proteins and each similar protein or peptide may share at least some activity. The TopPredII computer program predicts a potential transmembrane domain within the gg619_{—}2 protein sequence, centered around amino acid 188 of SEQ ID NO:23.

Clone “cl181_{—}3”

A polynucleotide of the present invention has been identified as clone “cl181_{—}3”. cl181_{—}3 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cl181_{—}3 is a full-length clone, including the entire coding sequence of a secreted protein (also referred to herein as “cl181_{—}3 protein”).

The nucleotide sequence of cl181_{—}3 as presently determined is reported in SEQ ID NO:35. What applicants presently believe to be the proper reading frame and the predicted amino acid sequence of the cl181_{—}3 protein corresponding to the foregoing nucleotide sequence is reported in SEQ ID NO:36. Amino acids 50 to 62 are a predicted leader/signal sequence, with the predicted mature amino acid sequence beginning at amino acid 63, or are a transmembrane domain.

The EcoRI/NotI restriction fragment obtainable from the deposit containing clone cl181_{—}3 should be approximately 2800 bp.

The nucleotide sequence disclosed herein for cl181_{—}3 was searched against the GenBank and GeneSeq nucleotide sequence databases using BLASTN/BLASTX and FASTA search protocols. cl181_{—}3 demonstrated at least some similarity with sequences identified as C16190 (Human aorta cDNA 5′-end GEN-241B04) and L36900 (Saccharomyces cerevisiae mitochondrion transfer RNA-Ser1 (tRNA-Ser) gene and varI gene, complete cds). Based upon sequence similarity, cl181_{—}3 proteins and each similar protein or peptide may share at least some activity. The TopPredII computer program predicts a potential transmembrane domain within the cl181_{—}3 protein sequence centered around amino acid 77 of SEQ ID NO:36.

Clone “cr1044_{—}1”

A polynucleotide of the present invention has been identified as clone “cr1044_{—}1”. cr1044_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cr1044_{—}1 is a full-length clone, including the entire coding sequence of a secreted protein (also referred to herein as “cr1044_{—}1 protein”).

The nucleotide sequence of cr1044_{—}1 as presently determined is reported in SEQ ID NO:37. What applicants presently believe to be the proper reading frame and the predicted amino acid sequence of the cr1044_{—}1 protein corresponding to the foregoing nucleotide sequence is reported in SEQ ID NO:38. Amino acids 158 to 170 of SEQ ID NO:36 are a predicted leader/signal sequence, with the predicted mature amino acid sequence beginning at amino acid 171 of SEQ ID NO:38, or are a transmembrane domain. Base pairs 175 to 237 of SEQ ID NO:37 are a possible intron; if this sequence were removed from SEQ ID NO:37, another potential cr1044_{—}1 reading frame and predicted amino acid sequence that could be encoded by basepairs 45 to 830 of SEQ ID NO:37 is reported in SEQ ID NO:64. Amino acids 7 to 19 of SEQ ID NO:31 are a predicted leader/signal sequence, with the predicted mature amino acid sequence beginning at amino acid 20 of SEQ ID NO:64, or are a transmembrane domain.

The EcoRI/NotI restriction fragment obtainable from the deposit containing clone cr1044_{—}1 should be approximately 3200 bp.

The nucleotide sequence disclosed herein for cr1044_{—}1 was searched against the GenBank and GeneSeq nucleotide sequence databases using BLASTN/BLASTX and FASTA search protocols. cr1044_{—}1 demonstrated at least some similarity with sequences identified as N99156 (zb81g04.s1 Soares senescent fibroblasts NbHSF Homo sapiens cDNA clone 310038 3′), Q46852 (clone of recombinant human kappa casein gene fragment), and T20727 (Human gene signature HUMGS01945). The predicted amino acid sequence disclosed herein for cr1044_{—}1 was searched against the GenPept and GeneSeq amino acid sequence databases using the BLASTX search protocol. The predicted cr1044_{—}1 protein demonstrated at least some similarity to sequences identified as M16279 (antigen [Homo sapiens]) and U82164 (human CD99 type 11). Based upon sequence similarity, cr1044_{—}1 proteins and each similar protein or peptide may share at least some activity.

Clone “cz251_{—}1”

A polynucleotide of the present invention has been identified as clone “cz251_{—}1”. cz251_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cz251_{—}1 is a full-length clone, including the entire coding sequence of a secreted protein (also referred to herein as “cz251_{—}1 protein”).

The nucleotide sequence of cz251_{—}1 as presently determined is reported in SEQ ID NO:39. What applicants presently believe to be the proper reading frame and the predicted amino acid sequence of the cz251_{—}1 protein corresponding to the foregoing nucleotide sequence is reported in SEQ ID NO:40.

The EcoRI/NotI restriction fragment obtainable from the deposit containing clone cz251_{—}1 should be approximately 1200 bp.

The nucleotide sequence disclosed herein for cz251_{—}1 was searched against the GenBank and GeneSeq nucleotide sequence databases using BLASTN/BLASTX and FASTA search protocols. cz251_{—}1 demonstrated at least some similarity with sequences identified as R55084 (yg87a06.rl Homo sapiens cDNA clone 40244 5′) and U00930 (Human clone C4E 1.63 (CAC)n/(GTG)n repeat-containing mRNA). The predicted amino acid sequence disclosed herein for cz251_{—}1 was searched against the GenPept and GeneSeq amino acid sequence databases using the BLASTX search protocol. The predicted cz251_{—}1 protein demonstrated at least some similarity to the sequence identified as Z68751 (F10G4.1 [Caenorhabditis elegans]). Based upon sequence similarity, cz251_{—}1 proteins and each similar protein or peptide may share at least some activity. The nucleotide sequence of cz251_{—}1 may contain CAAA-like repeats.

Clone “dd12_{—}7”

A polynucleotide of the present invention has been identified as clone “dd12_{—}7”. dd12_{—}7 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dd12_{—}7 is a full-length clone, including the entire coding sequence of a secreted protein (also referred to herein as “dd12_{—}7 protein”).

The nucleotide sequence of dd12_{—}7 as presently determined is reported in SEQ ID NO:41. What applicants presently believe to be the proper reading frame and the predicted amino acid sequence of the dd12_{—}7 protein corresponding to the foregoing nucleotide sequence is reported in SEQ ID NO:42.

The EcoRI/NotI restriction fragment obtainable from the deposit containing clone dd12_{—}7 should be approximately 1550 bp.

The nucleotide sequence disclosed herein for dd12_{—}7 was searched against the GenBank and GeneSeq nucleotide sequence databases using BLASTN/BLASTX and FASTA search protocols. dd12_{—}7 demonstrated at least some similarity with sequences identified as AA257999 (zs34a02.s1 Soares NbHTGBC Homo sapiens cDNA clone 687050 3′). Based upon sequence similarity, dd12_{—}7 proteins and each similar protein or peptide may share at least some activity.

Clone “fn191_{—}3”

A polynucleotide of the present invention has been identified as clone “fn191_{—}3”. fn191_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fn191_{—}3 is a full-length clone, including the entire coding sequence of a secreted protein (also referred to herein as “fn191_{—}3 protein”).

The nucleotide sequence of fn191_{—}3 as presently determined is reported in SEQ ID NO:43. What applicants presently believe to be the proper reading frame and the predicted amino acid sequence of the fn191_{—}3 protein corresponding to the foregoing nucleotide sequence is reported in SEQ ID NO:44. Amino acids 39 to 51 are a predicted leader/signal sequence, with the predicted mature amino acid sequence beginning at amino acid 52, or are a transmembrane domain.

The EcoRI/NotI restriction fragment obtainable from the deposit containing clone fn191_{—}3 should be approximately 3000 bp.

The nucleotide sequence disclosed herein for fn191_{—}3 was searched against the GenBank and GeneSeq nucleotide sequence databases using BLASTN/BLASTX and FASTA search protocols. fn191_{—}3 demonstrated at least some similarity with sequences identified as AA046787 (zk72f07.rl Soares pregnant uterus NbHPU Homo sapiens cDNA clone 488389 5′ similar to gb:L07077 ENOYL-COA HYDRATASE (HUMAN);contains Alu repetitive element), G13132 (human chromosome 7 STS sWSS3349; single read), T06013 (EST03902 Homo sapiens cDNA clone HFBDL25), T08594 (EST06486 Homo sapiens cDNA clone HIBBG72 5′ end), and W28342 (45g9 Human retina cDNA randomly primed sublibrary Homo sapiens cDNA). Based upon sequence similarity, fn191_{—}3 proteins and each similar protein or peptide may share at least some activity. The nucleotide sequence in the 5′ untranslated region of fn191_{—}3 may contain some repetitive elements.

Clone “gm196_{—}4”

A polynucleotide of the present invention has been identified as clone “gm196_{—}4”. gm196_{—}4 was isolated from a human adult uterus cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gm196_{—}4 is a full-length clone, including the entire coding sequence of a secreted protein (also referred to herein as “gm196_{—}4 protein”).

The nucleotide sequence of gm196_{—}4 as presently determined is reported in SEQ ID NO:45. What applicants presently believe to be the proper reading frame and the predicted amino acid sequence of the gm196_{—}4 protein corresponding to the foregoing nucleotide sequence is reported in SEQ ID NO:46. Another potential gm196_{—}4 reading frame and predicted amino acid sequence is encoded by basepairs 1364 to 2809 of SEQ ID NO:43 and is reported in SEQ ID NO:66. A frameshift in the nucleotide sequence of SEQ ID NO:45 could join the reading frames of SEQ ID NO:46 and SEQ ID NO:66. The TopPredII computer program predicts two potential transmembrane domains within the amino acid sequence of SEQ ID NO:66. Preferred fragments of the amino acid sequence of SEQ ID NO:66 comprise amino acids 1 to 163 or amino acids 236 to 245 of SEQ ID NO:66.

The EcoRI/NotI restriction fragment obtainable from the deposit containing clone gm196_{—}4 should be approximately 3000 bp.

The nucleotide sequence disclosed herein for gm196_{—}4 was searched against the GenBank and GeneSeq nucleotide sequence databases using BLASTN/BLASTX and FASTA search protocols. gm196_{—}4 demonstrated at least some similarity with sequences identified as AA233552 (zr43a10.s1 Soares NhHMPu S1 Homo sapiens cDNA clone 666138 3′), AB005666 (Homo sapiens mRNA for GTPase-activating protein, complete cds), F12887 (H. sapiens partial cDNA sequence; clone c-3fh09), T25078 (Human gene signature HUMGS07218), and T75264 (yc88g09.rl Homo sapiens cDNA clone 23008 5′). The predicted amino acid sequence disclosed herein for gm196_{—}4 was searched against the GenPept and GeneSeq amino acid sequence databases using the BLASTX search protocol. The predicted gm196_{—}4 protein demonstrated at least some similarity to the sequence identified as M64788 (GTPase activating protein [Homo sapiens]). Based upon sequence similarity, gm196_{—}4 proteins and each similar protein or peptide may share at least some activity.

Clone “gn141_{—}1”

A polynucleotide of the present invention has been identified as clone “gn114_{—}1”. gn114_{—}1 was isolated from a human adult blood (peripheral blood mononuclear cells treated with granulocyte-colony stimulating factor in vivo) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gn114_{—}1 is a full-length clone, including the entire coding sequence of a secreted protein (also referred to herein as “gn114_{—}1 protein”).

The nucleotide sequence of gn114_{—}1 as presently determined is reported in SEQ ID NO:47. What applicants presently believe to be the proper reading frame and the predicted amino acid sequence of the gn114_{—}1 protein corresponding to the foregoing nucleotide sequence is reported in SEQ ID NO:48. Amino acids 26 to 38 of SEQ ID NO:48 are a predicted leader/signal sequence, with the predicted mature amino acid sequence beginning at amino acid 39 of SEQ ID NO:48, or are a transmembrane domain.

The EcoRI/NotI restriction fragment obtainable from the deposit containing clone gn114_{—}1 should be approximately 1500 bp.

The nucleotide sequence disclosed herein for gn114_{—}1 was searched against the GenBank and GeneSeq nucleotide sequence databases using BLASTN/BLASTX and FASTA search protocols. gn114_{—}1 demonstrated at least some similarity with sequences identified as AA370547 (EST82206 Prostate gland I Homo sapiens cDNA 5′ end), C04732 (Human Heart cDNA, clone 3NHC3910), and C05361 (Human Heart cDNA, clone 3NHC2451). Based upon sequence similarity, gn114_{—}1 proteins and each similar protein or peptide may share at least some activity. The TopPredII computer program predicts an additional potential transmembrane domain within the gn114_{—}1 protein sequence centered around amino acid 90 of SEQ ID NO:48.

Clone “hj968_{—}2”

A polynucleotide of the present invention has been identified as clone “hj968_{—}2”. hj968_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. hj968_{—}2 is a full-length clone, including the entire coding sequence of a secreted protein (also referred to herein as “hj968_{—}2 protein”).

The nucleotide sequence of hj968_{—}2 as presently determined is reported in SEQ ID NO:49. What applicants presently believe to be the proper reading frame and the predicted amino acid sequence of the hj968_{—}2 protein corresponding to the foregoing nucleotide sequence is reported in SEQ ID NO:50. Amino acids 1 to 9 are a predicted leader/signal sequence, with the predicted mature amino acid sequence beginning at amino acid 10, or are a transmembrane domain.

The EcoRI/NotI restriction fragment obtainable from the deposit containing clone hj968_{—}2 should be approximately 1800 bp.

The nucleotide sequence disclosed herein for hj968_{—}2 was searched against the GenBank and GeneSeq nucleotide sequence databases using BLASTN/BLASTX and FASTA search protocols. hj968_{—}2 demonstrated at least some similarity with sequences identified as AA071746 (mf16h07.rl Life Tech mouse brain Mus musculus cDNA clone 405277 5′) and AA325286 (EST28500 Cerebellum II Homo sapiens cDNA 5′ end). The predicted amino acid sequence disclosed herein for hj968_{—}2 was searched against the GenPept and GeneSeq amino acid sequence databases using the BLASTX search protocol. The predicted hj968_{—}2 protein demonstrated at least some similarity to the sequence identified as U23528 (translated cosmid B0034.[Caenorhabditis elegans]). Based upon sequence similarity, hj968_{—}2 proteins and each similar protein or peptide may share at least some activity.

Clone “hk10_{—}3”

A polynucleotide of the present invention has been identified as clone “hk10_{—}3”. hk10_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. hk10_{—}3 is a full-length clone, including the entire coding sequence of a secreted protein (also referred to herein as “hk10_{—}3 protein”).

The nucleotide sequence of hk10_{—}3 as presently determined is reported in SEQ ID NO:51. What applicants presently believe to be the proper reading frame and the predicted amino acid sequence of the hk10_{—}3 protein corresponding to the foregoing nucleotide sequence is reported in SEQ ID NO:52. Amino acids 28 to 40 are a predicted leader/signal sequence, with the predicted mature amino acid sequence beginning at amino acid 41, or are a transmembrane domain.

The EcoRI/NotI restriction fragment obtainable from the deposit containing clone hk10_{—}3 should be approximately 1300 bp.

The nucleotide sequence disclosed herein for hk10_{—}3 was searched against the GenBank and GeneSeq nucleotide sequence databases using BLASTN/BLASTX and FASTA search protocols. hk10_{—}3 demonstrated at least some similarity with sequences identified as AA305975 (EST176966 Jurkat T-cells VI Homo sapiens cDNA 5′ end similar to S. cerevisiae hypothetical protein FAA3-BET1), F18016 (H.sapiens EST sequence 016-T), N36880 (yy37e07.s1 Homo sapiens cDNA clone 273444 3′), and R87757 (yo45a08.s1 Homo sapiens cDNA clone 180854 3′). The predicted amino acid sequence disclosed herein for hk10_{—}3 was searched against the GenPept and GeneSeq amino acid sequence databases using the BLASTX search protocol. The predicted hk10_{—}3 protein demonstrated at least some similarity to the sequence identified as Z38113 (CAI 0.21 [Saccharomyces cerevisiae]). Based upon sequence similarity, hk10_{—}3 proteins and each similar protein or peptide may share at least some activity.

Clone “hm236_{—}1”

A polynucleotide of the present invention has been identified as clone “hm236_{—}1”. hm236_{—}1 was isolated from a human adult testes (teratocarcinoma NCCIT) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. hm236_{—}1 is a full-length clone, including the entire coding sequence of a secreted protein (also referred to herein as “hm236_{—}1 protein”).

The nucleotide sequence of hm236_{—}1 as presently determined is reported in SEQ ID NO:53. What applicants presently believe to be the proper reading frame and the predicted amino acid sequence of the hm236_{—}1 protein corresponding to the foregoing nucleotide sequence is reported in SEQ ID NO:54.

The EcoRI/NotI restriction fragment obtainable from the deposit containing clone hm236_{—}1 should be approximately 1800 bp.

The nucleotide sequence disclosed herein for hm236_{—}1 was searched against the GenBank and GeneSeq nucleotide sequence databases using BLASTN/BLASTX and FASTA search protocols. hm236_{—}1 demonstrated at least some similarity with sequences identified as AA026169 (zk01b03.rl Soares pregnant uterus NbHPU Homo sapiens cDNA clone 469229 5′), AA046211 (zk77e04.s1 Soares pregnant uterus NbHPU Homo sapiens cDNA clone 488862 3′), AA223088 (PMY0368 KGla Lambda Zap Express cDNA Library Homo sapiens cDNA 5′), AB002368 and AC003010 (Human mRNA for KIAA0370 gene, partial cds), AC002399 (Human chromosome 16p11.2 BAC clone CIT987SK-A-481B3; HTGS phase 1, 18 unordered pieces, sequencing in progress), R86676 (ym86f03.rl Homo sapiens cDNA clone), and T21501 (Human gene signature HUMGS02874). The predicted amino acid sequence disclosed herein for hm236_{—}1 was searched against the GenPept and GeneSeq amino acid sequence databases using the BLASTX search protocol. The predicted hm236_{—}1 protein demonstrated at least some similarity to sequences identified as AB002368 (KIAA0370 [Homo sapiens]). Based upon sequence similarity, hm236_{—}1 proteins and each similar protein or peptide may share at least some activity.

Clone “do15_{—}4”

A polynucleotide of the present invention has been identified as clone “do15_{—}4”. do15_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. do15_{—}4 is a full-length clone, including the entire coding sequence of a secreted protein (also referred to herein as “do15_{—}4 protein”).

The nucleotide sequence of do15_{—}4 as presently determined is reported in SEQ ID NO:67. What applicants presently believe to be the proper reading frame and the predicted amino acid sequence of the do15_{—}4 protein corresponding to the foregoing nucleotide sequence is reported in SEQ ID NO:68. Amino acids 394 to 406 are a predicted leader/signal sequence, with the predicted mature amino acid sequence beginning at amino acid 407, or are a transmembrane domain.

The EcoRI/NotI restriction fragment obtainable from the deposit containing clone do15_{—}4 should be approximately 1900 bp.

The nucleotide sequence disclosed herein for do15_{—}4 was searched against the GenBank and GeneSeq nucleotide sequence databases using BLASTN/BLASTX and FASTA search protocols. do15_{—}4 demonstrated at least some similarity with sequences identified as AA113909 (zm80f12.rl Stratagene neuroepithelium (#937231) Homo sapiens cDNA clone 531983 5′), AA189888 (mu55h06.rl Soares mouse lymph node NbMLN Mus musculus cDNA clone 643355 5′), and U52052 (Human S6 A-8 mRNA expressed in chromosome 6-suppressed melanoma cells). Based upon sequence similarity, do15_{—}4 proteins and each similar protein or peptide may share at least some activity.

Clone “dx290_{—}1”

A polynucleotide of the present invention has been identified as clone “dx290_{—}1”. dx290_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dx290_{—}1 is a full-length clone, including the entire coding sequence of a secreted protein (also referred to herein as “dx290_{—}1 protein”).

The nucleotide sequence of dx290_{—}1 as presently determined is reported in SEQ ID NO:69. What applicants presently believe to be the proper reading frame and the predicted amino acid sequence of the dx290_{—}1 protein corresponding to the foregoing nucleotide sequence is reported in SEQ ID NO:70.

The EcoRI/NotI restriction fragment obtainable from the deposit containing clone dx290_{—}1 should be approximately 2300 bp.

The nucleotide sequence disclosed herein for dx290_{—}1 was searched against the GenBank and GeneSeq nucleotide sequence databases using BLASTN/BLASTX and FASTA search protocols. dx290_{—}1 demonstrated at least some similarity with the sequence identified as AA064383 (m147h02.rl Stratagene mouse testis (#937308) Mus musculus cDNA clone 515187 5′). Based upon sequence similarity, dx290_{—}1 proteins and each similar protein or peptide may share at least some activity.

Clone “ek390_{—}4”

A polynucleotide of the present invention has been identified as clone “ek390_{—}4”. ek390_{—}4 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ek390_{—}4 is a full-length clone, including the entire coding sequence of a secreted protein (also referred to herein as “ek390_{—}4 protein”).

The nucleotide sequence of ek390_{—}4 as presently determined is reported in SEQ ID NO:71. What applicants presently believe to be the proper reading frame and the predicted amino acid sequence of the ek390_{—}4 protein corresponding to the foregoing nucleotide sequence is reported in SEQ ID NO:72. Amino acids 25 to 37 are a predicted leader/signal sequence, with the predicted mature amino acid sequence beginning at amino acid 38, or are a transmembrane domain.

The EcoRI/NotI restriction fragment obtainable from the deposit containing clone ek390_{—}4 should be approximately 1000 bp.

The nucleotide sequence disclosed herein for ek390_{—}4 was searched against the GenBank and GeneSeq nucleotide sequence databases using BLASTN/BLASTX and FASTA search protocols. ek390_{—}4 demonstrated at least some similarity with sequences identified as AA075783 (zm89h02.rl Stratagene ovarian cancer (#937219) Homo sapiens cDNA clone 545139 5′), AA427538 (zw32g04.rl Soares ovary tumor NbHOT Homo sapiens cDNA clone 771030 5′), AA427539 (zw32g04.s1 Soares ovary tumor NbHOT Homo sapiens cDNA clone 771030 3′), AA453353 (zx47a06.rl Soares testis NHT Homo sapiens cDNA clone 795346 5′), C20637 (HUMGS0004639, Human Gene Signature, 3′-directed cDNA sequence), R74326 (y101c07.s1 Homo sapiens cDNA clone 156972 3′), R74420 (y101c07.rl Homo sapiens cDNA clone 156972 5′), T22914 (Human gene signature), U41197 (Human [TTTC] 10 short tandem repeat polymorphism UM65, D17S1340), and X58237 (Human mRNA for anti-lectin antibody epitope (clone p36/8-6)). Based upon sequence similarity, ek390_{—}4 proteins and each similar protein or peptide may share at least some activity. The TopPredII computer program predicts a potential transmembrane domain within the ek390_{—}4 protein sequence centered around amino acid 160 of SEQ ID NO:72. The nucleotide sequence of ek390_{—}4 indicates that it may contain GGGA repeat sequences.

Clone “er471_{—}7”

A polynucleotide of the present invention has been identified as clone “er471_{—}7”. er471_{—}7 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. er471_{—}7 is a full-length clone, including the entire coding sequence of a secreted protein (also referred to herein as “er471_{—}7 protein”).

The nucleotide sequence of er471_{—}7 as presently determined is reported in SEQ ID NO:73. What applicants presently believe to be the proper reading frame and the predicted amino acid sequence of the er471_{—}7 protein corresponding to the foregoing nucleotide sequence is reported in SEQ ID NO:74. Amino acids 74 to 86 are a predicted leader/signal sequence, with the predicted mature amino acid sequence beginning at amino acid 87, or are a transmembrane domain.

The EcoRI/NotI restriction fragment obtainable from the deposit containing clone er471_{—}7 should be approximately 2250 bp.

The nucleotide sequence disclosed herein for er471_{—}7 was searched against the GenBank and GeneSeq nucleotide sequence databases using BLASTN/BLASTX and FASTA search protocols. er471_{—}7 demonstrated at least some similarity with sequences identified as AA039137 (mi98h06.rl Soares mouse embryo NbME13.5 14.5 Mus musculus cDNA clone 474683 5′), AA066962 (mm38g05.rl Stratagene mouse melanoma (#937312) Mus musculus cDNA clone 523832 5′), AA189170 (zq47h05.s1 Stratagene hNT neuron (#937233) Homo sapiens cDNA clone 632889 3′), AA609188 (af12c10.s1 Soares testis NHT Homo sapiens cDNA clone 1031442 3′), and W07704 (zb02e02.rl Soares fetal lung NbHL19W Homo sapiens cDNA clone 300890 5′ similar to SW:YN66_YEAST P40164 HYPOTHETICAL 98.1 KD PROTEIN IN SPX19-GCR2 INTERGENIC REGION). The predicted amino acid sequence disclosed herein for er471_{—}7 was searched against the GenPept and GeneSeq amino acid sequence databases using the BLASTX search protocol. The predicted er471_{—}7 protein demonstrated at least some similarity to sequences identified as AF016448 (Cosmid F41E6 [Caenorhabditis elegans]) and L08407 (collagen type XVII [Mus musculus]). Based upon sequence similarity, er471_{—}7 proteins and each similar protein or peptide may share at least some activity. The TopPredII computer program predicts three potential transmembrane domains within the er471_{—}7 protein sequence, centered around amino acids 40, 80, and 110 of SEQ ID NO:74, respectively.

Clone “fs40_{—}3”

A polynucleotide of the present invention has been identified as clone “fs40_{—}3”. fs40_{—}3 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fs40_{—}3 is a full-length clone, including the entire coding sequence of a secreted protein (also referred to herein as “fs40_{—}3 protein”).

The nucleotide sequence of fs40_{—}3 as presently determined is reported in SEQ ID NO:75. What applicants presently believe to be the proper reading frame and the predicted amino acid sequence of the fs40_{—}3 protein corresponding to the foregoing nucleotide sequence is reported in SEQ ID NO:76.

The EcoRI/NotI restriction fragment obtainable from the deposit containing clone fs40_{—}3 should be approximately 1000 bp.

The nucleotide sequence disclosed herein for fs40_{—}3 was searched against the GenBank and GeneSeq nucleotide sequence databases using BLASTN/BLASTX and FASTA search protocols. fs40_{—}3 demonstrated at least some similarity with sequences identified as AA411142 (zt37g01.rl Soares ovary tumor NbHOT Homo sapiens cDNA clone 724560 5′), AA412527 (zu12a03.s1 Soares testis NHT Homo sapiens cDNA clone 731596 3′), AA565855 (nj32d09.s1 NCI_CGAP_AA1 Homo sapiens cDNA clone IMAGE:994193), H17042 (ym39f12.s1 Homo sapiens cDNA clone 50584 3′), and T33280 (EST57284 Homo sapiens cDNA 3′ end similar to None). Based upon sequence similarity, fs40_{—}3 proteins and each similar protein or peptide may share at least some activity. The TopPredII computer program predicts a potential transmembrane domain within the fs40_{—}3 protein sequence at the C-terminus of SEQ ID NO:76.

Clone “ga63_{—}6”

A polynucleotide of the present invention has been identified as clone “ga63_{—}6”. ga63_{—}6 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ga63_{—}6 is a full-length clone, including the entire coding sequence of a secreted protein (also referred to herein as “ga63_{—}6 protein”).

The nucleotide sequence of ga63_{—}6 as presently determined is reported in SEQ ID NO:77. What applicants presently believe to be the proper reading frame and the predicted amino acid sequence of the ga63_{—}6 protein corresponding to the foregoing nucleotide sequence is reported in SEQ ID NO:78. Amino acids 11 to 23 are a predicted leader/signal sequence, with the predicted mature amino acid sequence beginning at amino acid 24, or are a transmembrane domain.

The EcoRI/NotI restriction fragment obtainable from the deposit containing clone ga63_{—}6 should be approximately 2300 bp.

The nucleotide sequence disclosed herein for ga63_{—}6 was searched against the GenBank and GeneSeq nucleotide sequence databases using BLASTN/BLASTX and FASTA search protocols. ga63_{—}6 demonstrated at least some similarity with sequences identified as AA405433 (zu13h10.rl Soares testis NHT Homo sapiens cDNA clone 731779 5′similar to TR G474970 G474970 SP32 PRECURSOR), AA406076 (zu67c02.s1 Soares testis NHT Homo sapiens cDNA clone 743042 3′ similar to TR:G475021 G475021 SP32 PRECURSOR), AA424694 (zu13h10.s1 Soares testis NHT Homo sapiens cDNA clone 731779 3′ similar to TR G475021 G475021 SP32 PRECURSOR; contains element TAR1 repetitive element), D16200 (Pig mRNA for sp32, partial sequence), D16203 (Guinea pig mRNA for sp32, complete cds), and D17573 (Mouse mRNA for proacrosin-binding protein (sp32), complete cds). The predicted amino acid sequence disclosed herein for ga63_{—}6 was searched against the GenPept and GeneSeq amino acid sequence databases using the BLASTX search protocol. The predicted ga63_{—}6 protein demonstrated at least some similarity to sequences identified as D16200 (sp32 precursor [Sus scrofa]), and D17574 (alternative splicing product for proacrosin-binding protein (sp32) [Mus musculus]). The sp32 protein is found in the acrosomal vescicle of sperm, which is involved in egg-sperm fusion in fertilization. This protein is initially synthesized as a 61-kDa precursor protein with a putative signal peptide at the amino terminus. The carboxyl-terminal half of the precursor molecule corresponds to the mature sp32 protein. Thus, sp32 is produced by post-translational modification of the precursor. The binding of sp32 to proacrosin may be involved in packaging the acrosin zymogen into the acrosomal matrix. (Baba et al., 1994, J Biol. Chem. 269 (13): 10133-10140, which is incorporated by reference herein). Based upon sequence similarity, ga63_{—}6 proteins and each similar protein or peptide may share at least some activity.

Clone “gm335_{—}4”

A polynucleotide of the present invention has been identified as clone “gm335_{—}4”. gm335_{—}4 was isolated from a human adult uterus cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gm335_{—}4 is a full-length clone, including the entire coding sequence of a secreted protein (also referred to herein as “gm335_{—}4 protein”).

The nucleotide sequence of gm335_{—}4 as presently determined is reported in SEQ ID NO:79. What applicants presently believe to be the proper reading frame and the predicted amino acid sequence of the gm335_{—}4 protein corresponding to the foregoing nucleotide sequence is reported in SEQ ID NO:80. Amino acids 8 to 20 are a predicted leader/signal sequence, with the predicted mature amino acid sequence beginning at amino acid 21, or are a transmembrane domain.

The EcoRI/NotI restriction fragment obtainable from the deposit containing clone gm335_{—}4 should be approximately 800 bp.

The nucleotide sequence disclosed herein for gm335_{—}4 was searched against the GenBank and GeneSeq nucleotide sequence databases using BLASTN/BLASTX and FASTA search protocols. gm335_{—}4 demonstrated at least some similarity with sequences identified as AA055367 (zf20b05.rl Soares fetal heart NbHH19W Homo sapiens cDNA clone 377457 5′), AC002389 (Human DNA from chromosome 19 specific cosmid R28461, genomic sequence, complete sequence), W08522 (mb46h10.rl Soares mouse p3NMF19.5 Mus musculus cDNA clone 332515 5′), and X93916 (S.scrofa mRNA (clone VIB11; expressed sequence tag)). Based upon sequence similarity, gm335_{—}4 proteins and each similar protein or peptide may share at least some activity.

Clone “hy370_{—}9”

A polynucleotide of the present invention has been identified as clone “hy370_{—}9”. hy370_{—}9 was isolated from a human adult trachea cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. hy370_{—}9 is a full-length clone, including the entire coding sequence of a secreted protein (also referred to herein as “hy370_{—}9 protein”).

The nucleotide sequence of hy370_{—}9 as presently determined is reported in SEQ ID NO:81. What applicants presently believe to be the proper reading frame and the predicted amino acid sequence of the hy370_{—}9 protein corresponding to the foregoing nucleotide sequence is reported in SEQ ID NO:82. Amino acids 8 to 20 are a predicted leader/signal sequence, with the predicted mature amino acid sequence beginning at amino acid 21, or are a transmembrane domain.

The EcoRI/NotI restriction fragment obtainable from the deposit containing clone hy370_{—}9 should be approximately 1200 bp.

The nucleotide sequence disclosed herein for hy370_{—}9 was searched against the GenBank and GeneSeq nucleotide sequence databases using BLASTN/BLASTX and FASTA search protocols. hy370_{—}9 demonstrated at least some similarity with the sequence identified as AA763313 (vv89h07.rl Stratagene mouse skin (#937313) Mus musculus cDNA clone 1229629 5′). Based upon sequence similarity, hy370_{—}9 proteins and each similar protein or peptide may share at least some activity. The TopPredII computer program predicts an additional potential transmembrane domain within the hy370_{—}9 protein sequence centered around amino acid 140 of SEQ ID NO:82.

Clone “ie47_{—}4”

A polynucleotide of the present invention has been identified as clone “ie47_{—}4”. ie47_{—}4 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ie47_{—}4 is a full-length clone, including the entire coding sequence of a secreted protein (also referred to herein as “ie47_{—}4 protein”).

The nucleotide sequence of ie47_{—}4 as presently determined is reported in SEQ ID NO:83. What applicants presently believe to be the proper reading frame and the predicted amino acid sequence of the ie47_{—}4 protein corresponding to the foregoing nucleotide sequence is reported in SEQ ID NO:84. Amino acids 17 to 29 are a predicted leader/signal sequence, with the predicted mature amino acid sequence beginning at amino acid 30, or are a transmembrane domain.

The EcoRI/NotI restriction fragment obtainable from the deposit containing clone ie47_{—}4 should be approximately 2300 bp.

The nucleotide sequence disclosed herein for ie47_{—}4 was searched against the GenBank and GeneSeq nucleotide sequence databases using BLASTN/BLASTX and FASTA search protocols. ie47_{—}4 demonstrated at least some similarity with sequences identified as AA071953 (mf17h08.rl Life Tech mouse brain Mus musculus cDNA clone 405375 5′ similar to TR G304421 G304421 SILENCER ELEMENT), AA207250 (zq82d05.s1 Stratagene hNT neuron (#937233) Homo sapiens cDNA clone 648105 3′ similar to TR G304421 G304421 SILENCER ELEMENT), L14938 (Chicken SCG10 protein mRNA, complete cds), L20260 (Mouse SCG10 gene sequence), R49053 (yg58c05.s1 Homo sapiens cDNA clone 37017 3′), S82024 (SCG10 neuron-specific growth-associated protein/stathmin homolog [human, embryo, mRNA]), T25428 (Human gene signature HUMGS07594, T25428 standard; cDNA to mRNA), W54204 (md04a12.rl Soares mouse embryo NbME13.5 14.5 Mus musculus cDNA clone 367390 5′ similar to SW:SCGB_XENLA Q09002 SCG10 PROTEIN HOMOLOG A), X71433 (X. laevis SCG10 mRNA), and Z99916 (Human DNA sequence *** SEQUENCING IN PROGRESS *** from clone 221G9; HTGS phase 1). The predicted amino acid sequence disclosed herein for ie47_{—}4 was searched against the GenPept and GeneSeq amino acid sequence databases using the BLASTX search protocol. The predicted ie47_{—}4 protein demonstrated at least some similarity to sequences identified as L14938 (SCG10 protein [Gallus gallus]) and S82024 (SCG10 neuron-specific growth-associated protein/stathmin homolog [human, embryo, Peptide] [Homo sapiens]). SCG10 protein is considered to be a membrane-bound protein present in neural growth cones and developing neurons (Maucuer et al., 1993, J. Biol. Chem. 268: 16420-16429; Stein et al., 1988, Neuron 1:463-476; which are incorporated by reference herein). Based upon sequence similarity, ie47_{—}4 proteins and each similar protein or peptide may share at least some activity.

Clone “s195_{—}10”

A polynucleotide of the present invention has been identified as clone “s195_{—}10”. s195_{—}10 was isolated from a human adult neural tissue cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. s195_{—}10 is a full-length clone, including the entire coding sequence of a secreted protein (also referred to herein as “s195_{—}10 protein”).

The nucleotide sequence of s195_{—}10 as presently determined is reported in SEQ ID NO:85. What applicants presently believe to be the proper reading frame and the predicted amino acid sequence of the s195_{—}10 protein corresponding to the foregoing nucleotide sequence is reported in SEQ ID NO:86. Amino acids 35 to 47 are a predicted leader/signal sequence, with the predicted mature amino acid sequence beginning at amino acid 48, or are a transmembrane domain.

The EcoRI/NotI restriction fragment obtainable from the deposit containing clone s195_{—}10 should be approximately 3500 bp.

The nucleotide sequence disclosed herein for s195_{—}10 was searched against the GenBank and GeneSeq nucleotide sequence databases using BLASTN/BLASTX and FASTA search protocols. s195_{—}10 demonstrated at least some similarity with sequences identified as AA113800 (zn65b05.s1 Stratagene HeLa cell s3 937216 Homo sapiens cDNA clone 563025 3′ similar to TR:G600018 G600018 SSM4P), AA114062 (zn65b05.rl Stratagene HeLa cell s3 937216 Homo sapiens cDNA clone 563025 5′), AA280316 (zt10f06.s1 Soares NbHTGBC Homo sapiens cDNA clone 712739 3′), AF009301 (Homo sapiens TEB4 protein mRNA, complete cds), N70344 (za60f10.s1 Homo sapiens cDNA clone 296971 3′), R60474 (yh13g07.rl Homo sapiens cDNA clone 43058 5′), and T26266 (standard; cDNA to mRNA; 148 BP, Human gene signature HUMGS08505). The predicted amino acid sequence disclosed herein for s195_{—}10 was searched against the GenPept and GeneSeq amino acid sequence databases using the BLASTX search protocol. The predicted s195_{—}10 protein demonstrated at least some similarity to sequences identified as AF009301 (TEB4 protein [Homo sapiens]), X76715 (SSM4 gene product [Saccharomyces cerevisiae]), Z46861 (Ssm4p [Saccharomyces cerevisiae]), and Z47047 (Ssm4p [Saccharomyces cerevisiae]). Based upon sequence similarity, s195_{—}10 proteins and each similar protein or peptide may share at least some activity. The TopPredII computer program predicts eleven additional potential transmembrane domains within the s195_{—}10 protein sequence, centered around amino acids 130, 170, 210, 260, 320, 470, 520, 560, 600, 650, and 690 of SEQ ID NO:86, respectively. The nucleotide sequence of s195_{—}10 indicates that it may contain a simple GAA repeat region.

Clone “bf228_{—}14”

A polynucleotide of the present invention has been identified as clone “bf228_{—}14”. bf228_{—}14 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bf228_{—}14 is a full-length clone, including the entire coding sequence of a secreted protein (also referred to herein as “bf228_{—}14 protein”).

The nucleotide sequence of bf228_{—}14 as presently determined is reported in SEQ ID NO:97. What applicants presently believe to be the proper reading frame and the predicted amino acid sequence of the bf228_{—}14 protein corresponding to the foregoing nucleotide sequence is reported in SEQ ID NO:98. Amino acids 18 to 30 are a predicted leader/signal sequence, with the predicted mature amino acid sequence beginning at amino acid 31, or are a transmembrane domain.

The EcoRI/NotI restriction fragment obtainable from the deposit containing clone bf228_{—}14 should be approximately 1400 bp.

The nucleotide sequence disclosed herein for bf228_{—}14 was searched against the GenBank and GeneSeq nucleotide sequence databases using BLASTN/BLASTX and FASTA search protocols. bf228_{—}14 demonstrated at least some homology with sequences identified as AA069549 (zm52e03.s1 Stratagene fibroblast (#937212) Homo sapiens cDNA clone 529276 3′), H83278 (yq49h09.rl Homo sapiens cDNA clone 199169 5′), and N94898 (zb31b01.s1 Homo sapiens cDNA clone 305161 3′ similar to contains Alu repetitive element;contains element MSR1 repetitive element). Based upon homology, bf228_{—}14 proteins and each homologous protein or peptide may share at least some activity. The nucleotide sequence of bf228_{—}14 indicates that it may contain an Alu repetitive element.

Clone “bg249_{—}1”

A polynucleotide of the present invention has been identified as clone “bg249_{—}1”. bg249_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bg249_{—}1 is a full-length clone, including the entire coding sequence of a secreted protein (also referred to herein as “bg249_{—}1 protein”).

The nucleotide sequence of bg249_{—}1 as presently determined is reported in SEQ ID NO:99. What applicants presently believe to be the proper reading frame and the predicted amino acid sequence of the bg249_{—}1 protein corresponding to the foregoing nucleotide sequence is reported in SEQ ID NO:100.

The EcoRI/NotI restriction fragment obtainable from the deposit containing clone bg249_{—}1 should be approximately 2700 bp.

The nucleotide sequence disclosed herein for bg249_{—}1 was searched against the GenBank and GeneSeq nucleotide sequence databases using BLASTN/BLASTX and FASTA search protocols. bg249_{—}1 demonstrated at least some homology with sequences identified as AA151021 (z147c04.rl Soares pregnant uterus NbHPU Homo sapiens cDNA clone 505062 5′), AA278781 (zs79a01.rl Soares NbHTGBC Homo sapiens cDNA clone 703656 5′), R75099 (MDB1032R Mouse brain, Stratagene Mus musculus cDNA 5′end), and T06990 (EST04879 Homo sapiens cDNA clone HFBEB91). Based upon homology, bg249_{—}1 proteins and each homologous protein or peptide may share at least some activity.

Clone “bv286_{—}1”

A polynucleotide of the present invention has been identified as clone “bv286_{—}1”. bv286_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bv286_{—}1 is a full-length clone, including the entire coding sequence of a secreted protein (also referred to herein as “bv286_{—}1 protein”).

The nucleotide sequence of bv286_{—}1 as presently determined is reported in SEQ ID NO:101. What applicants presently believe to be the proper reading frame and the predicted amino acid sequence of the bv286_{—}1 protein corresponding to the foregoing nucleotide sequence is reported in SEQ ID NO:102. Amino acids 14 to 26 are a predicted leader/signal sequence, with the predicted mature amino acid sequence beginning at amino acid 27, or are a transmembrane domain.

The EcoRI/NotI restriction fragment obtainable from the deposit containing clone bv286_{—}1 should be approximately 550 bp.

The nucleotide sequence disclosed herein for bv286_{—}1 was searched against the GenBank and GeneSeq nucleotide sequence databases using BLASTN/BLASTX and FASTA search protocols. bv286_{—}1 demonstrated at least some homology with sequences identified as AA132163 (z138c07.rl Soares pregnant uterus NbHPU Homo sapiens cDNA clone 504204 5′ similar to WP:F55A11.1 CE05943 EF HAND DOMAINS), AA552888 (nk57h08.s1 NCI_CGAP Pr7 Homo sapiens cDNA clone IMAGE:1017663 similar to WP:F55A11.1 CE05943 EF HAND DOMAINS), and H12316 (yj11d07.s1 Homo sapiens cDNA clone 148429 3′ similar to SP:JSC027 JS0027 PROBABLE CALCIUM-BINDING PROTEIN). The predicted amino acid sequence disclosed herein for bv286_{—}1 was searched against the GenPept and GeneSeq amino acid sequence databases using the BLASTX search protocol. The predicted bv286_{—}1 protein demonstrated at least some identity with sequences identified as L22647 (prostaglandin receptor ep1 subtype [Homo sapiens]). Based upon homology, bv286_{—}1 proteins and each homologous protein or peptide may share at least some activity. The nucleotide sequence of bv286_{—}1 indicates that it may contain an Alu repetitive element.

Clone “co36_{—}1”

A polynucleotide of the present invention has been identified as clone “co36_{—}1”. co36_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. co36_{—}1 is a full-length clone, including the entire coding sequence of a secreted protein (also referred to herein as “co36_{—}1 protein”).

The nucleotide sequence of co36_{—}1 as presently determined is reported in SEQ ID NO:103. What applicants presently believe to be the proper reading frame and the predicted amino acid sequence of the co36_{—}1 protein corresponding to the foregoing nucleotide sequence is reported in SEQ ID NO:104.

The EcoRI/NotI restriction fragment obtainable from the deposit containing clone co36_{—}1 should be approximately 3300 bp.

The nucleotide sequence disclosed herein for co36_{—}1 was searched against the GenBank and GeneSeq nucleotide sequence databases using BLASTN/BLASTX and FASTA search protocols. No hits were found in the database.

Clone “cp116_{—}1”

A polynucleotide of the present invention has been identified as clone “cp116_{—}1”. cp116_{—}1 was isolated from a human adult salivary gland cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cp116_{—}1 is a full-length clone, including the entire coding sequence of a secreted protein (also referred to herein as “cp116_{—}1 protein”).

The nucleotide sequence of cp116_{—}1 as presently determined is reported in SEQ ID NO:105. What applicants presently believe to be the proper reading frame and the predicted amino acid sequence of the cp116_{—}1 protein corresponding to the foregoing nucleotide sequence is reported in SEQ ID NO:106. Amino acids 3 to 15 are a predicted leader/signal sequence, with the predicted mature amino acid sequence beginning at amino acid 16, or are a transmembrane domain.

The EcoRI/NotI restriction fragment obtainable from the deposit containing clone cp116_{—}1 should be approximately 1600 bp.

The nucleotide sequence disclosed herein for cp116_{—}1 was searched against the GenBank and GeneSeq nucleotide sequence databases using BLASTN/BLASTX and FASTA search protocols. No hits were found in the database.

Clone “cw1195_{—}2”

A polynucleotide of the present invention has been identified as clone “cw1195_{—}2”. cw1195_{—}2 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cw1195_{—}2 is a full-length clone, including the entire coding sequence of a secreted protein (also referred to herein as “cw1195_{—}2 protein”).

The nucleotide sequence of the 5′ portion of cw1195_{—}2 as presently determined is reported in SEQ ID NO:107. An additional internal nucleotide sequence from cw1195_{—}2 as presently determined is reported in SEQ ID NO:108. What applicants believe is the proper reading frame and the predicted amino acid sequence encoded by such internal sequence is reported in SEQ ID NO:109. Additional nucleotide sequence from the 3′ portion of cw1195_{—}2, including the polyA tail, is reported in SEQ ID NO:110.

The EcoRI/NotI restriction fragment obtainable from the deposit containing clone cw1195_{—}2 should be approximately 3300 bp.

The nucleotide sequence disclosed herein for cw1195_{—}2 was searched against the GenBank and GeneSeq nucleotide sequence databases using BLASTN/BLASTX and FASTA search protocols. cw1195_{—}2 demonstrated at least some homology with sequences identified as AA205460 (zq66f07.s1 Stratagene neuroepithelium (#937231) Homo sapiens cDNA clone 646597 3′) and AA362052 (EST71451 MCF7 cell line Homo sapiens cDNA 5′ end similar to EST containing Alu repeat). Based upon homology, cw1195_{—}2 proteins and each homologous protein or peptide may share at least some activity. The nucleotide sequence of cw1195_{—}2 indicates that it may contain an Alu repetitive element.

Clone “fh13_{—}10”

A polynucleotide of the present invention has been identified as clone “fh13_{—}10”. fh13_{—}10 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. fh13_{—}10 is a full-length clone, including the entire coding sequence of a secreted protein (also referred to herein as “fh13_{—}10 protein”).

The nucleotide sequence of fh13_{—}10 as presently determined is reported in SEQ ID NO:111. What applicants presently believe to be the proper reading frame and the predicted amino acid sequence of the fh13_{—}10 protein corresponding to the foregoing nucleotide sequence is reported in SEQ ID NO:112.

The EcoRI/NotI restriction fragment obtainable from the deposit containing clone fh13_{—}10 should be approximately 1400 bp.

The nucleotide sequence disclosed herein for fh13_{—}10 was searched against the GenBank and GeneSeq nucleotide sequence databases using BLASTN/BLASTX and FASTA search protocols. fh13_{—}10 demonstrated at least some homology with sequences identified as J00089 (Human Alu family interspersed repeat; clone BLUR6) and X00481 (Human non-alu family interspersed repeat). Based upon homology, fh13_{—}10 proteins and each homologous protein or peptide may share at least some activity. The nucleotide sequence of fh13_{—}10 indicates that it may contain a repetitive element.

Clone “gc57_{—}4”

A polynucleotide of the present invention has been identified as clone “gc57_{—}4”. gc57_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. gc57_{—}4 is a full-length clone, including the entire coding sequence of a secreted protein (also referred to herein as “gc57_{—}4 protein”).

The nucleotide sequence of gc57_{—}4 as presently determined is reported in SEQ ID NO:113. What applicants presently believe to be the proper reading frame and the predicted amino acid sequence of the gc57_{—}4 protein corresponding to the foregoing nucleotide sequence is reported in SEQ ID NO:114. Amino acids 10 to 22 are a predicted leader/signal sequence, with the predicted mature amino acid sequence beginning at amino acid 23, or are a transmembrane domain.

The EcoRI/NotI restriction fragment obtainable from the deposit containing clone gc57_{—}4 should be approximately 1900 bp.

The nucleotide sequence disclosed herein for gc57_{—}4 was searched against the GenBank and GeneSeq nucleotide sequence databases using BLASTN/BLASTX and FASTA search protocols. gc57_{—}4 demonstrated at least some homology with sequences identified as AA095328 (13005.seq.F Fetal heart, Lambda ZAP Express Homo sapiens cDNA 5′), AA126440 (zk94e02.s1 Soares pregnant uterus NbHPU Homo sapiens cDNA clone 490490 3′), and Z82195 (Human DNA sequence from clone J274L71). Based upon homology, gc57_{—}4 proteins and each homologous protein or peptide may share at least some activity. The nucleotide sequence of gc57_{—}4 indicates that it may contain an Alu repetitive element.

Clone “h1165_{—}3”

A polynucleotide of the present invention has been identified as clone “h1165_{—}3”. h1165_{—}3 was isolated from a human adult blood (peripheral blood mononuclear cells treated with phytohemagglutinin and phorbol meristate acetate and mixed lymphocyte reaction) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. h1165_{—}3 is a full-length clone, including the entire coding sequence of a secreted protein (also referred to herein as “h1165_{—}3 protein”).

The nucleotide sequence of h1165_{—}3 as presently determined is reported in SEQ ID NO:115. What applicants presently believe to be the proper reading frame and the predicted amino acid sequence of the h1165_{—}3 protein corresponding to the foregoing nucleotide sequence is reported in SEQ ID NO:116.

The EcoRI/NotI restriction fragment obtainable from the deposit containing clone h1165_{—}3 should be approximately 1250 bp.

The nucleotide sequence disclosed herein for h1165_{—}3 was searched against the GenBank and GeneSeq nucleotide sequence databases using BLASTN/BLASTX and FASTA search protocols. h1165_{—}3 demonstrated at least some homology with sequences identified as AA173098 (zp31d03.rl Stratagene neuroepithelium (#937231) Homo sapiens cDNA clone 611045 5′ similar to contains element TAR1 repetitive element), AA305139 (EST176159 Colon carcinoma (Caco-2) cell line II Homo sapiens cDNA 5′ end), AA426375 (zv54h02.s1 Soares testis NHT Homo sapiens cDNA clone 757491 3′), and N72370 (yv38c11.rl Homo sapiens cDNA clone 245012 5′). The predicted amino acid sequence disclosed herein for h1165_{—}3 was searched against the GenPept and GeneSeq amino acid sequence databases using the BLASTX search protocol. The predicted h1165_{—}3 protein demonstrated at least some identity with sequences identified as U58758 (coded for by C. elegans cDNA yk83a5.3; coded for by C. elegans cDNA yk83a5.5 [Caenorhabditis elegans]). Based upon homology, h1165_{—}3 proteins and each homologous protein or peptide may share at least some activity.

Clone “hb752_{—}1”

A polynucleotide of the present invention has been identified as clone “hb752_{—}1”. hb752_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. hb752_{—}1 is a full-length clone, including the entire coding sequence of a secreted protein (also referred to herein as “hb752_{—}1 protein”).

The nucleotide sequence of hb752_{—}1 as presently determined is reported in SEQ ID NO:117. What applicants presently believe to be the proper reading frame and the predicted amino acid sequence of the hb752_{—}1 protein corresponding to the foregoing nucleotide sequence is reported in SEQ ID NO:118. Amino acids 16 to 28 are a predicted leader/signal sequence, with the predicted mature amino acid sequence beginning at amino acid 29, or are a transmembrane domain.

The EcoRI/NotI restriction fragment obtainable from the deposit containing clone hb752_{—}1 should be approximately 1800 bp.

The nucleotide sequence disclosed herein for hb752_{—}1 was searched against the GenBank and GeneSeq nucleotide sequence databases using BLASTN/BLASTX and FASTA search protocols. hb752_{—}1 demonstrated at least some homology with sequences identified as AA100979 (zm26f09.s1 Stratagene pancreas (#937208) Homo sapiens cDNA clone 526793 3′), AA490528 (aa51g113s1 NCI_CGAP_GCB1 Homo sapiens cDNA clone 824516 3′), H65670 (yr72g12.rl Homo sapiens cDNA clone 210886 5′), H86790 (ys72c03.s1 Homo sapiens cDNA clone 220324 3′), N58917 (yy61f11.s1 Homo sapiens cDNA clone 278061 3′), and Z43307 (H. sapiens partial cDNA sequence; clone c-18g09). Based upon homology, hb752_{—}1 proteins and each homologous protein or peptide may share at least some activity.

Clone “bi127_{—}5”

A polynucleotide of the present invention has been identified as clone “bi127_{—}5”. bi127_{—}5 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bi127_{—}5 is a full-length clone, including the entire coding sequence of a secreted protein (also referred to herein as “bi127_{—}5 protein”).

The nucleotide sequence of bi127_{—}5 as presently determined is reported in SEQ ID NO:129. What applicants presently believe to be the proper reading frame and the predicted amino acid sequence of the bi127_{—}5 protein corresponding to the foregoing nucleotide sequence is reported in SEQ ID NO:130.

The EcoRI/NotI restriction fragment obtainable from the deposit containing clone bi127_{—}5 should be approximately 2500 bp.

The nucleotide sequence disclosed herein for bi127_{—}5 was searched against the GenBank and GeneSeq nucleotide sequence databases using BLASTN/BLASTX and FASTA search protocols. bi127_{—}5 demonstrated at least some similarity with sequences identified as AA055840 (zf20c06.s1 Soares fetal heart NbHH19W Homo sapiens cDNA clone 377482 3′), AA334304 (EST38496 Embryo, 9 week Homo sapiens cDNA 5′ end similar to similar to H. sapiens hypothetical protein, chromosome 3p21.1 gene sequence (GB:L13435)), AA399397 (zt59f11.rl Soares testis NHT Homo sapiens cDNA clone 726669 5′), AA576692 (nm73a07.s1 NCI_CGAP_Co9 Homo sapiens cDNA clone IMAGE:1073844), H01918 (yj29a07.s1 Homo sapiens cDNA clone 150132 3′), H94897 (yu57h08.s1 Homo sapiens cDNA clone 230271 3′), L13435 (Human chromosome 3p21.1 gene sequence), R85965 (yt66g02.s1 Soares retina N2b4HR Homo sapiens cDNA clone 275499 3′), and X95828 (H.sapiens DNA NotI jumping clone J32A032D). Based upon sequence similarity, bi127_{—}5 proteins and each similar protein or peptide may share at least some activity. The TopPredII computer program predicts a potential transmembrane domain within the bi127_{—}5 protein sequence centered around amino acid 15 of SEQ ID NO:130.

Clone “bl194_{—}2”

A polynucleotide of the present invention has been identified as clone “bl194_{—}2”. bl194_{—}2 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bl194_{—}2 is a full-length clone, including the entire coding sequence of a secreted protein (also referred to herein as “bl194_{—}2 protein”).

The nucleotide sequence of bl194_{—}2 as presently determined is reported in SEQ ID NO:130. What applicants presently believe to be the proper reading frame and the predicted amino acid sequence of the bl194_{—}2 protein corresponding to the foregoing nucleotide sequence is reported in SEQ ID NO:132. Amino acids 88 to 100 are a predicted leader/signal sequence, with the predicted mature amino acid sequence beginning at amino acid 101, or are a transmembrane domain.

The EcoRI/NotI restriction fragment obtainable from the deposit containing clone bl194_{—}2 should be approximately 1600 bp.

The nucleotide sequence disclosed herein for bl194_{—}2 was searched against the GenBank and GeneSeq nucleotide sequence databases using BLASTN/BLASTX and FASTA search protocols. bl194_{—}2 demonstrated at least some similarity with sequences identified as AA136931 (zn97f05.s1 Stratagene fetal retina 937202 Homo sapiens cDNA clone 566145 3′), AA148976 (zn99e10.rl Stratagene colon (#937204) Homo sapiens cDNA clone 566346 5′), AA148977 (zn99e10.s1 Stratagene colon (#937204) Homo sapiens cDNA clone 566346 3′), AA196293 (zp92g07.s1 Stratagene HeLa cell s3 937216 Homo sapiens cDNA clone 627708 3′), AA487754 (ab13e12.rl Stratagene lung (#937210) Homo sapiens cDNA clone 840718 5′), H01254 (yj27b02.rl Homo sapiens cDNA clone 149931 5′), H86324 (yt05f07.rl Homo sapiens cDNA clone 223429 5′), N23958 (yx71c02.s1 Homo sapiens cDNA clone 267170 3′), N31859 (yx71c02.rl Homo sapiens cDNA clone 267170 5′), R01674 (ye76b07.s1 Homo sapiens cDNA clone 123637 3′), and T78480 (yd68g08.s1 Homo sapiens cDNA clone 113438 3′). The predicted amino acid sequence disclosed herein for bl194_{—}2 was searched against the GenPept and GeneSeq amino acid sequence databases using the BLASTX search protocol. The predicted bl194_{—}2 protein demonstrated at least some similarity to the sequence identified as S75895 (NADH dehydrogenase subunit 2, ND2 [human, brain, Peptide Mitochondrial Partial Mutant, 79 aa] [Homo sapiens]). Based upon sequence similarity, bl194_{—}2 proteins and each similar protein or peptide may share at least some activity.

Clone “cc130_{—}1”

A polynucleotide of the present invention has been identified as clone “cc130_{—}1”. cc130_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cc130_{—}1 is a full-length clone, including the entire coding sequence of a secreted protein (also referred to herein as “cc1301 protein”).

The nucleotide sequence of cc130_{—}1 as presently determined is reported in SEQ ID NO:133. What applicants presently believe to be the proper reading frame and the predicted amino acid sequence of the cc130_{—}1 protein corresponding to the foregoing nucleotide sequence is reported in SEQ ID NO:134. Amino acids 7 to 19 are a predicted leader/signal sequence, with the predicted mature amino acid sequence beginning at amino acid 20, or are a transmembrane domain.

The EcoRI/NotI restriction fragment obtainable from the deposit containing clone cc130_{—}1 should be approximately 2500 bp.

The nucleotide sequence disclosed herein for cc130_{—}1 was searched against the GenBank and GeneSeq nucleotide sequence databases using BLASTN/BLASTX and FASTA search protocols. cc130_{—}1 demonstrated at least some similarity with sequences identified as T21181 (Human gene signature HUMGS02491), T70127 (yc17d06.rl Homo sapiens cDNA clone 80939 5′ similar to SP:BUTY_BOVIN P18892 BUTYROPHILIN PRECURSOR), T92875 (ye27h03.rl Homo sapiens cDNA clone 118997 5′), U39576 (Human butyrophilin precursor mRNA, complete cds), U90546 (Human butyrophilin (BTF4) mRNA, complete cds), and W69453 (zd45e02.s1 Soares fetal heart NbHH19W Homo sapiens cDNA clone 343610 3′). The predicted amino acid sequence disclosed herein for cc130_{—}1 was searched against the GenPept and GeneSeq amino acid sequence databases using the BLASTX search protocol. The predicted cc130_{—}1 protein demonstrated at least some similarity to sequences identified as M35551 (BOVBUTBT1_{—}1 Bovine butyrophilin mRNA, complete cds. [Bos taurus]), R71361 (Human truncated MOG), U39576 (butyrophilin precursor [Homo sapiens]), and U90546 (butyrophilin [Homo sapiens]). Butyrophilin may function in the secretion of milk-fat droplets and may act as a specific membrane-associated receptor for the association of cytoplasmic droplets with the apical plasma membrane. The subcellular location of butyrophilin is that of a Type I membrane protein. Butyrophilin also exhibits tissue specificity, being expressed in mammary tissue and secreted in association with the milk-fat-globule membrane during lactation. Butyrophilin is also homologous to MOG (myelinoligo dendrocyte protein) which is used to treat auto-immune diseases. Both butyrophilin and MOG are homologous in the same amino acids to an immunoglobulin variable region; this may indicate the existence of a protein-protein binding (receptor) site. Based upon sequence similarity, cc1301 proteins and each similar protein or peptide may share at least some activity. The TopPredII computer program predicts an additional potential transmembrane domain within the cc130_{—}1 protein sequence centered around amino acid 255 of SEQ ID NO:134.

Clone “ch582_{—}1”

A polynucleotide of the present invention has been identified as clone “ch582_{—}1”. ch582_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ch582_{—}1 is a full-length clone, including the entire coding sequence of a secreted protein (also referred to herein as “ch582_{—}1 protein”).

The nucleotide sequence of ch582_{—}1 as presently determined is reported in SEQ ID NO:135. What applicants presently believe to be the proper reading frame and the predicted amino acid sequence of the ch582_{—}1 protein corresponding to the foregoing nucleotide sequence is reported in SEQ ID NO:136. Amino acids 23 to 35 are a predicted leader/signal sequence, with the predicted mature amino acid sequence beginning at amino acid 36, or are a transmembrane domain.

The EcoRI/NotI restriction fragment obtainable from the deposit containing clone ch582_{—}1 should be approximately 2300 bp.

The nucleotide sequence of ch582_{—}1 indicates that it may contain one or more repetitive elements.

Clone “cq294_{—}14”

A polynucleotide of the present invention has been identified as clone “cq294_{—}14”. cq294_{—}14 was isolated from a human adult heart cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cq294_{—}14 is a full-length clone, including the entire coding sequence of a secreted protein (also referred to herein as “cq294_{—}14 protein”).

The nucleotide sequence of cq294_{—}14 as presently determined is reported in SEQ ID NO:137. What applicants presently believe to be the proper reading frame and the predicted amino acid sequence of the cq294_{—}14 protein corresponding to the foregoing nucleotide sequence is reported in SEQ ID NO:138.

The EcoRI/NotI restriction fragment obtainable from the deposit containing clone cq294_{—}14 should be approximately 1850 bp.

The nucleotide sequence disclosed herein for cq294_{—}14 was searched against the GenBank and GeneSeq nucleotide sequence databases using BLASTN/BLASTX and FASTA search protocols. cq294_{—}14 demonstrated at least some similarity with sequences identified as AA133962 (z134c12.s1 Soares pregnant uterus NbHPU Homo sapiens cDNA clone 503830 3′), AA447968 (zv83h10.s1 Soares total fetus Nb2HF8 9w Homo sapiens cDNA clone 760291 3′), N47086 (yy85c08.s1 Homo sapiens cDNA clone 280334 3′), R33663 (yh82g06.s1 Homo sapiens cDNA clone 136282 3′), R45544 (yg43g12.s1 Homo sapiens cDNA clone 35358 3′), R77637 (yi76h09.s1 Homo sapiens cDNA clone 145217 3′), and W37736 (zc10h10.rl Soares parathyroid tumor NbHPA Homo sapiens cDNA clone). Based upon sequence similarity, cq294_{—}14 proteins and each similar protein or peptide may share at least some activity. The TopPredII computer program predicts three potential transmembrane domains within the cq294_{—}14 protein sequence, centered around amino acids 15, 25, and 50 of SEQ ID NO:138, respectively. The nucleotide sequence of cq294_{—}14 indicates that it may contain one or more repetitive elements.

Clone “dd454_{—}1”

A polynucleotide of the present invention has been identified as clone “dd454_{—}1”. dd454_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dd454_{—}1 is a full-length clone, including the entire coding sequence of a secreted protein (also referred to herein as “dd454_{—}1 protein”).

The nucleotide sequence of dd454_{—}1 as presently determined is reported in SEQ ID NO:139. What applicants presently believe to be the proper reading frame and the predicted amino acid sequence of the dd454_{—}1 protein corresponding to the foregoing nucleotide sequence is reported in SEQ ID NO:140.

The EcoRI/NotI restriction fragment obtainable from the deposit containing clone dd454_{—}1 should be approximately 2300 bp.

The nucleotide sequence disclosed herein for dd454_{—}1 was searched against the GenBank and GeneSeq nucleotide sequence databases using BLASTN/BLASTX and FASTA search protocols. dd454_{—}1 demonstrated at least some similarity with sequences identified as AA393499 (zt73g06.rl Soares testis NHT Homo sapiens cDNA clone 728026 5′) and AA430063 (zw67a12.s1 Soares testis NHT Homo sapiens cDNA clone 781246 3′). Based upon sequence similarity, dd454_{—}1 proteins and each similar protein or peptide may share at least some activity.

Clone “du157_{—}12”

A polynucleotide of the present invention has been identified as clone “du157_{—}12”. du157_{—}12 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. du157_{—}12 is a full-length clone, including the entire coding sequence of a secreted protein (also referred to herein as “du157_{—}12 protein”).

The nucleotide sequence of du157_{—}12 as presently determined is reported in SEQ ID NO:141. What applicants presently believe to be the proper reading frame and the predicted amino acid sequence of the du157_{—}12 protein corresponding to the foregoing nucleotide sequence is reported in SEQ ID NO:142.

The EcoRI/NotI restriction fragment obtainable from the deposit containing clone du157_{—}12 should be approximately 4050 bp.

The nucleotide sequence disclosed herein for du157_{—}12 was searched against the GenBank and GeneSeq nucleotide sequence databases using BLASTN/BLASTX and FASTA search protocols. du157_{—}12 demonstrated at least some similarity with sequences identified as AA164862 (zq41g04.rl Stratagene hNT neuron (#937233) Homo sapiens cDNA clone 632310 5′), AA284379 (zs59a07.rl NCI_CGAP_GCB1 Homo sapiens cDNA clone IMAGE:701748 5′), and T22493 (Human gene signature HUMGS04104). Based upon sequence similarity, du157_{—}12 proteins and each similar protein or peptide may share at least some activity.

Clone “du372_{—}1”

A polynucleotide of the present invention has been identified as clone “du372_{—}1”. du372_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. du372_{—}1 is a full-length clone, including the entire coding sequence of a secreted protein (also referred to herein as “du372_{—}1 protein”).

The nucleotide sequence of du372_{—}1 as presently determined is reported in SEQ ID NO:143. What applicants presently believe to be the proper reading frame and the predicted amino acid sequence of the du372_{—}1 protein corresponding to the foregoing nucleotide sequence is reported in SEQ ID NO:145. Amino acids 69 to 81 are a predicted leader/signal sequence, with the predicted mature amino acid sequence beginning at amino acid 82, or are a transmembrane domain.

The EcoRI/NotI restriction fragment obtainable from the deposit containing clone du372_{—}1 should be approximately 1500 bp.

The nucleotide sequence disclosed herein for du372_{—}1 was searched against the GenBank and GeneSeq nucleotide sequence databases using BLASTN/BLASTX and FASTA search protocols. du372_{—}1 demonstrated at least some similarity with sequences identified as AA099051 (zn45c07.rl Stratagene HeLa cell s3 937216 Homo sapiens cDNA clone 550380 5′), AA424986 (zw06g01.rl Soares NhHMPu S1 Homo sapiens cDNA clone 768528 5′), AA480114 (zv41h05.s1 Soares ovary tumor NbHOT Homo sapiens cDNA clone 756249 3′), H73153 (yu26e 11.rl Homo sapiens cDNA clone 234956 5′), and H73629 (yu26f11.rl Homo sapiens cDNA clone 234957 5′). Based upon sequence similarity, du372_{—}1 proteins and each similar protein or peptide may share at least some activity.

Clone “ej90_{—}5”

A polynucleotide of the present invention has been identified as clone “ej90_{—}5”. ej90_{—}5 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ej90_{—}5 is a full-length clone, including the entire coding sequence of a secreted protein (also referred to herein as “ej90_{—}5 protein”).

The nucleotide sequence of ej90_{—}5 as presently determined is reported in SEQ ID NO:145. What applicants presently believe to be the proper reading frame and the predicted amino acid sequence of the ej90_{—}5 protein corresponding to the foregoing nucleotide sequence is reported in SEQ ID NO:146. Amino acids 8 to 20 are a predicted leader/signal sequence, with the predicted mature amino acid sequence beginning at amino acid 21, or are a transmembrane domain.

The EcoRI/NotI restriction fragment obtainable from the deposit containing clone ej90_{—}5 should be approximately 850 bp.

The nucleotide sequence disclosed herein for ej90_{—}5 was searched against the GenBank and GeneSeq nucleotide sequence databases using BLASTN/BLASTX and FASTA search protocols. ej90_{—}5 demonstrated at least some similarity with sequences identified as AA099387 (zk85e10.rl Soares pregnant uterus NbHPU Homo sapiens cDNA clone 489642 5′), AA099388 (zk85e10.s1 Soares pregnant uterus NbHPU Homo sapiens cDNA clone 489642 3′), AA256657 (zr85c06.rl Soares NhHMPu S1 Homo sapiens cDNA clone 682474 5′), and X851 11 (X.laevis mRNA for XEL-1). Based upon sequence similarity, ej90_{—}5 proteins and each similar protein or peptide may share at least some activity. The TopPredII computer program predicts an additional potential trans-membrane domain within the ej90_{—}5 protein sequence centered around amino acid 164 of SEQ ID NO:146.

Clone “ic2_{—}6”

A polynucleotide of the present invention has been identified as clone “ic2_{—}6”. ic2_{—}6 was isolated from a human adult retina (retinoblastoma WERI-Rb1) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ic2_{—}6 is a full-length clone, including the entire coding sequence of a secreted protein (also referred to herein as “ic2_{—}6 protein”).

The nucleotide sequence of ic2_{—}6 as presently determined is reported in SEQ ID NO:147. What applicants presently believe to be the proper reading frame and the predicted amino acid sequence of the ic2_{—}6 protein corresponding to the foregoing nucleotide sequence is reported in SEQ ID NO:148. Amino acids 5 to 17 are a predicted leader/signal sequence, with the predicted mature amino acid sequence beginning at amino acid 18, or are a transmembrane domain.

The EcoRI/NotI restriction fragment obtainable from the deposit containing clone ic2_{—}6 should be approximately 2000 bp.

The nucleotide sequence disclosed herein for ic2_{—}6 was searched against the GenBank and GeneSeq nucleotide sequence databases using BLASTN/BLASTX and FASTA search protocols. ic2_{—}6 demonstrated at least some similarity with sequences identified as AA104139 (mp03a12.rl Stratagene mouse heart (#937316) Mus musculus cDNA clone 568126 5′), N39195 (yv26e08.s1 Homo sapiens cDNA clone 243878 3′), and Z59762 (H.sapiens CpG DNA, clone 171h5, reverse read cpg171hS.rt1a). Based upon sequence similarity, ic2_{—}6 proteins and each similar protein or peptide may share at least some activity.

Clone “bn97_{—}1”

A polynucleotide of the present invention has been identified as clone “bn97_{—}1”. bn97_{—}1 was isolated from a human adult placenta cDNA library was identified as encoding a secreted or transmembrane protein on the basis of computer analysis of the amino acid sequence of the encoded protein. bn97_{—}1 is a full-length clone, including the entire coding sequence of a secreted protein (also referred to herein as “bn97_{—}1 protein”).

The nucleotide sequence of bn97_{—}1 as presently determined is reported in SEQ ID NO:159. What applicants presently believe to be the proper reading frame and the predicted amino acid sequence of the bn97_{—}1 protein corresponding to the foregoing nucleotide sequence is reported in SEQ ID NO:160. Amino acids 55 to 67 are a predicted leader/signal sequence, with the predicted mature amino acid sequence beginning at amino acid 68, or are a transmembrane domain.

The EcoRI/NotI restriction fragment obtainable from the deposit containing clone bn97_{—}1 should be approximately 1700 bp.

The nucleotide sequence disclosed herein for bn97_{—}1 was searched against the GenBank and GeneSeq nucleotide sequence databases using BLASTN/BLASTX and FASTA search protocols. bn97_{—}1 demonstrated at least some identity with sequences identified as AA046689 (zk72h06.s1 Soares pregnant uterus NbHPU Homo sapiens cDNA clone 488411 3′), D30934 (Human fetal-lung cDNA 5′-end sequence), R78820 (yi90b03.rl Homo sapiens cDNA clone 146477 5′), and R91687 (yq10h09.s1 Homo sapiens cDNA clone 196577 3′). The predicted amino acid sequence disclosed herein for bn97_{—}1 was searched against the GenPept and GeneSeq amino acid sequence databases using the BLASTX search protocol. The predicted bn97_{—}1 protein demonstrated at least some identity with sequences identified as Al 0431 (Hepatitis-B virus surface antigen P31). The bn97_{—}1 protein also shows some identity (30% identity, 50% conserved amino acids) to both bovine and human lectin-like receptor for oxidized LDL (low-density lipoprotein). While this homology is weak, it gets stronger (44% identity and 62% conserved amino acids) in the lectin-like domain. Further, the 3′ untranslated region of the bovine receptor has seven mRNA unstabilising sequences (ATTTA) and bn97_{—}1 has four in its 3′ untranslated region. This lectin-like receptor for oxidized LDL (designated LOX-1, Sawamura et al., 1997, Nature 386: 73-77) is an integral membrane protein which binds oxidized low-density lipoproteins, internalizes them into the endothelial cells and destroys them, thus playing a crucial role in the pathogenesis of atherosclerosis. Based upon identity, bn97_{—}1 proteins and each identical protein or peptide may share at least some activity.

Clone “bn268_{—}11”

A polynucleotide of the present invention has been identified as clone “bn268_{—}11”. bn268_{—}11 was isolated from a human adult placenta cDNA library was identified as encoding a secreted or transmembrane protein on the basis of computer analysis of the amino acid sequence of the encoded protein. bn268_{—}11 is a full-length clone, including the entire coding sequence of a secreted protein (also referred to herein as “bn268_{—}11 protein”).

The nucleotide sequence of bn268_{—}11 as presently determined is reported in SEQ ID NO:161. What applicants presently believe to be the proper reading frame and the predicted amino acid sequence of the bn268_{—}11 protein corresponding to the foregoing nucleotide sequence is reported in SEQ ID NO:162.

The EcoRI/NotI restriction fragment obtainable from the deposit containing clone bn268_{—}11 should be approximately 1050 bp.

The nucleotide sequence disclosed herein for bn268_{—}11 was searched against the GenBank and GeneSeq nucleotide sequence databases using BLASTN/BLASTX and FASTA search protocols. bn268_{—}11 demonstrated at least some identity with sequences identified as D62832 (Human aorta cDNA 5′-end GEN-330C09) and U20159 (Mus musculus 76 kDa tyrosine phosphoprotein SLP-76 mRNA, complete cds). The predicted amino acid sequence disclosed herein for bn268_{—}11 was searched against the GenPept and GeneSeq amino acid sequence databases using the BLASTX search protocol. The predicted bn268_{—}11 protein demonstrated at least some identity with sequences identified as D83171 (GDP-GTP exchange protein for Rho1p [Saccharomyces cerevisiae]). Based upon identity, bn268_{—}11 proteins and each identical protein or peptide may share at least some activity. The TopPredII computer program predicts a potential transmembrane domain within the bn268_{—}11 protein sequence centered around amino acid 33 of SEQ ID NO:162; this region may also function as a signal sequence.

Clone “cb96_{—}10”

A polynucleotide of the present invention has been identified as clone “cb96_{—}10”. cb96_{—}10 was isolated from a human fetal brain cDNA library was identified as encoding a secreted or transmembrane protein on the basis of computer analysis of the amino acid sequence of the encoded protein. cb96_{—}10 is a full-length clone, including the entire coding sequence of a secreted protein (also referred to herein as “cb96_{—}10 protein”).

The nucleotide sequence of cb96_{—}10 as presently determined is reported in SEQ ID NO:163. What applicants presently believe to be the proper reading frame and the predicted amino acid sequence of the cb96_{—}10 protein corresponding to the foregoing nucleotide sequence is reported in SEQ ID NO:164. Amino acids 74 to 86 are a predicted leader/signal sequence, with the predicted mature amino acid sequence beginning at amino acid 87, or are a transmembrane domain.

The EcoRI/NotI restriction fragment obtainable from the deposit containing clone cb96_{—}10 should be approximately 2100 bp.

The nucleotide sequence disclosed herein for cb96_{—}10 was searched against the GenBank and GeneSeq nucleotide sequence databases using BLASTN/BLASTX and FASTA search protocols. cb96_{—}10 demonstrated at least some identity with sequences identified as AA459012, AA459236, AA256744 (zs31h11.rl Soares NbHTGBC Homo sapiens cDNA clone 686853 5′), N54489 (yv40f07.s1 Soares fetal liver spleen 1NFLS Homo sapiens cDNA clone 245221 3′), and N57339 (yw81h07.rl Homo sapiens cDNA clone 258685 5′). The predicted amino acid sequence disclosed herein for cb96_{—}10 was searched against the GenPept and GeneSeq amino acid sequence databases using the BLASTX search protocol. The predicted cb96_{—}10 protein demonstrated at least some identity with sequences identified as X80036 (ascorbate peroxidase [Arabidopsis thaliana]). Based upon identity, cb96_{—}10 proteins and each identical protein or peptide may share at least some activity. The TopPredII computer program predicts seven potential transmembrane domains within the cb96_{—}10 protein sequence, centered around amino acid residues 25, 80, 125, 225, 300, 350, and 440 of SEQ ID NO:162. Therefore, cb96_{—}10 is likely to be an integral membrane protein with multiple helices in the membrane; it also contains the sequence motif of the actinin-type actin-binding domains that are believed to anchor actin to the cell membrane.

Clone “cb213_{—}11”

A polynucleotide of the present invention has been identified as clone “cb213_{—}11”. cb213_{—}11 was isolated from a human fetal brain cDNA library was identified as encoding a secreted or transmembrane protein on the basis of computer analysis of the amino acid sequence of the encoded protein. cb213_{—}11 is a full-length clone, including the entire coding sequence of a secreted protein (also referred to herein as “cb213_{—}11 protein”).

The nucleotide sequence of cb213_{—}11 as presently determined is reported in SEQ ID NO:165. What applicants presently believe to be the proper reading frame and the predicted amino acid sequence of the cb213_{—}11 protein corresponding to the foregoing nucleotide sequence is reported in SEQ ID NO:166. Amino acids 29 to 41 are a predicted leader/signal sequence, with the predicted mature amino acid sequence beginning at amino acid 42, or are a transmembrane domain.

The EcoRI/NotI restriction fragment obtainable from the deposit containing clone cb213_{—}11 should be approximately 2400 bp.

The nucleotide sequence disclosed herein for cb213_{—}11 was searched against the GenBank and GeneSeq nucleotide sequence databases using BLASTN/BLASTX and FASTA search protocols. cb213_{—}11 demonstrated at least some identity with sequences identified as AA332165 (EST36344 Embryo, 8 week I Homo sapiens cDNA 5′ end) and R34507 (g58a03.rl Homo sapiens cDNA clone 36801 5′). The predicted amino acid sequence disclosed herein for cb213_{—}11 was searched against the GenPept and GeneSeq amino acid sequence databases using the BLASTX search protocol. The predicted cb213_{—}11 protein demonstrated at least some identity with sequences identified as U39847 (A013 ankyrin [Caenorhabditis elegans]). Based upon identity, cb213_{—}11 proteins and each identical protein or peptide may share at least some activity.

Clone “cj457_{—}41”

A polynucleotide of the present invention has been identified as clone “cj457_{—}4”. cj457_{—}4 was isolated from a human fetal brain cDNA library was identified as encoding a secreted or transmembrane protein on the basis of computer analysis of the amino acid sequence of the encoded protein. cj457_{—}4 is a full-length clone, including the entire coding sequence of a secreted protein (also referred to herein as “cj457_{—}4 protein”).

The nucleotide sequence of cj457_{—}4 as presently determined is reported in SEQ ID NO:167. What applicants presently believe to be the proper reading frame and the predicted amino acid sequence of the cj457_{—}4 protein corresponding to the foregoing nucleotide sequence is reported in SEQ ID NO:168. Amino acids 11 to 23 are a predicted leader/signal sequence, with the predicted mature amino acid sequence beginning at amino acid 24, or are a transmembrane domain.

The EcoRI/NotI restriction fragment obtainable from the deposit containing clone cj457_{—}4 should be approximately 3350 bp.

The nucleotide sequence disclosed herein for cj457_{—}4 was searched against the GenBank and GeneSeq nucleotide sequence databases using BLASTN/BLASTX and FASTA search protocols. cj457_{—}4 demonstrated at least some identity with sequences identified as T92881 (ye22a10.s1 Homo sapiens cDNA clone 118458 3′) and T92488 (ye21g09.rl Homo sapiens cDNA clone 118432 5′). Based upon identity, cj457_{—}4 proteins and each identical protein or peptide may share at least some activity. The TopPredII computer program predicts a potential transmembrane domain within the cj457_{—}4 protein sequence, centered around amino acid 17 of SEQ ID NO:168; this region may also function as a signal sequence.

Clone “cz653_{—}11”

A polynucleotide of the present invention has been identified as clone “cz653_{—}11”. cz653_{—}11 was isolated from a human adult testes cDNA library was identified as encoding a secreted or transmembrane protein on the basis of computer analysis of the amino acid sequence of the encoded protein. cz653_{—}11 is a full-length clone, including the entire coding sequence of a secreted protein (also referred to herein as “cz653_{—}11 protein”).

The nucleotide sequence of cz653_{—}11 as presently determined is reported in SEQ ID NO:169. What applicants presently believe to be the proper reading frame and the predicted amino acid sequence of the cz653_{—}11 protein corresponding to the foregoing nucleotide sequence is reported in SEQ ID NO:170.

The EcoRI/NotI restriction fragment obtainable from the deposit containing clone cz653_{—}11 should be approximately 1300 bp.

The nucleotide sequence disclosed herein for cz653_{—}11 was searched against the GenBank and GeneSeq nucleotide sequence databases using BLASTN/BLASTX and FASTA search protocols. cz653_{—}11 demonstrated at least some identity with sequences identified as AA024740 (ze76c09.s1 Soares fetal heart NbHH19W Homo sapiens cDNA clone 364912 3′), AA203204 (zx57b04.rl Soares fetal liver spleen 1NFLS S1 Homo sapiens cDNA clone 446575 5′ similar to contains element MSR1 repetitive element), W72894 (zd59e06.s1 Soares fetal heart NbHH19W Homo sapiens cDNA clone 344962 3′), and W76099 (zd59e06.rl Soares fetal heart NbHH19W Homo sapiens cDNA clone 344962 5′). The predicted cz653_{—}11 demonstrated similarity to various WD-40 repeat containing proteins such as beta transducin-like protein (L28125) and coatomer, beta-prime subunit (AJ006523). The homology appears to be due to the presence of the Beta-transducin family Trp-Asp repeats signature (WD-40) beginning at residue 262 of SEQ ID NO:117. The WD-40 repeat has been thought to be a protein-protein interaction domain. Based upon identity, cz653_{—}11 proteins and each identical protein or peptide may share at least some activity. The TopPredII computer program predicts a potential transmembrane domain within the cz653_{—}11 protein sequence centered around amino acid 200 of SEQ ID NO:170.

Clone “dx138_{—}4”

A polynucleotide of the present invention has been identified as clone “dx138_{—}4”. dx138_{—}4 was isolated from a human adult testes cDNA library was identified as encoding a secreted or transmembrane protein on the basis of computer analysis of the amino acid sequence of the encoded protein. dx138_{—}4 is a full-length clone, including the entire coding sequence of a secreted protein (also referred to herein as “dx138_{—}4 protein”).

The nucleotide sequence of dx138_{—}4 as presently determined is reported in SEQ ID NO:171. What applicants presently believe to be the proper reading frame and the predicted amino acid sequence of the dx138_{—}4 protein corresponding to the foregoing nucleotide sequence is reported in SEQ ID NO:172. Amino acids 268 to 280 are a predicted leader/signal sequence, with the predicted mature amino acid sequence beginning at amino acid 281, or are a transmembrane domain.

The EcoRI/NotI restriction fragment obtainable from the deposit containing clone dx138_{—}4 should be approximately 2300 bp.

The nucleotide sequence disclosed herein for dx138_{—}4 was searched against the GenBank and GeneSeq nucleotide sequence databases using BLASTN/BLASTX and FASTA search protocols. dx138_{—}4 demonstrated at least some identity with sequences identified as AA108970 (m163a06.rl Stratagene mouse testis (#937308) Mus musculus cDNA clone 516658 5′), AA280976 (zs97f01.rl Soares NbHTGBC Homo sapiens cDNA clone 711577 5′ similar to contains Alu repetitive element), H99316 (yx23a03.s1 Homo sapiens cDNA clone 262540 3′), T36050 (EST96120 Homo sapiens cDNA 5′), X85637 (H.sapiens mRNA for expressed sequence tag, clone CAM tEST417 (A)), and Z22280 (H.sapiens DNA sequence). Based upon identity, dx138_{—}4 proteins and each identical protein or peptide may share at least some activity.

Clone “ij167_{—}5”

A polynucleotide of the present invention has been identified as clone “ij167_{—}5”. ij167_{—}5 was isolated from a human adult blood (peripheral blood mononuclear cells treated in vivo with G-CSF) cDNA library was identified as encoding a secreted or transmembrane protein on the basis of computer analysis of the amino acid sequence of the encoded protein. ij167_{—}5 is a full-length clone, including the entire coding sequence of a secreted protein (also referred to herein as “ij167_{—}5 protein”).

The nucleotide sequence of ij167_{—}5 as presently determined is reported in SEQ ID NO:173. What applicants presently believe to be the proper reading frame and the predicted amino acid sequence of the ij167_{—}5 protein corresponding to the foregoing nucleotide sequence is reported in SEQ ID NO:174. Amino acids 12 to 24 are a predicted leader/signal sequence, with the predicted mature amino acid sequence beginning at amino acid 25, or are a transmembrane domain.

The EcoRI/NotI restriction fragment obtainable from the deposit containing clone ij167_{—}5 should be approximately 1050 bp.

The nucleotide sequence disclosed herein for ij167_{—}5 was searched against the GenBank and GeneSeq nucleotide sequence databases using BLASTN/BLASTX and FASTA search protocols. ij167_{—}5 demonstrated at least some identity with sequences identified as N71115 (za87h10.s1 Homo sapiens cDNA clone 299587 3′), T85491 (yd78b01.rl Homo sapiens cDNA clone 114313 5′), W04374 (za43f06.rl Soares fetal liver spleen 1NFLS Homo sapiens cDNA clone 295331 5′), W05476 (za87h10.rl Soares fetal lung NbHL19W Homo sapiens cDNA clone 299587 5′), and W40146 (zb74d09.rl Soares fetal lung NbHL19W Homo sapiens cDNA clone 309329 5′). The predicted amino acid sequence disclosed herein for ij167_{—}5 was searched against the GenPept and GeneSeq amino acid sequence databases using the BLASTX search protocol. The predicted ij167_{—}5 protein demonstrated at least some identity with sequences identified as M96653 (adenylyl cyclase, type 6 [Mus musculus]). Based upon identity, ij167_{—}5 proteins and each identical protein or peptide may share at least some activity. The TopPredII computer program predicts a potential transmembrane domain within the ij167_{—}5 protein sequence, centered around amino acid 40 of SEQ ID NO:174.

Clone “bd107_{—}16”

A polynucleotide of the present invention has been identified as clone “bd10716”. bd107_{—}16 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bd107_{—}16 is a full-length clone, including the entire coding sequence of a secreted protein (also referred to herein as “bd107_{—}16 protein”).

The nucleotide sequence of bd107_{—}16 as presently determined is reported in SEQ ID NO:183. What applicants presently believe to be the proper reading frame and the predicted amino acid sequence of the bd107_{—}16 protein corresponding to the foregoing nucleotide sequence is reported in SEQ ID NO:184.

The EcoRI/NotI restriction fragment obtainable from the deposit containing clone bd107_{—}16 should be approximately 1500 bp.

The nucleotide sequence disclosed herein for bd107_{—}16 was searched against the GenBank and GeneSeq nucleotide sequence databases using BLASTN/BLASTX and FASTA search protocols. bd107_{—}16 demonstrated at least some similarity with sequences identified as AA261841 (zs17h09.rl NCI_CGAP_GCB1 Homo sapiens cDNA clone IMAGE:685505 5′), AA424094 (zv80d05.rl Soares total fetus Nb2HF8 9w Homo sapiens cDNA clone 759945 5′), AA449717 (zx09b06.s1 Soares total fetus Nb2HF8 9w Homo sapiens cDNA clone 785939 3′ similar to TR:E246888 E246888 CHROMOSOME XVI READING FRAME ORF YPL146C), AA875866 (ob34d08.s1 NCI_CGAP_Kid5 Homo sapiens cDNA clone IMAGE:1325583 3′ similar to TR:Q12080 Q12080 P2610), H80410 (yu97b09.rl Homo sapiens cDNA clone 241145 5′), N39747 (yx92h07.rl Homo sapiens cDNA clone 269245 5′), R97655 (yq59d12.rl Homo sapiens cDNA clone 200087 5′), T19822 (Human gene signature HUMGS00904; standard; cDNA to mRNA), and W68551 (zd36h03.rl Soares fetal heart NbHH19W Homo sapiens cDNA clone 342773 5′). The predicted amino acid sequence disclosed herein for bd107_{—}16 was searched against the GenPept and GeneSeq amino acid sequence databases using the BLASTX search protocol. The predicted bd107_{—}16 protein demonstrated at least some similarity to the sequence identified as U43703 (Lpi2p [Saccharomyces cerevisiae]). Based upon sequence similarity, bd107_{—}16 proteins and each similar protein or peptide may share at least some activity.

Clone “bm41_{—}7”

A polynucleotide of the present invention has been identified as clone “bm41_{—}7”. bm41_{—}7 was isolated from a human adult muscle cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bm41_{—}7 is a full-length clone, including the entire coding sequence of a secreted protein (also referred to herein as “bm41_{—}7 protein”).

The nucleotide sequence of bm41_{—}7 as presently determined is reported in SEQ ID NO:185. What applicants presently believe to be the proper reading frame and the predicted amino acid sequence of the bm41_{—}7 protein corresponding to the foregoing nucleotide sequence is reported in SEQ ID NO:186. Amino acids 15 to 27 are a predicted leader/signal sequence, with the predicted mature amino acid sequence beginning at amino acid 28, or are within a transmembrane domain.

The EcoRI/NotI restriction fragment obtainable from the deposit containing clone bm41_{—}7 should be approximately 1700 bp.

The nucleotide sequence disclosed herein for bm41_{—}7 was searched against the GenBank and GeneSeq nucleotide sequence databases using BLASTN/BLASTX and FASTA search protocols. bm41_{—}7 demonstrated at least some similarity with sequences identified as AF047439 (Homo sapiens unknown mRNA, complete cds), H44519 (yo74d10.rl Homo sapiens cDNA clone 183667 5′), N29833 (yw93d10.s1 Homo sapiens cDNA clone 259795 3′), T23021 (Human gene signature HUMGS04750; standard; cDNA to mRNA), W58059 (zd22f10.rl Soares fetal heart NbHH19W Homo sapiens cDNA clone), and Z78368 (H.sapiens mRNA, expressed sequence tag ICRFp507F18226, mRNA sequence). The predicted amino acid sequence disclosed herein for bm41_{—}7 was searched against the GenPept and GeneSeq amino acid sequence databases using the BLASTX search protocol. The predicted bm41_{—}7 protein demonstrated at least some similarity to the sequence identified as AF047439 (unknown [Homo sapiens]). Based upon sequence similarity, bm41_{—}7 proteins and each similar protein or peptide may share at least some activity. The TopPredII computer program predicts an additional potential trans-membrane domain within the bm41_{—}7 protein sequence centered around amino acid 252 of SEQ ID NO:186.

Clone “br342_{—}11”

A polynucleotide of the present invention has been identified as clone “br342_{—}11”. br342_{—}11 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. br342_{—}11 is a full-length clone, including the entire coding sequence of a secreted protein (also referred to herein as “br342_{—}11 protein”).

The nucleotide sequence of br342_{—}11 as presently determined is reported in SEQ ID NO:187. What applicants presently believe to be the proper reading frame and the predicted amino acid sequence of the br342_{—}11 protein corresponding to the foregoing nucleotide sequence is reported in SEQ ID NO:188. Amino acids 49 to 61 are a predicted leader/signal sequence, with the predicted mature amino acid sequence beginning at amino acid 62, or are within a transmembrane domain.

The EcoRI/NotI restriction fragment obtainable from the deposit containing clone br342_{—}11 should be approximately 1400 bp.

The nucleotide sequence disclosed herein for br342_{—}11 was searched against the GenBank and GeneSeq nucleotide sequence databases using BLASTN/BLASTX and FASTA search protocols. br342_{—}11 demonstrated at least some similarity with sequences identified as Z69722 (Human DNA sequence from cosmid U212C1, between markers DXS366 and DXS87 on chromosome X), Z93019 Human DNA sequence SEQUENCING IN PROGRESS *** from clone 49C23; HTGS phase 1; Human DNA sequence from PAC 49C23 on chromosome X contains malate dehydrogenase pseudogene and STS), and Z95126 (Human DNA sequence *** SEQUENCING IN PROGRESS *** from clone 30P20; HTGS phase 1; Human DNA sequence from PAC 30P20 on chromosome Xq21.1-Xq21.3. Contains set pseudogene, ESTs and STS). The predicted amino acid sequence disclosed herein for br342_{—}11 was searched against the GenPept and GeneSeq amino acid sequence databases using the BLASTX search protocol. The predicted br342_{—}11 protein demonstrated at least some similarity to sequences identified as D89049 (lectin-like oxidized LDL receptor [Bos taurus]) and R99586 (Low density lipoprotein receptor). Based upon sequence similarity, br342_{—}11 proteins and each similar protein or peptide may share at least some activity. The nucleotide sequence of br342_{—}11 indicates that it may contain one or more of the following repetitive elements: MER4A, MER4B.

Clone “ej258_{—}11”

A polynucleotide of the present invention has been identified as clone “ej258_{—}11”. ej258_{—}11 was isolated from a human adult placenta cDNA library using methods which are selective for cDNAs encoding, secreted proteins (see U.S. Pat. No. 5,536,637), or was identified as encoding a secreted or transmembrane protein on the basis of computer analysis of the amino acid sequence of the encoded protein. ej258_{—}11 is a full-length clone, including the entire coding sequence of a secreted protein (also referred to herein as “ej258_{—}11 protein”).

The nucleotide sequence of ej258_{—}11 as presently determined is reported in SEQ ID NO:189. What applicants presently believe to be the proper reading frame and the predicted amino acid sequence of the ej258_{—}11 protein corresponding to the foregoing nucleotide sequence is reported in SEQ ID NO:190.

The EcoRI/NotI restriction fragment obtainable from the deposit containing clone ej258_{—}11 should be approximately 670 bp.

The nucleotide sequence disclosed herein for ej258_{—}11 was searched against the GenBank and GeneSeq nucleotide sequence databases using BLASTN/BLASTX and FASTA search protocols. ej258_{—}11 demonstrated at least some similarity with sequences identified as AA217161 (mu86g11.rl Soares mouse lymph node NbMLN Mus musculus cDNA clone 652484 5′ similar to WP:F35H12.2 CE04511), AA330720 (EST34452 Embryo, 6 week I Homo sapiens cDNA 5′ end), AJ000649 (Oryctolagus cuniculus unknown differentially expressed mRNA), U17432 (Bos taurus beta-mannosidase mRNA, complete cds), U91321 (Human chromosome 16p13 BAC clone CIT987SK-363E6, complete sequence), Z74031 (Caenorhabditis elegans cosmid F32D8), and Z99127 (Human DNA sequence *** SEQUENCING IN PROGRESS *** from clone 102G20; HTGS phase 1; Human DNA sequence from PAC 102G20 on chromosome 1q24-q25. Contains ESTS, STSs and a predicted CpG island). The predicted amino acid sequence disclosed herein for ej258_{—}11 was searched against the GenPept and GeneSeq amino acid sequence databases using the BLASTX search protocol. The predicted ej258_{—}11 protein demonstrated at least some similarity to the sequence identified as U41540 (coded for by C. elegans cDNA yk42d12.5; coded for by C. elegans cDNA yk27e10.5; coded for by C. elegans cDNA cm08h6; coded for by C. elegans cDNA yk88e12.5). Based upon sequence similarity, ej258_{—}11 proteins and each similar protein or peptide may share at least some activity.

Clone “k232_{—}2x”

A polynucleotide of the present invention has been identified as clone “k232_{—}2x”. A cDNA clone was first isolated from a murine adult bone marrow cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. This murine cDNA was then used to isolate k232_{—}2x, a full-length human cDNA clone, including the entire coding sequence of a secreted protein (also referred to herein as “k232_{—}2x protein”).

The nucleotide sequence of k232_{—}2x as presently determined is reported in SEQ ID NO:191. What applicants presently believe to be the proper reading frame and the predicted amino acid sequence of the k232_{—}2x protein corresponding to the foregoing nucleotide sequence is reported in SEQ ID NO:192. Amino acids 4 to 16 are a predicted leader/signal sequence, with the predicted mature amino acid sequence beginning at amino acid 17, or are within a transmembrane domain.

The EcoRI/NotI restriction fragment obtainable from the deposit containing clone k232_{—}2x should be approximately 555 bp.

The nucleotide sequence disclosed herein for k232_{—}2x was searched against the GenBank and GeneSeq nucleotide sequence databases using BLASTN/BLASTX and FASTA search protocols. k232_{—}2x demonstrated at least some similarity with sequences identified as AA087828 (mn94b04.rl Stratagene mouse lung 937302 Mus musculus cDNA clone 551695 5′), AA095731 (15720.seq.F Fetal heart, Lambda ZAP Express Homo sapiens cDNA 5′), AA398859 (zt80e12.rl Soares testis NHT Homo sapiens cDNA clone 728686 5′), N78829 (zb17a05.s1 Homo sapiens cDNA clone 302288 3′), T21965 (Human gene signature HUMGS03508), and W17346 (zb18c05.rl Soares fetal lung NbHL19W Homo sapiens cDNA clone 302408 5′). Based upon sequence similarity, k232_{—}2x proteins and each similar protein or peptide may share at least some activity. The TopPredII computer program predicts two potential transmembrane domains within the k232_{—}2x protein sequence, one near the signal sequence and another near the C-terminus of SEQ ID NO:192.

Clone “lf307_{—}5”

A polynucleotide of the present invention has been identified as clone “lf307_{—}5”. lf307_{—}5 was isolated from a human adult spinal cord cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. lf307_{—}5 is a full-length clone, including the entire coding sequence of a secreted protein (also referred to herein as “lf307_{—}5 protein”).

The nucleotide sequence of the 5′ portion of lf307_{—}5 as presently determined is reported in SEQ ID NO:193. What applicants presently believe is the proper reading frame for the coding region is indicated in SEQ ID NO:194. The predicted amino acid sequence of the lf307_{—}5 protein corresponding to the foregoing nucleotide sequence is reported in SEQ ID NO:194. Additional nucleotide sequence from the 3′ portion of lf307_{—}5, including the polyA tail, is reported in SEQ ID NO:195.

The EcoRI/NotI restriction fragment obtainable from the deposit containing clone lf307_{—}5 should be approximately 1000 bp.

The nucleotide sequence disclosed herein for lf307_{—}5 was searched against the GenBank and GeneSeq nucleotide sequence databases using BLASTN/BLASTX and FASTA search protocols. lf307_{—}5 demonstrated at least some similarity with sequences identified as AA039895 (zk46a02.rl Soares pregnant uterus NbHPU Homo sapiens cDNA clone 485834 5′) and AA513783 (nh89a05.rl NCI_CGAP_Br1.1 Homo sapiens cDNA clone 965648). Based upon sequence similarity, lf307_{—}5 proteins and each similar protein or peptide may share at least some activity. The TopPredII computer program predicts a potential transmembrane domain within the lf307_{—}5 protein sequence centered around amino acid 50 of SEQ ID NO:194.

Clone “lr204_{—}1”

A polynucleotide of the present invention has been identified as clone “lr204_{—}1”. lr204_{—}1 was isolated from a human adult lymph node cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. lr204_{—}1 is a full-length clone, including the entire coding sequence of a secreted protein (also referred to herein as “lr204_{—}1 protein”).

The nucleotide sequence of lr204_{—}1 as presently determined is reported in SEQ ID NO:196. What applicants presently believe to be the proper reading frame and the predicted amino acid sequence of the lr204_{—}1 protein corresponding to the foregoing nucleotide sequence is reported in SEQ ID NO:197. Amino acids 29 to 41 are a predicted leader/signal sequence, with the predicted mature amino acid sequence beginning at amino acid 42, or are within a transmembrane domain.

The EcoRI/NotI restriction fragment obtainable from the deposit containing clone lr204_{—}1 should be approximately 900 bp.

The nucleotide sequence disclosed herein for lr204_{—}1 was searched against the GenBank and GeneSeq nucleotide sequence databases using BLASTN/BLASTX and FASTA search protocols. lr204_{—}1 demonstrated at least some similarity with sequences identified as N47763 (yy55e07.rl Homo sapiens cDNA clone 277476 5′) and N56875 (yy55e07.s1 Homo sapiens cDNA clone 277476 3′ similar to SW:CYTO_BOVIN P01035 CYSTATIN, COLOSTRUM). The predicted amino acid sequence disclosed herein for lr204_{—}1 was searched against the GenPept and GeneSeq amino acid sequence databases using the BLASTX search protocol. The predicted lr204_{—}1 protein demonstrated at least some similarity to sequences identified as M27891 (cystatin C [Homo sapiens]), P94392 (Sequence of complete recombinant cystatin C in E. coli), R43323 (Cystatin polypeptide), and X62412 (cystatin [unidentified]). Based upon sequence similarity, lr204_{—}1 proteins and each similar protein or peptide may share at least some activity. The predicted lr204_{—}1 protein contains a sequence highly conserved in all cystatins (QIVAG in human cystatin C, QIVKG in the predicted lr204_{—}1 protein). Cystatins are inhibitors of papain-like cysteine proteinases such as cathepsins. Cystatin C belongs to family 2 of the cystatin superfamily. The family 2 cystatins are secreted proteins of about 120 amino acids. All cystatins have important roles in processes involving cysteine proteinase activity like bone resorption. They are also implicated in a variety of diseases (e.g. sepsis, cancer metastasis, rheumatoid arthritis etc.) since they regulate potentially harmful proteinase activity. The predicted lr204_{—}1 protein appears to be a novel cystatin C related inhibitor of cysteine proteinases. The TopPredII computer program predicts a potential transmembrane domain within the lr204_{—}1 protein sequence near amino acid 42 of SEQ ID NO:197.

Clone “as20_{—}2”

A polynucleotide of the present invention has been identified as clone “as20_{—}2”. as20_{—}2 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. as20_{—}2 is a full-length clone, including the entire coding sequence of a secreted protein (also referred to herein as “as20_{—}2 protein”) and a poly(A) tail at its 3′ end.

The nucleotide sequence of as20_{—}2 as presently determined is reported in SEQ ID NO:205. What applicants presently believe to be the proper reading frame and the predicted amino acid sequence of the as20_{—}2 protein corresponding to the foregoing nucleotide sequence is reported in SEQ ID NO:206. Amino acids 17 to 29 are a predicted leader/signal sequence, with the predicted mature amino acid sequence beginning at amino acid 30, or are within a transmembrane domain.

The EcoRI/NotI restriction fragment obtainable from the deposit containing clone as20_{—}2 should be approximately 2100 bp.

The nucleotide sequence disclosed herein for as20_{—}2 was searched against the GenBank and GeneSeq nucleotide sequence databases using BLASTN/BLASTX and FASTA search protocols. as20_{—}2 demonstrated at least some similarity with sequences identified as AA192606 (zq01g04.s1 Stratagene muscle 937209 Homo sapiens cDNA clone 628470 3′), AF000657 (Arabidopsis thaliana BAC F19G10, complete sequence), T91778 (yd52c10.s1 Homo sapiens cDNA clone 111858 3′ similar to contains Alu repetitive element), and W26193 (22b2 Human retina cDNA randomly primed sublibrary Homo sapiens cDNA). Based upon sequence similarity, as20_{—}2 proteins and each similar protein or peptide may share at least some activity. The TopPredII computer program predicts a potential transmembrane domain within the as20_{—}2 protein sequence centered around amino acid 97 of SEQ ID NO:206.

Clone “bf227_{—}8”

A polynucleotide of the present invention has been identified as clone “bf227_{—}8”. bf227_{—}8 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bf227_{—}8 is a full-length clone, including the entire coding sequence of a secreted protein (also referred to herein as “bf227_{—}8 protein”) and a poly(A) tail at its 3′ end.

The nucleotide sequence of bf227_{—}8 as presently determined is reported in SEQ ID NO:207. What applicants presently believe to be the proper reading frame and the predicted amino acid sequence of the bf227_{—}8 protein corresponding to the foregoing nucleotide sequence is reported in SEQ ID NO:208.

The EcoRI/NotI restriction fragment obtainable from the deposit containing clone bf227_{—}8 should be approximately 1400 bp.

The nucleotide sequence disclosed herein for bf227_{—}8 was searched against the GenBank and GeneSeq nucleotide sequence databases using BLASTN/BLASTX and FASTA search protocols. bf227_{—}8 demonstrated at least some similarity with sequences identified as AA452345 (zx15c09.rl Soares total fetus Nb2HF8 9w Homo sapiens cDNA clone 786544 5′), N20474 (yx39d10.s1 Homo sapiens cDNA clone 264115 3′), N79685 (yz82a08.rl Homo sapiens cDNA clone 289526 5′), and W73775 (zd50d09.s1 Soares fetal heart NbHH19W Homo sapiens cDNA clone 344081 3′). The predicted amino acid sequence disclosed herein for bf227_{—}8 was searched against the GenPept and GeneSeq amino acid sequence databases using the BLASTX search protocol. The predicted bf227_{—}8 protein demonstrated at least some similarity to sequences identified as U11768 (coat protein [Grapevine fanleaf virus]). Based upon sequence similarity, bf227_{—}8 proteins and each similar protein or peptide may share at least some activity. The TopPredII computer program predicts a potential transmembrane domain within the bf227_{—}8 protein sequence centered around amino acid 22 of SEQ ID NO:208.

Clone “bh157_{—}7”

A polynucleotide of the present invention has been identified as clone “bh157_{—}7”. bh157_{—}7 was isolated from a human adult ovary cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. bh157_{—}7 is a full-length clone, including the entire coding sequence of a secreted protein (also referred to herein as “bh157_{—}7 protein”) and a poly(A) tail at its 3′ end.

The nucleotide sequence of bh157_{—}7 as presently determined is reported in SEQ ID NO:209. What applicants presently believe to be the proper reading frame and the predicted amino acid sequence of the bh157_{—}7 protein corresponding to the foregoing nucleotide sequence is reported in SEQ ID NO:210.

The EcoRI/NotI restriction fragment obtainable from the deposit containing clone bh157_{—}7 should be approximately 1250 bp.

The nucleotide sequence disclosed herein for bh157_{—}7 was searched against the GenBank and GeneSeq nucleotide sequence databases using BLASTN/BLASTX and FASTA search protocols. bh157_{—}7 demonstrated at least some similarity with sequences identified as AA312435 (EST183106 Jurkat T-cells VI Homo sapiens cDNA 5′ end) and T62753 (yc70g05.rl Homo sapiens cDNA clone 86072 5′). The predicted amino acid sequence disclosed herein for bh157_{—}7 was searched against the GenPept and GeneSeq amino acid sequence databases using the BLASTX search protocol. The predicted bh157_{—}7 protein demonstrated at least some similarity to sequences identified as X84037 (E-selectin ligand-1 [Mus musculus]). Based upon sequence similarity, bh157_{—}7 proteins and each similar protein or peptide may share at least some activity.

Clone “cg426_{—}8”

A polynucleotide of the present invention has been identified as clone “cg426_{—}8”. cg426_{—}8 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. cg426_{—}8 is a full-length clone, including the entire coding sequence of a secreted protein (also referred to herein as “cg426_{—}8 protein”) and a poly(A) tail at its 3′ end.

The nucleotide sequence of cg426_{—}8 as presently determined is reported in SEQ ID NO:211. What applicants presently believe to be the proper reading frame and the predicted amino acid sequence of the cg426_{—}8 protein corresponding to the foregoing nucleotide sequence is reported in SEQ ID NO:212. Amino acids 4 to 16 are a predicted leader/signal sequence, with the predicted mature amino acid sequence beginning at amino acid 17, or are within a transmembrane domain.

The EcoRI/NotI restriction fragment obtainable from the deposit containing clone cg426_{—}8 should be approximately 2500 bp.

The nucleotide sequence disclosed herein for cg426_{—}8 was searched against the GenBank and GeneSeq nucleotide sequence databases using BLASTN/BLASTX and FASTA search protocols. cg426_{—}8 demonstrated at least some similarity with sequences identified as AA523415 (ng30a08.s1 NCI_CGAP_Co3 Homo sapiens cDNA clone 936278 similar to contains element MER22 repetitive element), N58694 (yv64f11.rl Homo sapiens cDNA clone 247533 5′), and W78817 (zh51c03.rl Soares fetal liver spleen 1NFLS S1 Homo sapiens cDNA clone 415588 5′ similar to contains MER1.t3 MER1 repetitive element). Based upon sequence similarity, cg426_{—}8 proteins and each similar protein or peptide may share at least some activity. The TopPredII computer program predicts a potential transmembrane domain within the cg426_{—}8 protein sequence centered around amino acid 55 of SEQ ID NO:212. The nucleotide sequence of cg426_{—}8 may contain a MER repetitive element.

Clone “ck48_{—}12”

A polynucleotide of the present invention has been identified as clone “ck48_{—}12”. ck48_{—}12 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ck48_{—}12is a full-length clone, including the entire coding sequence of a secreted protein (also referred to herein as “ck48_{—}12 protein”) and a poly(A) tail at its 3′ end.

The nucleotide sequence of ck48_{—}12 as presently determined is reported in SEQ ID NO:213. What applicants presently believe to be the proper reading frame and the predicted amino acid sequence of the ck48_{—}12 protein corresponding to the foregoing nucleotide sequence is reported in SEQ ID NO:214. Amino acids 119 to 131 are a predicted leader/signal sequence, with the predicted mature amino acid sequence beginning at amino acid 132, or are within a transmembrane domain.

The EcoRI/NotI restriction fragment obtainable from the deposit containing clone ck48_{—}12 should be approximately 1350 bp.

The nucleotide sequence disclosed herein for ck48_{—}12 was searched against the GenBank and GeneSeq nucleotide sequence databases using BLASTN/BLASTX and FASTA search protocols. ck48_{—}12 demonstrated at least some similarity with sequences identified as AA064481 (m150a08.rl Stratagene mouse testis (#937308) Mus musculus cDNA clone 515414 5′) and AA397716 (zt87f10.rl Soares testis NHT Homo sapiens cDNA clone 729355 5′). Based upon sequence similarity, ck48_{—}12 proteins and each similar protein or peptide may share at least some activity. The ck48_{—}12 protein sequence may have a transmembrane domain at the carboxyl terminus of SEQ ID NO:214.

Clone “co1000_{—}1”

A polynucleotide of the present invention has been identified as clone “co1000_{—}1”. co1000_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. co1000_{—}1 is a full-length clone, including the entire coding sequence of a secreted protein (also referred to herein as “co1000_{—}1 protein”) and a poly(A) tail at its 3′ end.

The nucleotide sequence of co1000_{—}1 as presently determined is reported in SEQ ID NO:215. What applicants presently believe to be the proper reading frame and the predicted amino acid sequence of the co1000_{—}1 protein corresponding to the foregoing nucleotide sequence is reported in SEQ ID NO:216. Amino acids 14 to 26 are a predicted leader/signal sequence, with the predicted mature amino acid sequence beginning at amino acid 27, or are within a transmembrane domain.

The EcoRI/NotI restriction fragment obtainable from the deposit containing clone co1000_{—}1 should be approximately 1600 bp.

The nucleotide sequence disclosed herein for co1000_{—}1 was searched against the GenBank and GeneSeq nucleotide sequence databases using BLASTN/BLASTX and FASTA search protocols. co1000_{—}1 demonstrated at least some similarity with sequences identified as M77867 (EST01451 Homo sapiens cDNA clone HFBCA06 similar to Alu repetitive element). Based upon sequence similarity, co1000_{—}1 proteins and each similar protein or peptide may share at least some activity. The nucleotide sequence of co1000_{—}1 indicates that it may contain Alu and Mer4 repetitive elements.

Clone “ct489_{—}14”

A polynucleotide of the present invention has been identified as clone “ct489_{—}14”. ct489_{—}14 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. ct489_{—}14 is a full-length clone, including the entire coding sequence of a secreted protein (also referred to herein as “ct489_{—}14 protein”) and a poly(A) tail at its 3′ end.

The nucleotide sequence of ct489_{—}14 as presently determined is reported in SEQ ID NO:217. What applicants presently believe to be the proper reading frame and the predicted amino acid sequence of the ct489_{—}14 protein corresponding to the foregoing nucleotide sequence is reported in SEQ ID NO:218. Amino acids 12 to 24 are a predicted leader/signal sequence, with the predicted mature amino acid sequence beginning at amino acid 25, or are within a transmembrane domain.

The EcoRI/NotI restriction fragment obtainable from the deposit containing clone ct489_{—}14 should be approximately 1900 bp.

The nucleotide sequence disclosed herein for ct489_{—}14 was searched against the GenBank and GeneSeq nucleotide sequence databases using BLASTN/BLASTX and FASTA search protocols. ct489_{—}14 demonstrated at least some similarity with sequences identified as H21179 (yn66d09.s1 Homo sapiens cDNA clone 173393 3′), N99345 (IMAGE:59425 Homo sapiens cDNA clone 59425), and R89669 (ym97f05.rl Homo sapiens cDNA clone 166881 5′). The predicted amino acid sequence disclosed herein for ct489_{—}14 was searched against the GenPept and GeneSeq amino acid sequence databases using the BLASTX search protocol. The predicted ct489_{—}14 protein demonstrated at least some similarity to sequences identified as U23803 (heterogeneous ribonucleoprotein A0 [Homo sapiens]). Based upon sequence similarity, ct489_{—}14proteins and each similar protein or peptide may share at least some activity. The TopPredII computer program predicts a potential transmembrane domain within the ct489_{—}14 protein sequence centered around amino acid 280 of SEQ ID NO:217.

Clone “df821_{—}1”

A polynucleotide of the present invention has been identified as clone “df821_{—}1”. df821_{—}1 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. df821_{—}1 is a full-length clone, including the entire coding sequence of a secreted protein (also referred to herein as “df821_{—}1 protein”) and a poly(A) tail at its 3′ end.

The nucleotide sequence of df821_{—}1 as presently determined is reported in SEQ ID NO:219. What applicants presently believe to be the proper reading frame and the predicted amino acid sequence of the df821_{—}1 protein corresponding to the foregoing nucleotide sequence is reported in SEQ ID NO:220.

The EcoRI/NotI restriction fragment obtainable from the deposit containing clone df821_{—}1 should be approximately 800 bp.

The nucleotide sequence disclosed herein for df821_{—}1 was searched against the GenBank and GeneSeq nucleotide sequence databases using BLASTN/BLASTX and FASTA search protocols. df821_{—}1 demonstrated at least some similarity with sequences identified as AA311729 (EST182669 Jurkat T-cells VI Homo sapiens cDNA 5′ end similar to similar to hypothetical protein pIL2), M17412 (Rat growth and transformation-dependent mRNA, 3′ end), W01700 (za37a03.rl Soares fetal liver spleen 1NFLS Homo sapiens cDNA clone 294700 5′ similar to PIR A26882 A26882 pIL2 hypothetical protein-rat), W44481 (zc28g12.rl Soares senescent fibroblasts NbHSF Homo sapiens cDNA clone 323686 5′ similar to PIR:A26882 A26882 pIL2 hypothetical protein-rat), and W93991 (zd98b04.s1 Soares fetal heart NbHH19W Homo sapiens cDNA clone 357487 3′ similar to PIR A26882 A26882 pIL2 hypothetical protein-rat). The predicted amino acid sequence disclosed herein for df821_{—}1 was searched against the GenPept and GeneSeq amino acid sequence databases using the BLASTX search protocol. The predicted df821_{—}1 protein demonstrated at least some similarity to sequences identified as M17412 (growth and transformation dependent protein [Rattus norvegicus]). Based upon sequence similarity, df821_{—}1 proteins and each similar protein or peptide may share at least some activity. The TopPredII computer program predicts a potential transmembrane domain within the df821_{—}1 protein sequence centered around amino acid 110 of SEQ ID NO:220.

Clone “dy41_{—}2”

A polynucleotide of the present invention has been identified as clone “dy41_{—}2”. dy41_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. dy41_{—}2 is a full-length clone, including the entire coding sequence of a secreted protein (also referred to herein as “dy41_{—}2 protein”) and a poly(A) tail at its 3′ end.

The nucleotide sequence of dy41_{—}2 as presently determined is reported in SEQ ID NO:221. What applicants presently believe to be the proper reading frame and the predicted amino acid sequence of the dy41_{—}2 protein corresponding to the foregoing nucleotide sequence is reported in SEQ ID NO:222. Amino acids 32 to 44 are a predicted leader/signal sequence, with the predicted mature amino acid sequence beginning at amino acid 45, or are within a transmembrane domain.

The EcoRI/NotI restriction fragment obtainable from the deposit containing clone dy41_{—}2 should be approximately 1200 bp.

The nucleotide sequence disclosed herein for dy41_{—}2 was searched against the GenBank and GeneSeq nucleotide sequence databases using BLASTN/BLASTX and FASTA search protocols. dy41_{—}2 demonstrated at least some similarity with sequences identified as H02323 (yj40f04.s1 Homo sapiens cDNA clone 151231 3′). The predicted amino acid sequence disclosed herein for dy41_{—}2 was searched against the GenPept and GeneSeq amino acid sequence databases using the BLASTX search protocol. The predicted dy41_{—}2 protein demonstrated at least some similarity to sequences identified as D89050 (lectin-like oxidized LDL receptor [Homo sapiens]). Based upon sequence similarity, dy412 proteins and each similar protein or peptide may share at least some activity. The TopPredII computer program predicts a potential transmembrane domain within the dy412 protein sequence, from amino acid 40 to amino acid 60 of SEQ ID NO:222.

Deposit of Clones Clones bh389_{—}11, bk112_{—}15, bk200_{—}13, di386_{—}3, em397_{—}2, fh170_{—}7, fn53_{—}4, fq505_{—}4, fw13_{—}9, and gg619_{—}2 were deposited on Jun. 10, 1997 with the American Type Culture Collection (10801 University Boulevard, Manassas, Va. 20110-2209 U.S.A.) as an original deposit under the Budapest Treaty and were given the accession number ATCC 98451, from which each clone comprising a particular polynucleotide is obtainable. All restrictions on the availability to the public of the deposited material will be irrevocably removed upon the granting of the patent, except for the requirements specified in 37 C.F.R. §1.808(b), and the term of the deposit will comply with 37 C.F.R. §1.806.

Clones cl181_{—}3, cr1044_{—}1, cz251_{—}1, dd12_{—}7, fn191_{—}3, gm196_{—}4, gn114_{—}1, hj968_{—}2, hk10_{—}3, and hm236_{—}1 were deposited on Jun. 12, 1997 with the American Type Culture Collection (10801 University Boulevard, Manassas, Va. 20110-2209 U.S.A.) as an original deposit under the Budapest Treaty and were given the accession number ATCC 98456, from which each clone comprising a particular polynucleotide is obtainable. All restrictions on the availability to the public of the deposited material will be irrevocably removed upon the granting of the patent, except for the requirements specified in 37 C.F.R. §1.808(b), and the term of the deposit will comply with 37 C.F.R. §1.806.Clones do15_{—}4, dx290_{—}1, ek390_{—}4 er471_{—}7, fs40_{—}3, ga63_{—}6, gm335_{—}4, hy370_{—}9, ie47_{—}4, and s195_{—}10 were deposited on Jun. 19, 1997 with the American Type Culture Collection (10801 University Boulevard, Manassas, Va. 20110-2209 U.S.A.) as an original deposit under the Budapest Treaty and were given the accession number ATCC 98468, from which each clone comprising a particular polynucleotide is obtainable. All restrictions on the availability to the public of the deposited material will be irrevocably removed upon the granting of the patent, except for the requirements specified in 37 C.F.R. §1.808(b), and the term of the deposit will comply with 37 C.F.R. §1.806.

Clones bf228_{—}14, bg249_{—}1, bv286_{—}1, co36_{—}1, cp116_{—}1, cw1195_{—}2, fh13_{—}10, gc57_{—}4, h1165_{—}3 and hb752_{—}1 were deposited on Jul. 2, 1997 with the American Type Culture Collection as an original deposit under the Budapest Treaty and were given the accession number ATCC 98482, from which each clone comprising a particular polynucleotide is obtainable. All restrictions on the availability to the public of the deposited material will be irrevocably removed upon the granting of the patent, except for the requirements specified in 37 C.F.R. §1.808(b).

Clones bi127_{—}5, bl194_{—}2, cc1301, ch582_{—}1, cq294_{—}14, dd454_{—}1, du157_{—}9, du372_{—}1, ej90_{—}5, and ic2_{—}6 were deposited on Aug. 5, 1997 with the American Type Culture Collection (10801 University Boulevard, Manassas, Va. 20110-2209 U.S.A.) as an original deposit under the Budapest Treaty and were given the accession number ATCC 98501, from which each clone comprising a particular polynucleotide is obtainable. Clone du157_{—}12 is an additional isolate of clone du157_{—}9 and was deposited on Apr. 7, 1998 with the American Type Culture Collection (10801 University Boulevard, Manassas, Va. 20110-2209 U.S.A.) as an original deposit under the Budapest Treaty and was given the accession number ATCC 98724, from which the du157_{—}12 clone comprising a particular polynucleotide is obtainable. All restrictions on the availability to the public of the deposited material will be irrevocably removed upon the granting of the patent, except for the requirements specified in 37 C.F.R. §1.808(b), and the term of the deposit will comply with 37 C.F.R. §1.806.

Clones bn97_{—}1, bn268_{—}11, cb96_{—}10, cb213_{—}11, cj457_{—}4, cz653_{—}11, dx138_{—}4, and ij167_{—}5 were deposited on Sep. 4, 1997 with the American Type Culture Collection (10801 University Boulevard, Manassas, Va. 20110-2209 U.S.A.) as an original deposit under the Budapest Treaty and were given the accession number ATCC 98535, from which each clone comprising a particular polynucleotide is obtainable. All restrictions on the availability to the public of the deposited material will be irrevocably removed upon the granting of the patent, except for the requirements specified in 37 C.F.R. §1.808(b), and the term of the deposit will comply with 37 C.F.R. §1.806.

Clones bd107_{—}16 and bm41_{—}7 were deposited on Sep. 25, 1998 with the American Type Culture Collection (10801 University Boulevard, Manassas, Va. 20110-2209 U.S.A.) as an original deposit under the Budapest Treaty and were given the accession number ATCC 98898, from which each clone comprising a particular polynucleotide is obtainable. Clones bd107_{—}13, bm41_{—}3, br342_{—}11, ej258_{—}11, k232_{—}2x, lf307_{—}5, and lr204_{—}1 were deposited on Oct. 2, 1997 with the American Type Culture Collection as an original deposit under the Budapest Treaty and were given the accession number ATCC 98551, from which each clone comprising a particular polynucleotide is obtainable. Clones bd107_{—}13 and bm41_{—}3 are additional isolates of clones bd107_{—}16 and bm41_{—}7, respectively. All restrictions on the availability to the public of the deposited material will be irrevocably removed upon the granting of the patent, except for the requirements specified in 37 C.F.R. §1.808(b), and the term of the deposit will comply with 37 C.F.R. §1.806.

Clones as20_{—}2, bf227_{—}8, bh157_{—}7, cg426_{—}8, ck48_{—}12, co1000_{—}1, ct489_{—}14, df821_{—}1 and dy412 were deposited on Nov. 7, 1997 with the American Type Culture Collection (10801 University Boulevard, Manassas, Va. 20110-2209 U.S.A.) as an original deposit under the Budapest Treaty and were given the accession number ATCC 98580, from which each clone comprising a particular polynucleotide is obtainable. All restrictions on the availability to the public of the deposited material will be irrevocably removed upon the granting of the patent, except for the requirements specified in 37 C.F.R. §1.808(b), and the term of the deposit will comply with 37 C.F.R. §1.806.

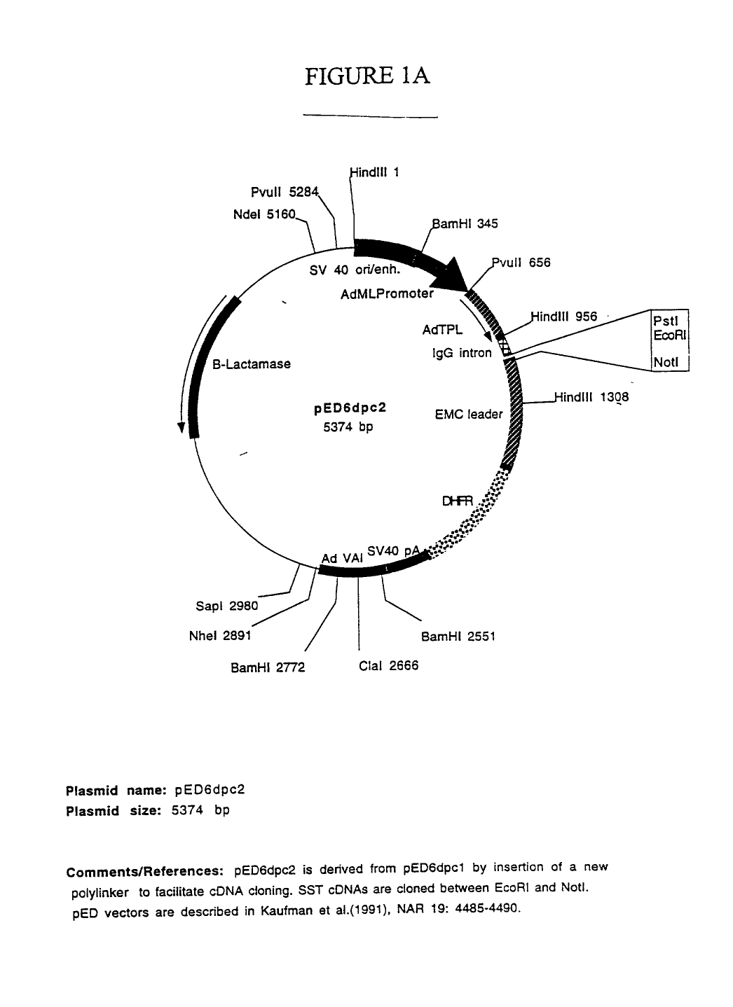

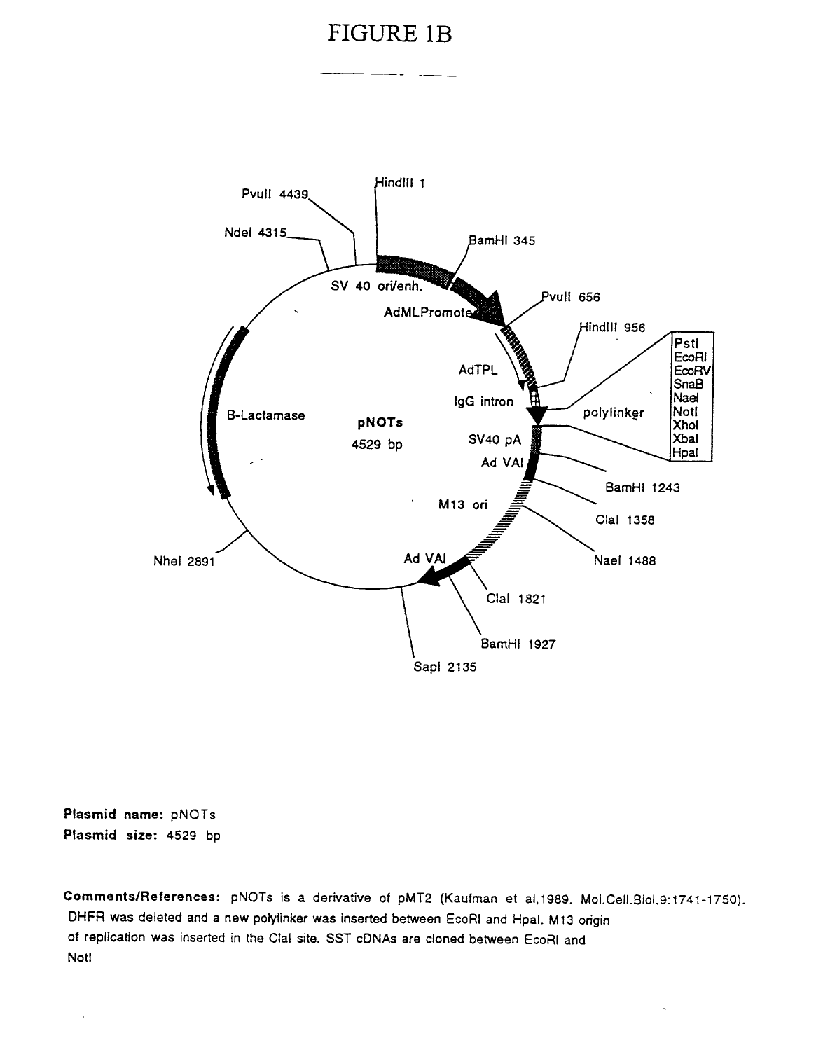

Each clone has been transfected into separate bacterial cells (E. coli) in this composite deposit. Each clone can be removed from the vector in which it was deposited by performing an EcoRI/NotI digestion (5′ site, EcoRI; 3′ site, NotI) to produce the appropriate fragment for such clone. Each clone was deposited in either the pED6 or pNOTs vector depicted in FIGS. 1A and 1B, respectively. The pED6dpc2 vector (“pED6”) was derived from pED6dpc1 by insertion of a new polylinker to facilitate cDNA cloning (Kaufman et al., 1991, Nudeic Acids Res. 19: 4485-4490); the pNOTs vector was derived from pMT2 (Kaufman et al., 1989, Mol. Cell. Biol. 9: 946-958) by deletion of the DHFR sequences, insertion of a new polylinker, and insertion of the M13 origin of replication in the ClaI site. In some instances, the deposited clone can become “flipped” (i.e., in the reverse orientation) in the deposited isolate. In such instances, the cDNA insert can still be isolated by digestion with EcoRI and NotI. However, NotI will then produce the 5′ site and EcoRI will produce the 3′ site for placement of the cDNA in proper orientation for expression in a suitable vector. The cDNA may also be expressed from the vectors in which they were deposited.

Bacterial cells containing a particular clone can be obtained from the composite deposit as follows:

An oligonucleotide probe or probes should be designed to the sequence that is known for that particular clone. This sequence can be derived from the sequences provided herein, or from a combination of those sequences. The sequence of an oligonucleotide probe that was used to isolate or to sequence each full-length clone is identified below, and should be most reliable in isolating the clone of interest.

| Clone | Probe Sequence |
|---|---|
| bh389_11 | SEQ ID NO:24 |
| bk112_15 | SEQ ID NO:25 |
| bk200_13 | SEQ ID NO:26 |
| di386_3 | SEQ ID NO:27 |
| em397_2 | SEQ ID NO:28 |
| fh170_7 | SEQ ID NO:29 |
| fn53_4 | SEQ ID NO:30 |
| fq505_4 | SEQ ID NO:31 |
| fw13_9 | SEQ ID NO:32 |
| gg619_2 | SEQ ID NO:33 |
| cl181_3 | SEQ ID NO:55 |
| cr1044_1 | SEQ ID NO:56 |
| cz251_1 | SEQ ID NO:57 |
| dd12_7 | SEQ ID NO:58 |
| fn191_3 | SEQ ID NO:59 |
| gm196_4 | SEQ ID NO:60 |
| gn114_1 | SEQ ID NO:61 |
| hj968_2 | SEQ ID NO:62 |
| hk10_3 | SEQ ID NO:63 |
| hm236_1 | SEQ ID NO:64 |
| do15_4 | SEQ ID NO:87 |
| dx290_1 | SEQ ID NO:88 |
| ek390_4 | SEQ ID NO:89 |
| er471_7 | SEQ ID NO:90 |
| fs40_3 | SEQ ID NO:91 |
| ga63_6 | SEQ ID NO:92 |
| gm335_4 | SEQ ID NO:93 |
| hy370_9 | SEQ ID NO:94 |
| ie47_4 | SEQ ID NO:95 |
| s195_10 | SEQ ID NO:96 |
| bf228_14 | SEQ ID NO:119 |
| bg249_1 | SEQ ID NO:120 |
| bv286_1 | SEQ ID NO:121 |
| co36_1 | SEQ ID NO:122 |
| cp116_1 | SEQ ID NO:123 |
| cw1195_2 | SEQ ID NO:124 |
| fh13_10 | SEQ ID NO:125 |
| gc57_4 | SEQ ID NO:126 |
| h1165_3 | SEQ ID NO:127 |
| hb752_1 | SEQ ID NO:128 |
| bi127_5 | SEQ ID NO:149 |
| bl194_2 | SEQ ID NO:150 |
| ccl30_1 | SEQ ID NO:151 |
| ch582_1 | SEQ ID NO:152 |
| cq294_14 | SEQ ID NO:153 |
| dd454_1 | SEQ ID NO:154 |
| du157_12 | SEQ ID NO:155 |
| du372_1 | SEQ ID NO:156 |
| ej90_5 | SEQ ID NO:157 |
| ic2_6 | SEQ ID NO:158 |
| bn97_1 | SEQ ID NO:175 |
| bn268_11 | SEQ ID NO:176 |
| cb96_10 | SEQ ID NO:177 |
| cb213_11 | SEQ ID NO:178 |
| cj457_4 | SEQ ID NO:179 |
| cz653_11 | SEQ ID NO:180 |
| dx138_4 | SEQ ID NO:181 |
| ij167_5 | SEQ ID NO:182 |
| bd107_16 | SEQ ID NO:198 |
| bm41_7 | SEQ ID NO:199 |
| br342_11 | SEQ ID NO:200 |
| ej258_11 | SEQ ID NO:201 |
| k232_2x | SEQ ID NO:202 |
| lf307_5 | SEQ ID NO:203 |
| lr204_1 | SEQ ID NO:204 |
| as20_2 | SEQ ID NO:223 |
| bf227_8 | SEQ ID NO:224 |
| bh157_7 | SEQ ID NO:225 |
| cg426_8 | SEQ ID NO:226 |
| ck48_12 | SEQ ID NO:227 |
| co1000_1 | SEQ ID NO:228 |
| ct489_14 | SEQ ID NO:229 |
| df821_1 | SEQ ID NO:230 |
| dy41_2 | SEQ ID NO:231 |

In the sequences listed above which include an N at position 2, that position is occupied in preferred probes/primers by a biotinylated phosphoaramidite residue rather than a nucleotide (such as, for example, that produced by use of biotin phosphoramidite (1-dimethoxytrityloxy-2-(N-biotinyl-4-aminobutyl)-propyl-3-O-(2-cyanoethyl)-(N,N-diisopropyl)-phosphoramadite) (Glen Research, cat. no. 10-1953)).

The design of the oligonucleotide probe should preferably follow these parameters:

(a) It should be designed to an area of the sequence which has the fewest ambiguous bases (“N's”), if any;

(b) It should be designed to have a Tm of approx. 80° C. (assuming 2° for each A or T and 4 degrees for each G or C).

The oligonucleotide should preferably be labeled with γ-³²P ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4e+6 dpm/pmole.

The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65° C. for 1 hour with gentle agitation in 6× SSC (20× stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). Preferably, the probe is then added to the hybridization mix at a concentration greater than or equal to 1e+6 dpm/mL. The filter is then preferably incubated at 65° C. with gentle agitation overnight. The filter is then preferably washed in 500 mL of 2× SSC/0.5% SDS at room temperature without agitation, preferably followed by 500 mL of 2× SSC/0.1% SDS at room temperature with gentle shaking for 15 minutes. A third wash with 0.1× SSC/0.5% SDS at 65° C. for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10, 773-778 (1992) and in R. S. McDowell, et al., J. Amer. Chem. Soc. 114 9245-9253 (1992), both of which are incorporated herein by reference. Such fragments may be fused to carrier molecules such as immunoglobulins for many purposes, including increasing the valency of protein binding sites. For example, fragments of the protein may be fused through “linker” sequences to the Fc portion of an immunoglobulin. For a bivalent form of the protein, such a fusion could be to the Fc portion of an IgG molecule. Other immunoglobulin isotypes may also be used to generate such fusions. For example, a protein-IgM fusion would generate a decavalent form of the protein of the invention.

The present invention also provides both full-length and mature forms of the disclosed proteins. The full-length form of the such proteins is identified in the sequence listing by translation of the nucleotide sequence of each disclosed clone. The mature form(s) of such protein may be obtained by expression of the disclosed full-length polynucleotide (preferably those deposited with ATCC) in a suitable mammalian cell or other host cell. The sequence(s) of the mature form(s) of the protein may also be determinable from the amino acid sequence of the full-length form.

The present invention also provides genes corresponding to the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1, incorporated by reference herein). In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614, 396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Where the protein of the present invention is membrane-bound (e.g., is a receptor), the present invention also provides for soluble forms of such protein. In such forms part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information.

Proteins and protein fragments of the present invention include proteins with amino acid sequence lengths that are at least 25%(more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous amino acids that shares at least 75% sequence identity (more preferably, at least 85% identity; most preferably at least 95% identity) with any such segment of any of the disclosed proteins.

Species homologues of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide. Preferably, polynucleotide species homologues have at least 60% sequence identity (more preferably, at least 75% identity; most preferably at least 90% identity) with the given polynucleotide, and protein species homologues have at least 30% sequence identity (more preferably, at least 45% identity; most preferably at least 60% identity) with the given protein, where sequence identity is determined by comparing the nucleotide sequences of the polynucleotides or the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from mammalian species. Most preferably, species homologues are those isolated from certain mammalian species such as, for example, Pan troglodytes, Gorilla gorilla, Pongo pygmaeus, Hylobates concolor, Macaca mulatta, Papio papio, Papio hamadryas, Cercopithecus aethiops, Cebus capucinus, Aotus trivirgatus, Sanguinus oedipus, Microcebus murinus, Mus musculus, Rattus norvegicus, Cricetulus griseus, Felis catus, Mustela vison, Canis familiaris, Oryctolagus cuniculus, Bos taurus, Ovis aries, Sus scrofa, and Equus caballus, for which genetic maps have been created allowing the identification of syntenic relationships between the genomic organization of genes in one species and the genomic organization of the related genes in another species (O'Brien and Seuánez, 1988, Ann. Rev. Genet. 22: 323-351; O'Brien et al., 1993, Nature Genetics 3:103-112; Johansson et al., 1995, Genomics 25: 682-690; Lyons et al., 1997, Nature Genetics 15: 47-56; O'Brien et al., 1997, Trends in Genetics 13(10): 393-399; Carver and Stubbs, 1997, Genome Research 7:1123-1137; all of which are incorporated by reference herein).

The invention also encompasses allelic variants of the disclosed polynucleotides or proteins; that is, naturally-occurring alternative forms of the isolated polynucleotides which also encode proteins which are identical or have significantly similar sequences to those encoded by the disclosed polynucleotides. Preferably, allelic variants have at least 60% sequence identity (more preferably, at least 75% identity; most preferably at least 90% identity) with the given polynucleotide, where sequence identity is determined by comparing the nucleotide sequences of the polynucleotides when aligned so as to maximize overlap and identity while minimizing sequence gaps. Allelic variants may be isolated and identified by making suitable probes or primers from the sequences provided herein and screening a suitable nucleic acid source from individuals of the appropriate species.

The invention also includes polynucleotides with sequences complementary to those of the polynucleotides disclosed herein.

The present invention also includes polynucleotides that hybridize under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

| | | Hybrid | | Wash |
|---|---|---|---|---|
| Stringency | Polynucleotide | Length | Hybridization Temperature and | Temperature |
| Condition | Hybrid | (bp)^{‡} | Buffer^{†} | and Buffer^{†} |
| A | DNA:DNA | ≧50 | 65° C.; 1 × SSC -or- | 65° C.; 0.3 × SSC |
| | | | 42° C.; 1 × SSC, 50% formamide | |
| B | DNA:DNA | <50 | T_{B}*; 1 × SSC | T_{B}*; 1 × SSC |
| C | DNA:RNA | ≦50 | 67° C.; 1 × SSC -or- | 67° C.; 0.3 × SSC |
| | | | 45° C.; 1 × SSC, 50% formamide | |
| D | DNA:RNA | <50 | T_{D}*; 1 × SSC | T_{D}*; 1 × SSC |
| E | RNA:RNA | ≧50 | 70° C.; 1 × SSC -or- | 70° C.; 0.3 × SSC |
| | | | 50° C.; 1 × SSC, 50% formamide | |
| F | RNA:RNA | <50 | T_{F}*; 1 × SSC | T_{F}*; 1 × SSC |
| G | DNA:DNA | ≧50 | 65° C.; 4 × SSC -or- | 65° C.; 1 × SSC |
| | | | 42° C.; 4 × SSC, 50% formamide | |
| H | DNA:DNA | <50 | T_{H}*; 4 × SSC | T_{H}*; 4 × SSC |
| I | DNA:RNA | ≧50 | 67° C.; 4 × SSC -or- | 67° C.; 1 × SSC |
| | | | 45° C.; 4 × SSC, 50% formamide | |
| J | DNA:RNA | <50 | T_{J}*; 4 × SSC | T_{J}*; 4 × SSC |
| K | RNA:RNA | ≧50 | 70° C.; 4 × SSC -or- | 67° C.; 1 × SSC |
| | | | 50° C.; 4 × SSC, 50% formamide | |
| L | RNA:RNA | <50 | T_{L}*; 2 × SSC | T_{L}*; 2 × SSC |
| M | DNA:DNA | ≧50 | 50° C.; 4 × SSC -or- | 50° C.; 2 × SSC |
| | | | 40° C.; 6 × SSC, 50% formamide | |
| N | DNA:DNA | <50 | T_{N}*; 6 × SSC | T_{N}*; 6 × SSC |
| O | DNA:RNA | ≧50 | 55° C.; 4 × SSC -or- | 55° C.; 2 × SSC |
| | | | 42° C.; 6 × SSC, 50% formamide | |
| P | DNA:RNA | <50 | T_{P}*; 6 × SSC | T_{P}*; 6 × SSC |
| Q | RNA:RNA | ≧50 | 60° C.; 4 × SSC -or- | 60° C.; 2 × SSC |
| | | | 45° C.; 6 × SSC, 50% formamide | |
| R | RNA:RNA | <50 | T_{R}*; 4 × SSC | T_{R}*; 4 × SSC |
| ^{‡}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{†}: SSPE (1 × SSPE is 0.15M NaCl, 10 mM NaH₂PO₄, and 1.25 mM EDTA, pH 7.4) can be substituted for SSC (1 × SSC is 0.15M NaCl and 15 mM sodium citrate) in the hybridization and wash buffers; washes are performed for 15 minutes after hybridization is complete. | | | | |
| *T_{B}-T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base | | | | |
| # pairs in length, Tₘ(° C.) = 81.5 + 16.6(log₁₀[Na⁺]) + 0.41(% G + C) − (600/N), where N is the number of bases in the hybrid, and [Na⁺] is the concentration of sodium ions in the hybridization buffer ([Na⁺] for 1 × SSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

The isolated polynucleotide of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.” The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLab (Beverly, Mass.), Pharmacia (Piscataway, N.J.) and InVitrogen, respectively. The protein can also be tagged with an epitope and subsequently purified by using a specific antibody directed to such epitope. One such epitope (“Flag”) is commercially available from Kodak (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The protein of the invention may also be expressed as a product of transgenic animals, e.g., as a component of the milk of transgenic cows, goats, pigs, or sheep which are characterized by somatic or germ cells containing a nucleotide sequence encoding the protein.

The protein may also be produced by known conventional chemical synthesis. Methods for constructing the proteins of the present invention by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

Uses and Biological Activity

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, those described in Gyuris et al., 1993, Cell 75: 791-803 and in Rossi et al., 1997, Proc. Natl. Acad. Sci. USA 94: 8405-8410, all of which are incorporated by reference herein) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J.E.e.a. Coligan eds. Vol 1 pp. 3.12.1-3.12.14, John Wiley and Sons, Toronto. 1994; and Measurement of mouse and human Interferon y, Schreiber, R. D. In Current Protocols in Immunology. J.E.e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J.E.e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6- Nordan, R. In Current Protocols in Immunology. J.E.e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11- Bennett, F., Giannotti, J., Clark, S.C. and Turner, K. J. In Current Protocols in Immunology. J.E.e.a. Coligan eds. Vol 1 pp. 6.15.1 John Wiley and Sons, Toronto. 1991; Measurement of mouse and human Interleukin 9- Ciarletta, A., Giannotti, J., Clark, S. C. and Turner, K. J. In Current Protocols in Immunology. J.E.e.a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β2 microglobulin protein or an MHC class II α chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowmanet al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J.E.e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad Sci. USA 88:7548-7551, 1991.

Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pps. 71-112 (Maibach, H I and Rovee, D T, eds.), Year Book Medical Publishers, Inc., Chicago, as modified by Eaglstein and Mertz, J. Invest. Dermatol 71:382-84 (1978).

Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin α family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-β group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selectins, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in:Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Cadherin/Tumor Invasion Suppressor Activity

Cadherins are calcium-dependent adhesion molecules that appear to play major roles during development, particularly in defining specific cell types. Loss or alteration of normal cadherin expression can lead to changes in cell adhesion properties linked to tumor growth and metastasis. Cadherin malfunction is also implicated in other human diseases, such as pemphigus vulgaris and pemphigus foliaceus (auto-immune blistering skin diseases), Crohn's disease, and some developmental abnormalities.

The cadherin superfamily includes well over forty members, each with a distinct pattern of expression. All members of the superfamily have in common conserved extracellular repeats (cadherin domains), but structural differences are found in other parts of the molecule. The cadherin domains bind calcium to form their tertiary structure and thus calcium is required to mediate their adhesion. Only a few amino acids in the first cadherin domain provide the basis for homophilic adhesion; modification of this recognition site can change the specificity of a cadherin so that instead of recognizing only itself, the mutant molecule can now also bind to a different cadherin. In addition, some cadherins engage in heterophilic adhesion with other cadherins.

E-cadherin, one member of the cadherin superfamily, is expressed in epithelial cell types. Pathologically, if E-cadherin expression is lost in a tumor, the malignant cells become invasive and the cancer metastasizes. Transfection of cancer cell lines with polynucleotides expressing E-cadherin has reversed cancer-associated changes by returning altered cell shapes to normal, restoring cells' adhesiveness to each other and to their substrate, decreasing the cell growth rate, and drastically reducing anchorage-independent cell growth. Thus, reintroducing E-cadherin expression reverts carcinomas to a less advanced stage. It is likely that other cadherins have the same invasion suppressor role in carcinomas derived from other tissue types. Therefore, proteins of the present invention with cadherin activity, and polynucleotides of the present invention encoding such proteins, can be used to treat cancer. Introducing such proteins or polynucleotides into cancer cells can reduce or eliminate the cancerous changes observed in these cells by providing normal cadherin expression.

Cancer cells have also been shown to express cadherins of a different tissue type than their origin, thus allowing these cells to invade and metastasize in a different tissue in the body. Proteins of the present invention with cadherin activity, and polynucleotides of the present invention encoding such proteins, can be substituted in these cells for the inappropriately expressed cadherins, restoring normal cell adhesive properties and reducing or eliminating the tendency of the cells to metastasize.

Additionally, proteins of the present invention with cadherin activity, and polynucleotides of the present invention encoding such proteins, can used to generate antibodies recognizing and binding to cadherins. Such antibodies can be used to block the adhesion of inappropriately expressed tumor-cell cadherins, preventing the cells from forming a tumor elsewhere. Such an anti-cadherin antibody can also be used as a marker for the grade, pathological type, and prognosis of a cancer, i.e. the more progressed the cancer, the less cadherin expression there will be, and this decrease in cadherin expression can be detected by the use of a cadherin-binding antibody.

Fragments of proteins of the present invention with cadherin activity, preferably a polypeptide comprising a decapeptide of the cadherin recognition site, and poly-nucleotides of the present invention encoding such protein fragments, can also be used to block cadherin function by binding to cadherins and preventing them from binding in ways that produce undesirable effects. Additionally, fragments of proteins of the present invention with cadherin activity, preferably truncated soluble cadherin fragments which have been found to be stable in the circulation of cancer patients, and polynucleotides encoding such protein fragments, can be used to disturb proper cell-cell adhesion.

Assays for cadherin adhesive and invasive suppressor activity include, without limitation, those described in: Hortsch et al. J Biol Chem 270 (32): 18809-18817, 1995; Miyaki et al. Cncogene 11: 2547-2552, 1995; Ozawa et al. Cell 63: 1033-1038, 1990.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Administration and Dosing

A protein of the present invention (from whatever source derived, including without limitation from recombinant and non-recombinant sources) may be used in a pharmaceutical composition when combined with a pharmaceutically acceptable carrier. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNF1, TNF1, TNF2, G-CSF, Meg-CSF, thrombopoietin, stem cell factor, and erythropoietin. The pharmaceutical composition may further contain other agents which either enhance the activity of the protein or compliment its activity or use in treatment. Such additional factors and/or agents may be included in the pharmaceutical composition to produce a synergistic effect with protein of the invention, or to minimize side effects. Conversely, protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent.

A protein of the present invention may be active in multimers (e.g., heterodimers or homodimers) or complexes with itself or other proteins. As a result, pharmaceutical compositions of the invention may comprise a protein of the invention in such multimeric or complexed form.

The pharmaceutical composition of the invention may be in the form of a complex of the protein(s) of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunolgobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of protein of the present invention is administered to a mammal having a condition to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with cytokine(s), lymphokine(s), other hematopoietic factor(s), thrombolytic or anti-thrombotic factors.

Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 ng to about 10 mg, more preferably about 0.1 μg to about 1 mg) of protein of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Protein of the invention may also be used to immunize animals to obtain polyclonal and monoclonal antibodies which specifically react with the protein. Such antibodies may be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides are known in the art, for example, as in R. P. Merrifield, J. Amer. Chem. Soc. 85, 2149-2154 (1963); J. L. Krstenansky, et al., FEBS Lett. 211, 10 (1987). Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropylmethylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the protein from the polymer matrix and to provide appropriate handling of the composition, yet not so much that the progenitor cells are prevented from infiltrating the matrix, thereby providing the protein the opportunity to assist the osteogenic activity of the progenitor cells.

In further compositions, proteins of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals and thoroughbred horses, in addition to humans, are desired patients for such treatment with proteins of the present invention.

The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA).

Cells may also be cultured ex vivo in the presence of proteins of the present invention in order to proliferate or to produce a desired effect on or activity in such cells. Treated cells can then be introduced in vivo for therapeutic purposes.

Patent and literature references cited herein are incorporated by reference as if fully set forth.

### Appendix

Clones bd107_{—}16, bm41_{—}7, br342_{—}11, ej258_{—}11, k232_{—}2x, lf307_{—}5 and lr204_{—}1 have been deposited with the American Type Culture Collection in this composite deposit, from which each clone comprising a particular polynucleotide is obtainable. Each clone has been transfected into separate bacterial cells (E. coli) in this composite deposit. The deposited cDNA can be removed from its vector by digestion with restriction enzymes (5′ site, EcoRI; 3′ site NotI). Bacterial cells containing a particular clone can be obtained from the composite deposit as follows:

An oligonucleotide probe should be designed to the sequence that is known for that particular clone. This sequence can be derived from the sequences provided in the attached Sequence Listing, or preferably, from a composite of those sequences. The sequence of an oligonucleotide probe that was used to isolate or to sequence each full-length clone is identified below, and should be most reliable in isolating the clone of interest.

| Clone | Probe Sequence¹ |
|---|---|
| bd107_16 | SEQ ID NO:198 |
| bm41_7 | SEQ ID NO:199 |
| br342_11 | SEQ ID NO:200 |
| ej258_11 | SEQ ID NO:201 |
| k232_2x | SEQ ID NO:202 |
| lf307_5 | SEQ ID NO:203 |
| lr204_1 | SEQ ID NO:204 |

¹References are to sequences in the Sequence Listing attached hereto.

The following SEQ ID NO:s in the attached Sequence Listing correspond to the indicated clone: bd107_{—}16: SEQ ID NO:183 ; bm41_{—}7: SEQ ID NO:185 ; br342_{—}11: SEQ ID NO:187 ; ej258_{—}11: SEQ ID NO:189 ; k232_{—}2x: SEQ ID NO:191 ; lf307_{—}5: SEQ ID NO:193, SEQ ID NO:195; lr204_{—}1: SEQ ID NO:196;

In the sequences listed above which include an N at position 2, that position is occupied in preferred probes/primers by a biotinylated phosphoaramidite residue rather than a nucleotide (such as, for example, that produced by use of biotin phosphoramidite (1-dimethoxytrityloxy-2-(N-biotinyl-4-aminobutyl)-propyl-3-O-(2-cyanoethyl)-(N,N-diisopropyl)-phosphoramadite) (Glen Research, cat. no. 10-1953)).

The design of the oligonucleotide probe should preferably follow these parameters:

(a) It should be designed to an area of the sequence which has the fewest ambiguous bases (“N's”), if any;

(b) It should be designed to have a Tm of approx. 80° C. (assuming 2° for each A or T and 4 degrees for each G or C).

The oligonucleotide should preferably be labelled with g-³²P ATP (specific activity 6000 Ci/mmole) and T4 polynucleotide kinase using commonly employed techniques for labelling oligonucleotides. Other labelling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4e+6 dpm/pmole.

The bacterial culture containing the pool of full-length clones should preferably be thawed and 100 μl of the stock used to inoculate a sterile culture flask containing 25 μml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65° C. for 1 hour with gentle agitation in 6× SSC (20× stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). Preferably, the probe is then added to the hybridization mix at a concentration greater than or equal to 1e+6 dpm/mL. The filter is then preferably incubated at 65° C. with gentle agitation overnight. The filter is then preferably washed in 500 mL of 2× SSC/0.5% SDS at room temperature without agitation, preferably followed by 500 mL of 2× SSC/0.1% SDS at room temperature with gentle shaking for 15 minutes. A third wash with 0.1× SSC/0.5% SDS at 65° C. for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing.

Once a particular clone has been isolated from the composite deposit, the cDNA can be removed from the vector in which it was deposited by performing an EcoRI/NotI digestion (5′ site, EcoRI; 3′ site, NotI). The resultant EcoRI/NotI fragments should have the following approximate sizes:

| Clone | Size |
|---|---|
| bd107_16 | 1500 bp |
| bm41_7 | 1700 bp |
| br342_11 | 1400 bp |
| ej258_11 | 670 bp |
| k232_2x | 555 bp |
| lf307_5 | 1000 bp |
| lr204_1 | 900 bp |

Clone Origin

All clones in the deposit are of human origin.

What is claimed is:

1. An isolated polynucleotide selected from the group consisting of:
(a) a polynucleotide comprising the nucleotide sequence of SEQ ID NO:1;
(b) a polynucleotide comprising the nucleotide sequence of SEQ ID NO:1 from nucleotide 12 to nucleotide 800;
(c) a polynucleotide comprising the nucleotide sequence of SEQ ID NO:1 from nucleotide 78 to nucleotide 800;
(d) a polynucleotide comprising the nucleotide sequence of SEQ ID NO:1 from nucleotide 1 to nucleotide 547;
(e) a polynucleotide comprising the nucleotide sequence of the full-length protein coding sequence of clone bh389_{—}11 deposited under accession number ATCC 98451;
(f) a polynucleotide encoding the full-length protein encoded by the cDNA insert of clone bh389_{—}11 deposited under accession number ATCC 98451;
(g) a polynucleotide comprising the nucleotide sequence of a mature protein coding sequence of clone bh389_{—}11 deposited under accession number ATCC 98451;
(h) a polynucleotide encoding a mature protein encoded by the cDNA insert of clone bh389_{—}11 deposited under accession number ATCC 98451;
(i) a polynucleotide encoding a protein comprising the amino acid sequence of SEQ ID NO:2;
(j) a polynucleotide encoding a protein comprising a fragment of the amino acid sequence of SEQ ID NO:2 having biological activity, the fragment comprising eight consecutive amino acids of SEQ ID NO:2;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

2. The polynucleotide of claim 1 wherein said polynucleotide is operably linked to at least one expression control sequence.

3. A host cell transformed with the polynucleotide of claim 2.

4. The host cell of claim 3, wherein said cell is a mammalian cell.

5. A process for producing a protein encoded by the polynucleotide of claim 2, which process comprises:
(a) growing a culture of the host cell of claim 3 in a suitable culture medium; and
(b) purifying said protein from the culture.

6. A protein produced according to the process of claim 5.

7. A protein comprising an amino acid sequence selected from the group consisting of:
(a) the amino acid sequence of SEQ ID NO:2;
(b) the amino acid sequence of SEQ ID NO:2 from amino acid 1 to amino acid 178;
(c) fragments of the amino acid sequence of SEQ ID NO:2 comprising eight consecutive amino acids of SEQ ID NO:2; and
(d) the amino acid sequence encoded by the cDNA insert of clone bh389_{—}11 deposited under accession number ATCC 98451;
the protein being substantially free from other mammalian proteins.

8. The protein of claim 7, wherein said protein comprises the amino acid sequence of SEQ ID NO:2.

9. The protein of claim 7, wherein said protein comprises the amino acid sequence of SEQ ID NO:2 from amino acid 1 to amino acid 178.

10. A composition comprising the protein of claim 7 and a pharmaceutically acceptable carrier.

11. An isolated gene corresponding to the cDNA sequence of SEQ ID NO:1.

12. An isolated polynucleotide selected from the group consisting of:
(a) a polynucleotide comprising the nucleotide sequence of SEQ ID NO:3;
(b) a polynucleotide comprising the nucleotide sequence of SEQ ID NO:3 from nucleotide 100 to nucleotide 882;
(c) a polynucleotide comprising the nucleotide sequence of SEQ ID NO:3 from nucleotide 635 to nucleotide 867;
(d) a polynucleotide comprising the nucleotide sequence of the full-length protein coding sequence of clone bk112_{—}15 deposited under accession number ATCC 9845;
(e) a polynucleotide encoding the full-length protein encoded by the cDNA insert of clone bk112_{—}15 deposited under accession number ATCC 98451;
(f) a polynucleotide comprising the nucleotide sequence of a mature protein coding sequence of clone bk112_{—}15 deposited under accession number ATCC 98451;
(g) a polynucleotide encoding a mature protein encoded by the cDNA insert of clone bk112_{—}15 deposited under accession number ATCC 98451;
(h) a polynucleotide encoding a protein comprising the amino acid sequence of SEQ ID NO:4;
(i) a polynucleotide encoding a protein comprising a fragment of the amino acid sequence of SEQ ID NO:4 having biological activity, the fragment comprising eight consecutive amino acids of SEQ ID NO:4;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

13. A protein comprising an amino acid sequence selected from the group consisting of:
(a) the amino acid sequence of SEQ ID NO:4;
(b) the amino acid sequence of SEQ ID NO:4 from amino acid 200 to amino acid 256;
(c) fragments of the amino acid sequence of SEQ ID NO:4 comprising eight consecutive amino acids of SEQ ID NO:4; and
(d) the amino acid sequence encoded by the cDNA insert of clone bk112_{—}15 deposited under accession number ATCC 98451;
the protein being substantially free from other mammalian proteins.

14. An isolated gene corresponding to the cDNA sequence of SEQ ID NO:3.

15. An isolated polynucleotide selected from the group consisting of:
(a) a polynucleotide comprising the nucleotide sequence of SEQ ID NO:5;
(b) a polynucleotide comprising the nucleotide sequence of SEQ ID NO:5 from nucleotide 245 to nucleotide 520;
(c) a polynucleotide comprising the nucleotide sequence of SEQ ID NO:5 from nucleotide 181 to nucleotide 527;
(d) a polynucleotide comprising the nucleotide sequence of the full-length protein coding sequence of clone bk200_{—}13 deposited under accession number ATCC 98451;
(e) a polynucleotide encoding the full-length protein encoded by the cDNA insert of clone bk200_{—}13 deposited under accession number ATCC 98451;
(f) a polynucleotide comprising the nucleotide sequence of a mature protein coding sequence of clone bk200_{—}13 deposited under accession number ATCC 98451;
(g) a polynucleotide encoding a mature protein encoded by the cDNA insert of clone bk200_{—}13 deposited under accession number ATCC 98451;
(h) a polynucleotide encoding a protein comprising the amino acid sequence of SEQ ID NO:6;
(i) a polynucleotide encoding a protein comprising a fragment of the amino acid sequence of SEQ ID NO:6 having biological activity, the fragment comprising eight consecutive amino acids of SEQ ID NO:6;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

16. A protein comprising an amino acid sequence selected from the group consisting of:
(a) the amino acid sequence of SEQ ID NO:6;
(b) fragments of the amino acid sequence of SEQ ID NO:6 comprising eight consecutive amino acids of SEQ ID NO:6; and
(c) the amino acid sequence encoded by the cDNA insert of clone bk200_{—}13 deposited under accession number ATCC 98451;
the protein being substantially free from other mammalian proteins.

17. An isolated gene corresponding to the cDNA sequence of SEQ ID NO:5.

18. An isolated polynucleotide selected from the group consisting of:
(a) a polynucleotide comprising the nucleotide sequence of SEQ ID NO:7;
(b) a polynucleotide comprising the nucleotide sequence of SEQ ID NO:7 from nucleotide 365 to nucleotide 784;
(c) a polynucleotide comprising the nucleotide sequence of SEQ ID NO:7 from nucleotide 518 to nucleotide 784;
(d) a polynucleotide comprising the nucleotide sequence of the full-length protein coding sequence of clone di386_{—}3 deposited under accession number ATCC 98451;
(e) a polynucleotide encoding the full-length protein encoded by the cDNA insert of clone di386_{—}3 deposited under accession number ATCC 98451;
(f) a polynucleotide comprising the nucleotide sequence of a mature protein coding sequence of clone di386_{—}3 deposited under accession number ATCC 98451;
(g) a polynucleotide encoding a mature protein encoded by the cDNA insert of clone di386_{—}3 deposited under accession number ATCC 98451;
(h) a polynucleotide encoding a protein comprising the amino acid sequence of SEQ ID NO:8;
(i) a polynucleotide encoding a protein comprising a fragment of the amino acid sequence of SEQ ID NO:8 having biological activity, the fragment comprising eight consecutive amino acids of SEQ ID NO:8;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

19. A protein comprising an amino acid sequence selected from the group consisting of:
(a) the amino acid sequence of SEQ ID NO:8;
(b) the amino acid sequence of SEQ ID NO:8 from amino acid 1 to amino acid 140;
(c) fragments of the amino acid sequence of SEQ ID NO:8 comprising eight consecutive amino acids of SEQ ID NO:8; and
(d) the amino acid sequence encoded by the cDNA insert of clone di386_{—}3 deposited under accession number ATCC 98451;
the protein being substantially free from other mammalian proteins.

20. An isolated gene corresponding to the cDNA sequence of SEQ ID NO:7 and SEQ ID NO:9.

21. An isolated polynucleotide selected from the group consisting of:
(a) a polynucleotide comprising the nucleotide sequence of SEQ ID NO:10;
(b) a polynucleotide comprising the nucleotide sequence of SEQ ID NO:10 from nucleotide 191 to nucleotide 781;
(c) a polynucleotide comprising the nucleotide sequence of SEQ ID NO:10 from nucleotide 56 to nucleotide 492;
(d) a polynucleotide comprising the nucleotide sequence of the full-length protein coding sequence of clone em397_{—}2 deposited under accession number ATCC 98451;
(e) a polynucleotide encoding the full-length protein encoded by the cDNA insert of clone em397_{—}2 deposited under accession number ATCC 98451;
(f) a polynucleotide comprising the nucleotide sequence of a mature protein coding sequence of clone em397_{—}2 deposited under accession number ATCC 98451;
(g) a polynucleotide encoding a mature protein encoded by the cDNA insert of clone em397_{—}2 deposited under accession number ATCC 98451;
(h) a polynucleotide encoding a protein comprising the amino acid sequence of SEQ ID NO:11;
(i) a polynucleotide encoding a protein comprising a fragment of the amino acid sequence of SEQ ID NO:11 having biological activity, the fragment comprising eight consecutive amino acids of SEQ ID NO:11;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

22. A protein comprising an amino acid sequence selected from the group consisting of:
(a) the amino acid sequence of SEQ ID NO:11;
(b) the amino acid sequence of SEQ ID NO:11 from amino acid 1 to amino acid 101;
(c) fragments of the amino acid sequence of SEQ ID NO:11 comprising eight consecutive amino acids of SEQ ID NO:11; and
(d) the amino acid sequence encoded by the cDNA insert of clone em397_{—}2 deposited under accession number ATCC 98451;
the protein being substantially free from other mammalian proteins.

23. An isolated gene corresponding to the cDNA sequence of SEQ ID NO:10.

24. An isolated polynucleotide selected from the group consisting of:
(a) a polynucleotide comprising the nucleotide sequence of SEQ ID NO:12;
(b) a polynucleotide comprising the nucleotide sequence of SEQ ID NO:12 from nucleotide 65 to nucleotide 1636;
(c) a polynucleotide comprising the nucleotide sequence of SEQ ID NO:12 from nucleotide 482 to nucleotide 1636;
(d) a polynucleotide comprising the nucleotide sequence of SEQ ID NO:12 from nucleotide 487 to nucleotide 1006;
(e) a polynucleotide comprising the nucleotide sequence of the full-length protein coding sequence of clone fh170_{—}7 deposited under accession number ATCC 98451;
(f) a polynucleotide encoding the full-length protein encoded by the cDNA insert of clone fh170_{—}7 deposited under accession number ATCC 98451;
(g) a polynucleotide comprising the nucleotide sequence of a mature protein coding sequence of clone fh170_{—}7 deposited under accession number ATCC 98451;
(h) a polynucleotide encoding a mature protein encoded by the cDNA insert of clone fh170_{—}7 deposited under accession number ATCC 98451;
(i) a polynucleotide encoding a protein comprising the amino acid sequence of SEQ ID NO:13;
(j) a polynucleotide encoding a protein comprising a fragment of the amino acid sequence of SEQ ID NO:13 having biological activity, the fragment comprising eight consecutive amino acids of SEQ ID NO:13;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

25. A protein comprising an amino acid sequence selected from the group consisting of:
(a) the amino acid sequence of SEQ ID NO:13;
(b) the amino acid sequence of SEQ ID NO:13 from amino acid 142 to amino acid 314;
(c) fragments of the amino acid sequence of SEQ ID NO:13 comprising eight consecutive amino acids of SEQ ID NO:13; and
(d) the amino acid sequence encoded by the cDNA insert of clone fh170_{—}7 deposited under accession number ATCC 98451;
the protein being substantially free from other mammalian proteins.

26. An isolated gene corresponding to the cDNA sequence of SEQ ID NO:12.

27. An isolated polynucleotide selected from the group consisting of:
(a) a polynucleotide comprising the nucleotide sequence of SEQ ID NO:15;
(b) a polynucleotide comprising the nucleotide sequence of SEQ ID NO:15 from nucleotide 41 to nucleotide 550;
(c) a polynucleotide comprising the nucleotide sequence of the full-length protein coding sequence of clone fn53_{—}4 deposited under accession number ATCC 98451;
(d) a polynucleotide encoding the full-length protein encoded by the cDNA insert of clone fn53_{—}4 deposited under accession number ATCC 98451;
(e) a polynucleotide comprising the nucleotide sequence of a mature protein coding sequence of clone fn53_{—}4 deposited under accession number ATCC 98451;
(f) a polynucleotide encoding a mature protein encoded by the cDNA insert of clone fn53_{—}4 deposited under accession number ATCC 98451;
(g) a polynucleotide encoding a protein comprising the amino acid sequence of SEQ ID NO:16;
(h) a polynucleotide encoding a protein comprising a fragment of the amino acid sequence of SEQ ID NO:16 having biological activity, the fragment comprising eight consecutive amino acids of SEQ ID NO:16;
(i) a polynucleotide which is an allelic variant of a polynucleotide of (a)-(f) above;
(j) a polynucleotide which encodes a species homologue of the protein of (g) or (h) above; and
(k) a polynucleotide that hybridizes under stringent conditions to any one of the polynucleotides specified in (a)-(h).

28. A protein comprising an amino acid sequence selected from the group consisting of:
(a) the amino acid sequence of SEQ ID NO:16;
(b) the amino acid sequence of SEQ ID NO:16 from amino acid 40 to amino acid 170;
(c) fragments of the amino acid sequence of SEQ ID NO:16 comprising eight consecutive amino acids of SEQ ID NO:16; and
(d) the amino acid sequence encoded by the cDNA insert of clone fn53_{—}4 deposited under accession number ATCC 98451;
the protein being substantially free from other mammalian proteins.

29. An isolated gene corresponding to the cDNA sequence of SEQ ID NO:15, SEQ ID NO:14, and SEQ ID NO:17.

30. An isolated polynucleotide selected from the group consisting of:
(a) a polynucleotide comprising the nucleotide sequence of SEQ ID NO:18;
(b) a polynucleotide comprising the nucleotide sequence of SEQ ID NO:18 from nucleotide 84 to nucleotide 404;
(c) a polynucleotide comprising the nucleotide sequence of SEQ ID NO:18 from nucleotide 78 to nucleotide 493;
(d) a polynucleotide comprising the nucleotide sequence of the full-length protein coding sequence of clone fq505_{—}4 deposited under accession number ATCC 98451;
(e) a polynucleotide encoding the full-length protein encoded by the cDNA insert of clone fq505_{—}4 deposited under accession number ATCC 98451;
(f) a polynucleotide comprising the nucleotide sequence of a mature protein coding sequence of clone fq505_{—}4 deposited under accession number ATCC 98451;
(g) a polynucleotide encoding a mature protein encoded by the cDNA insert of clone fq505_{—}4 deposited under accession number ATCC 98451;
(h) a polynucleotide encoding a protein comprising the amino acid sequence of SEQ ID NO:19;
(i) a polynucleotide encoding a protein comprising a fragment of the amino acid sequence of SEQ ID NO:19 having biological activity, the fragment comprising eight consecutive amino acids of SEQ ID NO:19;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

31. A protein comprising an amino acid sequence selected from the group consisting of:
(a) the amino acid sequence of SEQ ID NO:19;
(b) the amino acid sequence of SEQ ID NO:19 from amino acid 23 to amino acid 107;
(c) fragments of the amino acid sequence of SEQ ID NO:19 comprising eight consecutive amino acids of SEQ ID NO:19; and
(d) the amino acid sequence encoded by the cDNA insert of clone fq505_{—}4 deposited under accession number ATCC 98451;
the protein being substantially free from other mammalian proteins.

32. An isolated gene corresponding to the cDNA sequence of SEQ ID NO:18.

33. An isolated polynucleotide selected from the group consisting of:
(a) a polynucleotide comprising the nucleotide sequence of SEQ ID NO:20;
(b) a polynucleotide comprising the nucleotide sequence of SEQ ID NO:20 from nucleotide 1439 to nucleotide 1744;
(c) a polynucleotide comprising the nucleotide sequence of SEQ ID NO:20 from nucleotide 1241 to nucleotide 1754;
(d) a polynucleotide comprising the nucleotide sequence of the full-length protein coding sequence of clone fw13_{—}9 deposited under accession number ATCC 98451;
(e) a polynucleotide encoding the full-length protein encoded by the cDNA insert of clone fw13_{—}9 deposited under accession number ATCC 98451;
(f) a polynucleotide comprising the nucleotide sequence of a mature protein coding sequence of clone fw13_{—}9 deposited under accession number ATCC 98451;
(g) a polynucleotide encoding a mature protein encoded by the cDNA insert of clone fw13_{—}9 deposited under accession number ATCC 98451;
(h) a polynucleotide encoding a protein comprising the amino acid sequence of SEQ ID NO:21;
(i) a polynucleotide encoding a protein comprising a fragment of the amino acid sequence of SEQ ID NO:21 having biological activity, the fragment comprising eight consecutive amino acids of SEQ ID NO:21;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

34. A protein comprising an amino acid sequence selected from the group consisting of:
(a) the amino acid sequence of SEQ ID NO:21;
(b) the amino acid sequence of SEQ ID NO:21 from amino acid 1 to amino acid 57;
(c) fragments of the amino acid sequence of SEQ ID NO:21 comprising eight consecutive amino acids of SEQ ID NO:21; and
(d) the amino acid sequence encoded by the cDNA insert of clone fw13_{—}9 deposited under accession number ATCC 98451;
the protein being substantially free from other mammalian proteins.

35. An isolated gene corresponding to the cDNA sequence of SEQ ID NO:20.

36. An isolated polynucleotide selected from the group consisting of:
(a) a polynucleotide comprising the nucleotide sequence of SEQ ID NO:22;
(b) a polynucleotide comprising the nucleotide sequence of SEQ ID NO:22 from nucleotide 47 to nucleotide 919;
(c) a polynucleotide comprising the nucleotide sequence of SEQ ID NO:22 from nucleotide 124 to nucleotide 452;
(d) a polynucleotide comprising the nucleotide sequence of the full-length protein coding sequence of clone gg619_{—}2 deposited under accession number ATCC 98451;
(e) a polynucleotide encoding the full-length protein encoded by the cDNA insert of clone gg619_{—}2 deposited under accession number ATCC 98451;
(f) a polynucleotide comprising the nucleotide sequence of a mature protein coding sequence of clone gg619_{—}2 deposited under accession number ATCC 98451;
(g) a polynucleotide encoding a mature protein encoded by the cDNA insert of clone gg619_{—}2 deposited under accession number ATCC 98451;
(h) a polynucleotide encoding a protein comprising the amino acid sequence of SEQ ID NO:23;
(i) a polynucleotide encoding a protein comprising a fragment of the amino acid sequence of SEQ ID NO:23 having biological activity, the fragment comprising eight consecutive amino acids of SEQ ID NO:23;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

37. A protein comprising an amino acid sequence selected from the group consisting of:
(a) the amino acid sequence of SEQ ID NO:23;
(b) the amino acid sequence of SEQ ID NO:23 from amino acid 27 to amino acid 135;
(c) fragments of the amino acid sequence of SEQ ID NO:23 comprising eight consecutive amino acids of SEQ ID NO:23; and
(d) the amino acid sequence encoded by the cDNA insert of clone gg619_{—}2 deposited under accession number ATCC 98451;
the protein being substantially free from other mammalian proteins.

38. An isolated gene corresponding to the cDNA sequence of SEQ ID NO:22.

39. An isolated polynucleotide selected from the group consisting of:
(a) a polynucleotide comprising the nucleotide sequence of SEQ ID NO:35;
(b) a polynucleotide comprising the nucleotide sequence of SEQ ID NO:35 from nucleotide 2178 to nucleotide 2513;
(c) a polynucleotide comprising the nucleotide sequence of SEQ ID NO:35 from nucleotide 2364 to nucleotide 2513;
(d) a polynucleotide comprising the nucleotide sequence of SEQ ID NO:35 from nucleotide 1980 to nucleotide 2311;
(e) a polynucleotide comprising the nucleotide sequence of the full-length protein coding sequence of clone cl181_{—}3 deposited under accession number ATCC 98456;
(f) a polynucleotide encoding the full-length protein encoded by the cDNA insert of clone cl181_{—}3 deposited under accession number ATCC 98456;
(g) a polynucleotide comprising the nucleotide sequence of a mature protein coding sequence of clone cl181_{—}3 deposited under accession number ATCC 98456;
(h) a polynucleotide encoding a mature protein encoded by the cDNA insert of clone cl181_{—}3 deposited under accession number ATCC 98456;
(i) a polynucleotide encoding a protein comprising the amino acid sequence of SEQ ID NO:36;
(j) a polynucleotide encoding a protein comprising a fragment of the amino acid sequence of SEQ ID NO:36 having biological activity, the fragment comprising eight consecutive amino acids of SEQ ID NO:36;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

40. The polynucleotide of claim 38 wherein said polynucleotide is operably linked to at least one expression control sequence.

41. A host cell transformed with the polynucleotide of claim 39.

42. The host cell of claim 41, wherein said cell is a mammalian cell.

43. A process for producing a protein encoded by the polynucleotide of claim 39, which process comprises:
(a) growing a culture of the host cell of claim 41 in a suitable culture medium; and
(b) purifying said protein from the culture.

44. A protein produced according to the process of claim 43.

45. A protein comprising an amino acid sequence selected from the group consisting of:
(a) the amino acid sequence of SEQ ID NO:47;
(b) the amino acid sequence of SEQ ID NO:47 from amino acid 1 to amino acid 67;
(c) fragments of the amino acid sequence of SEQ ID NO:47 comprising eight consecutive amino acids of SEQ ID NO:47; and
(d) the amino acid sequence encoded by the cDNA insert of clone cl181_{—}3 deposited under accession number ATCC 98456;
the protein being substantially free from other mammalian proteins.

46. The protein of claim 45, wherein said protein comprises the amino acid sequence of SEQ ID NO:47.

47. The protein of claim 45, wherein said protein comprises the amino acid sequence of SEQ ID NO:36 from amino acid 1 to amino acid 67.

48. A composition comprising the protein of claim 45 and a pharmaceutically acceptable carrier.

49. An isolated gene corresponding to the cDNA sequence of SEQ ID NO:35.

50. An isolated polynucleotide selected from the group consisting of:
(a) a polynucleotide comprising the nucleotide sequence of SEQ ID NO:37;
(b) a polynucleotide comprising the nucleotide sequence of SEQ ID NO:37 from nucleotide 207 to nucleotide 893;
(c) a polynucleotide comprising the nucleotide sequence of SEQ ID NO:37 from nucleotide 1 to nucleotide 527;
(d) a polynucleotide comprising the nucleotide sequence of the full-length protein coding sequence of clone cr1044_{—}1 deposited under accession number ATCC 98456;
(e) a polynucleotide encoding the full-length protein encoded by the cDNA insert of clone cr1044_{—}1 deposited under accession number ATCC 98456;
(f) a polynucleotide comprising the nucleotide sequence of a mature protein coding sequence of clone cr1044_{—}1 deposited under accession number ATCC 98456;
(g) a polynucleotide encoding a mature protein encoded by the cDNA insert of clone cr1044_{—}1 deposited under accession number ATCC 98456;
(h) a polynucleotide encoding a protein comprising the amino acid sequence of SEQ ID NO:38;
(i) a polynucleotide encoding a protein comprising a fragment of the amino acid sequence of SEQ ID NO:38 having biological activity, the fragment comprising eight consecutive amino acids of SEQ ID NO:38;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

51. A protein comprising an amino acid sequence selected from the group consisting of:
(a) the amino acid sequence of SEQ ID NO:38;
(b) the amino acid sequence of SEQ ID NO:38 from amino acid 1 to amino acid 107;
(c) fragments of the amino acid sequence of SEQ ID NO:38 comprising eight consecutive amino acids of SEQ ID NO:38; and
(d) the amino acid sequence encoded by the cDNA insert of clone cr1044_{—}1 deposited under accession number ATCC 98456;
the protein being substantially free from other mammalian proteins.

52. An isolated gene corresponding to the cDNA sequence of SEQ ID NO:38.

53. An isolated polynucleotide selected from the group consisting of:
(a) a polynucleotide comprising the nucleotide sequence of SEQ ID NO:39;
(b) a polynucleotide comprising the nucleotide sequence of SEQ ID NO:39 from nucleotide 77 to nucleotide 400;
(c) a polynucleotide comprising the nucleotide sequence of SEQ ID NO:39 from nucleotide 118 to nucleotide 392;
(d) a polynucleotide comprising the nucleotide sequence of the full-length protein coding sequence of clone cz251_{—}1 deposited under accession number ATCC 98456;
(e) a polynucleotide encoding the full-length protein encoded by the cDNA insert of clone cz251_{—}1 deposited under accession number ATCC 98456;
(f) a polynucleotide comprising the nucleotide sequence of a mature protein coding sequence of clone cz251_{—}1 deposited under accession number ATCC 98456;
(g) a polynucleotide encoding a mature protein encoded by the cDNA insert of clone cz251_{—}1 deposited under accession number ATCC 98456;
(h) a polynucleotide encoding a protein comprising the amino acid sequence of SEQ ID NO:40;
(i) a polynucleotide encoding a protein comprising a fragment of the amino acid sequence of SEQ ID NO:40 having biological activity, the fragment comprising eight consecutive amino acids of SEQ ID NO:40;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

54. A protein comprising an amino acid sequence selected from the group consisting of:
(a) the amino acid sequence of SEQ ID NO:40;
(b) the amino acid sequence of SEQ ID NO:40 from amino acid 15 to amino acid 105;
(c) fragments of the amino acid sequence of SEQ ID NO:40 comprising eight consecutive amino acids of SEQ ID NO:40; and
(d) the amino acid sequence encoded by the cDNA insert of clone cz251_{—}1 deposited under accession number ATCC 98456;
the protein being substantially free from other mammalian proteins.

55. An isolated gene corresponding to the cDNA sequence of SEQ ID NO:39.

56. An isolated polynucleotide selected from the group consisting of:
(a) a polynucleotide comprising the nucleotide sequence of SEQ ID NO:41;
(b) a polynucleotide comprising the nucleotide sequence of SEQ ID NO:41 from nucleotide 13 to nucleotide 501;
(c) a polynucleotide comprising the nucleotide sequence of SEQ ID NO:41 from nucleotide 1 to nucleotide 506;
(d) a polynucleotide comprising the nucleotide sequence of the full-length protein coding sequence of clone dd12_{—}7 deposited under accession number ATCC 98456;
(e) a polynucleotide encoding the full-length protein encoded by the cDNA insert of clone dd12_{—}7 deposited under accession number ATCC 98456;
(f) a polynucleotide comprising the nucleotide sequence of a mature protein coding sequence of clone dd12_{—}7 deposited under accession number ATCC 98456;
(g) a polynucleotide encoding a mature protein encoded by the cDNA insert of clone dd12_{—}7 deposited under accession number ATCC 98456;
(h) a polynucleotide encoding a protein comprising the amino acid sequence of SEQ ID NO:42;
(i) a polynucleotide encoding a protein comprising a fragment of the amino acid sequence of SEQ ID NO:42 having biological activity, the fragment comprising eight consecutive amino acids of SEQ ID NO:42;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

57. A protein comprising an amino acid sequence selected from the group consisting of:
(a) the amino acid sequence of SEQ ID NO:42;
(b) fragments of the amino acid sequence of SEQ ID NO:42 comprising eight consecutive amino acids of SEQ ID NO:8; and
(c) the amino acid sequence encoded by the cDNA insert of clone dd12_{—}7 deposited under accession number ATCC 98456;
the protein being substantially free from other mammalian proteins.

58. An isolated gene corresponding to the cDNA sequence of SEQ ID NO:41.

59. An isolated polynucleotide selected from the group consisting of:
(a) a polynucleotide comprising the nucleotide sequence of SEQ ID NO:43;
(b) a polynucleotide comprising the nucleotide sequence of SEQ ID NO:43 from nucleotide 778 to nucleotide 1083;
(c) a polynucleotide comprising the nucleotide sequence of SEQ ID NO:43 from nucleotide 931 to nucleotide 1083;
(d) a polynucleotide comprising the nucleotide sequence of SEQ ID NO:43 from nucleotide 802 to nucleotide 1056;
(e) a polynucleotide comprising the nucleotide sequence of the full-length protein coding sequence of clone fn191_{—}3 deposited under accession number ATCC 98456;
(f) a polynucleotide encoding the full-length protein encoded by the cDNA insert of clone fn191_{—}3 deposited under accession number ATCC 98456;
(g) a polynucleotide comprising the nucleotide sequence of a mature protein coding sequence of clone fn191_{—}3 deposited under accession number ATCC 98456;
(h) a polynucleotide encoding a mature protein encoded by the cDNA insert of clone fn191_{—}3 deposited under accession number ATCC 98456;
(i) a polynucleotide encoding a protein comprising the amino acid sequence of SEQ ID NO:44;
(j) a polynucleotide encoding a protein comprising a fragment of the amino acid sequence of SEQ ID NO:44 having biological activity, the fragment comprising eight consecutive amino acids of SEQ ID NO:44;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

60. A protein comprising an amino acid sequence selected from the group consisting of:
(a) the amino acid sequence of SEQ ID NO:44;
(b) the amino acid sequence of SEQ ID NO:44 from amino acid 1 to amino acid 93;
(c) fragments of the amino acid sequence of SEQ ID NO:44 comprising eight consecutive amino acids of SEQ ID NO:44; and
(d) the amino acid sequence encoded by the cDNA insert of clone fn191_{—}3 deposited under accession number ATCC 98456;
the protein being substantially free from other mammalian proteins.

61. An isolated gene corresponding to the cDNA sequence of SEQ ID NO:43.

62. An isolated polynucleotide selected from the group consisting of:
(a) a polynucleotide comprising the nucleotide sequence of SEQ ID NO:45;
(b) a polynucleotide comprising the nucleotide sequence of SEQ ID NO:45 from nucleotide 390 to nucleotide 1355;
(c) a polynucleotide comprising the nucleotide sequence of SEQ ID NO:45 from nucleotide 1384 to nucleotide 1736;
(d) a polynucleotide comprising the nucleotide sequence of the full-length protein coding sequence of clone gm196_{—}4 deposited under accession number ATCC 98456;
(e) a polynucleotide encoding the full-length protein encoded by the cDNA insert of clone gm196_{—}4 deposited under accession number ATCC 98456;
(f) a polynucleotide comprising the nucleotide sequence of a mature protein coding sequence of clone gm196_{—}4 deposited under accession number ATCC 98456;
(g) a polynucleotide encoding a mature protein encoded by the cDNA insert of clone gm196_{—}4 deposited under accession number ATCC 98456;
(h) a polynucleotide encoding a protein comprising the amino acid sequence of SEQ ID NO:46;
(i) a polynucleotide encoding a protein comprising a fragment of the amino acid sequence of SEQ ID NO:46 having biological activity, the fragment comprising eight consecutive amino acids of SEQ ID NO:46;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

63. A protein comprising an amino acid sequence selected from the group consisting of:
(a) the amino acid sequence of SEQ ID NO:46;
(b) fragments of the amino acid sequence of SEQ ID NO:46 comprising eight consecutive amino acids of SEQ ID NO:46; and
(c) the amino acid sequence encoded by the cDNA insert of clone gm196_{—}4 deposited under accession number ATCC 98456;
the protein being substantially free from other mammalian proteins.

64. An isolated gene corresponding to the cDNA sequence of SEQ ID NO:45.

65. An isolated polynucleotide selected from the group consisting of:
(a) a polynucleotide comprising the nucleotide sequence of SEQ ID NO:47;
(b) a polynucleotide comprising the nucleotide sequence of SEQ ID NO:47 from nucleotide 879 to nucleotide 1391;
(c) a polynucleotide comprising the nucleotide sequence of SEQ ID NO:47 from nucleotide 519 to nucleotide 1074;
(d) a polynucleotide comprising the nucleotide sequence of the full-length protein coding sequence of clone gn114_{—}1 deposited under accession number ATCC 98456;
(e) a polynucleotide encoding the full-length protein encoded by the cDNA insert of clone gn114_{—}1 deposited under accession number ATCC 98456;
(f) a polynucleotide comprising the nucleotide sequence of a mature protein coding sequence of clone gn114_{—}1 deposited under accession number ATCC 98456;
(g) a polynucleotide encoding a mature protein encoded by the cDNA insert of clone gn114_{—}1 deposited under accession number ATCC 98456;
(h) a polynucleotide encoding a protein comprising the amino acid sequence of SEQ ID NO:48;
(i) a polynucleotide encoding a protein comprising a fragment of the amino acid sequence of SEQ ID NO:48 having biological activity, the fragment comprising eight consecutive amino acids of SEQ ID NO:48;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

66. A protein comprising an amino acid sequence selected from the group consisting of:
(a) the amino acid sequence of SEQ ID NO:48;
(b) the amino acid sequence of SEQ ID NO:48 from amino acid 1 to amino acid 65;
(c) fragments of the amino acid sequence of SEQ ID NO:48 comprising eight consecutive amino acids of SEQ ID NO:48; and
(d) the amino acid sequence encoded by the cDNA insert of clone gn114_{—}1 deposited under accession number ATCC 98456;
the protein being substantially free from other mammalian proteins.

67. An isolated gene corresponding to the cDNA sequence of SEQ ID NO:47.

68. An isolated polynucleotide selected from the group consisting of:
(a) a polynucleotide comprising the nucleotide sequence of SEQ ID NO:49;
(b) a polynucleotide comprising the nucleotide sequence of SEQ ID NO:49 from nucleotide 225 to nucleotide 1508;
(c) a polynucleotide comprising the nucleotide sequence of SEQ ID NO:49 from nucleotide 252 to nucleotide 1508;
(d) a polynucleotide comprising the nucleotide sequence of SEQ ID NO:15 from nucleotide 1 to nucleotide 302;
(e) a polynucleotide comprising the nucleotide sequence of the full-length protein coding sequence of clone hj968_{—}2 deposited under accession number ATCC 98456;
(f) a polynucleotide encoding the full-length protein encoded by the cDNA insert of clone hj968_{—}2 deposited under accession number ATCC 98456;
(g) a polynucleotide comprising the nucleotide sequence of a mature protein coding sequence of clone hj968_{—}2 deposited under accession number ATCC 98456;
(h) a polynucleotide encoding a mature protein encoded by the cDNA insert of clone hj968_{—}2 deposited under accession number ATCC 98456;
(i) a polynucleotide encoding a protein comprising the amino acid sequence of SEQ ID NO:50;
(j) a polynucleotide encoding a protein comprising a fragment of the amino acid sequence of SEQ ID NO:50 having biological activity, the fragment comprising eight consecutive amino acids of SEQ ID NO:50;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

69. A protein comprising an amino acid sequence selected from the group consisting of:
(a) the amino acid sequence of SEQ ID NO:50;
(b) the amino acid sequence of SEQ ID NO:50 from amino acid 1 to amino acid 26;
(c) fragments of the amino acid sequence of SEQ ID NO:50 comprising eight consecutive amino acids of SEQ ID NO:50; and
(d) the amino acid sequence encoded by the cDNA insert of clone hj968_{—}2 deposited under accession number ATCC 98456;
the protein being substantially free from other mammalian proteins.

70. An isolated gene corresponding to the cDNA sequence of SEQ ID NO:49.

71. An isolated polynucleotide selected from the group consisting of:
(a) a polynucleotide comprising the nucleotide sequence of SEQ ID NO:51;
(b) a polynucleotide comprising the nucleotide sequence of SEQ ID NO:51 from nucleotide 1113 to nucleotide 1274;
(c) a polynucleotide comprising the nucleotide sequence of SEQ ID NO:51 from nucleotide 1233 to nucleotide 1274;
(d) a polynucleotide comprising the nucleotide sequence of SEQ ID NO:51 from nucleotide 894 to nucleotide 1309;
(e) a polynucleotide comprising the nucleotide sequence of the full-length protein coding sequence of clone hk10_{—}3 deposited under accession number ATCC 98456;
(f) a polynucleotide encoding the full-length protein encoded by the cDNA insert of clone hk10_{—}3 deposited under accession number ATCC 98456;
(g) a polynucleotide comprising the nucleotide sequence of a mature protein coding sequence of clone hk10_{—}3 deposited under accession number ATCC 98456;
(h) a polynucleotide encoding a mature protein encoded by the cDNA insert of clone hk10_{—}3 deposited under accession number ATCC 98456;
(i) a polynucleotide encoding a protein comprising the amino acid sequence of SEQ ID NO:52;
(j) a polynucleotide encoding a protein comprising a fragment of the amino acid sequence of SEQ ID NO:52 having biological activity, the fragment comprising eight consecutive amino acids of SEQ ID NO:52;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

72. A protein comprising an amino acid sequence selected from the group consisting of:
(a) the amino acid sequence of SEQ ID NO:52;
(b) fragments of the amino acid sequence of SEQ ID NO:52 comprising eight consecutive amino acids of SEQ ID NO:52; and
(c) the amino acid sequence encoded by the cDNA insert of clone hk10_{—}3 deposited under accession number ATCC 98456;
the protein being substantially free from other mammalian proteins.

73. An isolated gene corresponding to the cDNA sequence of SEQ ID NO:52.

74. An isolated polynucleotide selected from the group consisting of:
(a) a polynucleotide comprising the nucleotide sequence of SEQ ID NO:53;
(b) a polynucleotide comprising the nucleotide sequence of SEQ ID NO:53 from nucleotide 96 to nucleotide 1145;
(c) a polynucleotide comprising the nucleotide sequence of SEQ ID NO:53 from nucleotide 109 to nucleotide 539;
(d) a polynucleotide comprising the nucleotide sequence of the full-length protein coding sequence of clone hm236_{—}1 deposited under accession number ATCC 98456;
(e) a polynucleotide encoding the full-length protein encoded by the cDNA insert of clone hm236_{—}1 deposited under accession number ATCC 98456;
(f) a polynucleotide comprising the nucleotide sequence of a mature protein coding sequence of clone hm236_{—}1 deposited under accession number ATCC 98456;
(g) a polynucleotide encoding a mature protein encoded by the cDNA insert of clone hm236_{—}1 deposited under accession number ATCC 98456;
(h) a polynucleotide encoding a protein comprising the amino acid sequence of SEQ ID NO:54;
(i) a polynucleotide encoding a protein comprising a fragment of the amino acid sequence of SEQ ID NO:54 having biological activity, the fragment comprising eight consecutive amino acids of SEQ ID NO:54;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

75. A protein comprising an amino acid sequence selected from the group consisting of:
(a) the amino acid sequence of SEQ ID NO:54;
(b) the amino acid sequence of SEQ ID NO:54 from amino acid 6 to amino acid 148;
(c) fragments of the amino acid sequence of SEQ ID NO:54 comprising eight consecutive amino acids of SEQ ID NO:54; and
(d) the amino acid sequence encoded by the cDNA insert of clone hm236_{—}1 deposited under accession number ATCC 98456;
the protein being substantially free from other mammalian proteins.

76. An isolated gene corresponding to the cDNA sequence of SEQ ID NO:54.

77. An isolated polynucleotide selected from the group consisting of:
(a) a polynucleotide comprising the nucleotide sequence of SEQ ID NO:67;
(b) a polynucleotide comprising the nucleotide sequence of SEQ ID NO:67 from nucleotide 185 to nucleotide 1600;
(c) a polynucleotide comprising the nucleotide sequence of SEQ ID NO:67 from nucleotide 1403 to nucleotide 1600;
(d) a polynucleotide comprising the nucleotide sequence of SEQ ID NO:67 from nucleotide 1 to nucleotide 850;
(e) a polynucleotide comprising the nucleotide sequence of the full-length protein coding sequence of clone do15_{—}4 deposited under accession number ATCC 98468;
(f) a polynucleotide encoding the full-length protein encoded by the cDNA insert of clone do15_{—}4 deposited under accession number ATCC 98468;
(g) a polynucleotide comprising the nucleotide sequence of a mature protein coding sequence of clone do15_{—}4 deposited under accession number ATCC 98468;
(h) a polynucleotide encoding a mature protein encoded by the cDNA insert of clone do15_{—}4 deposited under accession number ATCC 98468;
(i) a polynucleotide encoding a protein comprising the amino acid sequence of SEQ ID NO:68;
(j) a polynucleotide encoding a protein comprising a fragment of the amino acid sequence of SEQ ID NO:2 having biological activity, the fragment comprising eight consecutive amino acids of SEQ ID NO:2;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

78. The polynucleotide of claim 1 wherein said polynucleotide is operably linked to at least one expression control sequence.

79. A host cell transformed with the polynucleotide of claim 78.

80. The host cell of claim 79, wherein said cell is a mammalian cell.

81. A process for producing a protein encoded by the polynucleotide of claim 80, which process comprises:
(a) growing a culture of the host cell of claim 3 in a suitable culture medium; and
(b) purifying said protein from the culture.

82. A protein produced according to the process of claim 81.

82. A protein comprising an amino acid sequence selected from the group consisting of:
(a) the amino acid sequence of SEQ ID NO:68;
(b) the amino acid sequence of SEQ ID NO:68 from amino acid 1 to amino acid 222;
(c) fragments of the amino acid sequence of SEQ ID NO:68 comprising eight consecutive amino acids of SEQ ID NO:68; and
(d) the amino acid sequence encoded by the cDNA insert of clone do15_{—}4 deposited under accession number ATCC 98468;
the protein being substantially free from other mammalian proteins.

83. The protein of claim 82, wherein said protein comprises the amino acid sequence of SEQ ID NO:68.

84. The protein of claim 82, wherein said protein comprises the amino acid sequence of SEQ ID NO:68 from amino acid 1 to amino acid 222.

85. A composition comprising the protein of claim 82 and a pharmaceutically acceptable carrier.

86. An isolated gene corresponding to the cDNA sequence of SEQ ID NO:67.

87. An isolated polynucleotide selected from the group consisting of:
(a) a polynucleotide comprising the nucleotide sequence of SEQ ID NO:69;
(b) a polynucleotide comprising the nucleotide sequence of SEQ ID NO:69 from nucleotide 47 to nucleotide 2065;
(c) a polynucleotide comprising the nucleotide sequence of SEQ ID NO:69 from nucleotide 1086 to nucleotide 1848;
(d) a polynucleotide comprising the nucleotide sequence of the full-length protein coding sequence of clone dx290_{—}1 deposited under accession number ATCC 98468;
(e) a polynucleotide encoding the full-length protein encoded by the cDNA insert of clone dx290_{—}1 deposited under accession number ATCC 98468;
(f) a polynucleotide comprising the nucleotide sequence of a mature protein coding sequence of clone dx290_{—}1 deposited under accession number ATCC 98468;
(g) a polynucleotide encoding a mature protein encoded by the cDNA insert of clone dx290_{—}1 deposited under accession number ATCC 98468;
(h) a polynucleotide encoding a protein comprising the amino acid sequence of SEQ ID NO:70;
(i) a polynucleotide encoding a protein comprising a fragment of the amino acid sequence of SEQ ID NO:70 having biological activity, the fragment comprising eight consecutive amino acids of SEQ ID NO:70;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

88. A protein comprising an amino acid sequence selected from the group consisting of:
(a) the amino acid sequence of SEQ ID NO:70;
(b) the amino acid sequence of SEQ ID NO:70 from amino acid 312 to amino acid 600;
(c) fragments of the amino acid sequence of SEQ ID NO:70 comprising eight consecutive amino acids of SEQ ID NO:70; and
(d) the amino acid sequence encoded by the cDNA insert of clone dx290_{—}1 deposited under accession number ATCC 98468;
the protein being substantially free from other mammalian proteins.

89. An isolated gene corresponding to the cDNA sequence of SEQ ID NO:69.

90. An isolated polynucleotide selected from the group consisting of:
(a) a polynucleotide comprising the nucleotide sequence of SEQ ID NO:71;
(b) a polynucleotide comprising the nucleotide sequence of SEQ ID NO:71 from nucleotide 107 to nucleotide 724;
(c) a polynucleotide comprising the nucleotide sequence of SEQ ID NO:71 from nucleotide 218 to nucleotide 724;
(d) a polynucleotide comprising the nucleotide sequence of SEQ ID NO:71 from nucleotide 536 to nucleotide 866;
(e) a polynucleotide comprising the nucleotide sequence of the full-length protein coding sequence of clone ek390_{—}4 deposited under accession number ATCC 98468;
(f) a polynucleotide encoding the full-length protein encoded by the cDNA insert of clone ek390_{—}4 deposited under accession number ATCC 98468;
(g) a polynucleotide comprising the nucleotide sequence of a mature protein coding sequence of clone ek390_{—}4 deposited under accession number ATCC 98468;
(h) a polynucleotide encoding a mature protein encoded by the cDNA insert of clone ek390_{—}4 deposited under accession number ATCC 98468;
(i) a polynucleotide encoding a protein comprising the amino acid sequence of SEQ ID NO:72;
(j) a polynucleotide encoding a protein comprising a fragment of the amino acid sequence of SEQ ID NO:72 having biological activity, the fragment comprising eight consecutive amino acids of SEQ ID NO:72;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

91. A protein comprising an amino acid sequence selected from the group consisting of:
(a) the amino acid sequence of SEQ ID NO:72;
(b) the amino acid sequence of SEQ ID NO:72 from amino acid 6 to amino acid 92;
(c) fragments of the amino acid sequence of SEQ ID NO:72 comprising eight consecutive amino acids of SEQ ID NO:72; and
(d) the amino acid sequence encoded by the cDNA insert of clone ek390_{—}4 deposited under accession number ATCC 98468;
the protein being substantially free from other mammalian proteins.

92. An isolated gene corresponding to the cDNA sequence of SEQ ID NO:71.

93. An isolated polynucleotide selected from the group consisting of:
(a) a polynucleotide comprising the nucleotide sequence of SEQ ID NO:73;
(b) a polynucleotide comprising the nucleotide sequence of SEQ ID NO:73 from nucleotide 31 to nucleotide 1230;
(c) a polynucleotide comprising the nucleotide sequence of SEQ ID NO:73 from nucleotide 289 to nucleotide 1230;
(d) a polynucleotide comprising the nucleotide sequence of SEQ ID NO:73 from nucleotide 344 to nucleotide 1119;
(e) a polynucleotide comprising the nucleotide sequence of the full-length protein coding sequence of clone er471_{—}7 deposited under accession number ATCC 98468;
(f) a polynucleotide encoding the full-length protein encoded by the cDNA insert of clone er471_{—}7 deposited under accession number ATCC 98468;
(g) a polynucleotide comprising the nucleotide sequence of a mature protein coding sequence of clone er471_{—}7 deposited under accession number ATCC 98468;
(h) a polynucleotide encoding a mature protein encoded by the cDNA insert of clone er471_{—}7 deposited under accession number ATCC 98468;
(i) a polynucleotide encoding a protein comprising the amino acid sequence of SEQ ID NO:74;
(j) a polynucleotide encoding a protein comprising a fragment of the amino acid sequence of SEQ ID NO:74 having biological activity, the fragment comprising eight consecutive amino acids of SEQ ID NO:74;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

94. A protein comprising an amino acid sequence selected from the group consisting of:
(a) the amino acid sequence of SEQ ID NO:74;
(b) the amino acid sequence of SEQ ID NO:74 from amino acid 111 to amino acid 363;
(c) fragments of the amino acid sequence of SEQ ID NO:74 comprising eight consecutive amino acids of SEQ ID NO:74; and
(d) the amino acid sequence encoded by the cDNA insert of clone er471_{—}7 deposited under accession number ATCC 98468;
the protein being substantially free from other mammalian proteins.

95. An isolated gene corresponding to the cDNA sequence of SEQ ID NO:74.

96. An isolated polynucleotide selected from the group consisting of:
(a) a polynucleotide comprising the nucleotide sequence of SEQ ID NO:75;
(b) a polynucleotide comprising the nucleotide sequence of SEQ ID NO:75 from nucleotide 62 to nucleotide 322;
(c) a polynucleotide comprising the nucleotide sequence of SEQ ID NO:75 from nucleotide 571 to nucleotide 878;
(d) a polynucleotide comprising the nucleotide sequence of the full-length protein coding sequence of clone fs40_{—}3 deposited under accession number ATCC 98468;
(e) a polynucleotide encoding the full-length protein encoded by the cDNA insert of clone fs40_{—}3 deposited under accession number ATCC 98468;
(f) a polynucleotide comprising the nucleotide sequence of a mature protein coding sequence of clone fs40_{—}3 deposited under accession number ATCC 98468;
(g) a polynucleotide encoding a mature protein encoded by the cDNA insert of clone fs40_{—}3 deposited under accession number ATCC 98468;
(h) a polynucleotide encoding a protein comprising the amino acid sequence of SEQ ID NO:76;
(i) a polynucleotide encoding a protein comprising a fragment of the amino acid sequence of SEQ ID NO:76 having biological activity, the fragment comprising eight consecutive amino acids of SEQ ID NO:76;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

97. A protein comprising an amino acid sequence selected from the group consisting of:
(a) the amino acid sequence of SEQ ID NO:76;
(b) fragments of the amino acid sequence of SEQ ID NO:76 comprising eight consecutive amino acids of SEQ ID NO:76; and
(c) the amino acid sequence encoded by the cDNA insert of clone fs40_{—}3 deposited under accession number ATCC 98468;
the protein being substantially free from other mammalian proteins.

98. An isolated gene corresponding to the cDNA sequence of SEQ ID NO:75.

99. An isolated polynucleotide selected from the group consisting of:
(a) a polynucleotide comprising the nucleotide sequence of SEQ ID NO:77;
(b) a polynucleotide comprising the nucleotide sequence of SEQ ID NO:77 from nucleotide 43 to nucleotide 1671;
(c) a polynucleotide comprising the nucleotide sequence of SEQ ID NO:77 from nucleotide 112 to nucleotide 1671;
(d) a polynucleotide comprising the nucleotide sequence of SEQ ID NO:77 from nucleotide 224 to nucleotide 679;
(e) a polynucleotide comprising the nucleotide sequence of the full-length protein coding sequence of clone ga63_{—}6 deposited under accession number ATCC 98468;
(f) a polynucleotide encoding the full-length protein encoded by the cDNA insert of clone ga63_{—}6 deposited under accession number ATCC 98468;
(g) a polynucleotide comprising the nucleotide sequence of a mature protein coding sequence of clone ga63_{—}6 deposited under accession number ATCC 98468;
(h) a polynucleotide encoding a mature protein encoded by the cDNA insert of clone ga63_{—}6 deposited under accession number ATCC 98468;
(i) a polynucleotide encoding a protein comprising the amino acid sequence of SEQ ID NO:78;
(j) a polynucleotide encoding a protein comprising a fragment of the amino acid sequence of SEQ ID NO:78 having biological activity, the fragment comprising eight consecutive amino acids of SEQ ID NO:78;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

100. A protein comprising an amino acid sequence selected from the group consisting of:
(a) the amino acid sequence of SEQ ID NO:78;
(b) the amino acid sequence of SEQ ID NO:78 from amino acid 62 to amino acid 212;
(c) fragments of the amino acid sequence of SEQ ID NO:78 comprising eight consecutive amino acids of SEQ ID NO:78; and
(d) the amino acid sequence encoded by the cDNA insert of clone ga63_{—}6 deposited under accession number ATCC 98468;
the protein being substantially free from other mammalian proteins.

101. An isolated gene corresponding to the cDNA sequence of SEQ ID NO:77.

102. An isolated polynucleotide selected from the group consisting of:
(a) a polynucleotide comprising the nucleotide sequence of SEQ ID NO:79;
(b) a polynucleotide comprising the nucleotide sequence of SEQ ID NO:79 from nucleotide 17 to nucleotide 523;
(c) a polynucleotide comprising the nucleotide sequence of SEQ ID NO:79 from nucleotide 77 to nucleotide 523;
(d) a polynucleotide comprising the nucleotide sequence of SEQ ID NO:79 from nucleotide 1 to nucleotide 392;
(e) a polynucleotide comprising the nucleotide sequence of the full-length protein coding sequence of clone gm335_{—}4 deposited under accession number ATCC 98468;
(f) a polynucleotide encoding the full-length protein encoded by the cDNA insert of clone gm335_{—}4 deposited under accession number ATCC 98468;
(g) a polynucleotide comprising the nucleotide sequence of a mature protein coding sequence of clone gm335_{—}4 deposited under accession number ATCC 98468;
(h) a polynucleotide encoding a mature protein encoded by the cDNA insert of clone gm335_{—}4 deposited under accession number ATCC 98468;
(i) a polynucleotide encoding a protein comprising the amino acid sequence of SEQ ID NO:80;
(j) a polynucleotide encoding a protein comprising a fragment of the amino acid sequence of SEQ ID NO:80 having biological activity, the fragment comprising eight consecutive amino acids of SEQ ID NO:80;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

103. A protein comprising an amino acid sequence selected from the group consisting of:
(a) the amino acid sequence of SEQ ID NO:80;
(b) the amino acid sequence of SEQ ID NO:80 from amino acid 1 to amino acid 125;
(c) fragments of the amino acid sequence of SEQ ID NO:80 comprising eight consecutive amino acids of SEQ ID NO:80; and
(d) the amino acid sequence encoded by the cDNA insert of clone gm335_{—}4 deposited under accession number ATCC 98468;
the protein being substantially free from other mammalian proteins.

104. An isolated gene corresponding to the cDNA sequence of SEQ ID NO:79.

105. An isolated polynucleotide selected from the group consisting of:
(a) a polynucleotide comprising the nucleotide sequence of SEQ ID NO:81;
(b) a polynucleotide comprising the nucleotide sequence of SEQ ID NO:81 from nucleotide 2 to nucleotide 991;
(c) a polynucleotide comprising the nucleotide sequence of SEQ ID NO:81 from nucleotide 62 to nucleotide 991;
(d) a polynucleotide comprising the nucleotide sequence of SEQ ID NO:81 from nucleotide 2 to nucleotide 504;
(e) a polynucleotide comprising the nucleotide sequence of the full-length protein coding sequence of clone hy370_{—}9 deposited under accession number ATCC 98468;
(f) a polynucleotide encoding the full-length protein encoded by the cDNA insert of clone hy370_{—}9 deposited under accession number ATCC 98468;
(g) a polynucleotide comprising the nucleotide sequence of a mature protein coding sequence of clone hy370_{—}9 deposited under accession number ATCC 98468;
(h) a polynucleotide encoding a mature protein encoded by the cDNA insert of clone hy370_{—}9 deposited under accession number ATCC 98468;
(i) a polynucleotide encoding a protein comprising the amino acid sequence of SEQ ID NO:82;
(j) a polynucleotide encoding a protein comprising a fragment of the amino acid sequence of SEQ ID NO:82 having biological activity, the fragment comprising eight consecutive amino acids of SEQ ID NO:82;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

106. A protein comprising an amino acid sequence selected from the group consisting of:
(a) the amino acid sequence of SEQ ID NO:82;
(b) the amino acid sequence of SEQ ID NO:82 from amino acid 1 to amino acid 167;
(c) fragments of the amino acid sequence of SEQ ID NO:82 comprising eight consecutive amino acids of SEQ ID NO:16; and
(d) the amino acid sequence encoded by the cDNA insert of clone hy370_{—}9 deposited under accession number ATCC 98468;
the protein being substantially free from other mammalian proteins.

107. An isolated gene corresponding to the cDNA sequence of SEQ ID NO:81.

108. An isolated polynucleotide selected from the group consisting of:
(a) a polynucleotide comprising the nucleotide sequence of SEQ ID NO:83;
(b) a polynucleotide comprising the nucleotide sequence of SEQ ID NO:83 from nucleotide 77 to nucleotide 616;
(c) a polynucleotide comprising the nucleotide sequence of SEQ ID NO:83 from nucleotide 164 to nucleotide 616;
(d) a polynucleotide comprising the nucleotide sequence of SEQ ID NO:83 from nucleotide 1 to nucleotide 415;
(e) a polynucleotide comprising the nucleotide sequence of the full-length protein coding sequence of clone ie47_{—}4 deposited under accession number ATCC 98468;
(f) a polynucleotide encoding the full-length protein encoded by the cDNA insert of clone ie47_{—}4 deposited under accession number ATCC 98468;
(g) a polynucleotide comprising the nucleotide sequence of a mature protein coding sequence of clone ie47_{—}4 deposited under accession number ATCC 98468;
(h) a polynucleotide encoding a mature protein encoded by the cDNA insert of clone ie47_{—}4 deposited under accession number ATCC 98468;
(i) a polynucleotide encoding a protein comprising the amino acid sequence of SEQ ID NO:83;
(j) a polynucleotide encoding a protein comprising a fragment of the amino acid sequence of SEQ ID NO:83 having biological activity, the fragment comprising eight consecutive amino acids of SEQ ID NO:83;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

109. A protein comprising an amino acid sequence selected from the group consisting of:
(a) the amino acid sequence of SEQ ID NO:84;
(b) the amino acid sequence of SEQ ID NO:84 from amino acid 1 to amino acid 113;
(c) fragments of the amino acid sequence of SEQ ID NO:84 comprising eight consecutive amino acids of SEQ ID NO:84; and
(d) the amino acid sequence encoded by the cDNA insert of clone ie47_{—}4 deposited under accession number ATCC 98468;
the protein being substantially free from other mammalian proteins.

110. An isolated gene corresponding to the cDNA sequence of SEQ ID NO:84.

111. An isolated polynucleotide selected from the group consisting of:
(a) a polynucleotide comprising the nucleotide sequence of SEQ ID NO:85;
(b) a polynucleotide comprising the nucleotide sequence of SEQ ID NO:85 from nucleotide 564 to nucleotide 2813;
(c) a polynucleotide comprising the nucleotide sequence of SEQ ID NO:85 from nucleotide 705 to nucleotide 2813;
(d) a polynucleotide comprising the nucleotide sequence of SEQ ID NO:85 from nucleotide 793 to nucleotide 1628;
(e) a polynucleotide comprising the nucleotide sequence of the full-length protein coding sequence of clone s195_{—}10 deposited under accession number ATCC 98468;
(f) a polynucleotide encoding the full-length protein encoded by the cDNA insert of clone s195_{—}10 deposited under accession number ATCC 98468;
(g) a polynucleotide comprising the nucleotide sequence of a mature protein coding sequence of clone s195_{—}10 deposited under accession number ATCC 98468;
(h) a polynucleotide encoding a mature protein encoded by the cDNA insert of clone s195_{—}10 deposited under accession number ATCC 98468;
(i) a polynucleotide encoding a protein comprising the amino acid sequence of SEQ ID NO:86;
(j) a polynucleotide encoding a protein comprising a fragment of the amino acid sequence of SEQ ID NO:86 having biological activity, the fragment comprising eight consecutive amino acids of SEQ ID NO:86;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

112. A protein comprising an amino acid sequence selected from the group consisting of:
(a) the amino acid sequence of SEQ ID NO:86;
(b) the amino acid sequence of SEQ ID NO:86 from amino acid 78 to amino acid 355;
(c) fragments of the amino acid sequence of SEQ ID NO:86 comprising eight consecutive amino acids of SEQ ID NO:86; and
(d) the amino acid sequence encoded by the cDNA insert of clone s195_{—}10 deposited under accession number ATCC 98468;
the protein being substantially free from other mammalian proteins.

113. An isolated gene corresponding to the cDNA sequence of SEQ ID NO:85.

114. A composition comprising an isolated polynucleotide selected from the group consisting of:
(a) a polynucleotide comprising the nucleotide sequence of SEQ ID NO:97;
(b) a polynucleotide comprising the nucleotide sequence of SEQ ID NO:97 from nucleotide 516 to nucleotide 797;
(c) a polynucleotide comprising the nucleotide sequence of SEQ ID NO:97 from nucleotide 606 to nucleotide 797;
(d) a polynucleotide comprising the nucleotide sequence of SEQ ID NO:97 from nucleotide 1 to nucleotide 773;
(e) a polynucleotide comprising the nucleotide sequence of the full-length protein coding sequence of clone bf228_{—}14 deposited under accession number ATCC 98482;
(f) a polynucleotide encoding the full-length protein encoded by the cDNA insert of clone bf228_{—}14 deposited under accession number ATCC 98482;
(g) a polynucleotide comprising the nucleotide sequence of the mature protein coding sequence of clone bf228_{—}14 deposited under accession number ATCC 98482;
(h) a polynucleotide encoding the mature protein encoded by the cDNA insert of clone bf228_{—}14 deposited under accession number ATCC 98482;
(i) a polynucleotide encoding a protein comprising the amino acid sequence of SEQ ID NO:98;
(j) a polynucleotide encoding a protein comprising a fragment of the amino acid sequence of SEQ ID NO:98 having biological activity;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

115. A composition of claim 1 wherein said polynucleotide is operably linked to an expression control sequence.

116. A host cell transformed with a composition of claim 115.

117. The host cell of claim 116, wherein said cell is a mammalian cell.

118. A process for producing a protein, which comprises:
(a) growing a culture of the host cell of claim 117 in a suitable culture medium; and
(b) purifying the protein from the culture.

119. A protein produced according to the process of claim 118.

120. The protein of claim 115 comprising a mature protein.

121. A composition comprising a protein, wherein said protein comprises an amino acid sequence selected from the group consisting of:
(a) the amino acid sequence of SEQ ID NO:98;
(b) the amino acid sequence of SEQ ID NO:98 from amino acid 1 to amino acid 86;
(c) fragments of the amino acid sequence of SEQ ID NO:98; and
(d) the amino acid sequence encoded by the cDNA insert of clone bf228_{—}14 deposited under accession number ATCC 98482;
the protein being substantially free from other mammalian proteins.

122. The composition of claim 121, wherein said protein comprises the amino acid sequence of SEQ ID NO:98.

123. The composition of claim 121, wherein said protein comprises the amino acid sequence of SEQ ID NO:98 from amino acid 1 to amino acid 86.

124. The composition of claim 121, further comprising a pharmaceutically acceptable carrier.

125. A method for preventing, treating or ameliorating a medical condition which comprises administering to a mammalian subject a therapeutically effective amount of a composition of claim 121.

126. An isolated gene corresponding to the cDNA sequence of SEQ ID NO:97.

127. A composition comprising an isolated polynucleotide selected from the group consisting of:
(a) a polynucleotide comprising the nucleotide sequence of SEQ ID NO:99;
(b) a polynucleotide comprising the nucleotide sequence of SEQ ID NO:99 from nucleotide 137 to nucleotide 1240;
(c) a polynucleotide comprising the nucleotide sequence of SEQ ID NO:99 from nucleotide 1 to nucleotide 1153;
(d) a polynucleotide comprising the nucleotide sequence of the full-length protein coding sequence of clone bg249_{—}1 deposited under accession number ATCC 98482;
(e) a polynucleotide encoding the full-length protein encoded by the cDNA insert of clone bg249_{—}1 deposited under accession number ATCC 98482;
(f) a polynucleotide comprising the nucleotide sequence of the mature protein coding sequence of clone bg249_{—}1 deposited under accession number ATCC 98482;
(g) a polynucleotide encoding the mature protein encoded by the cDNA insert of clone bg249_{—}1 deposited under accession number ATCC 98482;
(h) a polynucleotide encoding a protein comprising the amino acid sequence of SEQ ID NO:100;
(i) a polynucleotide encoding a protein comprising a fragment of the amino acid sequence of SEQ ID NO:100 having biological activity;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

128. A composition comprising a protein, wherein said protein comprises an amino acid sequence selected from the group consisting of:
(a) the amino acid sequence of SEQ ID NO:100;
(b) the amino acid sequence of SEQ ID NO:100 from amino acid 1 to amino acid 339;
(c) fragments of the amino acid sequence of SEQ ID NO:100; and
(d) the amino acid sequence encoded by the cDNA insert of clone bg249_{—}1 deposited under accession number ATCC 98482;
the protein being substantially free from other mammalian proteins.

129. An isolated gene corresponding to the cDNA sequence of SEQ ID NO:99.

130. A composition comprising an isolated polynucleotide selected from the group consisting of:
(a) a polynucleotide comprising the nucleotide sequence of SEQ ID NO:101;
(b) a polynucleotide comprising the nucleotide sequence of SEQ ID NO:101 from nucleotide 26 to nucleotide 301;
(c) a polynucleotide comprising the nucleotide sequence of SEQ ID NO:101 from nucleotide 104 to nucleotide 301;
(d) a polynucleotide comprising the nucleotide sequence of SEQ ID NO:101 from nucleotide 1 to nucleotide 119;
(e) a polynucleotide comprising the nucleotide sequence of the full-length protein coding sequence of clone bv286_{—}1 deposited under accession number ATCC 98482;
(f) a polynucleotide encoding the full-length protein encoded by the cDNA insert of clone bv286_{—}1 deposited under accession number ATCC 98482;
(g) a polynucleotide comprising the nucleotide sequence of the mature protein coding sequence of clone bv286_{—}1 deposited under accession number ATCC 98482;
(h) a polynucleotide encoding the mature protein encoded by the cDNA insert of clone bv286_{—}1 deposited under accession number ATCC 98482;
(i) a polynucleotide encoding a protein comprising the amino acid sequence of SEQ ID NO:102;
(j) a polynucleotide encoding a protein comprising a fragment of the amino acid sequence of SEQ ID NO:102 having biological activity;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

131. A composition comprising a protein, wherein said protein comprises an amino acid sequence selected from the group consisting of:
(a) the amino acid sequence of SEQ ID NO:102;
(b) the amino acid sequence of SEQ ID NO:102 from amino acid 1 to amino acid 31;
(c) fragments of the amino acid sequence of SEQ ID NO:102; and
(d) the amino acid sequence encoded by the cDNA insert of clone bv286_{—}1 deposited under accession number ATCC 98482;
the protein being substantially free from other mammalian proteins.

132. An isolated gene corresponding to the cDNA sequence of SEQ ID NO:101.

133. A composition comprising an isolated polynucleotide selected from the group consisting of:
(a) a polynucleotide comprising the nucleotide sequence of SEQ ID NO:103;
(b) a polynucleotide comprising the nucleotide sequence of SEQ ID NO:103 from nucleotide 663 to nucleotide 755;
(c) a polynucleotide comprising the nucleotide sequence of SEQ ID NO:103 from nucleotide 1 to nucleotide 850;
(d) a polynucleotide comprising the nucleotide sequence of the full-length protein coding sequence of clone co36_{—}1 deposited under accession number ATCC 98482;
(e) a polynucleotide encoding the full-length protein encoded by the cDNA insert of clone co36_{—}1 deposited under accession number ATCC 98482;
(f) a polynucleotide comprising the nucleotide sequence of the mature protein coding sequence of clone co36_{—}1 deposited under accession number ATCC 98482;
(g) a polynucleotide encoding the mature protein encoded by the cDNA insert of clone co36_{—}1 deposited under accession number ATCC 98482;
(h) a polynucleotide encoding a protein comprising the amino acid sequence of SEQ ID NO:104;
(i) a polynucleotide encoding a protein comprising a fragment of the amino acid sequence of SEQ ID NO:104 having biological activity;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

134. A composition comprising a protein, wherein said protein comprises an amino acid sequence selected from the group consisting of:
(a) the amino acid sequence of SEQ ID NO:104;
(b) the amino acid sequence of SEQ ID NO:104 from amino acid 1 to amino acid 22;
(c) fragments of the amino acid sequence of SEQ ID NO:104; and
(d) the amino acid sequence encoded by the cDNA insert of clone co36_{—}1 deposited under accession number ATCC 98482;
the protein being substantially free from other mammalian proteins.

135. An isolated gene corresponding to the cDNA sequence of SEQ ID NO:103.

136. A composition comprising an isolated polynucleotide selected from the group consisting of:
(a) a polynucleotide comprising the nucleotide sequence of SEQ ID NO:105;
(b) a polynucleotide comprising the nucleotide sequence of SEQ ID NO:105 from nucleotide 127 to nucleotide 783;
(c) a polynucleotide comprising the nucleotide sequence of SEQ ID NO:105 from nucleotide 172 to nucleotide 783;
(d) a polynucleotide comprising the nucleotide sequence of SEQ ID NO:105 from nucleotide 7 to nucleotide 462;
(e) a polynucleotide comprising the nucleotide sequence of the full-length protein coding sequence of clone cp116_{—}1 deposited under accession number ATCC 98482;
(f) a polynucleotide encoding the full-length protein encoded by the cDNA insert of clone cp116_{—}1 deposited under accession number ATCC 98482;
(g) a polynucleotide comprising the nucleotide sequence of the mature protein coding sequence of clone cp116_{—}1 deposited under accession number ATCC 98482;
(h) a polynucleotide encoding the mature protein encoded by the cDNA insert of clone cp116_{—}1 deposited under accession number ATCC 98482;
(i) a polynucleotide encoding a protein comprising the amino acid sequence of SEQ ID NO:106;
(j) a polynucleotide encoding a protein comprising a fragment of the amino acid sequence of SEQ ID NO:106 having biological activity;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

137. A composition comprising a protein, wherein said protein comprises an amino acid sequence selected from the group consisting of:
(a) the amino acid sequence of SEQ ID NO:106;
(b) the amino acid sequence of SEQ ID NO:106 from amino acid 1 to amino acid 112;
(c) fragments of the amino acid sequence of SEQ ID NO:106; and
(d) the amino acid sequence encoded by the cDNA insert of clone cp116_{—}1 deposited under accession number ATCC 98482;
the protein being substantially free from other mammalian proteins.

138. An isolated gene corresponding to the cDNA sequence of SEQ ID NO:105.

139. A composition comprising an isolated polynucleotide selected from the group consisting of:
(a) a polynucleotide comprising the nucleotide sequence of SEQ ID NO:108;
(b) a polynucleotide comprising the nucleotide sequence of SEQ ID NO:108 from nucleotide 231 to nucleotide 533;
(c) a polynucleotide comprising the nucleotide sequence of the full-length protein coding sequence of clone cw1195_{—}2 deposited under accession number ATCC 98482;
(d) a polynucleotide encoding the full-length protein encoded by the cDNA insert of clone cw1195_{—}2 deposited under accession number ATCC 98482;
(e) a polynucleotide comprising the nucleotide sequence of the mature protein coding sequence of clone cw1195_{—}2 deposited under accession number ATCC 98482;
(f) a polynucleotide encoding the mature protein encoded by the cDNA insert of clone cw1195_{—}2 deposited under accession number ATCC 98482;
(g) a polynucleotide encoding a protein comprising the amino acid sequence of SEQ ID NO:109;
(h) a polynucleotide encoding a protein comprising a fragment of the amino acid sequence of SEQ ID NO:109 having biological activity;
(i) a polynucleotide which is an allelic variant of a polynucleotide of (a)-(f) above;
(j) a polynucleotide which encodes a species homologue of the protein of (g) or (h) above; and
(k) a polynucleotide capable of hybridizing under stringent conditions to any one of the polynucleotides specified in (a)-(h).

140. A composition comprising a protein, wherein said protein comprises an amino acid sequence selected from the group consisting of:
(a) the amino acid sequence of SEQ ID NO:109;
(b) fragments of the amino acid sequence of SEQ ID NO:109; and
(c) the amino acid sequence encoded by the cDNA insert of clone cw1195_{—}2 deposited under accession number ATCC 98482;
the protein being substantially free from other mammalian proteins.

141. An isolated gene corresponding to the cDNA sequence of SEQ ID NO:108, SEQ ID NO:107 or SEQ ID NO:110.

142. A composition comprising an isolated polynucleotide selected from the group consisting of:
(a) a polynucleotide comprising the nucleotide sequence of SEQ ID NO:111;
(b) a polynucleotide comprising the nucleotide sequence of SEQ ID NO:111 from nucleotide 645 to nucleotide 782;
(c) a polynucleotide comprising the nucleotide sequence of SEQ ID NO:111 from nucleotide 10 to nucleotide 773;
(d) a polynucleotide comprising the nucleotide sequence of the full-length protein coding sequence of clone fh13_{—}10 deposited under accession number ATCC 98482;
(e) a polynucleotide encoding the full-length protein encoded by the cDNA insert of clone fh13_{—}10 deposited under accession number ATCC 98482;
(f) a polynucleotide comprising the nucleotide sequence of the mature protein coding sequence of clone fh13_{—}10 deposited under accession number ATCC 98482;
(g) a polynucleotide encoding the mature protein encoded by the cDNA insert of clone fh13_{—}10 deposited under accession number ATCC 98482;
(h) a polynucleotide encoding a protein comprising the amino acid sequence of SEQ ID NO:112;
(i) a polynucleotide encoding a protein comprising a fragment of the amino acid sequence of SEQ ID NO:112 having biological activity;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

143. A composition comprising a protein, wherein said protein comprises an amino acid sequence selected from the group consisting of:
(a) the amino acid sequence of SEQ ID NO:112;
(b) the amino acid sequence of SEQ ID NO:112 from amino acid 1 to amino acid 43;
(c) fragments of the amino acid sequence of SEQ ID NO:112; and
(d) the amino acid sequence encoded by the cDNA insert of clone fh13_{—}10 deposited under accession number ATCC 98482;
the protein being substantially free from other mammalian proteins.

144. An isolated gene corresponding to the cDNA sequence of SEQ ID NO:111.

145. A composition comprising an isolated polynucleotide selected from the group consisting of:
(a) a polynucleotide comprising the nucleotide sequence of SEQ ID NO:113;
(b) a polynucleotide comprising the nucleotide sequence of SEQ ID NO:113 from nucleotide 94 to nucleotide 216;
(c) a polynucleotide comprising the nucleotide sequence of SEQ ID NO:113 from nucleotide 160 to nucleotide 216;
(d) a polynucleotide comprising the nucleotide sequence of SEQ ID NO:113 from nucleotide 20 to nucleotide 193;
(e) a polynucleotide comprising the nucleotide sequence of the full-length protein coding sequence of clone gc57_{—}4 deposited under accession number ATCC 98482;
(f) a polynucleotide encoding the full-length protein encoded by the cDNA insert of clone gc57_{—}4 deposited under accession number ATCC 98482;
(g) a polynucleotide comprising the nucleotide sequence of the mature protein coding sequence of clone gc57_{—}4 deposited under accession number ATCC 98482;
(h) a polynucleotide encoding the mature protein encoded by the cDNA insert of clone gc57_{—}4 deposited under accession number ATCC 98482;
(i) a polynucleotide encoding a protein comprising the amino acid sequence of SEQ ID NO:114;
(j) a polynucleotide encoding a protein comprising a fragment of the amino acid sequence of SEQ ID NO:114 having biological activity;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

146. A composition comprising a protein, wherein said protein comprises an amino acid sequence selected from the group consisting of:
(a) the amino acid sequence of SEQ ID NO:114;
(b) the amino acid sequence of SEQ ID NO:114 from amino acid 1 to amino acid 33;
(c) fragments of the amino acid sequence of SEQ ID NO:114; and
(d) the amino acid sequence encoded by the cDNA insert of clone gc57_{—}4 deposited under accession number ATCC 98482;
the protein being substantially free from other mammalian proteins.

147. An isolated gene corresponding to the cDNA sequence of SEQ ID NO:113.

148. A composition comprising an isolated polynucleotide selected from the group consisting of:
(a) a polynucleotide comprising the nucleotide sequence of SEQ ID NO:115;
(b) a polynucleotide comprising the nucleotide sequence of SEQ ID NO:115 from nucleotide 2 to nucleotide 943;
(c) a polynucleotide comprising the nucleotide sequence of SEQ ID NO:115 from nucleotide 2 to nucleotide 670;
(d) a polynucleotide comprising the nucleotide sequence of the full-length protein coding sequence of clone h1165_{—}3 deposited under accession number ATCC 98482;
(e) a polynucleotide encoding the full-length protein encoded by the cDNA insert of clone h1165_{—}3 deposited under accession number ATCC 98482;
(f) a polynucleotide comprising the nucleotide sequence of the mature protein coding sequence of clone h1165_{—}3 deposited under accession number ATCC 98482;
(g) a polynucleotide encoding the mature protein encoded by the cDNA insert of clone h1165_{—}3 deposited under accession number ATCC 98482;
(h) a polynucleotide encoding a protein comprising the amino acid sequence of SEQ ID NO:116;
(i) a polynucleotide encoding a protein comprising a fragment of the amino acid sequence of SEQ ID NO:116 having biological activity;
(j) a polynucleotide which is an allelic variant of a polynucleotide of (a)-(g) above;
(k) a polynucleotide which encodes a species homologue of the protein of (h) or (i) above; and
(l) a polynucleotide capable of hybridizing under stringent conditions to any one of the polynucleotides specified in (a)-(i).

149. A composition comprising a protein, wherein said protein comprises an amino acid sequence selected from the group consisting of:
(a) the amino acid sequence of SEQ ID NO:116;
(b) the amino acid sequence of SEQ ID NO:116 from amino acid 1 to amino acid 223;
(c) fragments of the amino acid sequence of SEQ ID NO:116; and
(d) the amino acid sequence encoded by the cDNA insert of clone h1165_{—}3 deposited under accession number ATCC 98482;
the protein being substantially free from other mammalian proteins.

150. An isolated gene corresponding to the cDNA sequence of SEQ ID NO:115.

151. A composition comprising an isolated polynucleotide selected from the group consisting of:
(a) a polynucleotide comprising the nucleotide sequence of SEQ ID NO:117;
(b) a polynucleotide comprising the nucleotide sequence of SEQ ID NO:117 from nucleotide 1242 to nucleotide 1457;
(c) a polynucleotide comprising the nucleotide sequence of SEQ ID NO:117 from nucleotide 1326 to nucleotide 1457;
(d) a polynucleotide comprising the nucleotide sequence of SEQ ID NO:117 from nucleotide 869 to nucleotide 1544;
(e) a polynucleotide comprising the nucleotide sequence of the full-length protein coding sequence of clone hb752_{—}1 deposited under accession number ATCC 98482;
(f) a polynucleotide encoding the full-length protein encoded by the cDNA insert of clone hb752_{—}1 deposited under accession number ATCC 98482;
(g) a polynucleotide comprising the nucleotide sequence of the mature protein coding sequence of clone hb752_{—}1 deposited under accession number ATCC 98482;
(h) a polynucleotide encoding the mature protein encoded by the cDNA insert of clone hb752_{—}1 deposited under accession number ATCC 98482;
(i) a polynucleotide encoding a protein comprising the amino acid sequence of SEQ ID NO:118;
(j) a polynucleotide encoding a protein comprising a fragment of the amino acid sequence of SEQ ID NO:118 having biological activity;
(k) a polynucleotide which is an allelic variant of a polynucleotide of (a)-(h) above;
(l) a polynucleotide which encodes a species homologue of the protein of (i) or (j) above; and
(m) a polynucleotide capable of hybridizing under stringent conditions to any one of the polynucleotides specified in (a)-(j).

152. A composition comprising a protein, wherein said protein comprises an amino acid sequence selected from the group consisting of:
(a) the amino acid sequence of SEQ ID NO:118;
(b) the amino acid sequence of SEQ ID NO:118 from amino acid 1 to amino acid 69;
(c) fragments of the amino acid sequence of SEQ ID NO:118; and
(d) the amino acid sequence encoded by the cDNA insert of clone hb752_{—}1 deposited under accession number ATCC 98482;
the protein being substantially free from other mammalian proteins.

153. An isolated gene corresponding to the cDNA sequence of SEQ ID NO:117.

154. An isolated polynucleotide selected from the group consisting of:
(a) a polynucleotide comprising the nucleotide sequence of SEQ ID NO:129;
(b) a polynucleotide comprising the nucleotide sequence of SEQ ID NO:129 from nucleotide 864 to nucleotide 1340;
(c) a polynucleotide comprising the nucleotide sequence of SEQ ID NO:129 from nucleotide 1 to nucleotide 1175;
(d) a polynucleotide comprising the nucleotide sequence of the full-length protein coding sequence of clone bi127_{—}5 deposited under accession number ATCC 98501;
(e) a polynucleotide encoding the full-length protein encoded by the cDNA insert of clone bi127_{—}5 deposited under accession number ATCC 98501;
(f) a polynucleotide comprising the nucleotide sequence of a mature protein coding sequence of clone bi127_{—}5 deposited under accession number ATCC 98501;
(g) a polynucleotide encoding a mature protein encoded by the cDNA insert of clone bi127_{—}5 deposited under accession number ATCC 98501;
(h) a polynucleotide encoding a protein comprising the amino acid sequence of SEQ ID NO:130;
(i) a polynucleotide encoding a protein comprising a fragment of the amino acid sequence of SEQ ID NO:130 having biological activity, the fragment comprising eight consecutive amino acids of SEQ ID NO:130;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

155. The polynucleotide of claim 154 wherein said polynucleotide is operably linked to at least one expression control sequence.

156. A host cell transformed with the polynucleotide of claim 155.

157. The host cell of claim 3, wherein said cell is a mammalian cell.

158. A process for producing a protein encoded by the polynucleotide of claim 155, which process comprises:
(a) growing a culture of the host cell of claim 156 in a suitable culture medium; and
(b) purifying said protein from the culture.

159. A protein produced according to the process of claim 158.

160. A protein comprising an amino acid sequence selected from the group consisting of:
(a) the amino acid sequence of SEQ ID NO:130;
(b) the amino acid sequence of SEQ ID NO:130 from amino acid 1 to amino acid 104;
(c) fragments of the amino acid sequence of SEQ ID NO:130 comprising eight consecutive amino acids of SEQ ID NO:130; and
(d) the amino acid sequence encoded by the cDNA insert of clone bi127_{—}5 deposited under accession number ATCC 98501;
the protein being substantially free from other mammalian proteins.

161. The protein of claim 160, wherein said protein comprises the amino acid sequence of SEQ ID NO:130.

162. The protein of claim 160, wherein said protein comprises the amino acid sequence of SEQ ID NO:130 from amino acid 1 to amino acid 104.

163. A composition comprising the protein of claim 160 and a pharmaceutically acceptable carrier.

164. An isolated gene corresponding to the cDNA sequence of SEQ ID NO:129.

165. An isolated polynucleotide selected from the group consisting of:
(a) a polynucleotide comprising the nucleotide sequence of SEQ ID NO:131;
(b) a polynucleotide comprising the nucleotide sequence of SEQ ID NO:131 from nucleotide 46 to nucleotide 738;
(c) a polynucleotide comprising the nucleotide sequence of SEQ ID NO:131 from nucleotide 346 to nucleotide 738;
(d) a polynucleotide comprising the nucleotide sequence of SEQ ID NO:131 from nucleotide 688 to nucleotide 1425;
(e) a polynucleotide comprising the nucleotide sequence of the full-length protein coding sequence of clone bl194_{—}2 deposited under accession number ATCC 98501;
(f) a polynucleotide encoding the full-length protein encoded by the cDNA insert of clone bl194_{—}2 deposited under accession number ATCC 98501;
(g) a polynucleotide comprising the nucleotide sequence of a mature protein coding sequence of clone bl194_{—}2 deposited under accession number ATCC 98501;
(h) a polynucleotide encoding a mature protein encoded by the cDNA insert of clone bl194_{—}2 deposited under accession number ATCC 98501;
(i) a polynucleotide encoding a protein comprising the amino acid sequence of SEQ ID NO:132;
(j) a polynucleotide encoding a protein comprising a fragment of the amino acid sequence of SEQ ID NO:132 having biological activity, the fragment comprising eight consecutive amino acids of SEQ ID NO:132;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

166. A protein comprising an amino acid sequence selected from the group consisting of:
(a) the amino acid sequence of SEQ ID NO:132;
(b) the amino acid sequence of SEQ ID NO:132 from amino acid 1 to amino acid 171;
(c) fragments of the amino acid sequence of SEQ ID NO:132 comprising eight consecutive amino acids of SEQ ID NO:132; and
(d) the amino acid sequence encoded by the cDNA insert of clone bl194_{—}2 deposited under accession number ATCC 98501;
the protein being substantially free from other mammalian proteins.

167. An isolated gene corresponding to the cDNA sequence of SEQ ID NO:131.

168. An isolated polynucleotide selected from the group consisting of:
(a) a polynucleotide comprising the nucleotide sequence of SEQ ID NO:133;
(b) a polynucleotide comprising the nucleotide sequence of SEQ ID NO:133 from nucleotide 234 to nucleotide 1235;
(c) a polynucleotide comprising the nucleotide sequence of SEQ ID NO:133 from nucleotide 291 to nucleotide 1235;
(d) a polynucleotide comprising the nucleotide sequence of SEQ ID NO:133 from nucleotide 209 to nucleotide 1050;
(e) a polynucleotide comprising the nucleotide sequence of the full-length protein coding sequence of clone cc130_{—}1 deposited under accession number ATCC 98501;
(f) a polynucleotide encoding the full-length protein encoded by the cDNA insert of clone cc130_{—}1 deposited under accession number ATCC 98501;
(g) a polynucleotide comprising the nucleotide sequence of a mature protein coding sequence of clone cc130_{—}1 deposited under accession number ATCC 98501;
(h) a polynucleotide encoding a mature protein encoded by the cDNA insert of clone cc130_{—}1 deposited under accession number ATCC 98501;
(i) a polynucleotide encoding a protein comprising the amino acid sequence of SEQ ID NO:134;
(j) a polynucleotide encoding a protein comprising a fragment of the amino acid sequence of SEQ ID NO:134 having biological activity, the fragment comprising eight consecutive amino acids of SEQ ID NO:134;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

169. A protein comprising an amino acid sequence selected from the group consisting of:
(a) the amino acid sequence of SEQ ID NO:134;
(b) the amino acid sequence of SEQ ID NO:134 from amino acid 1 to amino acid 272;
(c) fragments of the amino acid sequence of SEQ ID NO:134 comprising eight consecutive amino acids of SEQ ID NO:134; and
(d) the amino acid sequence encoded by the cDNA insert of clone cc130_{—}1 deposited under accession number ATCC 98501;
the protein being substantially free from other mammalian proteins.

170. An isolated gene corresponding to the cDNA sequence of SEQ ID NO:133.

171. An isolated polynucleotide selected from the group consisting of:
(a) a polynucleotide comprising the nucleotide sequence of SEQ ID NO:135;
(b) a polynucleotide comprising the nucleotide sequence of SEQ ID NO:135 from nucleotide 1554 to nucleotide 1784;
(c) a polynucleotide comprising the nucleotide sequence of SEQ ID NO:135 from nucleotide 1659 to nucleotide 1784;
(d) a polynucleotide comprising the nucleotide sequence of SEQ ID NO:7 from nucleotide 1508 to nucleotide 1865;
(e) a polynucleotide comprising the nucleotide sequence of the full-length protein coding sequence of clone ch582_{—}1 deposited under accession number ATCC 98501;
(f) a polynucleotide encoding the full-length protein encoded by the cDNA insert of clone ch582_{—}1 deposited under accession number ATCC 98501;
(g) a polynucleotide comprising the nucleotide sequence of a mature protein coding sequence of clone ch582_{—}1 deposited under accession number ATCC 98501;
(h) a polynucleotide encoding a mature protein encoded by the cDNA insert of clone ch582_{—}1 deposited under accession number ATCC 98501;
(i) a polynucleotide encoding a protein comprising the amino acid sequence of SEQ ID NO:136;
(j) a polynucleotide encoding a protein comprising a fragment of the amino acid sequence of SEQ ID NO:136 having biological activity, the fragment comprising eight consecutive amino acids of SEQ ID NO:136;
(k) a polynucleotide which is an allelic variant of a polynucleotide of (a)-(h) above;
(l) a polynucleotide which encodes a species homologue of the protein of (i) or above; and
(m) a polynucleotide that hybridizes under stringent conditions to any one of the polynucleotides specified in (a)-(j).

172. A protein comprising an amino acid sequence selected from the group consisting of:
(a) the amino acid sequence of SEQ ID NO:136;
(b) fragments of the amino acid sequence of SEQ ID NO:136 comprising eight consecutive amino acids of SEQ ID NO:136; and
(c) the amino acid sequence encoded by the cDNA insert of clone ch582_{—}1 deposited under accession number ATCC 98501;
the protein being substantially free from other mammalian proteins.

173. An isolated gene corresponding to the cDNA sequence of SEQ ID NO:135.

174. An isolated polynucleotide selected from the group consisting of:
(a) a polynucleotide comprising the nucleotide sequence of SEQ ID NO:137;
(b) a polynucleotide comprising the nucleotide sequence of SEQ ID NO:137 from nucleotide 1375 to nucleotide 1605;
(c) a polynucleotide comprising the nucleotide sequence of SEQ ID NO:137 from nucleotide 1107 to nucleotide 1539;
(d) a polynucleotide comprising the nucleotide sequence of the full-length protein coding sequence of clone cq294_{—}14 deposited under accession number ATCC 98501;
(e) a polynucleotide encoding the full-length protein encoded by the cDNA insert of clone cq294_{—}14 deposited under accession number ATCC 98501;
(f) a polynucleotide comprising the nucleotide sequence of a mature protein coding sequence of clone cq294_{—}14 deposited under accession number ATCC 98501;
(g) a polynucleotide encoding a mature protein encoded by the cDNA insert of clone cq294_{—}14 deposited under accession number ATCC 98501;
(h) a polynucleotide encoding a protein comprising the amino acid sequence of SEQ ID NO:138;
(i) a polynucleotide encoding a protein comprising a fragment of the amino acid sequence of SEQ ID NO:138 having biological activity, the fragment comprising eight consecutive amino acids of SEQ ID NO:138;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

175. A protein comprising an amino acid sequence selected from the group consisting of:
(a) the amino acid sequence of SEQ ID NO:138;
(b) the amino acid sequence of SEQ ID NO:138 from amino acid 1 to amino acid 55;
(c) fragments of the amino acid sequence of SEQ ID NO:138 comprising eight consecutive amino acids of SEQ ID NO:138; and
(d) the amino acid sequence encoded by the cDNA insert of clone cq294_{—}14 deposited under accession number ATCC 98501;
the protein being substantially free from other mammalian proteins.

176. An isolated gene corresponding to the cDNA sequence of SEQ ID NO:137.

177. An isolated polynucleotide selected from the group consisting of:
(a) a polynucleotide comprising the nucleotide sequence of SEQ ID NO:139;
(b) a polynucleotide comprising the nucleotide sequence of SEQ ID NO:139 from nucleotide 66 to nucleotide 1880;
(c) a polynucleotide comprising the nucleotide sequence of SEQ ID NO:139 from nucleotide 1 to nucleotide 581;
(d) a polynucleotide comprising the nucleotide sequence of the full-length protein coding sequence of clone dd454_{—}1 deposited under accession number ATCC 98501;
(e) a polynucleotide encoding the full-length protein encoded by the cDNA insert of clone dd454_{—}1 deposited under accession number ATCC 98501;
(f) a polynucleotide comprising the nucleotide sequence of a mature protein coding sequence of clone dd454_{—}1 deposited under accession number ATCC 98501;
(g) a polynucleotide encoding a mature protein encoded by the cDNA insert of clone dd454_{—}1 deposited under accession number ATCC 98501;
(h) a polynucleotide encoding a protein comprising the amino acid sequence of SEQ ID NO:140;
(i) a polynucleotide encoding a protein comprising a fragment of the amino acid sequence of SEQ ID NO:140 having biological activity, the fragment comprising eight consecutive amino acids of SEQ ID NO:140;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

178. A protein comprising an amino acid sequence selected from the group consisting of:
(a) the amino acid sequence of SEQ ID NO:140;
(b) the amino acid sequence of SEQ ID NO:140 from amino acid 1 to amino acid 172;
(c) fragments of the amino acid sequence of SEQ ID NO:140 comprising eight consecutive amino acids of SEQ ID NO:140; and
(d) the amino acid sequence encoded by the cDNA insert of clone dd454_{—}1 deposited under accession number ATCC 98501;
the protein being substantially free from other mammalian proteins.

179. An isolated gene corresponding to the cDNA sequence of SEQ ID NO:139.

180. An isolated polynucleotide selected from the group consisting of:
(a) a polynucleotide comprising the nucleotide sequence of SEQ ID NO:141;
(b) a polynucleotide comprising the nucleotide sequence of SEQ ID NO:141 from nucleotide 462 to nucleotide 3170;
(c) a polynucleotide comprising the nucleotide sequence of SEQ ID NO:141 from nucleotide 1188 to nucleotide 1517;
(d) a polynucleotide comprising the nucleotide sequence of the full-length protein coding sequence of clone du157_{—}12 deposited under accession number ATCC 98724;
(e) a polynucleotide encoding the full-length protein encoded by the cDNA insert of clone du157_{—}12 deposited under accession number ATCC 98724;
(f) a polynucleotide comprising the nucleotide sequence of a mature protein coding sequence of clone du157_{—}12 deposited under accession number ATCC 98724;
(g) a polynucleotide encoding a mature protein encoded by the cDNA insert of clone du157_{—}12 deposited under accession number ATCC 98724;
(h) a polynucleotide encoding a protein comprising the amino acid sequence of SEQ ID NO:142;
(i) a polynucleotide encoding a protein comprising a fragment of the amino acid sequence of SEQ ID NO:142 having biological activity, the fragment comprising eight consecutive amino acids of SEQ ID NO:142;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

181. A protein comprising an amino acid sequence selected from the group consisting of:
(a) the amino acid sequence of SEQ ID NO:142;
(b) the amino acid sequence of SEQ ID NO:14 from amino acid 251 to amino acid 352;
(c) fragments of the amino acid sequence of SEQ ID NO:142 comprising eight consecutive amino acids of SEQ ID NO:142; and
(d) the amino acid sequence encoded by the cDNA insert of clone du157_{—}12 deposited under accession number ATCC 98724;
the protein being substantially free from other mammalian proteins.

182. An isolated gene corresponding to the cDNA sequence of SEQ ID NO:141.

183. An isolated polynucleotide selected from the group consisting of:
(a) a polynucleotide comprising the nucleotide sequence of SEQ ID NO:143;
(b) a polynucleotide comprising the nucleotide sequence of SEQ ID NO:143 from nucleotide 865 to nucleotide 1158;
(c) a polynucleotide comprising the nucleotide sequence of SEQ ID NO:143 from nucleotide 1108 to nucleotide 1158;
(d) a polynucleotide comprising the nucleotide sequence of SEQ ID NO:143 from nucleotide 1 to nucleotide 764;
(e) a polynucleotide comprising the nucleotide sequence of the full-length protein coding sequence of clone du372_{—}1 deposited under accession number ATCC 98501;
(f) a polynucleotide encoding the full-length protein encoded by the cDNA insert of clone du372_{—}1 deposited under accession number ATCC 98501;
(g) a polynucleotide comprising the nucleotide sequence of a mature protein coding sequence of clone du372_{—}1 deposited under accession number ATCC 98501;
(h) a polynucleotide encoding a mature protein encoded by the cDNA insert of clone du372_{—}1 deposited under accession number ATCC 98501;
(i) a polynucleotide encoding a protein comprising the amino acid sequence of SEQ ID NO:144;
(j) a polynucleotide encoding a protein comprising a fragment of the amino acid sequence of SEQ ID NO:144 having biological activity, the fragment comprising eight consecutive amino acids of SEQ ID NO:144;
(k) a polynucleotide which is an allelic variant of a polynucleotide of (a)-(h) above;
(l) a polynucleotide which encodes a species homologue of the protein of (i) or 0(j) above; and
(m) a polynucleotide that hybridizes under stringent conditions to any one of the polynucleotides specified in (a)-(j).

184. A protein comprising an amino acid sequence selected from the group consisting of:
(a) the amino acid sequence of SEQ ID NO:144;
(b) the amino acid sequence of SEQ ID NO:144 from amino acid 69 to amino acid 98;
(c) fragments of the amino acid sequence of SEQ ID NO:144 comprising eight consecutive amino acids of SEQ ID NO:144; and
(d) the amino acid sequence encoded by the cDNA insert of clone du372_{—}1 deposited under accession number ATCC 98501;
the protein being substantially free from other mammalian proteins.

185. An isolated gene corresponding to the cDNA sequence of SEQ ID NO:143.

186. An isolated polynucleotide selected from the group consisting of:
(a) a polynucleotide comprising the nucleotide sequence of SEQ ID NO:145;
(b) a polynucleotide comprising the nucleotide sequence of SEQ ID NO:145 from nucleotide 32 to nucleotide 586;
(c) a polynucleotide comprising the nucleotide sequence of SEQ ID NO:145 from nucleotide 92 to nucleotide 586;
(d) a polynucleotide comprising the nucleotide sequence of SEQ ID NO:145 from nucleotide 1 to nucleotide 481;
(e) a polynucleotide comprising the nucleotide sequence of the full-length protein coding sequence of clone ej90_{—}5 deposited under accession number ATCC 98501;
(f) a polynucleotide encoding the full-length protein encoded by the cDNA insert of clone ej90_{—}5 deposited under accession number ATCC 98501;
(g) a polynucleotide comprising the nucleotide sequence of a mature protein coding sequence of clone ej90_{—}5 deposited under accession number ATCC 98501;
(h) a polynucleotide encoding a mature protein encoded by the cDNA insert of clone ej90_{—}5 deposited under accession number ATCC 98501;
(i) a polynucleotide encoding a protein comprising the amino acid sequence of SEQ ID NO:146;
(j) a polynucleotide encoding a protein comprising a fragment of the amino acid sequence of SEQ ID NO:146 having biological activity, the fragment comprising eight consecutive amino acids of SEQ ID NO:146;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

187. A protein comprising an amino acid sequence selected from the group consisting of:
(a) the amino acid sequence of SEQ ID NO:146;
(b) the amino acid sequence of SEQ ID NO:146 from amino acid 1 to amino acid 150;
(c) fragments of the amino acid sequence of SEQ ID NO:146 comprising eight consecutive amino acids of SEQ ID NO:146; and
(d) the amino acid sequence encoded by the cDNA insert of clone ej90_{—}5 deposited under accession number ATCC 98501;
the protein being substantially free from other mammalian proteins.

188. An isolated gene corresponding to the cDNA sequence of SEQ ID NO:145.

189. An isolated polynucleotide selected from the group consisting of:
(a) a polynucleotide comprising the nucleotide sequence of SEQ ID NO:147;
(b) a polynucleotide comprising the nucleotide sequence of SEQ ID NO:147 from nucleotide 281 to nucleotide 1786;
(c) a polynucleotide comprising the nucleotide sequence of SEQ ID NO:147 from nucleotide 332 to nucleotide 1786;
(d) a polynucleotide comprising the nucleotide sequence of SEQ ID NO:147 from nucleotide 1 to nucleotide 574;
(e) a polynucleotide comprising the nucleotide sequence of the full-length protein coding sequence of clone ic2_{—}6 deposited under accession number ATCC 98501;
(f) a polynucleotide encoding the full-length protein encoded by the cDNA insert of clone ic2_{—}6 deposited under accession number ATCC 98501;
(g) a polynucleotide comprising the nucleotide sequence of a mature protein coding sequence of clone ic2_{—}6 deposited under accession number ATCC 98501;
(h) a polynucleotide encoding a mature protein encoded by the cDNA insert of clone ic2_{—}6 deposited under accession number ATCC 98501;
(i) a polynucleotide encoding a protein comprising the amino acid sequence of SEQ ID NO:148;
(j) a polynucleotide encoding a protein comprising a fragment of the amino acid sequence of SEQ ID NO:148 having biological activity, the fragment comprising eight consecutive amino acids of SEQ ID NO:148;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

190. A protein comprising an amino acid sequence selected from the group consisting of:
(a) the amino acid sequence of SEQ ID NO:148;
(b) the amino acid sequence of SEQ ID NO:148 from amino acid 1 to amino acid 98;
(c) fragments of the amino acid sequence of SEQ ID NO:148 comprising eight consecutive amino acids of SEQ ID NO:148; and
(d) the amino acid sequence encoded by the cDNA insert of clone ic2_{—}6 deposited under accession number ATCC 98501;
the protein being substantially free from other mammalian proteins.

191. An isolated gene corresponding to the cDNA sequence of SEQ ID NO:147.

192. An isolated polynucleotide selected from the group consisting of:
(a) a polynucleotide comprising the nucleotide sequence of SEQ ID NO:159;
(b) a polynucleotide comprising the nucleotide sequence of SEQ ID NO:159 from nucleotide 69 to nucleotide 908;
(c) a polynucleotide comprising the nucleotide sequence of SEQ ID NO:159 from nucleotide 270 to nucleotide 908;
(d) a polynucleotide comprising the nucleotide sequence of the full-length protein coding sequence of clone bn97_{—}1 deposited under accession number ATCC 98535;
(e) a polynucleotide encoding the full-length protein encoded by the cDNA insert of clone bn97_{—}1 deposited under accession number ATCC 98535;
(f) a polynucleotide comprising the nucleotide sequence of a mature protein coding sequence of clone bn97_{—}1 deposited under accession number ATCC 98535;
(g) a polynucleotide encoding a mature protein encoded by the cDNA insert of clone bn97_{—}1 deposited under accession number ATCC 98535;
(h) a polynucleotide encoding a protein comprising the amino acid sequence of SEQ ID NO:160;
(i) a polynucleotide encoding a protein comprising a fragment of the amino acid sequence of SEQ ID NO:160 having biological activity, the fragment comprising eight consecutive amino acids of SEQ ID NO:160;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

193. The polynucleotide of claim 191 wherein said polynucleotide is operably linked to at least one expression control sequence.

194. A host cell transformed with the polynucleotide of claim 192.

195. The host cell of claim 194, wherein said cell is a mammalian cell.

196. A process for producing a protein encoded by the polynucleotide of claim 193, which process comprises:
(a) growing a culture of the host cell of claim 3 in a suitable culture medium; and
(b) purifying said protein from the culture.

197. A protein produced according to the process of claim 196.

198. A protein comprising an amino acid sequence selected from the group consisting of:
(a) the amino acid sequence of SEQ ID NO:160;
(b) the amino acid sequence of SEQ ID NO:160 from amino acid 1 to amino acid 83;
(c) fragments of the amino acid sequence of SEQ ID NO:160 comprising eight consecutive amino acids of SEQ ID NO:160; and
(d) the amino acid sequence encoded by the cDNA insert of clone bn97_{—}1 deposited under accession number ATCC 98535;
the protein being substantially free from other mammalian proteins.

199. The protein of claim 198, wherein said protein comprises the amino acid sequence of SEQ ID NO:2.

200. The protein of claim 198, wherein said protein comprises the amino acid sequence of SEQ ID NO:2 from amino acid 1 to amino acid 83.

201. A composition comprising the protein of claim 198 and a pharmaceutically acceptable carrier.

202. An isolated gene corresponding to the cDNA sequence of SEQ ID NO:1.

203. An isolated polynucleotide selected from the group consisting of:
(a) a polynucleotide comprising the nucleotide sequence of SEQ ID NO:161;
(b) a polynucleotide comprising the nucleotide sequence of SEQ ID NO:161 from nucleotide 562 to nucleotide 777;
(c) a polynucleotide comprising the nucleotide sequence of SEQ ID NO:161 from nucleotide 236 to nucleotide 673;
(d) a polynucleotide comprising the nucleotide sequence of the full-length protein coding sequence of clone bn268_{—}11 deposited under accession number ATCC 98535;
(e) a polynucleotide encoding the full-length protein encoded by the cDNA insert of clone bn268_{—}11 deposited under accession number ATCC 98535;
(f) a polynucleotide comprising the nucleotide sequence of a mature protein coding sequence of clone bn268_{—}11 deposited under accession number ATCC 98535;
(g) a polynucleotide encoding a mature protein encoded by the cDNA insert of clone bn268_{—}11 deposited under accession number ATCC 98535;
(h) a polynucleotide encoding a protein comprising the amino acid sequence of SEQ ID NO:162;
(i) a polynucleotide encoding a protein comprising a fragment of the amino acid sequence of SEQ ID NO:162 having biological activity, the fragment comprising eight consecutive amino acids of SEQ ID NO:162;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

204. A protein comprising an amino acid sequence selected from the group consisting of:
(a) the amino acid sequence of SEQ ID NO:162;
(b) the amino acid sequence of SEQ ID NO:162 from amino acid 1 to amino acid 37;
(c) fragments of the amino acid sequence of SEQ ID NO:162 comprising eight consecutive amino acids of SEQ ID NO:162; and
(d) the amino acid sequence encoded by the cDNA insert of clone bn268_{—}11 deposited under accession number ATCC 98535;
the protein being substantially free from other mammalian proteins.

205. An isolated gene corresponding to the cDNA sequence of SEQ ID NO:161.

206. An isolated polynucleotide selected from the group consisting of:
(a) a polynucleotide comprising the nucleotide sequence of SEQ ID NO:161;
(b) a polynucleotide comprising the nucleotide sequence of SEQ ID NO:161 from nucleotide 286 to nucleotide 1686;
(c) a polynucleotide comprising the nucleotide sequence of SEQ ID NO:161 from nucleotide 544 to nucleotide 1686;
(d) a polynucleotide comprising the nucleotide sequence of SEQ ID NO:161 from nucleotide 365 to nucleotide 1160;
(e) a polynucleotide comprising the nucleotide sequence of the full-length protein coding sequence of clone cb96_{—}10 deposited under accession number ATCC 98535;
(f) a polynucleotide encoding the full-length protein encoded by the cDNA insert of clone cb96_{—}10 deposited under accession number ATCC 98535;
(g) a polynucleotide comprising the nucleotide sequence of a mature protein coding sequence of clone cb96_{—}10 deposited under accession number ATCC 98535;
(h) a polynucleotide encoding a mature protein encoded by the cDNA insert of clone cb96_{—}10 deposited under accession number ATCC 98535;
(i) a polynucleotide encoding a protein comprising the amino acid sequence of SEQ ID NO:164;
(j) a polynucleotide encoding a protein comprising a fragment of the amino acid sequence of SEQ ID NO:164 having biological activity, the fragment comprising eight consecutive amino acids of SEQ ID NO:164;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

207. A protein comprising an amino acid sequence selected from the group consisting of:
(a) the amino acid sequence of SEQ ID NO:164;
(b) the amino acid sequence of SEQ ID NO:164 from amino acid 28 to amino acid 395;
(c) fragments of the amino acid sequence of SEQ ID NO:164 comprising eight consecutive amino acids of SEQ ID NO:164; and
(d) the amino acid sequence encoded by the cDNA insert of clone cb96_{—}10 deposited under accession number ATCC 98535;
the protein being substantially free from other mammalian proteins.

208. An isolated gene corresponding to the cDNA sequence of SEQ ID NO:163.

209. An isolated polynucleotide selected from the group consisting of:
(a) a polynucleotide comprising the nucleotide sequence of SEQ ID NO:165;
(b) a polynucleotide comprising the nucleotide sequence of SEQ ID NO:165 from nucleotide 99 to nucleotide 1049;
(c) a polynucleotide comprising the nucleotide sequence of SEQ ID NO:165 from nucleotide 222 to nucleotide 1049;
(d) a polynucleotide comprising the nucleotide sequence of SEQ ID NO:165 from nucleotide 632 to nucleotide 998;
(e) a polynucleotide comprising the nucleotide sequence of the full-length protein coding sequence of clone cb2_{—}311 deposited under accession number ATCC 98535;
(f) a polynucleotide encoding the full-length protein encoded by the cDNA insert of clone cb213_{—}11 deposited under accession number ATCC 98535;
(g) a polynucleotide comprising the nucleotide sequence of a mature protein coding sequence of clone cb213_{—}11 deposited under accession number ATCC 98535;
(h) a polynucleotide encoding a mature protein encoded by the cDNA insert of clone cb213_{—}11 deposited under accession number ATCC 98535;
(i) a polynucleotide encoding a protein comprising the amino acid sequence of SEQ ID NO:166;
(j) a polynucleotide encoding a protein comprising a fragment of the amino acid sequence of SEQ ID NO:166 having biological activity, the fragment comprising eight consecutive amino acids of SEQ ID NO:166;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

210. A protein comprising an amino acid sequence selected from the group consisting of:
(a) the amino acid sequence of SEQ ID NO:166;
(b) the amino acid sequence of SEQ ID NO:166 from amino acid 187 to amino acid 300;
(c) fragments of the amino acid sequence of SEQ ID NO:166 comprising eight consecutive amino acids of SEQ ID NO:166; and
(d) the amino acid sequence encoded by the cDNA insert of clone cb213_{—}11 deposited under accession number ATCC 98535;
the protein being substantially free from other mammalian proteins.

211. An isolated gene corresponding to the cDNA sequence of SEQ ID NO:165.

212. An isolated polynucleotide selected from the group consisting of:
(a) a polynucleotide comprising the nucleotide sequence of SEQ ID NO:167;
(b) a polynucleotide comprising the nucleotide sequence of SEQ ID NO:167 from nucleotide 3003 to nucleotide 3137;
(c) a polynucleotide comprising the nucleotide sequence of SEQ ID NO:167 from nucleotide 3072 to nucleotide 3137;
(d) a polynucleotide comprising the nucleotide sequence of SEQ ID NO:167 from nucleotide 2713 to nucleotide 3114;
(e) a polynucleotide comprising the nucleotide sequence of the full-length protein coding sequence of clone cj457_{—}4 deposited under accession number ATCC 98535;
(f) a polynucleotide encoding the full-length protein encoded by the cDNA insert of clone cj457_{—}4 deposited under accession number ATCC 98535;
(g) a polynucleotide comprising the nucleotide sequence of a mature protein coding sequence of clone cj457_{—}4 deposited under accession number ATCC 98535;
(h) a polynucleotide encoding a mature protein encoded by the cDNA insert of clone cj457_{—}4 deposited under accession number ATCC 98535;
(i) a polynucleotide encoding a protein comprising the amino acid sequence of SEQ ID NO:168;
(j) a polynucleotide encoding a protein comprising a fragment of the amino acid sequence of SEQ ID NO:168 having biological activity, the fragment comprising eight consecutive amino acids of SEQ ID NO:168;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

213. A protein comprising an amino acid sequence selected from the group consisting of:
(a) the amino acid sequence of SEQ ID NO:168;
(b) the amino acid sequence of SEQ ID NO:168 from amino acid 1 to amino acid 37;
(c) fragments of the amino acid sequence of SEQ ID NO:168 comprising eight consecutive amino acids of SEQ ID NO:168; and
(d) the amino acid sequence encoded by the cDNA insert of clone cj457_{—}4 deposited under accession number ATCC 98535;
the protein being substantially free from other mammalian proteins.

214. An isolated gene corresponding to the cDNA sequence of SEQ ID NO:167.

215. An isolated polynucleotide selected from the group consisting of:
(a) a polynucleotide comprising the nucleotide sequence of SEQ ID NO:169;
(b) a polynucleotide comprising the nucleotide sequence of SEQ ID NO:169 from nucleotide 284 to nucleotide 1357;
(c) a polynucleotide comprising the nucleotide sequence of SEQ ID NO:169 from nucleotide 603 to nucleotide 1233;
(d) a polynucleotide comprising the nucleotide sequence of the full-length protein coding sequence of clone cz653_{—}11 deposited under accession number ATCC 98535;
(e) a polynucleotide encoding the full-length protein encoded by the cDNA insert of clone cz653_{—}11 deposited under accession number ATCC 98535;
(f) a polynucleotide comprising the nucleotide sequence of a mature protein coding sequence of clone cz653_{—}11 deposited under accession number ATCC 98535;
(g) a polynucleotide encoding a mature protein encoded by the cDNA insert of clone cz653_{—}11 deposited under accession number ATCC 98535;
(h) a polynucleotide encoding a protein comprising the amino acid sequence of SEQ ID NO:170;
(i) a polynucleotide encoding a protein comprising a fragment of the amino acid sequence of SEQ ID NO:170 having biological activity, the fragment comprising eight consecutive amino acids of SEQ ID NO:170;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

216. A protein comprising an amino acid sequence selected from the group consisting of:
(a) the amino acid sequence of SEQ ID NO:170;
(b) the amino acid sequence of SEQ ID NO:170 from amino acid 147 to amino acid 358;
(c) fragments of the amino acid sequence of SEQ ID NO:170 comprising eight consecutive amino acids of SEQ ID NO:170; and
(d) the amino acid sequence encoded by the cDNA insert of clone cz653_{—}11 deposited under accession number ATCC 98535;
the protein being substantially free from other mammalian proteins.

217. An isolated gene corresponding to the cDNA sequence of SEQ ID NO:169.

218. An isolated polynucleotide selected from the group consisting of:
(a) a polynucleotide comprising the nucleotide sequence of SEQ ID NO:171;
(b) a polynucleotide comprising the nucleotide sequence of SEQ ID NO:171 from nucleotide 621 to nucleotide 1763;
(c) a polynucleotide comprising the nucleotide sequence of SEQ ID NO:171 from nucleotide 1461 to nucleotide 1763;
(d) a polynucleotide comprising the nucleotide sequence of the full-length protein coding sequence of clone dx138_{—}4 deposited under accession number ATCC 98535;
(e) a polynucleotide encoding the full-length protein encoded by the cDNA insert of clone dx138_{—}4 deposited under accession number ATCC 98535;
(f) a polynucleotide comprising the nucleotide sequence of a mature protein coding sequence of clone dx138_{—}4 deposited under accession number ATCC 98535;
(g) a polynucleotide encoding a mature protein encoded by the cDNA insert of clone dx138_{—}4 deposited under accession number ATCC 98535;
(h) a polynucleotide encoding a protein comprising the amino acid sequence of SEQ ID NO:172;
(i) a polynucleotide encoding a protein comprising a fragment of the amino acid sequence of SEQ ID NO:172 having biological activity, the fragment comprising eight consecutive amino acids of SEQ ID NO:172;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

219. A protein comprising an amino acid sequence selected from the group consisting of:
(a) the amino acid sequence of SEQ ID NO:172;
(b) the amino acid sequence of SEQ ID NO:172 from amino acid 83 to amino acid 229
(c) fragments of the amino acid sequence of SEQ ID NO:172 comprising eight consecutive amino acids of SEQ ID NO:172; and
(d) the amino acid sequence encoded by the cDNA insert of clone dx138_{—}4 deposited under accession number ATCC 98535;
the protein being substantially free from other mammalian proteins.

220. An isolated gene corresponding to the cDNA sequence of SEQ ID NO:171.

221. An isolated polynucleotide selected from the group consisting of:
(a) a polynucleotide comprising the nucleotide sequence of SEQ ID NO:173;
(b) a polynucleotide comprising the nucleotide sequence of SEQ ID NO:173 from nucleotide 119 to nucleotide 295;
(c) a polynucleotide comprising the nucleotide sequence of SEQ ID NO:173 from nucleotide 191 to nucleotide 295;
(d) a polynucleotide comprising the nucleotide sequence of the full-length protein coding sequence of clone ij167_{—}5 deposited under accession number ATCC 98535;
(e) a polynucleotide encoding the full-length protein encoded by the cDNA insert of clone ij167_{—}5 deposited under accession number ATCC 98535;
(f) a polynucleotide comprising the nucleotide sequence of a mature protein coding sequence of clone ij167_{—}5 deposited under accession number ATCC 98535;
(g) a polynucleotide encoding a mature protein encoded by the cDNA insert of clone ij167_{—}5 deposited under accession number ATCC 98535;
(h) a polynucleotide encoding a protein comprising the amino acid sequence of SEQ ID NO:174;
(i) a polynucleotide encoding a protein comprising a fragment of the amino acid sequence of SEQ ID NO:174 having biological activity, the fragment comprising eight consecutive amino acids of SEQ ID NO:174;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

222. A protein comprising an amino acid sequence selected from the group consisting of:
(a) the amino acid sequence of SEQ ID NO:174;
(b) the amino acid sequence of SEQ ID NO:174 from amino acid 1 to amino acid 26;
(c) fragments of the amino acid sequence of SEQ ID NO:174 comprising eight consecutive amino acids of SEQ ID NO:174; and
(d) the amino acid sequence encoded by the cDNA insert of clone ij167_{—}5 deposited under accession number ATCC 98535;
the protein being substantially free from other mammalian proteins.

223. An isolated gene corresponding to the cDNA sequence of SEQ ID NO:173.

224. An isolated polynucleotide selected from the group consisting of:
(a) a polynucleotide comprising the nucleotide sequence of SEQ ID NO:183;
(b) a polynucleotide comprising the nucleotide sequence of SEQ ID NO:183 from nucleotide 25 to nucleotide 1458;
(c) a polynucleotide comprising the nucleotide sequence of SEQ ID NO:183 from nucleotide 21 to nucleotide 730;
(d) a polynucleotide comprising the nucleotide sequence of the full-length protein coding sequence of clone bd107_{—}16 deposited under accession number ATCC 98898;
(e) a polynucleotide encoding the full-length protein encoded by the cDNA insert of clone bd107_{—}16 deposited under accession number ATCC 98898;
(f) a polynucleotide comprising the nucleotide sequence of a mature protein coding sequence of clone bd107_{—}16 deposited under accession number ATCC 98898;
(g) a polynucleotide encoding a mature protein encoded by the cDNA insert of clone bd107_{—}16 deposited under accession number ATCC 98898;
(h) a polynucleotide encoding a protein comprising the amino acid sequence of SEQ ID NO:184;
(i) a polynucleotide encoding a protein comprising a fragment of the amino acid sequence of SEQ ID NO:184 having biological activity, the fragment comprising eight consecutive amino acids of SEQ ID NO:184;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

225. The polynucleotide of claim 224 wherein said polynucleotide is operably linked to at least one expression control sequence.

226. A host cell transformed with the polynucleotide of claim 225.

227. The host cell of claim 226, wherein said cell is a mammalian cell.

228. A process for producing a protein encoded by the polynucleotide of claim 225, which process comprises:
(a) growing a culture of the host cell of claim 226 in a suitable culture medium; and
(b) purifying said protein from the culture.

229. A protein produced according to the process of claim 228.

230. A protein comprising an amino acid sequence selected from the group consisting of:
(a) the amino acid sequence of SEQ ID NO:184;
(b) the amino acid sequence of SEQ ID NO:184 from amino acid 2 to amino acid 118;
(c) fragments of the amino acid sequence of SEQ ID NO:184 comprising eight consecutive amino acids of SEQ ID NO:184; and
(d) the amino acid sequence encoded by the cDNA insert of clone bd107_{—}16 deposited under accession number ATCC 98898;
the protein being substantially free from other mammalian proteins.

231. The protein of claim 230, wherein said protein comprises the amino acid sequence of SEQ ID NO:184.

232. The protein of claim 230, wherein said protein comprises the amino acid sequence of SEQ ID NO:184 from amino acid 2 to amino acid 118.

233. A composition comprising the protein of claim 230 and a pharmaceutically acceptable carrier.

234. An isolated gene corresponding to the cDNA sequence of SEQ ID NO:183.

235. An isolated polynucleotide selected from the group consisting of:
(a) a polynucleotide comprising the nucleotide sequence of SEQ ID NO:185;
(b) a polynucleotide comprising the nucleotide sequence of SEQ ID NO:185 from nucleotide 6 to nucleotide 977;
(c) a polynucleotide comprising the nucleotide sequence of SEQ ID NO:185 from nucleotide 87 to nucleotide 977;
(d) a polynucleotide comprising the nucleotide sequence of SEQ ID NO:3 from nucleotide 8 to nucleotide 630;
(e) a polynucleotide comprising the nucleotide sequence of the full-length protein coding sequence of clone bm41_{—}7 deposited under accession number ATCC 98898;
(f) a polynucleotide encoding the full-length protein encoded by the cDNA insert of clone bm41_{—}7 deposited under accession number ATCC 98898;
(g) a polynucleotide comprising the nucleotide sequence of a mature protein coding sequence of clone bm41_{—}7 deposited under accession number ATCC 98898;
(h) a polynucleotide encoding a mature protein encoded by the cDNA insert of clone bm41_{—}7 deposited under accession number ATCC 98898;
(i) a polynucleotide encoding a protein comprising the amino acid sequence of SEQ ID NO:186;
(j) a polynucleotide encoding a protein comprising a fragment of the amino acid sequence of SEQ ID NO:186 having biological activity, the fragment comprising eight consecutive amino acids of SEQ ID NO:186;
(k) a polynucleotide which is an allelic variant of a polynucleotide of (a)-(h) above;
(l) a polynucleotide which encodes a species homologue of the protein of (i) or (j) above; and
(m) a polynucleotide that hybridizes under stringent conditions to any one of the polynucleotides specified in (a)-(j).

236. A protein comprising an amino acid sequence selected from the group consisting of:
(a) the amino acid sequence of SEQ ID NO:186;
(b) the amino acid sequence of SEQ ID NO:186 from amino acid 211 to amino acid 315;
(c) fragments of the amino acid sequence of SEQ ID NO:186 comprising eight consecutive amino acids of SEQ ID NO:186; and
(d) the amino acid sequence encoded by the cDNA insert of clone bm41_{—}7 deposited under accession number ATCC 98898;
the protein being substantially free from other mammalian proteins.

237. An isolated gene corresponding to the cDNA sequence of SEQ ID NO:185.

238. An isolated polynucleotide selected from the group consisting of:
(a) a polynucleotide comprising the nucleotide sequence of SEQ ID NO:187;
(b) a polynucleotide comprising the nucleotide sequence of SEQ ID NO:187 from nucleotide 168 to nucleotide 962;
(c) a polynucleotide comprising the nucleotide sequence of SEQ ID NO:187 from nucleotide 351 to nucleotide 962;
(d) a polynucleotide comprising the nucleotide sequence of the full-length protein coding sequence of clone br342_{—}11 deposited under accession number ATCC 98551;
(e) a polynucleotide encoding the full-length protein encoded by the cDNA insert of clone br342_{—}11 deposited under accession number ATCC 98551;
(f) a polynucleotide comprising the nucleotide sequence of a mature protein coding sequence of clone br342_{—}11 deposited under accession number ATCC 98551;
(g) a polynucleotide encoding a mature protein encoded by the cDNA insert of clone br342_{—}11 deposited under accession number ATCC 98551;
(h) a polynucleotide encoding a protein comprising the amino acid sequence of SEQ ID NO:188;
(i) a polynucleotide encoding a protein comprising a fragment of the amino acid sequence of SEQ ID NO:188 having biological activity, the fragment comprising eight consecutive amino acids of SEQ ID NO:188;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

239. A protein comprising an amino acid sequence selected from the group consisting of:
(a) the amino acid sequence of SEQ ID NO:188;
(b) the amino acid sequence of SEQ ID NO:188 from amino acid 1 to amino acid 78;
(c) fragments of the amino acid sequence of SEQ ID NO:188 comprising eight consecutive amino acids of SEQ ID NO:188; and
(d) the amino acid sequence encoded by the cDNA insert of clone br342_{—}11 deposited under accession number ATCC 98551;
the protein being substantially free from other mammalian proteins.

240. An isolated gene corresponding to the cDNA sequence of SEQ ID NO:187.

241. An isolated polynucleotide selected from the group consisting of:
(a) a polynucleotide comprising the nucleotide sequence of SEQ ID NO:189;
(b) a polynucleotide comprising the nucleotide sequence of SEQ ID NO:189 from nucleotide 134 to nucleotide 493;
(c) a polynucleotide comprising the nucleotide sequence of the full-length protein coding sequence of clone ej258_{—}11 deposited under accession number ATCC 98551;
(d) a polynucleotide encoding the full-length protein encoded by the cDNA insert of clone ej258_{—}11 deposited under accession number ATCC 98551;
(e) a polynucleotide comprising the nucleotide sequence of a mature protein coding sequence of clone ej258_{—}11 deposited under accession number ATCC 98551;
(f) a polynucleotide encoding a mature protein encoded by the cDNA insert of clone ej25811 deposited under accession number ATCC 98551;
(g) a polynucleotide encoding a protein comprising the amino acid sequence of SEQ ID NO:190;
(h) a polynucleotide encoding a protein comprising a fragment of the amino acid sequence of SEQ ID NO:190 having biological activity, the fragment comprising eight consecutive amino acids of SEQ ID NO:190;
(i) a polynucleotide which is an allelic variant of a polynucleotide of (a)-(f) above;
(j) a polynucleotide which encodes a species homologue of the protein of (g) or (h) above; and
(k) a polynucleotide that hybridizes under stringent conditions to any one of the polynucleotides specified in (a)-(h).

242. A protein comprising an amino acid sequence selected from the group consisting of:
(a) the amino acid sequence of SEQ ID NO:190;
(b) the amino acid sequence of SEQ ID NO:190 from amino acid 1 to amino acid 64;
(c) fragments of the amino acid sequence of SEQ ID NO:190 comprising eight consecutive amino acids of SEQ ID NO:190; and
(d) the amino acid sequence encoded by the cDNA insert of clone ej258_{—}11 deposited under accession number ATCC 98551;
the protein being substantially free from other mammalian proteins.

243. An isolated gene corresponding to the cDNA sequence of SEQ ID NO:189.

244. An isolated polynucleotide selected from the group consisting of:
(a) a polynucleotide comprising the nucleotide sequence of SEQ ID NO:191.
(b) a polynucleotide comprising the nucleotide sequence of SEQ ID NO:191 from nucleotide 14 to nucleotide 406;
(c) a polynucleotide comprising the nucleotide sequence of SEQ ID NO:191 from nucleotide 62 to nucleotide 406;
(d) a polynucleotide comprising the nucleotide sequence of the full-length protein coding sequence of clone k232_{—}2x deposited under accession number ATCC 98551;
(e) a polynucleotide encoding the full-length protein encoded by the cDNA insert of clone k232_{—}2x deposited under accession number ATCC 98551;
(f) a polynucleotide comprising the nucleotide sequence of a mature protein coding sequence of clone k232_{—}2x deposited under accession number ATCC 98551;
(g) a polynucleotide encoding a mature protein encoded by the cDNA insert of clone k232_{—}2x deposited under accession number ATCC 98551;
(h) a polynucleotide encoding a protein comprising the amino acid sequence of SEQ ID NO:192;
(i) a polynucleotide encoding a protein comprising a fragment of the amino acid sequence of SEQ ID NO:192 having biological activity, the fragment comprising eight consecutive amino acids of SEQ ID NO:192;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

245. A protein comprising an amino acid sequence selected from the group consisting of:
(a) the amino acid sequence of SEQ ID NO:192;
(b) the amino acid sequence of SEQ ID NO:192 from amino acid 1 to amino acid 81;
(c) fragments of the amino acid sequence of SEQ ID NO:192 comprising eight consecutive amino acids of SEQ ID NO:192; and
(d) the amino acid sequence encoded by the cDNA insert of clone k232_{—}2x deposited under accession number ATCC 98551;
the protein being substantially free from other mammalian proteins.

246. An isolated gene corresponding to the cDNA sequence of SEQ ID NO:191.

247. An isolated polynucleotide selected from the group consisting of:
(a) a polynucleotide comprising the nucleotide sequence of SEQ ID NO:193;
(b) a polynucleotide comprising the nucleotide sequence of SEQ ID NO:193 from nucleotide 580 to nucleotide 816;
(c) a polynucleotide comprising the nucleotide sequence of the full-length protein coding sequence of clone lf307_{—}5 deposited under accession number ATCC 98551;
(d) a polynucleotide encoding the full-length protein encoded by the cDNA insert of clone lf307_{—}5 deposited under accession number ATCC 98551;
(e) a polynucleotide comprising the nucleotide sequence of a mature protein coding sequence of clone lf307_{—}5 deposited under accession number ATCC 98551;
(f) a polynucleotide encoding a mature protein encoded by the cDNA insert of clone lf307_{—}5 deposited under accession number ATCC 98551;
(g) a polynucleotide encoding a protein comprising the amino acid sequence of SEQ ID NO:194;
(h) a polynucleotide encoding a protein comprising a fragment of the amino acid sequence of SEQ ID NO:194 having biological activity, the fragment comprising eight consecutive amino acids of SEQ ID NO:194;
(i) a polynucleotide which is an allelic variant of a polynucleotide of (a)-(f) above;
(j) a polynucleotide which encodes a species homologue of the protein of (g) or (h) above; and
(k) a polynucleotide that hybridizes under stringent conditions to any one of the polynucleotides specified in (a)-(h).

248. A protein comprising an amino acid sequence selected from the group consisting of:
(a) the amino acid sequence of SEQ ID NO:194;
(b) fragments of the amino acid sequence of SEQ ID NO:194 comprising eight consecutive amino acids of SEQ ID NO:194; and
(c) the amino acid sequence encoded by the cDNA insert of clone lf307_{—}5 deposited under accession number ATCC 98551;
the protein being substantially free from other mammalian proteins.

249. An isolated gene corresponding to the cDNA sequence of SEQ ID NO:193 and SEQ ID NO:195.

250. An isolated polynucleotide selected from the group consisting of:
(a) a polynucleotide comprising the nucleotide sequence of SEQ ID NO:196;
(b) a polynucleotide comprising the nucleotide sequence of SEQ ID NO:196 from nucleotide 127 to nucleotide 627;
(c) a polynucleotide comprising the nucleotide sequence of SEQ ID NO:196 from nucleotide 250 to nucleotide 627;
(d) a polynucleotide comprising the nucleotide sequence of the full-length protein coding sequence of clone lr204_{—}1 deposited under accession number ATCC 98551;
(e) a polynucleotide encoding the full-length protein encoded by the cDNA insert of clone lr204_{—}1 deposited under accession number ATCC 98551;
(f) a polynucleotide comprising the nucleotide sequence of a mature protein coding sequence of clone lr204_{—}1 deposited under accession number ATCC 98551;
(g) a polynucleotide encoding a mature protein encoded by the cDNA insert of clone lr204_{—}1 deposited under accession number ATCC 98551;
(h) a polynucleotide encoding a protein comprising the amino acid sequence of SEQ ID NO:197;
(i) a polynucleotide encoding a protein comprising a fragment of the amino acid sequence of SEQ ID NO:197 having biological activity, the fragment comprising eight consecutive amino acids of SEQ ID NO:197;
(j) a polynucleotide which is an allelic variant of a polynucleotide of (a)-(g) above;
(k) a polynucleotide which encodes a species homologue of the protein of (h) or (i) above; and
(l) a polynucleotide that hybridizes under stringent conditions to any one of the polynucleotides specified in (a)-(i).

251. A protein comprising an amino acid sequence selected from the group consisting of:
(a) the amino acid sequence of SEQ ID NO:197;
(b) the amino acid sequence of SEQ ID NO:197 from amino acid 23 to amino acid 106;
(c) fragments of the amino acid sequence of SEQ ID NO:197 comprising eight consecutive amino acids of SEQ ID NO:197; and
(d) the amino acid sequence encoded by the cDNA insert of clone lr204_{—}1 deposited under accession number ATCC 98551;
the protein being substantially free from other mammalian proteins.

252. An isolated gene corresponding to the cDNA sequence of SEQ ID NO:196.

253. An isolated polynucleotide selected from the group consisting of:
(a) a polynucleotide comprising the nucleotide sequence of SEQ ID NO:207;
(b) a polynucleotide comprising the nucleotide sequence of SEQ ID NO:207 from nucleotide 946 to nucleotide 1095;
(c) a polynucleotide comprising the nucleotide sequence of the full-length protein coding sequence of clone bf227_{—}8 deposited under accession number ATCC 98580;
(d) a polynucleotide encoding the full-length protein encoded by the cDNA insert of clone bf227_{—}8 deposited under accession number ATCC 98580;
(e) a polynucleotide comprising the nucleotide sequence of the mature protein coding sequence of clone bf227_{—}8 deposited under accession number ATCC 98580;
(f) a polynucleotide encoding the mature protein encoded by the cDNA insert of clone bf227_{—}8 deposited under accession number ATCC 98580;
(g) a polynucleotide encoding a protein comprising the amino acid sequence of SEQ ID NO:208;
(h) a polynucleotide encoding a protein comprising a fragment of the amino acid sequence of SEQ ID NO:208 having biological activity;
(i) a polynucleotide which is an allelic variant of a polynucleotide of (a)-(f) above;
(j) a polynucleotide which encodes a species homologue of the protein of (g) or (h) above; and
(k) a polynucleotide capable of hybridizing under stringent conditions to any one of the polynucleotides specified in (a)-(h).

254. The polynucleotide of claim 253 wherein said polynucleotide is operably linked to at least one expression control sequence.

255. A host cell transformed with the polynucleotide of claim 254.

256. The host cell of claim 255, wherein said cell is a mammalian cell.

257. A process for producing a protein encoded by the polynucleotide of claim 254, which process comprises:
(a) growing a culture of the host cell of claim 255 in a suitable culture medium; and
(b) purifying said protein from the culture.

258. A protein produced according to the process of claim 257.

259. An isolated polynucleotide encoding the protein of claim 258.

260. A protein comprising an amino acid sequence selected from the group consisting of:
(a) the amino acid sequence of SEQ ID NO:208;
(b) the amino acid sequence of SEQ ID NO:208 from amino acid 1 to amino acid 34;
(c) fragments of the amino acid sequence of SEQ ID NO:208; and
(d) the amino acid sequence encoded by the cDNA insert of clone bf227_{—}8 deposited under accession number ATCC 98580;
the protein being substantially free from other mammalian proteins.

261. The protein of claim 260, wherein said protein comprises the amino acid sequence of SEQ ID NO:208.

262. The protein of claim 261, wherein said protein comprises the amino acid sequence of SEQ ID NO:4 from amino acid 1 to amino acid 34.

263. A composition comprising the protein of claim 261 and a pharmaceutically acceptable carrier.

264. A method for preventing, treating or ameliorating a medical condition which comprises administering to a mammalian subject a therapeutically effective amount of a composition of claim 263.

265. A process for producing an isolated polynucleotide, wherein the process is selected from the group consisting of:
(a) a process comprising the steps of:
(i) preparing one or more polynucleotide probes that hybridize in 6× SSC at 65 degrees C. to a nucleotide sequence selected from the group consisting of:
(aa) SEQ ID NO:207, but excluding the poly(A) tail at the 3′ end of SEQ ID NO:207; and
(ab) the nucleotide sequence of the cDNA insert of clone bf227_{—}8 deposited under ATCC 98580;
(ii) hybridizing said probe(s) to human DNA; and
(iii) isolating the DNA polynucleotide detected with the probe(s); and
(b) a process comprising the steps of:
(i) preparing one or more polynucleotide primers that hybridize in 6× SSC at 65 degrees C. to a nucleotide sequence selected from the group consisting of:
(ba) SEQ ID NO:207, but excluding the poly(A) tail at the 3′ end of SEQ ID NO:207; and
(bb) the nucleotide sequence of the cDNA insert of clone bf227_{—}8 deposited under ATCC 98580;
(ii) hybridizing said primer(s) to human DNA;
(iii) amplifying human DNA sequences; and
(iv) isolating the polynucleotide product of step (b) (iii).

266. An isolated polynucleotide produced according to the process of claim 265, wherein the nucleotide sequence of said isolated polynucleotide corresponds to the cDNA sequence of SEQ ID NO:207, and extends contiguously from a nucleotide sequence corresponding to the 5′ end of SEQ ID NO:207 to a nucleotide sequence corresponding to the 3′ end of SEQ ID NO:207 but excluding the poly(A) tail at the 3′ end of SEQ ID NO:207.

267. An isolated polynucleotide produced according to the process of claim 265, wherein the nucleotide sequence of said isolated polynucleotide corresponds to the cDNA sequence of SEQ ID NO:207 from nucleotide 946 to nucleotide 1095, and extends contiguously from a nucleotide sequence corresponding to the 5′ end of said sequence of SEQ ID NO:207 from nucleotide 946 to nucleotide 1095, to a nucleotide sequence corresponding to the 3′ end of said sequence of SEQ ID NO:207 from nucleotide 946 to nucleotide 1095.

268. A composition comprising an isolated polynucleotide selected from the group consisting of:
(a) a polynucleotide comprising the nucleotide sequence of SEQ ID NO:209;
(b) a polynucleotide comprising the nucleotide sequence of SEQ ID NO:209 from nucleotide 183 to nucleotide 911;
(c) a polynucleotide comprising the nucleotide sequence of the full-length protein coding sequence of clone bh157_{—}7 deposited under accession number ATCC 98580;
(d) a polynucleotide encoding the full-length protein encoded by the cDNA insert of clone bh157_{—}7 deposited under accession number ATCC 98580;
(e) a polynucleotide comprising the nucleotide sequence of the mature protein coding sequence of clone bh157_{—}7 deposited under accession number ATCC 98580;
(f) a polynucleotide encoding the mature protein encoded by the cDNA insert of clone bh157_{—}7 deposited under accession number ATCC 98580;
(g) a polynucleotide encoding a protein comprising the amino acid sequence of SEQ ID NO:210;
(h) a polynucleotide encoding a protein comprising a fragment of the amino acid sequence of SEQ ID NO:210 having biological activity;
(i) a polynucleotide which is an allelic variant of a polynucleotide of (a)-(f) above;
(j) a polynucleotide which encodes a species homologue of the protein of (g) or (h) above; and
(k) a polynucleotide capable of hybridizing under stringent conditions to any one of the polynucleotides specified in (a)-(h).

269. A composition comprising a protein, wherein said protein comprises an amino acid sequence selected from the group consisting of:
(a) the amino acid sequence of SEQ ID NO:210;
(b) the amino acid sequence of SEQ ID NO:210 from amino acid 1 to amino acid 76;
(c) fragments of the amino acid sequence of SEQ ID NO:210; and
(d) the amino acid sequence encoded by the cDNA insert of clone bh157_{—}7 deposited under accession number ATCC 98580;
the protein being substantially free from other mammalian proteins.
